(19) United States
(12) Patent Application Publication
(10) Pub. No.: US 2025/0268821 A1
(43) Pub. Date: Aug. 28, 2025

(54) NINTEDANIB AND NINTEDANIB COMBINATION DRY POWDER COMPOSITIONS AND USES

(71) Applicant: AVALYN PHARMA INC., Cambridge, MA (US)

(72) Inventors: Mark William Surber, San Diego, CA (US); Stephen Pham, Seattle, WA (US)

(21) Appl. No.: 18/974,588

(22) Filed: Dec. 9, 2024

Related U.S. Application Data
(63) Continuation of application No. 18/961,095, filed on Nov. 26, 2024.
(63) Continuation of application No. PCT/US2023/023770, filed on May 26, 2023.
(60) Provisional application No. 63/425,928, filed on Nov. 16, 2022.
(60) Provisional application No. 63/346,856, filed on May 28, 2022.

(51) Int. Cl. A61K 9/00; A61K 31/496; A61K 47/18; A61K 47/24

(52) U.S. Cl. CPC A61K 9/0075; A61K 31/496; A61K 47/183; A61K 47/24

(57) ABSTRACT

The invention includes dry powder nintedanib formulation for dispersion and inhalation administration including salts thereof, indolinone derivative or salts thereof and in fixed dose combinations with carrier agents and other active ingredients. The aerosol delivery combination formulation may be administered as an inhaled aerosol over a few actuations or by two or more actuations. Each dose may be administered one or more times daily on a regular or interval daily dosing regimen. The special formulation parameters of the invention include the selection of the salt for complexation with the form of nintedanib used for an isolated dry powder along with particle size distributions and combination with a force control agent for improved aerosol delivery. Methods of the invention include therapeutically effective doses of the described invention used to treat interstitial lung disease.

### BACKGROUND OF THE INVENTION

A number of pulmonary diseases such as interstitial lung disease (ILD; and sub-class diseases therein), cancer (lung cancer; and sub-class diseases therein), fibrotic indications of the lungs, kidney, heart and eye, viral infections and diseases of the central nervous system are current areas of unmet clinical need.

In fibrosis, scarring serves a valuable healing role following injury. However, tissue may become progressively scarred following more chronic, repeated and or idiopathic injuries resulting in abnormal function. In the case of idiopathic pulmonary fibrosis (IPF), progressive pulmonary fibrosis (PPF); and other subclasses of ILD, if a sufficient proportion of the lung becomes scarred respiratory failure can occur. In any case, progressive scarring may result from a recurrent series of insults to different regions of the organ or a failure to halt the repair process after the injury has healed. In such cases the scarring process becomes uncontrolled and deregulated. In some forms of fibrosing disease scarring remains localized to a limited region, but in others it can affect a more diffuse and extensive area resulting in direct or associated organ failure.

In epithelial injury, epithelial cells are triggered to release several pro-fibrotic mediators, including the potent fibroblast growth factors transforming growth factor-beta (TGF-beta), tumor necrosis factor (TNF), platelet derived growth factor (PDGF), endothelin, other cytokines, metalloproteinases and the coagulation mediator tissue factor. Importantly, the triggered epithelial cell becomes vulnerable to apoptosis, and together with an apparent inability to restore the epithelial cell layer are the most fundamental abnormalities in fibrotic disease.

In conditions such as diseases, physiological responses characterized by control of pro-fibrotic factors with indolinone derivative, such as nintedanib is beneficial to attenuate and/or reverse fibrosis, treat cancer, or central nervous system disease. Therapeutic strategies exploiting such indolinone derivative and/or nintedanib effects in these and other indications are contemplated herein.

Despite the development of a number of promising therapies, a number pulmonary diseases such as interstitial lung disease (ILD; and sub-class diseases therein), cancer, vascular and many viral infectious disease remain unmet clinical needs. Additionally, a number of extrapulmonary diseases may also benefit from inhaled delivery of nintedanib or drug-drug combinations therein, together with formulations specifically designed to take advantage of inhaled device performance parameters. However, development of advanced nintedanib and combination formulations for delivery by inhalation carries a number of challenges that have not been completely overcome.

### BRIEF SUMMARY OF THE INVENTION

Special design considerations for nintedanib impact a number of parameters that are critical for developing an inhaled therapeutic product. By selective manipulation of formulation parameters and aerosol device parameters, the target organ dose, pharmacokinetic profile, and safety profile can be improved to increase efficacy, safety and maximize patient compliance. Described herein are compositions of nintedanib or salt thereof, and indolinone derivatives or salt thereof that are suitable for inhalation delivery to the lungs, central nervous system and/or systemic compartment and methods of use.

The invention includes dry powder formulation for dispersion and inhalation administration comprising nintedanib or salt thereof, or a indolinone derivative or salt thereof and one or more carrier or bulking agents. The bulk powder composition may also contain one or more force control agents from about 0.01% to about 20% of the bulk composition. Force control agents may be leucine, trileucine, lecithin, magnesium stearate, sodium stearate, sucrose stearate, fine lactose, polyvinylpyrrolidone, ethyl cellulose, Pluronic F-68, Cremophor RH 40, glyceryl monostearate, and polyethylene glycol 6000. Additionally, the bulk powder composition may include inorganic salts, e.g., sodium chloride, magnesium chloride, calcium chloride, potassium chloride, sodium bromide, potassium bromide, magnesium bromide and calcium bromide and combinations thereof as a stabilizing agent or a secondary excipient from about 0.01% to about 20% of the bulk composition. The bulk powder composition may also contain anion from about 0.001% to about 10% of the bulk composition. The anion may be bromide or chloride. The bulk powder composition may also contain a taste masking agent from about 0.001% to about 10% of the bulk composition. The task masking agent may be saccharin or other agent common for use in the art. The bulk powder composition may also include a sugar as a bulking agent or a stabilizing agent, such as lactose, mannitol, trehalose, dextrose. The bulk powder composition may also contain a amino acid from about 0.01% to about 20% of the bulk composition.

The invention also includes dry powder formulation for dispersion and inhalation administration comprising nintedanib or salt thereof, or an indolinone derivative or salt thereof in a fixed dose combination with Pirfenidone or pyridine analog. In this combination formulation, nintedanib or salt thereof, or an indolinone derivative or salt thereof is included in an amount from about 0.0001 mg to about 200 mg, and Pirfenidone or pyridine analog is included in an amount from about 1 mg to about 200 mg. carrier.

The invention also includes dry powder formulation for dispersion and inhalation administration comprising nintedanib or salt thereof, or a indolinone derivative or salt thereof in a fixed dose combination with a PDE4 inhibitor. In this combination formulation, nintedanib or salt thereof, or a indolinone derivative or salt thereof is included in an amount from about 0.0001 mg to about 200 mg, and the PDE4 inhibitor is included in an amount from about 0.01 mg to about 40 mg. carrier

The invention also includes dry powder formulation for dispersion and inhalation administration comprising nintedanib or salt thereof, or a indolinone derivative or salt thereof in a fixed dose combination with a prostacyclin analog. In this combination formulation, nintedanib or salt thereof, or a indolinone derivative or salt thereof is included in an amount from about 0.0001 mg to about 200 mg, and the prostacyclin analog is included in an amount from about 0.001 mg to about 10 mg carrier. The dry powder formulation may be administered as an inhaled aerosol created from a nintedanib or salt thereof, or a indolinone derivative or salt thereof dosing amount ranging from about 0.0001 mg to about 200 mg, and prostacyclin analog ranging from about 0.001 mg to about 10 mg per unit dose or single actuation. The combination formulation dose may be administered as an inhaled aerosol over a few actuations or by two or more actuations. Each dose may be administered one or more times daily on a regular or interval daily dosing regimen.

The special formulation parameters of the invention include the selection of the salt for complexation with the form of nintedanib used for an isolated dry powder. Preferred salts include esylate, mesylate, hydrochloride, and hydrobromide. The total delivery dose is from about 0.0001 mg to about 200 mg of nintedanib in the dry powder formulation described herein.

The invention includes a kit comprising: a unit dosage of a dry powder of nintedanib or salt thereof, as described herein in a container that is adapted for use with a dry powder inhaler for dispersion and resulting powder aerosol inhalation. Such compositions may also include combinations with Pirfenidone or pyridine analog. Such compositions may also include combinations with a phosphodiesterase 4 (PDE4) inhibitor. Such compositions may also include combinations with a prostacyclin analog.

Moreover, the physicochemical properties of the resulting aerosol created by the compositions and methods of the present invention are an important part of the therapeutic utility of the present invention because the specially selected formulation design parameters, together with dry powder dispersion by the dry powder inhaler structures as described below, yield an aerosol powder cloud that has uniquely advantageous properties for delivery of the active ingredient to a pulmonary compartment that is tailored to the pharmacodynamic absorption of the active pharmaceutical ingredient in the pulmonary organ. Administration of the dispersed dry powder, either as nintedanib particles, co-precipitate of nintedanib particles, combination of Nintedanib in either form and optional inclusion of a PDE4 inhibitor or prostacyclin yields an aerosol in the form of a cloud or dry messed containing the nintedanib or indolinone of salt thereof, or nintedanib or indolinone of salt thereof particles that have a mass median aerodynamic diameter less than [X] mean diameter less than about 5.0 μm and component dry particles of the Pirfenidone, PDE 4 inhibitor or prostacyclin having the mean particle size diameter as described herein. Accordingly, the aerosol can include a single API, a dual combination of Nintedanib, indolinone or salt thereof, a double combination of Nintedanib and Pirfenidone, or a triple or quadruple combination further including Pirfenidone and prostacyclin. The aerosol particles produced from a final bulk formulation placed in a dry powder inhaler, are formulated as the specially designed powder containing nintedanib or indolinone or salt at from about 0.0001 mg to about 200 mg. Alternatively, the aerosol particles produced from a final bulk formulation placed in a dry powder inhaler, formulated as the specially designed powder containing nintedanib or indolinone or salt thereof at from about 0.01 mg to about 100 mg. Alternatively, the aerosol particles produced from a final bulk formulation placed in a dry powder inhaler, formulated as the specially designed powder containing nintedanib or indolinone or salt thereof at from about 0.01 mg to about 50 mg, and present alone or in combination as described above. [Such compositions may also include combinations with Pirfenidone or pyridine analog in an amount of 1 mg to 200 mg within particles having a mean diameter less than about 5 μm. Such compositions may also include combinations with a PDE4 inhibitor in an amount of 0.01 mg to 40 mg within particles having a mean diameter less than about 5 μm. Such compositions may also include combinations with a prostacyclin analog in an amount of 0.001 mg to 10 mg within particles having a mean diameter less than about 5 μm. The preferred weight ratios are 0.0001:100 to 200:0.01 nintedanib/Pirfenidone ratio, 0.0001:40 to 200:0.01 nintedanib/PDE4 inhibitor, and 0.0001:10 to 200:0.001 nintedanib/prostacyclin. In some embodiments, administration of nintedanib or indolinone or salt thereof in combination with Pirfenidone or pyridine analog, by inhalation has reduced gastrointestinal and liver side-effects when compared to add-on oral administration of nintedanib and Pirfenidone. Reducing these side-effects increases patient safety, maximizes patient compliance, avoids dose reduction and/or stoppage protocols, and enables either local lung dose escalation or combination ratio optimization for additional efficacy otherwise not possible treating with the two oral products.

These and other aspects of the invention will be evident upon reference to the following detailed description. All of the U.S. patents, U.S. patent application publications, U.S. patent applications, foreign patents, foreign patent applications and non-patent publications referred to in this specification, are incorporated herein by reference in their entirety, as if each was incorporated individually.

### Certain Terminology

The term “mg” refers to milligram.

The term “μg” refers to microgram.

The term “microM” refers to micromolar.

As used herein, the term “about” is used synonymously with the term “approximately.” Illustratively, the use of the term “about” with regard to a certain therapeutically effective pharmaceutical dose indicates that values slightly outside the cited values, e.g., plus or minus 0.1% to 10%, which are also effective and safe.

As used herein, the terms “comprising,” “including,” “such as,” and “for example” are used in their open, non-limiting sense.

The terms “administration” or “administering” and “delivery” or “delivery” refer to a method of giving to a human a dosage of a therapeutic or prophylactic formulation, such as an nintedanib or salt thereof formulation described herein, for example as an anti-inflammatory, anti-fibrotic and/or anti-demyelination pharmaceutical composition, or for other purposes. The preferred delivery method or method of administration can vary depending on various factors, e.g., the components of the pharmaceutical composition, the desired site at which the formulation is to be introduced, delivered or administered, the site where therapeutic benefit is sought, or the proximity of the initial delivery site to the downstream diseased organ (e.g., aerosol delivery to the lung for absorption and secondary delivery to the heart, kidney, liver, central nervous system or other diseased destination).

The terms “pulmonary administration” or “inhalation” or “pulmonary delivery” and other related terms refer to a method of delivering to a human a dosage of a therapeutic or prophylactic formulation by a route such that the desired therapeutic or prophylactic agent is delivered to the lungs of a human.

The term “actuation” of “actuations” refers to triggering the device to release a metered amount of a drug formulation.

The term “abnormal liver function” may manifest as abnormalities in levels of biomarkers of liver function, including alanine transaminase, aspartate transaminase, bilirubin, and/or alkaline phosphatase, and is an indicator of drug-induced liver injury. See FDA Draft Guidance for Industry. Drug-Induced Liver Injury: Premarketing Clinical Evaluation, October 2007.

The term “base” refers to the active molecule itself that may exist with or without a corresponding salt. The description “base within the salt form” refers to the active molecule itself within a corresponding salt from. Active molecule weights and weight percentages described herein refer to the “base” or the “base within the salt form” and may be readily adjusted for the equivalent weight or weight percentages based on the individual salt species selected for the salt form of the base.

“Grade 2 liver function abnormalities” include elevations in alanine transaminase (ALT), aspartate transaminase (AST), alkaline phosphatase (ALP), or gamma-glutamyl transferase (GGT) greater than 2.5-times and less than or equal to 5-times the upper limit of normal (ULN). Grade 2 liver function abnormalities also include elevations of bilirubin levels greater than 1.5-times and less than or equal to 3-times the ULN.

“Gastrointestinal adverse events” include but are not limited to any one or more of the following: dyspepsia, nausea, diarrhea, gastroesophageal reflux disease (GERD) and vomiting.

A “carrier” or “excipient” is a compound or material used to facilitate administration of the compound, for example, to increase the solubility of the compound. Solid carriers include, e.g., starch, lactose, dicalcium phosphate, sucrose, and kaolin. In addition, various adjuvants such as are commonly used in the art may be included. These and other such compounds are described in the literature, e.g., in the Merck Index, Merck & Company, Rahway, NJ. Considerations for the inclusion of various components in pharmaceutical compositions are described, e.g., in Gilman et al. (Eds.) (1990); Goodman and Gilman's: The Pharmacological Basis of Therapeutics, 8^{th} Ed., Pergamon Press.

A “diagnostic” as used herein is a compound, method, system, or device that assists in the identification and characterization of a health or disease state. The diagnostic can be used in standard assays as is known in the art.

The term “bulking agent” refers to excipients used in pharmaceutical preparations to provide a matrix to carry the drug which are normally present in low quantities.

The term “force control agents” refers to excipients used in pharmaceutical preparations to decrease the adhesion between drug and carrier particles in adhesive mixtures for inhalation and hence to increase drug detachment during inhalation.

The terms “D10, D50 and D90” refer to volume-based diameters of particles at the 10th, 50th and 90th percentile.

The term “carrier-free blend” refers to dry powder inhalation blends that do not utilize an inert excipient to aid in the dispersion of drug particles.

The term “carrier blend” refers to dry powder inhalation blends that utilize an inert excipient to reduce cohesion force between drug particles and thereby aids in the dispersion of drug particles.

The term “shell former” refers to excipients used in spray dry powder preparation to form the outer shell to enable hollow or porous particles to form.

The term “glass former” refers to excipients used in spray dry powder preparation to prevent spray dried particles converting from amorphous form to crystalline form The term “ex vivo” refers to experimentation or manipulation done in or on living tissue in an artificial environment outside the organism.

The term “low resistance” refers to a dry powder inhalation device whereby about 100 liters per minute is required to generate the 4 kPa pressure drop required to actuate and disperse dry powder formulation contained therein.

The term “medium resistance” refers to a dry powder inhalation device whereby about 85 liters per minute is required to generate the 4 kPa pressure drop required to actuate and disperse dry powder formulation contained therein.

The term “high resistance” refers to a dry powder inhalation device whereby about 60 liters per minute is required to generate the 4 kPa pressure drop required to actuate and disperse dry powder formulation contained therein.

“Solvate” refers to the compound formed by the interaction of a solvent and nintedanib or an indolinone derivative compound, a metabolite, or salt thereof. Suitable solvates are pharmaceutically acceptable solvates including hydrates.

By “therapeutically effective amount” or “pharmaceutically effective amount” is meant nintedanib or a indolinone or salt that are useful in treatment of humans in therapeutically effective amounts and that produce the desired therapeutic effect as judged by clinical trial results and/or model animal pulmonary fibrosis, lung transplant rejection-associated chronic lung allograft dysfunction (CLAD) and restrictive allograft syndrome (RAS), cardiac fibrosis, kidney fibrosis, hepatic fibrosis, heart or kidney toxicity, cancer or disease resulting from active, previous or latent viral infection. When the invention comprises a dry powder combinations of two or more APIs, the term “therapeutically and synergistically effective” means that the addition of the second or additional plurality of APIs provides a superior therapeutic effect compared to the Nintedanib dose alone. In such aspects of the invention, the therapeutic advantage may be determined from independent assessment of the therapeutic contribution from the one or plurality of APIs for from a combination delivered together from a dry powder inhaler as an admixture of the therapeutic dry powder individual and combined components. Thus, a dose of dry powder pirfenidone that is “therapeutically and synergistically effective” in combination with the nintedanib dose yields a measurable advantage over the Nintedanib dose in isolation. Under some circumstances, the therapist putatively and synergistically effective dose is greater than the sum of the individual efficacy of each individual API.

A “therapeutic effect” relieves, to some extent, one or more of the symptoms associated with inflammation, fibrosis and/or demyelination. This includes slowing the progression of, or preventing or reducing additional inflammation, fibrosis and/or demyelination. For idiopathic pulmonary fibrosis (IPF), progressive pulmonary fibrosis (PPF) and restrictive allograft syndrome (RAS), a “therapeutic effect” is defined as a reduced level or rate of decline in forced vital capacity (FVC), and/or a patient-reported improvement in quality of life and/or a statistically significant increase in or stabilization of exercise tolerance and associated blood-oxygen saturation, reduced decline in baseline forced vital capacity, decreased incidence in acute exacerbations, increase in progression-free survival, increased time-to-death or disease progression, and/or reduced lung fibrosis. For CLAD, a “therapeutic effect” is defined as a reduced decline in forced expiratory volume in one second (FEV1), For cardiac fibrosis, a “therapeutic effect” is defined as a patient-reported improvement in quality of life and/or a statistically significant improvement in cardiac function, reduced fibrosis, reduced cardiac stiffness, reduced or reversed valvular stenosis, reduced incidence of arrhythmias and/or reduced atrial or ventricular remodeling. For kidney fibrosis, a “therapeutic effect” is defined as a patient-reported improvement in quality of life and/or a statistically significant improvement in glomerular filtration rate and associated markers. For hepatic fibrosis, a “therapeutic effect” is defined as a patient-reported improvement in quality of life and/or a statistically significant lowering of elevated aminotransferases (e.g., AST and ALT), alkaline phosphatases, gamma-glutamyl transferase, bilirubin, prothrombin time, globulins, as well as reversal of thrombocytopenia, leukopenia and neutropenia and coagulation defects. Further a potential reversal of imaging, endoscopic or other pathological findings. For disease resulting from active, previous or latent viral infection, a “therapeutic effect” is defined as a patient-reported improvement in quality of life and/or a statistically significant reduction in viral load, improved exercise capacity and associated blood-oxygen saturation, FEV1 and/or FVC, a slowed or halted progression in the same, progression-free survival, increased time-to-death or disease progression, and/or reduced incidence or acute exacerbation or reduction in neurologic symptoms. The term “prophylactic treatment” refers to treating a patient who is not yet diseased but who is susceptible to, or otherwise at risk of, a particular disease, or who is diseased but whose condition does not worsen while being treated with the pharmaceutical compositions described herein. The term “therapeutic treatment” refers to administering treatment to a patient already suffering from a disease. Thus, in preferred embodiments, treating is the administration to a mammal (either for therapeutic or prophylactic purposes) of therapeutically effective amounts of nintedanib or an indolinone derivative compound.

The term “fine particle fraction” is the proportion of aerosolized particles that are less than or equal to 5 microns in diameter.

The term “respirable delivered dose” or “fine particle dose” is the amount of drug particles inhaled during the inspiratory phase of the breath simulator that is equal to or less than 5 microns.

“Lung Deposition” as used herein, refers to the fraction of the nominal dose of an active pharmaceutical ingredient (API) that is deposited on the inner surface of the lungs.

“Nominal dose,” or “loaded dose” refers to the amount of drug that is placed in the dry powder inhaler prior to administration to a human. The amount of powder containing the nominal dose is referred to as the “fill amount.”

“Dispersion” refers to the process of scattering or diffusing the dry powder formulation fill amount into a respirable fine drug particle fraction by aerodynamic means.

“Enhanced pharmacokinetic profile” means an improvement in some pharmacokinetic parameter. Pharmacokinetic parameters that may be improved include, AUC last, AUC(0-∞) Tmax, and optionally a Cmax. The enhanced pharmacokinetic profile may be measured quantitatively by comparing a pharmacokinetic parameter obtained for a nominal dose of an active pharmaceutical ingredient (API) administered with one type of inhalation device with the same pharmacokinetic parameter obtained with oral administration of a composition of the same active pharmaceutical ingredient (API).

“Respiratory condition,” as used herein, refers to a disease or condition that is physically manifested in the respiratory tract, including, but not limited to, pulmonary fibrosis, cancer, disease resulting from active, previous or latent viral infection, bronchitis, chronic bronchitis, or emphysema.

“Drug absorption” or simply “absorption” typically refers to the process of movement of drug from site of delivery of a drug across a barrier into a blood vessel or the site of action, e.g., a drug being absorbed in the pulmonary capillary beds of the alveoli.

### BRIEF DESCRIPTION OF THE DRAWINGS

FIG. 1 is an initial scanning electron micrograph (SEM) of the hydrobromide salt of micronized nintedanib.

FIG. 2 is a scanning electron micrograph (SEM of the hydrobromide salt of micronized nintedanib after 5 months.

FIG. 3 is an initial XRPD of micronized nintedanib HBr.

FIG. 4 is an XRPD of nintedanib HBr—1 month 25°/60 RH and 40°/75 RH and after 5 months.

FIG. 5 are DSC of nintedanib HBr at an initial, 1 month 40°/75 RH and after 5 months intervals.

FIG. 6 are DVS Adsorption/desorption cycles for Trehalose:Leucine:NHBr 80:10:10 at an initial, one months, two months, and three months intervals

FIG. 7 are DVS Adsorption/desorption cycles for Lactose:Leucine:NHBr 80:10:10 at an initial and one month interval.

FIG. 8 are DVS Adsorption/desorption cycles for Trehalose:NHBr 90:10 at one month.

FIG. 9 are DVS Adsorption/desorption cycles for Lactose:NHBr 90:10 at an initial and one month interval.

FIG. 10 are DVS Adsorption/desorption cycles for Lactose:Leucine:NHBr 70:20:10 at 10 initial, one month, two months, and three months intervals.

FIG. 11 are DSC for Trehalose:leucine:NHBr 80:10:10 wt % at 10 initial, one month, two months, and three months intervals.

FIG. 12 is DSC for Lactose:leucine:NHBr 80:10:10 wt % at 10 initial and one month intervals.

FIG. 13 is DSC for Trehalose:NHBr 90:10 wt % at a one month interval.

FIG. 14 are DSC for Lactose:NHBr 90:10 wt % at an initial and one month interval.

FIG. 15 are DSC for Lactose:leucine:NHBr 70:20:10 wt % at 10 initial, one month, two months, and three months intervals.

FIGS. 16A and 16B show the change in blood glucose levels in sheep on two separate occasions (A and B); sheep were given oral glucose in the absence of a preceding dose of CuSO₄ (glucose), or following CuSO₄ administration (CuSO₄+glucose). Data are means SD for n=4 sheep; **p<0.01; ****p<0.0001.

FIG. 17 shows the spike in blood glucose levels confirming effective reticular groove closure following CuSO₄ administration (CuSO₄+glucose). In the absence of CuSO₄ (assessed at a separate time) there was no ‘spike’ in blood glucose levels observed. Data are means±SD for n=10 sheep; ****p<0.0001.

FIGS. 18A, 18B, 18C, 18D, 18E, 18F and 18G show a lung function assessment in sheep pre- and post-treatment 1-4. (A) Transpulmonary pressure (index of airway resistance), (B) Dynamic compliance index, (C) Ventilation, (D) Tidal volume, (E) Breath frequency, (F) Inspiratory flow and (G) Expiratory flow. White bars=pre-dose; Grey bars=post-dose. Data shown: mean±SEM for n=6 sheep; *p<0.05; one-way ANOVA and Sidak's multiple comparisons.

FIG. 19 shows sheep epithelial lining fluid (ELF) pharmacokinetics—Dry powder vs. nebulized inhaled nintedanib.

FIG. 20 shows sheep plasma pharmacokinetics—Dry powder vs. nebulized inhaled nintedanib

FIG. 21 is a scanning electron micrograph (SEM) micronized fine nintedanib hydrobromide salt HBr.

FIG. 22 is an x-ray diffraction pattern for nintedanib hydrobromide salt.

### DETAILED DESCRIPTION OF THE INVENTION

### Nintedanib and Indolinone Derivative Compounds—Therapeutic Utility

The indolinone derivative for use in a indolinone derivative formulation as described herein comprises nintedanib (methyl (3Z)-3-[[4-[methyl-[2-(4-methylpiperazin-1-yl)acetyl]amino]anilino]-phenylmethylidene]-2-oxo-1H-indole-6-carboxylate) or a salt thereof.

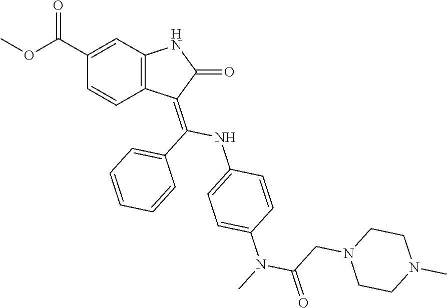

Other indolinone derivative compounds, or salts thereof, may be used in place of nintedanib. Indolinone derivative compounds include, but are not limited to, those compounds that are structurally similar to nintedanib and have the same type of biological activity as nintedanib. Indolinone derivative compounds include modifications to the nintedanib molecule that are foreseeable based on substitution of chemical moieties that preserve the Structure Activity Relationship (SAR) of nintedanib based on the interaction of nintedanib, or the subject derivative as specific and selective inhibitor of certain tyrosine kinases as described below. Indolinone derivative compounds include, but are not limited to, those compounds described in U.S. Pat. Nos. 6,762,180 and 7,119,093.

Nintedanib inhibits a broad range of kinases at pharmacologically relevant concentrations. Examples of targeted kinases include all three VEGFR subtypes (VEGFR-1, IC50 34 nM; VEGFR-2, IC50 21 nM; VEGFR-3, IC50 13 nM), FGFR types (FGFR-1, IC50 69 nM; FGFR-2, IC50 37 nM; FGFR-3, IC50 108 nM; FGFR-4, IC50 610 nM), and PDGFR-α (IC50, 59 nM) and PDGFR-β (IC50, 65 nM). The ability of nintedanib to simultaneously target these three, distinct proangiogenic receptor classes may enhance its antitumor effects and overcome pathways of resistance to VEGF- and VEGFR-2-targeted agents. Nintedanib also inhibited Flt-3 and members of the Src-family (Src, Lyn, and Lck), which may have therapeutic potential for conditions such as hematologic diseases.

IPF and PPF are a chronic and progressive, fibrotic lung diseases associated with a short median survival post diagnosis of 2-3 years due to a lack of effective therapies. Both IPF and PPF are characterized by uncontrolled fibroblast/myofibroblast proliferation and differentiation, and excessive collagen deposition within the lung interstitium and alveolar space, leading to symptoms of cough and dyspnea, and ultimately to respiratory failure.

In some embodiments, administration of nintedanib or indolinone or salt thereof, by inhalation has reduced gastrointestinal and liver side-effects when compared to oral administration. Reducing these side-effects increases patient safety, maximizes patient compliance, avoids dose reduction and/or stoppage protocols, and enables local lung dose escalation for additional efficacy otherwise not possible with the oral product.

In some embodiments, administration of nintedanib or indolinone or salt thereof in combination with pirfenidone or pyridine analog, by inhalation has reduced gastrointestinal and liver side-effects when compared to add-on oral administration of nintedanib and pirfenidone. Reducing these side-effects increases patient safety, maximizes patient compliance, avoids dose reduction and/or stoppage protocols, and enables either local lung dose escalation or combination ratio optimization for additional efficacy otherwise not possible treating with the two oral products. In such cases, the invention includes a combination of the dry powders disposed in a dry powder inhalation device such that when dispersed, the combination yields a dry powder combined aerosol comprising more than one API for inhalation.

The specially formulated nintedanib or indolinone dry powder for dispersion and inhaled administration are used in methods of treatment of lung disease in a human. The methods are applied to diseases including, not limited to, pulmonary fibrosis, idiopathic pulmonary fibrosis, progressive pulmonary fibrosis, radiation induced fibrosis, silicosis, asbestos induced pulmonary or pleural fibrosis, acute lung injury, acute respiratory distress syndrome (ARDS), sarcoidosis, usual interstitial pneumonia (UIP), cystic fibrosis, Chronic lymphocytic leukemia (CLL)-associated fibrosis, Hamman-Rich syndrome, Caplan syndrome, coal worker's pneumoconiosis, cryptogenic fibrosing alveolitis, obliterative bronchiolitis, chronic bronchitis, emphysema, pneumonitis, lung cancer, Wegner's granulamatosis, scleroderma-associated lung fibrosis, systemic sclerosis-associated interstitial lung disease (SSc-ILD), silicosis, interstitial lung disease, asbestos induced pulmonary and/or pleural fibrosis. In some methods the primary, lung disease is lung fibrosis (i.e. pulmonary fibrosis), while in other methodologies the fibrosis is a comorbidity of a separate disease such as cancer or is the result of a prior infection or surgery, including particularly chronic lung allograft dysfunction (CLAD), and including restrictive allograft syndrome (RAS).

### Pirfenidone and Pyridone Analog Compounds—Therapeutic Utility

For the invention described herein, Pirfenidone or pyridone analog thereof, are selected from 1-Phenyl-2-(1H)pyridone, 5-methyl-1-phenyl-1,2-dihydropyridin-2-one, 5-methyl-1-(4-methylphenyl)-2-(1H)-pyridone, 5-Methyl-1-(2′-pyridyl)-2-(1H)pyridone, 6-Methyl-1-phenyl-3-(1H)pyridone, 6-Methyl-1-phenyl-2-(1H)pyridone, 5-Methyl-1-p-tolyl-3-(1H)pyridone, 5-Methyl-1-phenyl-3-(1H)pyridone, 5-Methyl-1-p-tolyl-2-(1H)pyridone, 5-Ethyl-1-phenyl-2-(1H)pyridone, 5-Ethyl-1-phenyl-3-(1H)pyridone, and 4-Methyl-1-phenyl-3-(1H)pyridine, including deuterated forms.

In epithelial injury, epithelial cells are triggered to release several pro-inflammatory and pro-fibrotic mediators, including interleukin-1β, the potent fibroblast growth factors transforming growth factor-beta (TGF-beta), tumor necrosis factor (TNF), platelet derived growth factor (PDGF), endothelin, other cytokines, metalloproteinases and the coagulation mediator tissue factor. Importantly, the triggered epithelial cell becomes vulnerable to apoptosis, and together with an apparent inability to restore the epithelial cell layer are the most fundamental abnormalities in fibrotic disease.

In conditions such as diseases, physiological responses characterized by control of pro-inflammatory and pro-fibrotic factors with pyridone analog, such as Pirfenidone are beneficial treating or preventing fibrosis, inflammation, or transplant rejection. The mechanism of action for pyridone analogs, such as Pirfenidone is to regulate production of cytokines and growth factors. These effects may directly result from direct Pirfenidone exposure or may reflect secondary effects related to modulation of a single molecular target. In either event, Pirfenidone modulation of cytokines, growth factors and markers of oxidative stress demonstrate that the anti-fibrotic effects observed in vivo are associated with regulation of pathways relevant to ongoing fibrosis and provide support for the observed anti-fibrotic effects. Pirfenidone has been approved as an oral therapy for the treatment of idiopathic pulmonary fibrosis. See U.S. Pat. Nos. 10,092,552, 9,770,443, 10,028,966, 10,105,356, and 11,123,290, specifically incorporated by reference herein.

### PDE4 Inhibitors and Subtype Compounds—Therapeutic Utility

Phosphodiesterases (PDEs) mediate the hydrolysis of the second messengers, cyclic adenosine monophosphate (cAMP) or cyclic guanosine monophosphate (cGMP). PDEs are coded by 11 gene superfamilies containing multiple genes (coding for subtypes A, B, C, etc.) that also give rise to alternative mRNA-splicing variants leading to approximately 100 PDE isoforms.

The PDE4 subtypes A-D are encoded by different genes, PDE4A, B, C, and D, with post-translational processing resulting in N-terminal variant groups (long, short, and super-short form) according to the presence or absence of upstream conserved regions 1 and 2 (UCR1 or UCR2) N-terminal domains. PDE signaling is highly compartmentalized as PDE4 subtypes can integrate into macromolecular complexes known as signalosomes.

PDE4 has traditionally been implicated in the regulation of inflammation and the modulation of immunocompetent cells, and the three selective PDE4 inhibitors currently available support a beneficial role for PDE4 inhibitors in inflammatory and/or autoimmune diseases. Evidence points to the involvement of a range of immune cells and inflammatory responses in pulmonary fibrosis.

The first-in-class PDE4 inhibitor, oral roflumilast (Daliresp®, Daxas®) reduces the risk of COPD exacerbations in patients with severe COPD associated with chronic bronchitis and a history of exacerbations. Another compound, oral apremilast (Otezla®), is effective treating psoriatic arthritis and plaque psoriasis. A third PDE4 inhibitor, crisaborole (Eucrisa®) is effective treating mild-to-moderate atopic dermatitis.

The general anti-inflammatory potential of PDE4 inhibition and use in various inflammatory and immune-mediated diseases has been described. However, PDE4 may also play an important role in fibrosis. Roflumilast, apremilast and crisaborole each hold potential as PDE4 inhibitors to be effective in treating fibrotic diseases. Additionally, BI 1015550 (PDE4B inhibitor) has also shown promise. For purposes on this invention, PDE4 inhibitor includes Roflumilast, Apremilast, Crisaborole, BI 1015550, CHF6001, Ronomilast, Oglemilast, GSK256066, YM976, GS5759, GPD-1116, MEM1414, RPL554, Asp3258, E6005, GW842470X, OPA-15406, Leo-29102, DRM02, Pefcalcitol, HFP034, CBS3995, MK0873, Revamilast, NCS 613, FCPR03, HT-0712, MK0952, API-4, ASP9831, including deuterated forms.

### Prostacyclin Analogs and Subtype Compounds—Therapeutic Utility

Prostacyclin analogs promote vasodilation of pulmonary and systemic arterial vascular beds and inhibit platelet aggregation. In addition to its effects on the pulmonary vasculature, data indicate prostacyclin analogs have antifibrotic properties. By non-limiting example, prostacyclin analogs include Selexipag, Epoprostenol, Iloprost, Treprostinil. Specifically, these have been shown to have dose-dependent prevention of fibroblast proliferation to decrease extracellular matrix composition via a TGF-β1 and PDGF-BB antagonism in human peripheral lung fibroblasts, inhibit extracellular matrix-deposition by fibroblasts by both cyclic adenosine monophosphate (cAMP)-dependent and non-dependent mechanisms in human peripheral lung fibroblasts, suppress profibrotic fibroblast activity and the synthesis and deposition of collagen and fibronectin in mice, with anti-inflammatory processes mediated by NK-κB signaling, and suppress profibrotic fibroblast activity through Yes-associated protein (YAP)/transcriptional coactivator with PDZ-binding motif (TAZ) inhibition from prostacyclin (IP) receptor activation. Further, clinical results indicate prostacyclin analogs may benefit IPF patients. For purposes on this invention and by non-limiting example, prostacyclin analogs include Treprostinil, Iloprost, Epoprostinol and Berapost, including deuterated forms.

### Pulmonary Fibrosis

A method for treating or preventing progression of pulmonary disease, including progressive pulmonary fibrosis, such as idiopathic pulmonary fibrosis comprises administering nintedanib or indolinone or salt thereof or in combination with Pirfenidone or pyridone analog or PDE4 inhibitor or prostacyclin analog to a middle to lower respiratory tract of a patient having or suspected of having pulmonary disease through oral inhalation of a dry powder aerosol. A method of treating or preventing progression of interstitial pulmonary fibrosis and includes patients who are being mechanically ventilated. As noted above, administration of the dispersed dry powder, either as nintedanib particles, co-precipitate of nintedanib particles, combination of Nintedanib in either form and optional inclusion of a PDE4 inhibitor or prostacyclin yields an aerosol in the form of a cloud or dry messed containing the nintedanib or indolinone of salt thereof, or nintedanib or indolinone of salt thereof particles that have a mean diameter less than about 5.0 μm and component dry particles of the Pirfenidone, PDE 4 inhibitor or prostacyclin having the mean particle size diameter as described herein. Accordingly, the aerosol can include a single API, a dual combination of nintedanib, indolinone or salt thereof, a double combination of Nintedanib and Pirfenidone, or a triple or quadruple combination further including Pirfenidone and prostacyclin. In such cases, the method of the invention includes the therapeutically and/or therapeutically synergistic combination of the dry powders disposed in a dry powder inhalation device such that when dispersed, the combination yields a dry powder combined aerosol comprising more than one API for inhalation.

A method for treating or preventing progression pulmonary fibrosis, such as idiopathic pulmonary fibrosis (IPF), comprises administering nintedanib or indolinone or salt thereof or in combination with Pirfenidone or pyridone analog to a middle to lower respiratory tract of a subject having or suspected IPF through oral inhalation of a dry powder aerosol comprising nintedanib or salt thereof or in combination with Pirfenidone.

A method for treating or preventing progression of progressive pulmonary fibrosis (PPF), comprising administering nintedanib or indolinone or salt thereof or in combination with Pirfenidone or pyridone analog to a middle to lower respiratory tract of a subject having or suspected PPF through oral inhalation of a dry powder aerosol comprising nintedanib or salt thereof or in combination with Pirfenidone.

A method for treating or preventing progression of systemic sclerosis associated interstitial lung disease (SSc-ILD), comprising administering nintedanib or indolinone or salt thereof or in combination with Pirfenidone or pyridone analog to a middle to lower respiratory tract of a subject having or suspected of having SSc-ILD through oral inhalation of a dry powder aerosol comprising nintedanib or salt thereof or in combination with Pirfenidone.

A method for treating or preventing progression of bronchiolitis obliterans, comprising administering nintedanib or indolinone or salt thereof or in combination with Pirfenidone or pyridone analog to a middle to lower respiratory tract of a patient having or suspected of having bronchiolitis obliterans through oral inhalation of a dry powder aerosol comprising nintedanib or salt thereof or in combination with Pirfenidone.

A method for treating or preventing progression of chronic lung allograft dysfunction, comprising administering nintedanib or indolinone salt thereof or in combination with Pirfenidone or pyridone analog to a middle to lower respiratory tract of a patient having or suspected of having restrictive allograft syndrome through oral inhalation of a dry powder aerosol comprising nintedanib or salt thereof or in combination with Pirfenidone.

A method for treating or preventing progression of restrictive allograft syndrome, comprising administering nintedanib indolinone salt thereof or in combination with Pirfenidone or pyridone analog to a middle to lower respiratory tract of a patient having or suspected of having restrictive allograft syndrome through oral inhalation of a dry powder aerosol comprising nintedanib or salt thereof or in combination with Pirfenidone.

IPF as described herein refers to “idiopathic pulmonary fibrosis” and is in some embodiments a chronic disease that manifests over several years and is characterized by scar tissue within the lungs, in the absence of known provocation. Exercise-induced breathlessness and chronic dry cough may be the prominent symptoms. IPF belongs to a family of lung disorders known as the interstitial lung diseases (ILD) or, more accurately, the diffuse parenchymal lung diseases. Within this broad category of diffuse lung diseases, IPF belongs to the subgroup known as idiopathic interstitial pneumonia (IIP). There are seven distinct IIPs, differentiated by specific clinical features and pathological patterns. IPF is the most common form of IIP. It is associated with the pathologic pattern known as usual interstitial pneumonia (UIP); for that reason, IPF is often referred to as IPF/UIP. IPF is usually fatal, with an average survival of approximately three years from the time of diagnosis. There is no single test for diagnosing pulmonary fibrosis; several different tests including chest x-ray, HRCT, pulmonary function test, exercise testing, bronchoscopy and lung biopsy are used in conjunction with the methods described herein.

Idiopathic pulmonary fibrosis (also known as cryptogenic fibrosing alveolitis) is the most common form of interstitial lung disease and may be characterized by chronic progressive pulmonary parenchymal fibrosis. It is a progressive clinical syndrome with unknown etiology; the outcome is frequently fatal as no effective therapy exists. In some embodiments, nintedanib inhibits fibroblast proliferation and differentiation related to collagen synthesis, inhibits the production and activity of TGF-beta, reduces production of fibronectiv and connective tissue growth factor, inhibits TNF-alpha and I-CAM, increase production of IL-10, and/or reduces levels of platelet-derived growth factor (PDGF) A and B in bleomycin-induced lung fibrosis. The methods and compositions described herein may provide tolerability and usefulness in patients with advanced idiopathic pulmonary fibrosis and other lung diseases. In some embodiments, nintedanib methods and compositions described herein may provide tolerability and usefulness in patients with mild to moderate idiopathic pulmonary fibrosis. Increased patient survival, enhanced vital capacity, reduced episodes of acute exacerbation (compared to placebo), and/or slowed disease progression are observed following treatment with the compositions of the invention.

PPF as described herein refers to “progressive pulmonary fibrosis”. Like IPF, PPF is a chronic disease that manifests over several years and is characterized by scar tissue within the lungs. Exercise-induced breathlessness and chronic dry cough may be the prominent symptoms. PPF also belongs to the family of interstitial lung diseases (ILD) or, more accurately, the diffuse parenchymal lung diseases. PPF explicitly excludes idiopathic pulmonary fibrosis. IPF is defined as ILD with no known cause which is associated with the histological or radiological pattern of Usual Interstitial Pneumonia (UIP). There are specific criteria for PPF, largely comprising worsening symptoms together with increasing radiological fibrosis or impairment of respiratory physiology over time in non-IPF ILD. Despite the aforementioned histological and radiological differences with IPF, its natural history is similar. PPF is usually fatal, with an average survival of approximately 3-5 years from the time of diagnosis. There is no single test for diagnosing PPF; several different tests including chest x-ray, HRCT, pulmonary function test, exercise testing, bronchoscopy and lung biopsy are used in conjunction with the methods described herein.

Exemplary fibrotic lung diseases for the treatment or prevention using the methods described herein include, but are not limited to, idiopathic pulmonary fibrosis, progressive pulmonary fibrosis, systemic sclerosis-associated interstitial lung disease, pulmonary fibrosis secondary to transplant rejection such as bronchiolitis obliterans and restrictive allograft syndrome, systemic inflammatory disease such as rheumatoid arthritis, scleroderma, lupus, cryptogenic fibrosing alveolitis, radiation induced fibrosis, sarcoidosis, scleroderma, chronic asthma, silicosis, asbestos induced pulmonary or pleural fibrosis, acute lung injury and acute respiratory distress (including bacterial pneumonia induced, trauma induced, viral pneumonia induced, ventilator induced, non-pulmonary sepsis induced, and aspiration induced).

Where the methods of the invention are applied to treatments or preventing progression of pulmonary cancer, the disorder includes lung carcinoid tumors or bronchial carcinoids, primary or secondary lung cancers resulting from metastatic disease, including non-small cell lung cancer, bronchioloalveolar carcinoma, sarcoma, and lymphoma.

Methods of the invention include treatment or prophylaxis of patients identified as having gastrointestinal stromal tumors, relapsed or refractory Ph-positive Acute lymphoblastic leukemia (ALL), myelodysplastic/myeloproliferative diseases associated with platelet-derived growth factor receptor gene re-arrangements, aggressive systemic macrocytosis (ASM) (without or an unknown D816V c-KIT mutation), hyper eosinophilic syndrome (HES) and/or chronic eosinophilic leukemia (CEL) who have the FIP1L1-PDGFRα fusion kinase (CHIC2 allele deletion) or FIP1L1-PDGFR-alpha fusion kinase negative or unknown, or unresectable, recurrent and/or metastatic dermatofibrosarcoma protuberans, and combinations thereof.

### Lung Transplant Rejection

Lung transplant rejection initially manifests as Chronic Lung Allograft Dysfunction (CLAD) and is the major cause of mortality. The major feature is bronchiolitis obliterans. The rate of decline in lung function when severe averaging about 7-fold higher than seen in a patient with idiopathic pulmonary fibrosis (IPF). Some CLAD patients (approximately 30%) develop Restrictive Allograft Syndrome (RAS) which carries a worse prognosis. In these patients there is loss of both FVC, forced vital capacity creating restrictive pulmonary function. The pathophysiology is similar to IPF with progressive interstitial fibrosis.

A method for treating or preventing progression of pulmonary disease, comprising administering nintedanib or indolinone or salt thereof to a middle to lower respiratory tract of a patient having or suspected of having pulmonary disease through oral inhalation of a dry powder aerosol. The method includes treating or preventing progression of Chronic Lung Allograft Dysfunction (CLAD) as a manifestation of lung transplant rejection. The method includes delivery to patients who are being mechanically ventilated. The method also includes administration of nintedanib or indolinone or salt thereof and pirfenidone or pyridone analog in combination.

A method for treating or preventing progression of pulmonary disease, comprising administering nintedanib or indolinone or salt thereof to a middle to lower respiratory tract of a patient having or suspected of having pulmonary disease through oral inhalation of a dry powder aerosol. The method includes treating or preventing progression of bronchiolitis obliterans as a manifestation of lung transplant rejection. The method includes delivery to patients who are being mechanically ventilated. The method also includes administration of nintedanib or indolinone or salt thereof and Pirfenidone or pyridone analog in combination.

A method for treating or preventing progression of pulmonary disease, comprising administering nintedanib or indolinone or salt thereof to a middle to lower respiratory tract of a patient having or suspected of having pulmonary disease through oral inhalation of a dry powder aerosol. The method includes treating or preventing progression of Restrictive Allograft Syndrome (RAS) as a manifestation of lung transplant rejection. The method includes delivery to patients who are being mechanically ventilated. The method also includes administration of nintedanib or indolinone or salt thereof and Pirfenidone or pyridone analog in combination.

### Cardiac Fibrosis

A method for treating or preventing progression of an extrapulmonary disease, comprising administering nintedanib or indolinone or salt thereof to lower respiratory tract of a patient having or suspected of having cardiac fibrosis through oral inhalation of a dry powder aerosol, wherein cardiac fibrosis includes remodeling of cardiac tissue observed in chronic hypertension and may involve myocyte hypertrophy as well as fibrosis, an increased and non-uniform deposition of extracellular matrix proteins. The extracellular matrix connects myocytes, aligns contractile elements, prevents overextending and disruption of myocytes, transmits force and provides tensile strength to prevent rupture. Fibrosis occurs in many models of hypertension leading to an increased diastolic stiffness, a reduction in cardiac function and an increased risk of arrhythmias. If fibrosis rather than myocyte hypertrophy is the critical factor in impaired cardiovascular function, then reversal of cardiac fibrosis facilitates return of normal cardiac function. The method also includes administration of nintedanib or indolinone or salt thereof and pirfenidone or pyridone analog in combination.

The term “cardiac fibrosis” by non-limiting example relates to remodeling associated with or resulting from viral or bacterial infection, surgery, Duchenne muscular dystrophy, radiation therapy, chemotherapy, transplant rejection and chronic hypertension where myocyte hypertrophy as well as fibrosis is involved and an increased and non-uniform deposition of extracellular matrix proteins occurs. Fibrosis occurs in many models of hypertension leading to an increased diastolic stiffness, a reduction in cardiac function, an increased risk of arrhythmias and impaired cardiovascular function.

### Cancer

A method for treating or preventing progression of lung cancer, comprising administering nintedanib or indolinone or salt thereof to the respiratory tract of a patient having or suspected of having lung cancer through oral inhalation of a dry powder aerosol, wherein the lung cancer includes lung carcinoid tumors or bronchial carcinoids, primary or secondary lung cancers resulting from metastatic disease, including non-small cell lung cancer, bronchioloalveolar carcinoma, sarcoma, and lymphoma. The method also includes administration of nintedanib or indolinone or salt thereof and pirfenidone or pyridone analog in combination.

Lung cancer mortality is high, and annual lung cancer deaths equal prostate, breast, colon, and rectum cancers combined. Despite the advancement in knowledge on molecular mechanisms and the introduction of multiple new therapeutic lung cancer agents, the dismal 5-year survival rate (11-15%) remains relatively unaltered. This reflects the limited available knowledge on factors promoting oncogenic transformation to and proliferation of malignant cells.

We now know that tumor growth is not determined only by malignant cells, because interactions between cancer cells and the stromal compartment have major impacts on cancer growth and progression. Aggressive malignant cells are clever at exploiting the tumor microenvironment: tumor cells can (1) reside in the stroma and transform it, (2) alter the surrounding connective tissue, and (3) modify the metabolism of resident cells, thus yielding a stroma, which is permissive rather than defensive.

Beyond overcoming the microenvironmental control by the host, key characteristics of cancer cells is their ability to invade the tissue and metastasize distantly. For invasion and metastasis, the concerted interactions between fibroblasts, immune cells, and angiogenic cells and factors are essential.

The tumor stroma basically consists of (1) the nonmalignant cells of the tumor such as CAFs, specialized mesenchymal cell types distinctive to each tissue environment, innate and adaptive immune cells, and vasculature with endothelial cells and pericytes and (2) the extracellular matrix (ECM) consisting of structural proteins (collagen and elastin), specialized proteins (fibrillin, fibronectin, and elastin), and proteoglycans. Angiogenesis is central for cancer cell growth and survival and has hitherto been the most successful among stromal targets in anticancer therapy. Initiation of angiogenesis requires matrix metalloproteinase (MMP) induction leading to degradation of the basement membrane, sprouting of endothelial cells, and regulation of pericyte attachment. However, CAFs play an important role in synchronizing these events through the expression of numerous ECM molecules and growth factors, including transforming growth factor (TGF)-β, vascular endothelial growth factor (VEGF), and fibroblast growth factor (FGF2).

The normal tissue stroma is essential for maintenance and integrity of epithelial tissues and contains a multitude of cells that collaborate to sustain normal tissue homeostasis. There is a continuous and bilateral molecular crosstalk between normal epithelial cells and cells of the stromal compartment, mediated through direct cell-cell contacts or by secreted molecules. Thus, minor changes in one compartment may cause dramatic alterations in the whole system.

A similarity exists between stroma from wounds and tumors, because both entities had active angiogenesis and numerous proliferating fibroblasts secreting a complex ECM, all on a background of fibrin deposition. Consequently, the tumor stroma has been commonly referred to as activated or reactive stroma.

A genetic alteration during cancer development, leading to a malignant cell, will consequently change the stromal host compartment to establish a permissive and supportive environment for the cancer cell. During early stages of tumor development and invasion, the basement membrane is degraded, and the activated stroma, containing fibroblasts, inflammatory infiltrates, and newly formed capillaries, comes into direct contact with the tumor cells. The basement membrane matrix also modifies cytokine interactions between cancer cells and fibroblasts. These cancer-induced alterations in the stroma will contribute to cancer invasion. Animal studies have shown that both wounding and activated stroma provides oncogenic signals to facilitate tumorigenesis. Although normal stroma in most organs contains a minimal number of fibroblasts in association with physiologic ECM, the activated stroma is associated with more ECM-producing fibroblasts, enhanced vascularity, and increased ECM production. This formation of a specific tumor stroma type at sites of active tumor cell invasion is considered an integral part of the tumor invasion and has been termed as tumor stromatogenesis.

The expansion of the tumor stroma with a proliferation of fibroblasts and dense deposition of ECM is termed a desmoplastic reaction. It is secondary to malignant growth and can be separated from alveolar collapse, which does not show neither activated fibroblasts nor the dense collagen/ECM. Morphologically this is termed desmoplasia and was initially conceived as a defense mechanism to prevent tumor growth, but data have shown that in established tumors, this process, quite oppositely, participates in several aspects of tumor progression, such as angiogenesis, migration, invasion, and metastasis. The latter studies show that fibroblasts and tumor cells can enhance local tissue growth and cancer progression through secreting ECM and degrading components of ECM within the tumor stroma. This is in part related to the release of substances sequestered in the ECM, such as VEGF, and cleavage of products from ECM proteins as a response to secretion of carcinoma-associated MMPs.

Profibrotic growth factors, released by cancer cells, such as TGF-β, platelet-derived growth factor (PDGF), and FGF2 govern the volume and composition of the tumor stroma as they are all key mediators of fibroblast activation and tissue fibrosis. PDGF and FGF2 play significant roles in angiogenesis as well.

In tumors, activated fibroblasts are termed as peritumoral fibroblasts or carcinoma-associated fibroblasts (CAFs). CAFs, like activated fibroblasts, are highly heterogeneous and believed to derive from the same sources as activated fibroblasts. The main progenitor seems to be the locally residing fibroblast, but they may also derive from pericytes and smooth muscle cells from the vasculature, from bone marrow-derived mesenchymal cells, or by epithelial or endothelial mesenchymal transition. The term CAF is rather ambiguous because of the various origins from which these cells are derived, as is the difference between activated fibroblasts and CAFs. There are increasing evidence for epigenetic and possibly genetic distinctions between CAFs and normal fibroblasts. CAFs can be recognized by their expression of α-smooth muscle actin, but due to heterogeneity α-smooth muscle actin expression alone will not identify all CAFs. Hence, other used CAF markers are fibroblast-specific protein 1, fibroblast activation protein (FAP), and PDGF receptor (PDGFR) α/β.

In response to tumor growth, fibroblasts are activated mainly by TGF-β, chemokines such as monocyte chemotactic protein 1, and ECM-degrading agents such as MMPs. Although normal fibroblasts in several in vitro studies have demonstrated an inhibitory effect on cancer progression, today, there is solid evidence for a cancer-promoting role of CAFs. In breast carcinomas, as much as 80% of stromal fibroblasts are considered to have this activated phenotype (CAFs).

CAFs promote malignant growth, angiogenesis, invasion, and metastasis. The roles of CAFS and their potential as targets for cancer therapy have been studied in xenografts models, and evidence from translational studies has revealed a prognostic significance of CAFs in several carcinoma types.

In the setting of tumor growth, CAFs are activated and highly synthetic, secreting, for example, collagen type I and IV, extra domain A-fibronectin, heparin sulfate proteoglycans, secreted protein acidic and rich in cysteine, tenascin-C, connective tissue growth factors, MMPs, and plasminogen activators. In addition to secreting growth factors and cytokines, which affect cell motility, CAFs are an important source for ECM-degrading proteases such as MMPs that play several important roles in tumorigenesis. Through degradation of ECM, MMPs can, depending on substrate, promote tumor growth, invasion, angiogenesis, recruitment of inflammatory cells, and metastasis. Besides, a number of proinflammatory cytokines seem to be activated by MMPs.

After injection of B16M melanoma cells in mice, the formation of liver metastases was associated with an early activation of stellate cells (fibroblast-like) in the liver, as these seemed important for creating a metastatic niche and promoting angiogenesis. MMPs have also been linked to tumor angiogenesis in various in vivo models. CAFs, when coinjected into mice, facilitated the invasiveness of otherwise noninvasive cancer cells. Furthermore, xenografts containing CAFs apparently grow faster than xenografts infused with normal fibroblasts.

At CAF recruitment and accumulation in the tumor stroma, these cells will actively communicate with cancer cells, epithelial cells, endothelial cells, pericytes, and inflammatory cells through secretion of several growth factors, cytokines, and chemokines. CAFs provide potent oncogenic molecules such as TGF-β and hepatocyte growth factor (HGF).

TGF-β is a pleiotropic growth factor expressed by both cancer and stromal cells. TGF-β is, in the normal and premalignant cells, a suppressor of tumorigenesis, but as cancer cells progress, the antiproliferative effect is lost, and instead, TGF-β promotes tumorigenesis by inducing differentiation into an invasive phenotype. TGF-β may also instigate cancer progression through escape from immunosurveillance, and increased expression of TGF-β correlate strongly with the accumulation of fibrotic desmoplastic tissue and cancer progression. Recently, a small molecule inhibitor of TGF-β receptor type I was reported to inhibit the production of connective tissue growth factor by hepatocellular carcinoma (HCC) cells, resulting in reduced stromal component of the HCCs. Inhibition of the TGF-β receptor aborted the crosstalk between HCCs and CAFs and consequently avoided tumor proliferation, invasion, and metastasis. HGF belongs to the plasminogen family and is tethered to ECM in a precursor form. It binds to the high-affinity receptor c-met, and overexpression or constant oncogenic c-Met signaling lead to proliferation, invasion, and metastasis.

PDGFs are regulators of fibroblasts and pericytes and play important roles in tumor progression. It is a chemotactic and growth factor for mesenchymal and endothelial cells. It has a limited autocrine role in tumor cell replication, but is a potential player, in a paracrine fashion, and in tumor stroma development. It induces the proliferation of activated fibroblasts and possibly recruits CAFs indirectly by stimulation of TGF-β release from macrophages.

A tumor cannot develop without the parallel expansion of a tumor stroma. Although we still do not comprehend the exact mechanisms regulating fibroblast activation and their accumulation in cancer, the available evidence points to the possibility that the tumor stroma or CAFs are candidate targets for cancer treatment.

CAFs and MMPs have been considered two of the key regulators of epithelial-derived tumors representing potential new targets for integrative therapies, affecting both the transformed and non-transformed components of the tumor environment. As commented earlier, the experience with MMP inhibitors have so far been unsuccessful. Evidence that CAFs are epigenetically and possibly also genetically distinct from normal fibroblasts is beginning to define these cells as potential targets for anticancer therapy. FAP, expressed in more than 90% of epithelial carcinomas, emerged early as a promising candidate for targeting CAFs, and the potential therapeutic benefit of its inhibition was reviewed recently. In preclinical studies, abrogation of FAP attenuates tumor growth and significantly enhance tumor tissue uptake of anticancer drugs. In a phase I study, where patients with FAP-positive advanced carcinomas (colorectal cancer and NSCLC) were treated with FAP-antibody, the antibody bound specifically to tumor sites, but no objective responses were observed.

The consistent and repeated findings of cancer cells that readily undergo invasion and metastasis in response to TGF-β have pointed to the need of novel anticancer agents targeting the oncogenic activities of TGF-β. A large number of anti-TGF-β antibodies and TGF-β-receptor I kinases have been tested preclinically during the past decade. Because of the lack of success, targeting of the TGF-β signaling system still remains elusive. It should be noted that both protumoral and antitumoral effects have been assigned to TGF-β, and the multifunctional nature of TGF-β apparently represents the greatest barrier to effectively target this ligand, its receptor, or downstream effectors.

### Combinations

By non-limiting example, nintedanib or indolinone or salt thereof, are administered in a dosage regimen that includes in a fixed combination, co-administered, administered sequentially, or co-prescribed with a PDE4 inhibitor for the treatment of interstitial lung disease.

By non-limiting example, nintedanib or indolinone or salt thereof, are administered in a dosage regimen that includes in fixed combination, co-administered, administered sequentially, or co-prescribed with a prostacyclin analog for the treatment of interstitial lung disease.

By non-limiting example, nintedanib or indolinone or salt thereof, are administered in a dosage regimen that includes in fixed combination, co-administered, administered sequentially, or co-prescribed with pirfenidone or pyridine analog for the treatment of interstitial lung disease.

A promising approach to treat cancer is the administration of “cocktail therapy” or “cocktail prophylaxis” where the method is comprised of co-administering or sequentially administering inhaled nintedanib or indolinone or salt thereof with agents targeting cancer, including but not limited to gefitinib (Iressa, also known as ZD1839), Erlotinib (also known as Tarceva), Bortezomib (originally codenamed PS-341; marketed as Velcade® and Bortecad®), Janus kinase inhibitors, ALK inhibitors, PARP inhibitors (Iniparib; BSI 201); PI3K inhibitors, Apatinib (YN968D1), Selumetinib, Salinomycin, Abitrexate (methotrexate), Abraxane (Paclitaxel Albumin-stabilized Nanoparticle Formulation), Afatinib Dimaleate, Alimta (pemetrexed disodium), Avastin (Bevacizumab), Carboplatin, Cisplatin, Crizotinib, Erlotinib Hydrochloride, Folex (methotrexate), Folex PFS (methotrexate), Gefitinib Gilotrif (afatinib dimaleate), Gemcitabine Hydrochloride, Gemzar (gemcitabine hydrochloride), Iressa (Gefitinib), Methotrexate, Methotrexate LPF (methotrexate), Mexate (methotrexate), Mexate-AQ (methotrexate), Paclitaxel, Paclitaxel Albumin-stabilized Nanoparticle Formulation, Paraplat (carboplatin), Paraplatin (carboplatin), Pemetrexed Disodium, Platinol (cisplatin), Platinol-AQ (Cisplatin), Tarceva (Erlotinib Hydrochloride), Taxol (Paclitaxel), and Xalkori (Crizotinib). In such cases, the invention includes a combination of the dry powders disposed in a dry powder inhalation device such that when dispersed, the combination yields a dry powder combined aerosol comprising more than one API for inhalation and the resulting effect is therapeutically effective and/or synergistically and therapeutically effective.

Combinations approved for non-small cell lung cancer may include: Carboplatin-Taxol and Gemcitabline-Cisplatin.

Drugs approved for small cell lung cancer may include: Abitrexate (methotrexate), Etopophos (etoposide phosphate), Etoposide, Etoposide Phosphate, Folex (methotrexate), Folex PFS (methotrexate), Hycamtin (topotecan hydrochloride), Methotrexate, Methotrexate LPF (methotrexate), Mexate (methotrexate), Mexate-AQ (methotrexate), Toposar (etoposide), Topotecan Hydrochloride, and VePesid (etoposide).

### Pharmaceutical Dose, Formulation and Packaging

Selection of a particular nintedanib composition or indolinone or salt thereof, is accompanied by the selection of a specially designed product packaging and configuration that maximizes the therapeutic utility of the particular composition. Factors to be considered in selecting packaging may include, for example, intrinsic product stability, whether the formulation may be subject to lyophilization, device selection (e.g., dry-powder inhaler), and/or packaging form (e.g., dry powder formulations in a vial, capsule or blister pack).

In one preferred embodiment, the compositions will take the form of a unit dosage form such as vial, capsule or blister pack containing a dry powder, or other composition and thus the composition may contain, along with the active ingredient, a carrier or bulking agent such as lactose, mannitol, or the like; a lubricant such as magnesium stearate or the like; and/or a binder such as starch, gum acacia, polyvinyl pyrrolidine, gelatin, cellulose, cellulose derivatives or the like.

Nintedanib or indolinone or salt thereof compound formulations or combinations as described herein can be separated into two groups; those of simple formulation or complex formulations providing taste-masking for improved tolerability, stability and tolerability, immediate or sustained-release, and/or area-under-the-curve (AUC) shape-enhancing properties. Simple formulations may include dry powder inhaled nintedanib or indolinone formulations alone or with either water soluble or organic soluble non-encapsulating excipients with or without a carrier agent such as lactose.

Complex formulations containing active ingredient may include nintedanib or indolinone alone or combinations described herein with active ingredient encapsulated or complexed with water-soluble excipients such as lipids, liposomes, cyclodextrins, microencapsulation, and emulsions dry powder formulations for administration using a dry powder inhaler of nintedanib or indolinone alone or combinations described herein as a co-crystal/co-precipitate/spray dried complex or mixture with low-water soluble excipients/salts in dry powder form with or without a carrier agent such as lactose. Specific methods for simple and complex formulation preparation are described herein.

Compositions of the invention include each dose consisting of about 0.05 mg to about 100 mg nintedanib or indolinone compound. The nintedanib base or indolinone compound base, or nintedanib base or indolinone base within a nintedanib salt or indolinone salt thereof dose may be about 0.01 mg, about 0.05 mg, about 0.1 mg, about 0.2 mg, about 0.3 mg, about 0.4 mg, about 0.5 mg, about 0.6 mg, about 0.7 mg, about 0.8 mg, about 0.9 mg, about 1.0 mg, about 2.0 mg, about 3.0 mg, about 4.0 mg, about 5.0 mg, about 6.0 mg, about 7.0 mg, about 8.0 mg, about 9.0 mg, about 10.0 mg, about 15 mg, about 20 mg, about 25 mg, about 50 mg, about 75 mg or about 100 mg nintedanib or indolinone compound in 0.01 mg increments. Compositions of the invention may further include each dose consisting of a fine particle fraction between 10% and 100% with increment units of 1%. By nonlimiting example, a fine particle fraction more than about 10%, more than about 15%, more than about 20%, more than about 25%, more than about 30%, more than about 35%, more than about 40%, more than about 45%, more than about 50%, more than about 55%, more than about 60%, more than about 65%, more than about 70%, more than about 75%, more than about 80%, more than about 85%, more than about 90%, more than about 95%, and about 100%. Compositions of the invention may further include each dose consisting of a fine particle dose between about 0.001 mg to about 100 mg nintedanib base or indolinone compound base, or nintedanib base or indolinone base within a nintedanib salt or indolinone salt thereof. By nonlimiting example, about 0.001 mg, about 0.005 mg, about 0.01 mg, and about 0.05 mg in 0.01 mg increments. By further example, fine particle dose may be about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 35 mg, about 40 mg, about 45 mg, about 50 mg, about 60 mg, about 70 mg, about 80 mg, about 90 mg, and about 100 mg in 0.1 mg increments.

By nonlimiting example, a preferred embodiment contains micronized nintedanib or salt thereof, including the hydrobromide salt in solid particles with a particle size distribution defined as having a D10 between about 0.1 μm and about 2 μm, a D50 between about 1 μm and about 3 μm, and a D90 between about 1.5 μm and about 5 μm at a formulation content between about 1% and about 20% on a weight by weight basis. The preferred embodiment may further contain lactose with a particle size distribution defined as having a D10 between about 5 μm to about 15 μm, a D50 between about 50 μm to about 100 μm, and a D90 between about 120 μm to about 160 μm at a formulation content between about 60% and about 99% on a weight by weight basis. The preferred embodiment may further contains lactose fines with a particle size distribution defined as having a D50 less than about 5 μm and a D90 less than about 10 μm at a formulation content between more than 0% and about 20% on a weight by weight basis. On a weight-by-weight basis, this composition may further contain one or more of the following force control agents at about 0.1% to about 20% leucine, trileucine, magnesium stearate, sodium stearate and lecithin. Composition may be packaged in capsules, blister well or metered device reservoir, each consisting from about 1 mg to about 40 mg of the preferred dry powder formulation composition for administration using a dry powder inhaler. The preferred formulation described herein enables a high emitted dose from medium and high-resistance dry powder inhalation devices. For clarity, medium and high resistance devices are designed to require lower inhalation flow rates to actuate and disperse dry powder formulation dosages and are more well-suited for a human with pulmonary disease and reduced lung function whose inhalation flow rates may otherwise be insufficient to efficiently actuate and disperse the dry powder dose for inhalation administration from a low resistance device. Each dose may be administered in one, two, three, four or up to 20 inhalation puffs per dose, wherein each inhalation puff represents administration of the contents from a single capsule, single blister well or a single metered device reservoir dose. Each dose may be provided one time, two times, three times or four times daily for a maximum daily dose of 200 mg nintedanib.

Methods of the invention include treating a person suffering from an interstitial lung disease by administering an inhaled dry powder dose consisting of about 0.05 mg to about 100 mg nintedanib base or indolinone compound base, or nintedanib base or indolinone base within a nintedanib salt or indolinone salt thereof. The nintedanib base or indolinone compound base, or nintedanib base or indolinone base within a nintedanib salt or indolinone salt thereof dose may be about 0.01 mg, about 0.05 mg, about 0.1 mg, about 0.2 mg, about 0.3 mg, about 0.4 mg, about 0.5 mg, about 0.6 mg, about 0.7 mg, about 0.8 mg, about 0.9 mg, about 1.0 mg, about 2.0 mg, about 3.0 mg, about 4.0 mg, about 5.0 mg, about 6.0 mg, about 7.0 mg, about 8.0 mg, about 9.0 mg, about 10.0 mg, about 15 mg, about 20 mg, about 25 mg, about 50 mg, about 75 mg or about 100 mg nintedanib or indolinone compound in 0.01 mg increments. Methods of the invention include treating a person suffering from an interstitial lung disease with an inhaled dry powder nintedanib or indolinone analog composition wherein each dose consists of a fine particle fraction between 10% and 100% with increment units of 1%. By nonlimiting example, a fine particle fraction more than about 10%, more than about 15%, more than about 20%, more than about 25%, more than about 30%, more than about 35%, more than about 40%, more than about 45%, more than about 50%, more than about 55%, more than about 60%, more than about 65%, more than about 70%, more than about 75%, more than about 80%, more than about 85%, more than about 90%, more than about 95%, and about 100%. Methods of the invention include treating a person suffering from an interstitial lung disease with an inhaled dry powder composition wherein the fine particle dose is between about 0.001 mg to about 100 mg nintedanib base or indolinone compound base, or nintedanib base or indolinone base within a nintedanib salt or indolinone salt thereof. By nonlimiting example, about 0.001 mg, about 0.005 mg, about 0.01 mg, and about 0.05 mg in 0.01 mg increments. By further example, fine particle dose may be about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 35 mg, about 40 mg, about 45 mg, about 50 mg, about 60 mg, about 70 mg, about 80 mg, about 90 mg, and about 100 mg in 0.1 mg increments. Methods of the invention include treating a person suffering from an interstitial lung disease with an inhaled dry powder nintedanib or indolinone analogy composition once per day, twice per day, three times per day, four times per day or five times per day, wherein each dose consists of about 0.05 mg to about 100 mg nintedanib or indolinone compound, with a fine particle fraction between about 10% and 100%, delivering a fine particle dose between about 0.001 mg to about 100 mg nintedanib or indolinone analog.

Compositions of the invention also include a nintedanib or indolinone analog and Pirfenidone or pyridone analog combination dry powder. By nonlimiting example, each nintedanib or indolinone analog dose within the combination consists of about 0.01 mg to about 100 mg nintedanib base or indolinone compound base, or nintedanib base or indolinone base within a nintedanib salt or indolinone salt thereof. The nintedanib base or indolinone compound base, or nintedanib base or indolinone base within a nintedanib salt or indolinone salt thereof dose within the combination may be about 0.01 mg, about 0.05 mg, about 0.1 mg, about 0.2 mg, about 0.3 mg, about 0.4 mg, about 0.5 mg, about 0.6 mg, about 0.7 mg, about 0.8 mg, about 0.9 mg, about 1.0 mg, about 2.0 mg, about 3.0 mg, about 4.0 mg, about 5.0 mg, about 6.0 mg, about 7.0 mg, about 8.0 mg, about 9.0 mg, about 10.0 mg, about 15 mg, about 20 mg, about 25 mg, about 50 mg, about 75 mg or about 100 mg nintedanib or indolinone compound in 0.01 mg increments. By nonlimiting example, each Pirfenidone or pyridone analog dose within the combination consists of about 5 mg to about 200 mg Pirfenidone or pyridone analog compound. The Pirfenidone or pyridone analog dose within the combination may be about 5 mg, about 10 mg, about 15 mg, about 20 mg, about 25 mg, about 30 mg, about 35 mg, about 35 mg, about 40 mg, about 45 mg, about 50 mg, about 60 mg, about 65 mg, about 70 mg, about 75 mg, about 80 mg, about 85 mg, about 90 mg, about 95 mg, or about 100 mg Pirfenidone or pyridone analog. Compositions of the combination invention may further include each drug dose that, when dispersed into a dry aerosol for inhalation by a dry powder inhalation device, is comprised of a fine particle fraction between 10% and 100% with increment units of 1%. Accordingly, the invention includes the combination of the dry powder inhalation device By nonlimiting example, a fine particle fraction of each drug more than about 10%, more than about 15%, more than about 20%, more than about 25%, more than about 30%, more than about 35%, more than about 40%, more than about 45%, more than about 50%, more than about 55%, more than about 60%, more than about 65%, more than about 70%, more than about 75%, more than about 80%, more than about 85%, more than about 90%, more than about 95%, and about 100%. Compositions of the combination invention may further include each dose consisting of a fine particle dose between about 0.001 mg to about 100 mg nintedanib or indolinone analog and 5 mg to about 100 mg Pirfenidone or pyridone analog compound. By example the fine particle dose for the nintedanib base or indolinone compound base, or nintedanib base or indolinone base within a nintedanib salt or indolinone salt thereof from the combination may be about 0.001 mg, about 0.01 mg, about 0.05 mg. about 0.1 mg, about 0.5 mg, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 35 mg, about 40 mg, about 45 mg, about 50 mg, about 60 mg, about 70 mg, about 80 mg, about 90 mg, and about 100 mg nintedanib base or indolinone compound base, or nintedanib base or indolinone base within a nintedanib salt or indolinone salt thereof in 0.1 mg increments. By example the fine particle dose for the Pirfenidone or pyridone analog compound from the combination may be about 0.5 mg, about 1 mg, about 2.5 mg, about 5 mg, about 10 mg, about 15 mg, about 20 mg, about 25 mg, about 30 mg, about 35 mg, about 35 mg, about 40 mg, about 45 mg, about 50 mg, about 60 mg, about 65 mg, about 70 mg, about 75 mg, about 80 mg, about 85 mg, about 90 mg, about 95 mg, or about 100 mg Pirfenidone or pyridone analog. As noted above, the invention includes a combination of the dry powders disposed in a dry powder inhalation device such that when dispersed, the combination yields a dry powder combined aerosol comprising more than one API for inhalation.

Compositions ratios in mg:mg nintedanib or indolinone compound to Pirfenidone or pyridone analog will be about 1:1000, about 1:900, about 1:800, about 1:700, about 1:600, about 1:500, about 1:400, about 1:300, about 1:250, about 1:200, about 1:100, about 1:75, about 1:50, about 1:25, about 1:20, about 1:10, about 1:5, about 1:2.5, about 1:1, about 2:1, about 3:1 and about 4:1.

Methods of the invention include optimizing the co-formulated combination nintedanib or indolinone compound and Pirfenidone or pyridone analog ratio to circumvent a co-formulation chemical interaction or physiologic effect that increases the rate that inhalation delivered nintedanib or indolinone compound is eliminated from the lung to the plasma compared to that of nintedanib or indolinone delivered without co-formulated Pirfenidone or pyridone analog. By non-limiting example, 2:100 nintedanib:Pirfenidone mg:mg ratio reduces the pulmonary and increases the plasma nintedanib Cmax about 30-50%. To maximize the pulmonary residence time of inhaled nintedanib in co-formulated combination with Pirfenidone, it is desired to reduce this pharmacokinetic effect. By non-limiting example, this undesired pharmacokinetic effect is minimized by reducing the Pirfenidone content to less than 100 mg per dose with a nintedanib:Pirfenidone content ratio to between 1:30 and 1:100. By another non-limiting example, this undesired pharmacokinetic effect is minimized by reducing the Pirfenidone dose to less than 100 mg, while maintaining a 1:20 to 1:50 mg:mg nintedanib:Pirfenidone content ratio. By another non-limiting example, this undesired pharmacokinetic effect is minimized by increasing the nintedanib co-formulation content such that the resulting mg:mg nintedanib:Pirfenidone content ratio is less than 1:50.

Methods of the invention include optimizing the combination regiment for nintedanib or indolinone compound and Pirfenidone or pyridone analog ratio to improve therapeutic benefit, including efficacy, safety, tolerability and compliance. By non-limiting example, 100 mg Pirfenidone exists at the upper range of Pirfenidone tolerability as a nebulized, stand-alone solution and is near the upper threshold of that possible for a compliant and well-tolerated dry powder product. By non-limiting example, it is predicted the efficacy of this nintedanib or indolinone compound and Pirfenidone or pyridone analog co-formulated dry powder product will be greater than either active ingredient alone. To maximize the safety/tolerability/compliance-to-efficacy relationship, reducing the amount of overall administered dry powder increases compliance and increases both safety and tolerability of the combination product. Taking advantage of the added effect of co-formulating with nintedanib or indolinone compound, the amount of Pirfenidone or pyridone analog in the co-formulated dry powder product may be reduced, while maintaining the overall added benefit of administering both nintedanib or indolinone and Pirfenidone or pyridone analog to a patient. By non-limiting example, this desired outcome is created by reducing the Pirfenidone dose to less than 100 mg, while maintaining a 1:25 to 1:500 weight:weight nintedanib:Pirfenidone content ratio. Methods of the invention include treating a person suffering from an interstitial lung disease by administering a nintedanib or indolinone analog and Pirfenidone or pyridone analog combination dry powder by means of a dry powder inhaler known to those who are skilled in the art, wherein each nintedanib or indolinone analog dose within the combination consists of about 0.05 mg to about 100 mg nintedanib or indolinone compound and about 5 mg to about 100 mg Pirfenidone or pyridone analog compound. Methods of the invention include treating a person suffering from an interstitial lung disease with an inhaled dry powder nintedanib or indolinone analog and Pirfenidone or pyridone analog combination composition wherein each dose consists of a fine particle fraction between 10% and 100%, wherein the resulting fine particle dose is between about 0.001 mg to about 200 mg nintedanib or indolinone analog and between about 5 mg to about 200 mg Pirfenidone or pyridone analog. Methods of the invention include treating a person suffering from an interstitial lung disease with an inhaled dry powder nintedanib or indolinone analogy and Pirfenidone or pyridone analog combination composition once per day, twice per day, three times per day, four times per day or five times per day, wherein each dose consists of about 0.05 mg to about 200 mg nintedanib or indolinone compound and between about 5 mg to about 200 200 mg Pirfenidone or pyridone analog compound, with a fine particle fraction between about 10% and 100%, delivering a fine particle dose between about 0.001 mg to about 200 mg nintedanib or indolinone analog and between about 5 mg to about 200 mg Pirfenidone or pyridone analog. In preferred embodiments, the dose consists of 0.05 mg to 25 mg, 0.05 mg to 50 mg, 0.05 mg to 100 mg and up to 200 mg as described above.

Compositions of the invention also include a nintedanib or indolinone analog and PDE4 inhibitor combination dry powder. By nonlimiting example, each nintedanib or indolinone analog dose within the combination consists of about 0.05 mg to about 100 mg nintedanib base or indolinone compound base, or nintedanib base or indolinone base within a nintedanib salt or indolinone salt thereof. The nintedanib base or indolinone compound base, or nintedanib base or indolinone base within a nintedanib salt or indolinone salt thereof dose within the combination may be about 0.01 mg, about 0.05 mg, about 0.1 mg, about 0.2 mg, about 0.3 mg, about 0.4 mg, about 0.5 mg, about 0.6 mg, about 0.7 mg, about 0.8 mg, about 0.9 mg, about 1.0 mg, about 2.0 mg, about 3.0 mg, about 4.0 mg, about 5.0 mg, about 6.0 mg, about 7.0 mg, about 8.0 mg, about 9.0 mg, about 10.0 mg, about 15 mg, about 20 mg, about 25 mg, about 50 mg, about 75 mg or about 100 mg nintedanib base or indolinone compound base, or nintedanib base or indolinone base within a nintedanib salt or indolinone salt thereof in 0.01 mg increments. By nonlimiting example, each PDE4 inhibitor dose within the combination consists of about 0.01 mg to about 100 mg PDE4 inhibitor compound. The PDE4 inhibitor dose within the combination may be about 0.01 mg, about 0.05 mg, about 0.1 mg, about 0.2 mg, about 0.3 mg, about 0.4 mg, about 0.5 mg, about 0.6 mg, about 0.7 mg, about 0.8 mg, about 0.9 mg, about 1.0 mg, about 2.0 mg, about 3.0 mg, about 4.0 mg, about 5.0 mg, about 6.0 mg, about 7.0 mg, about 8.0 mg, about 9.0 mg, about 10.0 mg, about 15 mg, about 20 mg, about 25 mg, about 50 mg, about 75 mg or about 100 mg PDE4 inhibitor compound. Compositions of the combination invention may further include each drug dose consists of a fine particle fraction between 10% and 100% with increment units of 1%. By nonlimiting example, a fine particle fraction of each drug more than about 10%, more than about 15%, more than about 20%, more than about 25%, more than about 30%, more than about 35%, more than about 40%, more than about 45%, more than about 50%, more than about 55%, more than about 60%, more than about 65%, more than about 70%, more than about 75%, more than about 80%, more than about 85%, more than about 90%, more than about 95%, and about 100%. Compositions of the combination invention may further include each dose consisting of a fine particle dose between about 0.001 mg to about 100 mg nintedanib base or indolinone compound base, or nintedanib base or indolinone base within a nintedanib salt or indolinone salt thereof and 0.001 mg to about 100 mg PDE4 inhibitor compound. By example for the nintedanib or indolinone analog, a fine particle dose from the combination may be about 0.001, about 0.01, about 0.1 mg, about 0.5 mg, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 35 mg, about 40 mg, about 45 mg, about 50 mg, about 60 mg, about 70 mg, about 80 mg, about 90 mg, and about 100 mg nintedanib base or indolinone compound base, or nintedanib base or indolinone base within a nintedanib salt or indolinone salt thereof in 0.1 mg increments. By example for the PDE4 inhibitor fine particle dose from the combination may be about 0.001, about 0.01, about 0.1 mg, about 0.5 mg, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 35 mg, about 40 mg, about 45 mg, about 50 mg, about 60 mg, about 70 mg, about 80 mg, about 90 mg, and about 100 mg PDE4 inhibitor compound in 0.1 mg increments.

Compositions of the invention include ratios in mg:mg nintedanib or indolinone compound to PDE4 inhibitor will be about 1:1000, about 1:900, about 1:800, about 1:700, about 1:600, about 1:500, about 1:400, about 1:300, about 1:250, about 1:200, about 1:100, about 1:75, about 1:50, about 1:25, about 1:20, about 1:10, about 1:5, about 1:2.5, about 1:1, about 2:1, about 3:1 and about 4:1.

Methods of the invention include optimizing the co-formulated combination nintedanib or indolinone compound and PDE4 inhibitor ratio to improve therapeutic benefit. By non-limiting example, it is predicted the efficacy of this nintedanib or indolinone compound PDE4 inhibitor co-formulated dry powder product will be greater than either active ingredient alone. To maximize the safety/tolerability/compliance-to-efficacy relationship, reducing the amount of overall administered dry powder increases compliance and increases both safety and tolerability of the combination product. Taking advantage of the added effect, both the amount of co-formulating with nintedanib or indolinone compound and PDE4 inhibitor in the co-formulated dry powder product may be reduced, while maintaining the overall added benefit of administering both nintedanib or indolinone and PDE4 inhibitor to a patient. By non-limiting example, the ratio is optimized by reducing the PDE4 inhibitor content to less than 40 mg per dose with a nintedanib:PDE4 inhibitor content ratio to between 1:4 and 1:400 on a mg:mg basis. By non-limiting example, the ratio is optimized by reducing the PDE4 inhibitor content to less than 30 mg per dose with a nintedanib:PDE4 inhibitor content ratio to between 1:4 and 1:400 on a mg:mg basis. By non-limiting example, the ratio is optimized by reducing the PDE4 inhibitor content to less than 20 mg per dose with a nintedanib:PDE4 inhibitor content ratio to between 1:4 and 1:400 on a mg:mg basis. By non-limiting example, the ratio is optimized by reducing the PDE4 inhibitor content to less than 10 mg per dose with a nintedanib:PDE4 inhibitor content ratio to between 1:4 and 1:400 on a mg:mg basis. By non-limiting example, the ratio is optimized by reducing the PDE4 inhibitor content to less than 5 mg per dose with a nintedanib:PDE4 inhibitor content ratio to between 1:4 and 1:400 on a mg:mg basis. By non-limiting example, the ratio is optimized by reducing the PDE4 inhibitor content to less than 1 mg per dose with a nintedanib:PDE4 inhibitor content ratio to between 1:4 and 1:400 on a mg:mg basis.

Methods of the invention include treating a person suffering from an interstitial lung disease by administering a nintedanib or indolinone analog and a PDE4 inhibitor combination dry powder, wherein each nintedanib or indolinone analog dose within the combination consists of about 0.05 mg to about 100 mg nintedanib base or indolinone compound base, or nintedanib base or indolinone base within a nintedanib salt or indolinone salt thereof and about 0.01 mg to about 100 mg PDE4 inhibitor compound. Methods of the invention include treating a person suffering from an interstitial lung disease with an inhaled dry powder nintedanib or indolinone analog and PDE4 inhibitor combination composition wherein each dose consists of a fine particle fraction between 10% and 100%, wherein the resulting fine particle dose is between about 0.001 mg to about 100 mg nintedanib base or indolinone compound base, or nintedanib base or indolinone base within a nintedanib salt or indolinone salt thereof and between about 0.001 mg to about 100 mg PDE4 inhibitor. Methods of the invention include treating a person suffering from an interstitial lung disease with an inhaled dry powder nintedanib or indolinone analogy and pirfenidone or pyridone analog combination composition once per day, twice per day, three times per day, four times per day or five times per day, wherein each dose consists of about 0.05 mg to about 100 mg nintedanib base or indolinone compound base, or nintedanib base or indolinone base within a nintedanib salt or indolinone salt thereof and between about 0.01 mg to about 100 mg PDE4 inhibitor compound, with a fine particle fraction between about 10% and 100%, delivering a fine particle dose between about 0.001 mg to about 100 mg nintedanib base or indolinone compound base, or nintedanib base or indolinone base within a nintedanib salt or indolinone salt thereof and between about 0.001 mg to about 100 mg PDE4 inhibitor compound.

Compositions of the invention also include a nintedanib or indolinone analog and prostacyclin analog combination dry powder. By nonlimiting example, each nintedanib or indolinone analog dose within the combination consists of about 0.05 mg to about 100 mg nintedanib base or indolinone compound base, or nintedanib base or indolinone base within a nintedanib salt or indolinone salt thereof. The nintedanib base or indolinone compound base, or nintedanib base or indolinone base within a nintedanib salt or indolinone salt thereof dose may be about 0.01 mg, about 0.05 mg, about 0.1 mg, about 0.2 mg, about 0.3 mg, about 0.4 mg, about 0.5 mg, about 0.6 mg, about 0.7 mg, about 0.8 mg, about 0.9 mg, about 1.0 mg, about 2.0 mg, about 3.0 mg, about 4.0 mg, about 5.0 mg, about 6.0 mg, about 7.0 mg, about 8.0 mg, about 9.0 mg, about 10.0 mg, about 15 mg, about 20 mg, about 25 mg, about 50 mg, about 75 mg or about 100 mg nintedanib base or indolinone compound base, or nintedanib base or indolinone base within a nintedanib salt or indolinone salt thereof in 0.01 mg increments. By nonlimiting example, each prostacyclin analog dose within the combination consists of about 0.001 mg to about 10 mg prostacyclin analog compound. The prostacyclin analog dose within the combination may be about 0.001 mg, about 0.005 mg, about 0.01 mg, about 0.015 mg, about 0.020 mg, about 0.025 mg, about 0.030 mg, about 0.035 mg, about 0.040 mg, about 0.045 mg, about 0.050 mg, about 0.055 mg, about 0.060 mg, about 0.065 mg, about 0.070 mg, about 0.075 mg, about 0.080 mg, about 0.085 mg, about 0.090 mg, about 0.095 mg, about 0.1 mg, about 0.15, about 0.2 mg, about 0.25 mg, about 0.3 mg, about 0.35 mg, about 0.4 mg, about 0.45 mg, about 0.5 mg, about 0.55 mg, about 0.6 mg, about 0.65 mg, about 0.7 mg, about 0.75 mg, about 0.8 mg, about 0.85 mg, about 0.9 mg, about 0.95 mg, about 1.0 mg, about 1.1 mg, about 1.5 mg, about 2 mg, about 3 mg, about 4 mg, about 5 mg, and about 10 mg in 0.001 mg increments prostacyclin analog compound. Compositions of the combination invention may further include each drug dose consists of a fine particle fraction between 10% and 100% with increment units of 1%. By nonlimiting example, a fine particle fraction of each drug more than about 10%, more than about 15%, more than about 20%, more than about 25%, more than about 30%, more than about 35%, more than about 40%, more than about 45%, more than about 50%, more than about 55%, more than about 60%, more than about 65%, more than about 70%, more than about 75%, more than about 80%, more than about 85%, more than about 90%, more than about 95%, and about 100%. Compositions of the combination invention may further include each dose consisting of a fine particle dose between about 0.001 mg to about 100 mg nintedanib base or indolinone compound base, or nintedanib base or indolinone base within a nintedanib salt or indolinone salt thereof and about 0.0001 mg to about 10 mg prostacyclin analog compound. By example, a nintedanib base or indolinone compound base, or nintedanib base or indolinone base within a nintedanib salt or indolinone salt thereof fine particle dose from the combination maybe about 0.001, about 0.01, about 0.1 mg, about 0.5 mg, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 35 mg, about 40 mg, about 45 mg, about 50 mg, about 60 mg, about 70 mg, about 80 mg, about 90 mg, and about 100 mg nintedanib base or indolinone compound base, or nintedanib base or indolinone base within a nintedanib salt or indolinone salt thereof in 0.1 mg increments. By nonlimiting example, the prostacyclin analog fine particle dose from the combination may be about 0.0001 mg, about 0.0005 mg, about 0.001 mg, about 0.0015 mg, about 0.0020 mg, about 0.0025 mg, about 0.0030 mg, about 0.0035 mg, about 0.0040 mg, about 0.0045 mg, about 0.0050 mg, about 0.0055 mg, about 0.0060 mg, about 0.0065 mg, about 0.0070 mg, about 0.0075 mg, about 0.0080 mg, about 0.0085 mg, about 0.0090 mg, about 0.0095 mg, about 0.01 mg, about 0.015, about 0.02 mg, about 0.025 mg, about 0.03 mg, about 0.035 mg, about 0.04 mg, about 0.045 mg, about 0.05 mg, about 0.055 mg, about 0.06 mg, about 0.065 mg, about 0.07 mg, about 0.075 mg, about 0.08 mg, about 0.085 mg, about 0.09 mg, about 0.095 mg, about 0.1 mg, about 0.2 mg, about 0.3 mg, about 0.4 mg, about 0.5 mg, about 0.6 mg, about 0.7 mg, about 0.8 mg, about 0.9 mg, about 1.0 mg about 1.5 mg, about 2 mg, about 3 mg, about 4 mg, about 5 mg, and about 10 mg in 0.0001 mg increments prostacyclin analog compound. A “combination” of the invention includes a combination of the dry powders disposed in a dry powder inhalation device such that when dispersed, the combination yields a dry powder combined aerosol comprising more than one API for inhalation.

Compositions of the invention include ratios in mg:mg nintedanib or indolinone compound to prostacyclin analog will be about 1,000,000:1, about 100,000:1, about 10,000:1, about 1,000:1, about 800:1, about 700:1, about 600:1, about 500:1, about 400:1, about 300:1, about 250:1, about 200:1, about 150:1, about 100:1, about 75:1, about 50:1, about 25:1, about 20:1, about 10:1, about 5:1, about 1:1, about 2:1, about 3:1 and about 10:1.

Methods of the invention include optimizing the co-formulated combination nintedanib or indolinone compound and prostacyclin analog ratio to improve therapeutic benefit. By non-limiting example, it is predicted the efficacy of this nintedanib or indolinone compound prostacyclin analog co-formulated dry powder product will be greater than either active ingredient alone. To maximize the safety/tolerability/compliance-to-efficacy relationship, reducing the amount of overall administered dry powder increases compliance and increases both safety and tolerability of the combination product. Taking advantage of the added effect, both the amount of co-formulating with nintedanib or indolinone compound and prostacyclin analog in the co-formulated dry powder product may be reduced, while maintaining the overall added benefit of administering both nintedanib or indolinone and prostacyclin analog to a patient. By non-limiting example, the ratio is optimized by reducing the prostacyclin analog content to less than 0.04 mg per dose with a nintedanib:prostacyclin analog content ratio to between 1:4 and 250:1 on a mg:mg basis. By non-limiting example, the ratio is optimized by reducing the prostacyclin analog content to less than 0.02 mg per dose with a nintedanib:prostacyclin analog content ratio to between 1:4 and 250:1 on a mg:mg basis. By non-limiting example, the ratio is optimized by reducing the prostacyclin analog content to less than 0.018 mg per dose with a nintedanib:prostacyclin analog content ratio to between 1:4 and 250:1 on a mg:mg basis. By non-limiting example, the ratio is optimized by reducing the prostacyclin analog content to less than 0.015 mg per dose with a nintedanib:prostacyclin analog content ratio to between 1:4 and 250:1 on a mg:mg basis. By non-limiting example, the ratio is optimized by reducing the prostacyclin analog content to less than 0.01 mg per dose with a nintedanib:prostacyclin analog content ratio to between 1:4 and 250:1 on a mg:mg basis. By non-limiting example, the ratio is optimized by reducing the prostacyclin analog content to less than 0.005 mg per dose with a nintedanib:prostacyclin analog content ratio to between 1:4 and 250:1 on a mg:mg basis.

Methods of the invention include treating a person suffering from an interstitial lung disease by administering a nintedanib or indolinone analog and a prostacyclin analog combination dry powder, wherein each nintedanib or indolinone analog dose within the combination consists of about 0.05 mg to about 100 mg nintedanib or indolinone compound and about 0.001 mg to about 10 mg prostacyclin analog compound. Methods of the invention include treating a person suffering from an interstitial lung disease with an inhaled dry powder nintedanib or indolinone analog and prostacyclin analog combination composition wherein each dose consists of a fine particle fraction between 10% and 100%, wherein the resulting fine particle dose is between about 0.001 mg to about 100 mg nintedanib or indolinone analog and between about 0.0001 mg to about 10 mg prostacyclin analog compound. Methods of the invention include treating a person suffering from an interstitial lung disease with an inhaled dry powder nintedanib or indolinone analogy and pirfenidone or pyridone analog combination composition once per day, twice per day, three times per day, four times per day or five times per day, wherein each dose consists of about 0.05 mg to about 100 mg nintedanib or indolinone compound and between about 0.001 mg to about 10 mg prostacyclin analog compound, with a fine particle fraction between about 10% and 100%, delivering a fine particle dose between about 0.001 mg to about 100 mg nintedanib or indolinone analog and between about 0.0001 mg to about 10 mg prostacyclin analog compound.

In other embodiments, nintedanib or indolinone or combination described herein includes a taste-masking agent including sugar, saccharin (e.g., sodium saccharin), sweetener or other compound or agent that beneficially affects taste, after-taste, perceived unpleasant saltiness, sourness or bitterness, or that reduces the tendency of an oral or inhaled formulation to irritate a recipient (e.g., by causing coughing or sore throat or other undesired side effect, such as may reduce the delivered dose or adversely influence patient compliance with a prescribed therapeutic regimen). Certain taste-masking agents may form complexes with the nintedanib or indolinone or salt thereof.

In another embodiment, a salt form of nintedanib or indolinone counterion of the salt form of nintedanib or indolinone is acetate, acetonide, alanine, aluminum, arginine, ascorbate, asparagine, aspartic acid, benzathine, benzoate, besylate, bisulfate, bisulfite, bitartrate, bromide (including bromide and hydrobromide), calcium, carbonate, camphorsulfonate, cetylpridinium, chloride (including chloride and hydrochloride), chlortheophyllinate, cholinate, cysteine, deoxycholate, diethanolamine, diethylamine, diphosphate, diproprionate, disalicylate, edetate, edisylate, estolate, ethylamine, ethylenediamine, ethandisulfonate, esylate, esylate hydroxide, gluceptate, gluconate, glucuronate, glutamic acid, glutamine, glycine, hippurate, histidine, hydrobromide, hydrochloride, hydroxide, iodide, isethionate, isoleucine, lactate, lactobionate, laurylsulfate, leucine, lysine, magnesium, mandelate, meglumine, mesylate, metabisulfate, metabisulfite, methionine, methylbromide, methylsulfate, methyl p-hydroxybenzoate, mucate, naphthoate, napsylate, nitrate, nitrite, octadecanoate, oleate, ornithine, oxalate, pamoate, pentetate, phenylalanine, phosphate, piperazine, polygalacturonate, potassium, procaine, proline, propionate, propyl p-hydroxybenzoate, saccharin, salicylate, selenocysteine, serine, silver, sodium, sorbitan, magnesium stearate, sodium stearate, succinate, sulfate, sulfite, sulfosalicylate, tartrate, threonine, tosylate, triethylamine, triethiodide, trifluoroacetate, trioleate, tromethamine, tryptophan, tyrosine, valerate, valine, xinafoate, or zinc.

The nintedanib salt form or indolinone salt form is prepared as a chloride or bromide salt form.

In some embodiments, described herein is a kit comprising: a unit dosage of a dry powder formulation of nintedanib or indolinone or salt thereof, as described herein in a container that is adapted for use in a dry powder inhalation device.

In some embodiments, described herein is a kit comprising: a unit dosage of a dry powder formulation of nintedanib or indolinone or salt thereof in combination with pirfenidone, as described herein in a container that is adapted for use in a dry powder inhalation device.

In some embodiments, described herein is a kit comprising: a unit dosage of a dry powder formulation of nintedanib or indolinone or salt thereof in combination with a PDE4 inhibitor, as described herein in a container that is adapted for use in a dry powder inhalation device.

In some embodiments, described herein is a kit comprising: a unit dosage of a dry powder formulation of nintedanib or indolinone or salt thereof in combination with a prostacyclin analog, as described herein in a container that is adapted for use in a dry powder inhalation device.

An aerosol comprising a plurality of dry powder particles has a mass median aerodynamic diameter (MMAD) less than about 5.0 μm. In some embodiments, at least 20% of the dry powder particles in the aerosol have a diameter less than about 5 μm.

The invention includes a dry powder formulation comprising nintedanib or salt thereof, or a indolinone derivative or salt thereof, at concentrations of 0.1% w/w to about 100% w/w in a finely divided form having mass median diameters of 0.5 micrometers to 10 micrometers. The nintedanib or salts thereof, or an indolinone or salt thereof and optionally one or more carrier excipients (e.g. lactose, mannitol, sucrose, glucose, trehalose) at about 10% to about 99.99% to improve handling, dispensing, metering and dispersion of the drug. The formulation may optionally contain one or more slipping agents (e.g., L-leucine, trileucine, sodium stearate, magnesium stearate) at a concentration of about 0.1% w/w to about 10% w/w to reduce inter-particulate adhesion, improve powder flowability and reduce moisture effects. The formulations may be prepared by physical blending of nintedanib or salt thereof, with the aforementioned excipients. Alternatively, the dry powder formulation may form by precipitation techniques that include spray drying, vacuum drying, solvent extraction, controlled precipitation, emulsification or lyophilization. For these formulations, in addition to the excipients mentioned above for blended dry powder formulations, these may contain phospholipids (e.g., dipalmitoyl phosphatidylcholine, distearoylphosphatidylcholine, diarachidoylphosphatidyl-choline dibehenoylphosphatidylcholine, diphosphatidyl glycerol) at 10% w/w to about 99.9% w/w to act as emulsifying agent and bulking agent. Optionally the formulation of the present invention may also include a biocompatible, preferably biodegradable polymer, copolymer, or blend or other combination thereof at about 0.1% w/w 99.9% w/w. Examples of polymers include but not limited to polylactides, polylactide-glycosides, cyclodextrins, polyacrylates, methylcellulose, carboxymethylcellulose, polyvinyl alcohols, polyanhydrides, polylactams, polyvinyl pyrrolidones, polysaccharides (dextran, starches, chitin, chitosan, etc.), hyaluronic acid, proteins, (albumin, collagen, gelatin, etc.). The dry powder can be packaged as unit dose in blister pack or capsules at fill weights of 0.01 mg to 100 mg. Alternatively, the dry powder formulation can be packaged in a device reservoir that meters 0.01 mg to 100 mg at the point of use.

The invention includes a dry powder formulation comprising nintedanib or salt thereof, or an indolinone derivative or salt thereof and Pirfenidone, at a combined drug concentration of 0.1% w/w to about 100% w/w in a finely divided form that, when dispersed through a dry powder inhaler, having a mass median aerodynamic diameter less than 5 microns. The nintedanib or salts thereof, or a indolinone or salt thereof and Pirfenidone and optionally one or more carrier excipients (e.g. lactose, mannitol, sucrose, glucose, trehalose) at about 0.1% to about 99.99% to improve stability, handling, dispensing, metering and dispersion of the dry powder. The formulation may optionally contain one or more slipping agents (e.g., L-leucine, trileucine, sodium stearate, magnesium stearate) at a concentration of about 0.1% w/w to about 10% w/w to reduce inter-particulate adhesion, improve powder flowability and reduce moisture loss or uptake. The formulations may be prepared by physical blending of nintedanib or salt thereof and Pirfenidone, with the aforementioned excipients. Alternatively, the dry powder formulation may form by precipitation techniques that include spray drying, vacuum drying, solvent extraction, controlled precipitation, emulsification or lyophilization. For these formulations, in addition to the excipients mentioned above for blended dry powder formulations, these may contain phospholipids (e.g., dipalmitoyl phosphatidylcholine, distearoylphosphatidylcholine, diarachidoylphosphatidyl-choline dibehenoylphosphatidyl-choline, diphosphatidyl glycerol) at 0.1% w/w to about 99.9% w/w to act as emulsifying agent and bulking agent. Optionally the formulation of the present invention may also include a biocompatible, preferably biodegradable polymer, copolymer, or blend or other combination thereof at about 0.1% w/w 99.9% w/w. Examples of polymers include but not limited to polylactides, polylactide-glycosides, cyclodextrins, polyacrylates, methylcellulose, carboxymethylcellulose, polyvinyl alcohols, polyanhydrides, polylactams, polyvinyl pyrrolidones, polysaccharides (dextran, starches, chitin, chitosan, etc.), hyaluronic acid, proteins, (albumin, collagen, gelatin, etc.). The dry powder can be packaged as unit dose in blister pack or capsules at fill weights of 0.01 mg to 100 mg. Alternatively, the dry powder formulation can be packaged in a device reservoir that meters 0.01 mg to 200 mg at the point of use.

The invention includes a dry powder formulation comprising nintedanib or salt thereof, or an indolinone derivative or salt thereof and a PDE4 inhibitor, at a combined drug concentration of 0.1% w/w to about 100% w/w in a finely divided form having a mass median aerodynamic diameter less than 5 microns. The nintedanib or salts thereof, or a indolinone or salt thereof and PDE4 inhibitor and optionally one or more carrier excipients (e.g. lactose, mannitol, sucrose, glucose, trehalose) at about 10% to about 99.99% to improve handling, dispensing, metering and dispersion of the drug. The formulation may optionally contain one or more slipping agents (e.g., L-leucine, trileucine, sodium stearate, magnesium stearate) at a concentration of about 0.1% w/w to about 10% w/w to reduce inter-particulate adhesion, improve powder flowability and reduce moisture effects. The formulations may be prepared by physical blending of nintedanib or salt thereof and PDE4 inhibitor, with the aforementioned excipients. Alternatively, the dry powder formulation may form by precipitation techniques that include spray drying, vacuum drying, solvent extraction, controlled precipitation, emulsification or lyophilization. For these formulations, in addition to the excipients mentioned above for blended dry powder formulations, these may contain phospholipids (e.g., dipalmitoyl phosphatidylcholine, distearoylphosphatidylcholine, diarachidoylphosphatidylcholine dibehenoylphosphatidyl-choline, diphosphatidyl glycerol) at 10% w/w to about 99.9% w/w to act as emulsifying agent and bulking agent. Optionally the formulation of the present invention may also include a biocompatible, preferably biodegradable polymer, copolymer, or blend or other combination thereof at about 0.1% w/w 99.9% w/w. Examples of polymers include but not limited to polylactides, polylactide-glycolides, cyclodextrins, polyacrylates, methylcellulose, carboxymethylcellulose, polyvinyl alcohols, polyanhydrides, polylactams, polyvinyl pyrrolidones, polysaccharides (dextrans, starches, chitin, chitosan, etc.), hyaluronic acid, proteins, (albumin, collagen, gelatin, etc.). The dry powder can be packaged as unit dose in blister pack or capsules at fill weights of 0.01 mg to 100 mg. Alternatively, the dry powder formulation can be packaged in a device reservoir that meters 0.01 mg to 100 mg at the point of use.

The invention includes a dry powder formulation comprising nintedanib or salt thereof, or a indolinone derivative or salt thereof and a prostacyclin analog, at a combined drug concentration of 0.1% w/w to about 100% w/w in a finely divided form having a mass median aerodynamic diameter less than 5 microns. The nintedanib or salts thereof, or a indolinone or salt thereof and prostacyclin analog and optionally one or more carrier excipients (e.g. lactose, mannitol, sucrose, glucose, trehalose) at about 10% to about 99.99% to improve handling, dispensing, metering and dispersion of the drug. The formulation may optionally contain one or more slipping agents (e.g., L-leucine, trileucine, sodium stearate, magnesium stearate) at a concentration of about 0.1% w/w to about 10% w/w to reduce inter-particulate adhesion, improve powder flowability and reduce moisture effects. The formulations may be prepared by physical blending of nintedanib or salt thereof and prostacyclin analog, with the aforementioned excipients. Alternatively, the dry powder formulation may form by precipitation techniques that include spray drying, vacuum drying, solvent extraction, controlled precipitation, emulsification or lyophilization. For these formulations, in addition to the excipients mentioned above for blended dry powder formulations, these may contain phospholipids (e.g., dipalmitoyl phosphatidylcholine, distearoylphosphatidylcholine, diarachidoylphosphatidylcholine dibehenoylphosphatidyl-choline, diphosphatidyl glycerol) at 10% w/w to about 99.9% w/w to act as emulsifying agent and bulking agent. Optionally the formulation of the present invention may also include a biocompatible, preferably biodegradable polymer, copolymer, or blend or other combination thereof at about 0.1% w/w 99.9% w/w. Examples of polymers include but not limited to polylactides, polylactide-glycolides, cyclodextrins, polyacrylates, methylcellulose, carboxymethylcellulose, polyvinyl alcohols, polyanhydrides, polylactams, polyvinyl pyrrolidones, polysaccharides (dextrans, starches, chitin, chitosan, etc.), hyaluronic acid, proteins, (albumin, collagen, gelatin, etc.). The dry powder can be packaged as unit dose in blister pack or capsules at fill weights of 0.01 mg to 100 mg. Alternatively, the dry powder formulation can be packaged in a device reservoir that meters 0.01 mg to 100 mg at the point of use.

### Methods to Treat or Prevent Disease

For purposes of the methods described herein, a indolinone, salt or derivative thereof compound, most preferably nintedanib salt is formulated and administered using a dry powder inhalation device producing a particle size distribution optimized for delivery of the aerosol to the pulmonary compartment. In some embodiments, nintedanib or an indolinone derivative compound or salt thereof is formulated as a pharmaceutical composition suitable for aerosol formation, dose for indication, deposition location, pulmonary or extra-respiratory therapeutic action, good taste, manufacturing and storage stability, and patient safety and tolerability. The methods include steps for performing an admixture of solutions contained in a multi-container system that separates the active pharmaceutical ingredient (API) from other solutions prior to or immediately following placement into a nebulizer for aerosol administration.

For purposes of the methods described herein, a indolinone, salt or derivative thereof compound, most preferably nintedanib salt is co-formulated and administered in combination with Pirfenidone using a dry powder inhalation device producing a particle size distribution optimized for delivery of the aerosol to the pulmonary compartment. In some embodiments, nintedanib or an indolinone derivative compound or salt thereof and Pirfenidone is formulated as a pharmaceutical composition suitable for dry powder dispersion and inhalation, dose for indication, deposition location, pulmonary delivery for pulmonary, or extra-respiratory therapeutic action, good taste, manufacturing and storage stability, and patient safety and tolerability. The steps of the method of administration or for preparing the dosages described herein include preparing a dry admixture of powders contained in a multi-container mixture or in a container system holding multiple components that separate any individual or subset of active pharmaceutical ingredients (APIs) from other component compounds prior to delivery or placement in the dry powder inhalation device.

For purposes of the methods described herein, a indolinone, salt or derivative thereof compound, most preferably nintedanib salt is co-formulated and administered in combination with a PDE4 inhibitor using a dry powder inhalation device producing a particle size distribution optimized for delivery of the aerosol to the pulmonary compartment. In some embodiments, nintedanib or an indolinone derivative compound or salt thereof and PDE4 inhibitor is formulated as a pharmaceutical composition suitable for dry powder dispersion and inhalation, dose for indication, deposition location, pulmonary delivery for pulmonary, or extra-respiratory therapeutic action, good taste, manufacturing and storage stability, and patient safety and tolerability. The methods include steps for performing an admixture of solutions contained in a multi-container system that separates the active pharmaceutical ingredient (API) from other solutions prior to or immediately following placement into a nebulizer for aerosol administration.

For purposes of the methods described herein, a indolinone, salt or derivative thereof compound, most preferably nintedanib salt is co-formulated and administered in combination with a prostacyclin analog using a dry powder inhalation device producing a particle size distribution optimized for delivery of the aerosol to the pulmonary compartment. In some embodiments, nintedanib or an indolinone derivative compound or salt thereof and prostacyclin analog is formulated as a pharmaceutical composition suitable for dry powder dispersion and inhalation, dose for indication, deposition location, pulmonary delivery for pulmonary, or extra-respiratory therapeutic action, good taste, manufacturing and storage stability, and patient safety and tolerability. The methods include steps for performing an admixture of solutions contained in a multi-container system that separates the active pharmaceutical ingredient (API) from other solutions prior to or immediately following placement into a nebulizer for aerosol administration. The dry powder administration step is performed in less than about 10 inhalation events, less than about 8 inhalation events, less than about 5 inhalation events, less than about 2 inhalation events, or 1 inhalation events.

In the methods described herein, the aerosol comprises particles having a mass median aerodynamic diameter from about 1 micron to about 5 microns, from about 2 microns to about 5 microns, from about 3 microns to about 5 microns, from about 4 microns to about 5 microns. The inhaling step delivers a dose of a least 0.0001 mg nintedanib or indolinone or salt thereof, at least 0.001 mg, at least 0.01 mg, at least 0.1 mg, at least 0.5 mg at least 1.0 mg, at least 2.0 mg, at least 4.0 mg, at least 10 mg, at least 25 mg, at least 50 mg, at least 100 mg nintedanib or indolinone or salt thereof. When nintedanib or indolinone or salt thereof is co-formulated and administered in combination with Pirfenidone, the Pirfenidone component contains a dose of at least 1 mg Pirfenidone, at least 5 mg Pirfenidone, at least 10 mg Pirfenidone, at least 15 mg Pirfenidone, at least 20 mg Pirfenidone, at least 25 mg Pirfenidone, at least 30 mg Pirfenidone, at least 40 mg Pirfenidone, at least 50 mg Pirfenidone, at least 60 mg Pirfenidone, at least 70 mg Pirfenidone, at least 80 mg Pirfenidone, at least 90 mg Pirfenidone or at least 100 mg Pirfenidone. When nintedanib or indolinone or salt thereof is co-formulated and administered in combination with a PDE4 inhibitor, the PDE4 inhibitor component is at least 0.1 mg PDE4 inhibitor, at least 0.5 mg PDE4 inhibitor, at least 1 mg PDE4 inhibitor, at least 2.5 mg PDE4 inhibitor, at least 5 mg PDE4 inhibitor, at least 7.5 mg PDE4 inhibitor, at least 10 mg PDE4 inhibitor, at least 15 mg PDE4 inhibitor, at least 20 mg PDE4 inhibitor, at least 30 mg PDE4 inhibitor or at least 40 mg PDE4 inhibitor. When nintedanib or indolinone or salt thereof is co-formulated and administered in combination with a prostacyclin analog, the prostacyclin analog component is at least 0.001 mg prostacyclin analog, at least 0.001 mg, about 0.005 mg, about 0.01 mg, about 0.015 mg, about 0.020 mg, about 0.025 mg, about 0.030 mg, about 0.035 mg, about 0.040 mg, about 0.045 mg, about 0.050 mg, about 0.055 mg, about 0.060 mg, about 0.065 mg, about 0.070 mg, about 0.075 mg, about 0.080 mg, about 0.080 mg, about 0.085 mg, about 0.090 mg, about 0.095 mg, about 0.1 mg, about 0.2 mg, about 0.3 mg, about 0.4 mg, about 0.5 mg, about 0.6 mg, about 0.7 mg, about 0.8 mg, about 0.9 mg, about 1.0 mg, about 5 mg, about 10 mg prostacyclin analog.

In one aspect, described herein is a method for the treatment methods include of administering nintedanib or indolinone or salt thereof, to treat a patient, wherein the patient avoids abnormal liver function exhibited by a grade 2 or higher abnormality following oral administration in one or more biomarkers of liver function after nintedanib or indolinone or salt thereof, administration, comprising administering to said patient nintedanib or indolinone or salt thereof, at doses less than 1056 mg per day. “Grade 2 liver function abnormalities” include elevations in alanine transaminase (ALT), aspartate transaminase (AST), alkaline phosphatase (ALP), or gamma-glutamyl transferase (GGT) greater than 2.5-times and less than or equal to 5-times the upper limit of normal (ULN). Grade 2 liver function abnormalities also include elevations of bilirubin levels greater than 1.5-times and less than or equal to 3-times the ULN. One or more biomarkers of liver function is selected from the group consisting of alanine transaminase, aspartate transaminase, bilirubin, and alkaline phosphatase. The method further comprises the step of measuring one or more biomarkers of liver function. The blood nintedanib or indolinone Cmax following inhaled administration of nintedanib or indolinone or salt thereof, is less than 40.0 ng/mL. The blood nintedanib or indolinone Cmax following administration of nintedanib or indolinone or salt thereof, is less than 20.0 ng/mL, less than 10.0 ng/mL, less than 5.0 ng/mL.

The methods of administering nintedanib or indolinone or salt thereof, include the avoidance of nausea, diarrhea, headaches, leg aches/cramps, fluid retention, visual disturbances, itchy rash, lowered resistance to infection, bruising or bleeding, loss of appetite, weight gain, reduced number of blood cells (neutropenia, thrombocytopenia, anemia), headache, edema, congestive cardiac failure observed following oral administration, comprising administering to said patient inhaled nintedanib or indolinone or salt thereof at doses less than 100 mg per day.

The methods of the invention also include a maximum dose level of less than or equal to about 100 mg per day of nintedanib or salt thereof is delivered to the patient by inhalation. In some embodiments, less than or equal to about 50 mg, less than or equal to about 25 mg, less than or equal to about 10 mg, less than or equal to about 5 mg, less than or equal to about 2 mg, less than or equal to about 1 mg per day of nintedanib or indolinone is delivered to the patient by inhalation as one dose per day, two doses per day, three doses a day, four doses a day, five doses a day, six doses a day or greater than six doses per day, and may be administered daily, every other day, every third day, every fourth day, every fifth day, every sixth day or weekly, every other week, every third week or monthly.

Methods of treatment include as prophylaxis against interstitial lung disease (ILD) by administering nintedanib or indolinone or salt thereof to a subject having or suspected to have interstitial lung disease. Interstitial lung disease includes those described above and all conditions of idiopathic interstitial pneumonias as defined by American Thoracic Society/European Respiratory Society international multidisciplinary consensus classification of the idiopathic interstitial pneumonias, AM. J. Respir. Crit. Care Med. 165, 277-304 (2002) (incorporated herein by reference).

The therapeutic method may also include a diagnostic step, such as identifying a subject with or suspected of having ILD. The method further sub-classifies into idiopathic pulmonary fibrosis based on extent of disease, progression of disease, rate of advancement, or response to any existing therapy. The delivered amount of aerosol nintedanib or indolinone or salt thereof compound (or salt thereof) formulation is sufficient to provide acute, sub-acute, or chronic symptomatic relief, slowing of fibrosis progression, halting fibrosis progression, reversing fibrotic damage, and/or subsequent increase in survival and/or improved quality of life.

The therapeutic method may also include a diagnostic step of identifying a subject with or suspected of having fibrosis in other tissues, by non-limiting example in the heart, liver, kidney or skin and the therapeutic amount of dry powder aerosol nintedanib or indolinone or salt thereof compound is sufficient to provide acute, sub-acute, or chronic symptomatic relief, slowing of fibrosis progression, halting fibrosis progression, reversing fibrotic damage, and/or subsequent increase in survival and/or improved quality of life.

The therapeutic method may also include a diagnostic step identifying a subject with or suspected of having multiple sclerosis and the therapeutic method comprises administering dry powder aerosol nintedanib or indolinone or salt thereof sufficient to provide acute, sub-acute, or chronic symptomatic relief, slowing of demyelination progression, halting demyelination progression, reversing demyelinated damage, and/or subsequent increase in survival and/or improved quality of life.

Therapeutic treatment methods include administering a therapeutically effective aerosol doses to a patient wherein the dosage is calculated, titrated, or measured to establish or maintain therapeutically effective threshold drug concentrations in the lung and/or targeted downstream tissue, which may be measured as drug levels in epithelial lining fluid (ELF), sputum, lung tissue, bronchial lavage fluid (BAL), or by deconvolution of blood concentrations through pharmacokinetic analysis. One embodiment includes the use of aerosol administration, delivering high or titrated concentration drug exposure directly to the affected tissue for treatment of pulmonary fibrosis and inflammation associated with ILD (including idiopathic pulmonary fibrosis) in animals and humans. Peak lung ELF levels achieved following aerosol administration to the lung will be between 100 ng/mL epithelial lining fluid to about 20,000 ng/mL epithelial lining fluid nintedanib or indolinone compound.

As a non-limiting example, in a preferred embodiment, a indolinone derivative compound as provided herein (e.g., nintedanib) formulated to permit dry powder inhaled aerosol administration to supply effective concentrations or amounts to produce and maintain threshold drug concentrations in the blood and/or lung, which may be measured as drug levels in epithelial lining fluid (ELF), sputum, lung tissue, bronchial lavage fluid (BAL), or by deconvolution of blood concentrations through pharmacokinetic analysis that absorb to the pulmonary vasculature producing drug levels sufficient for extra-pulmonary therapeutics, maintenance or prophylaxis. Therapeutic treatment methods include the use of inhaled dry powder aerosol administration, delivering high concentration drug exposure in the pulmonary vasculature and subsequent tissues and associated vasculature for treatment, maintenance and/or prophylaxis of, but not limited to cardiac fibrosis, kidney fibrosis, hepatic fibrosis, heart or kidney toxicity, or multiple sclerosis. Peak tissue-specific plasma levels (e.g., heart, kidney and liver) or cerebral spinal fluid levels (e.g. central nervous system) achieved following aerosol administration to the lung following oral inhalation or to the lung or nasal cavity following intra-nasal administration will be between 0.01 ng/mL and about 50 ng/mL nintedanib or indolinone or salt thereof. Peak lung epithelial lining fluid levels achieved following inhaled dry powder administration to the lung are between 100 ng/mL epithelial lining fluid and about 20,000 ng/mL epithelial lining fluid nintedanib or indolinone.

As a non-limiting example, an indolinone derivative compound remains at the therapeutically effective concentration at the site of pulmonary pathology, suspected pulmonary pathology, and/or site of pulmonary absorption into the pulmonary vasculature for at least about 10 seconds, at least 1 minute, at least about a 5 minute period, at least about a 10 min period, at least about a 20 min period, at least about a 30 min period, at least about a 1 hour period, at least a 2 hour period, at least about a 4 hour period, at least an 8 hour period, at least a 12 hour period, at least a 24 hour period, at least a 48 hour period, at least a 72 hour period, or at least one week. The effective nintedanib or indolinone or salt thereof concentration is sufficient to cause a therapeutic effect and the effect may be localized or broad-acting to or from the site of pulmonary pathology.

Delivery sites such as a pulmonary epithelial lining fluid, nasal cavity or sinus, the an nintedanib or indolinone or salt thereof compound formulation as provided herein is administered in one or more administrations so as to achieve a respirable delivered dose (RDD) daily of nintedanib or indolinone or salt thereof of at least about 0.0001 mg to about 100 mg, including all integral values therein such as 0.0001, 0.001, 0.006, 0.01, 0.02, 0.4, 0.6, 0.8, 1, 1.2, 1.4, 1.6, 1.8, 2, 3, 4, 5, 6, 10, 15, 20, 25, 30, 35, 40, 45, 50, 60, 70, 80, 90, 100 milligrams. Daily nintedanib or indolinone compound RDD levels remain consistent across optimized dose and ratio co-formulated combinations with pirfenidone or pyridone analog, or PDE4 inhibitor or prostacyclin analog.

Delivery sites such as a pulmonary site, nasal cavity or sinus, the an nintedanib or indolinone or salt thereof compound formulation as provided herein is administered in one or more administrations so as to achieve a respirable delivered dose daily of nintedanib or indolinone or salt thereof of at least about 0.0001 mg to about 100 mg, including all integral values therein such as 0.0001, 0.001, 0.006, 0.01, 0.02, 0.4, 0.6, 0.8, 1, 1.2, 1.4, 1.6, 1.8, 2, 3, 4, 5, 6, 10, 15, 20, 25, 30, 35, 40, 45, 50, 60, 70, 80, 90 or 100 milligrams, and pirfenidone or pyridone analog in co-formulated combination with nintedanib or indolinone compound as provided herein is administered in one or more administrations so as to achieve a respirable delivered dose daily of pirfenidone or pyridone analog of at least about 0.0001 mg to about 100 mg, including all integral values therein such as 0.0001, 0.001, 0.006, 0.01, 0.02, 0.4, 0.6, 0.8, 1, 1.2, 1.4, 1.6, 1.8, 2, 3, 4, 5, 6, 10, 15, 20, 25, 30, 35, 40, 45, 50, 60, 70, 80, 90, 100 milligrams, and PDE4 inhibitor in co-formulated combination with nintedanib or indolinone compound as provided herein is administered in one or more administrations so as to achieve a respirable delivered dose daily of PDE4 inhibitor of at least about 0.001 mg to about 100 mg, including all integral values therein such as 0.001, 0.005, 0.01, 0.02, 0.4, 0.6, 0.8, 1, 1.2, 1.4, 1.6, 1.8, 2, 3, 4, 5, 6, 10, 15, 20, 25, 30, 35, 40, 45, 50, 60, 70, 80, 90, 100 milligrams, and prostacyclin analog in co-formulated combination with nintedanib or indolinone compound as provided herein is administered in one or more administrations so as to achieve a respirable delivered dose daily of prostacyclin analog of at least about 0.0001 mg to about 10 mg, including all integral values therein such as 0.0001, 0.0005, 0.001, 0.005, 0.01, 0.02, 0.4, 0.6, 0.8, 1, 1.2, 1.4, 1.6, 1.8, 2, 3, 4, 5, 6, 10 milligrams.

### Manufacture

The fixed dose combination formulation of nintedanib or a indolinone derivative compound with pirfenidone or a pyridine analog, or a PDE4 inhibitor, or a prostacyclin analog using any suitable method. The fixed dose combination formulation of nintedanib or an indolinone derivative compound with the compounds listed above can be in a form of ready-to-use inhalation solution to be delivered as aerosols by a nebulizer or a dry powder formulation to be delivered as aerosols by a dry powder inhaler device. Inhalation solution can be prepared by dissolution of the APIs and suitable excipients (e.g., buffer, osmolality adjusting agents, permeant ion adjusting agents, taste/tolerability adjusting agents), sterile filtered and aseptically filled into suitable container closure systems (e.g., low density polyethylene ampules, Type I glass ampules). For preparation of respirable dry powders and particles, many suitable methods conventional in the art such as mixing of micronized APIs, blending of APIs with carrier particles (e.g., lactose), spray drying, spray freezing and methods that involve the use of supercritical fluid (e.g., CO₂) or perfluorocarbons (e.g., perfluorooctyl bromide). These methods can be employed under conditions that result in the formation of respirable particles with desired aerodynamic properties (e.g., mass median aerodynamic diameter, geometric standard deviation). If desired, respirable dry particles with desired properties, such as size and density, can be selected using suitable methods, such as sieving or cyclone separation.

### Ready-to-Use Inhalation Solution Formulations

Ready-to-Use inhalation solutions of fixed dose combination of nintedanib or an indolinone derivative compound with pirfenidone or a pyridine analog can be manufactured using well established mixing equipment. Excipients that include buffering agents, pH adjusting agents, osmolality adjusting agents, and taste masking agents are sequentially added and mixed to dissolve one at a time. Nintedanib or salt thereof are added to the solution and mixed to dissolve. Nintedanib or salt thereof can also be pre-wetted with a wetting agent such as propylene glycol prior to adding into the solution to facilitate its dissolution. Pirfenidone or pyridone analog are then added and mix to dissolve. To facilitate the dissolution of nintedanib or an indolinone derivative compound with pirfenidone or a pyridine analog, the solution may be heated to 40-50° C. for a finite time until the APIs are completely dissolved. The formulation is adjusted to the target pH then filtered into a holding tank for bioburden reduction before sterile filtered and aseptically filled into suitable containers such as low density polyethylene ampules or clear cyclic olefin vials or Type I USP glass vials.

### Carrier-Free Blend Formulations

In the simplest form of dry powder inhalation formulation, the indolinone derivative compound, most preferably nintedanib as disclosed herein, can be formulated as a carrier-free dry powder in combination with Pirfenidone by simple blending of the two APIs. Nintedanib or salt thereof and Pirfenidone or pyridone analog are first micronized to a desirable size using one or combinations of the following methods: trituration, jet milling, ball milling, sieving or any other suitable method. The mass median aerodynamic diameter in this embodiment could range from 0.5 to 10 microns, preferably from 1 to 5 microns and most preferably 2-3 microns. Micronized nintedanib or salt thereof and micronized Pirfenidone or pyridone analog can be blended together in appropriate ratios ranging from 1:2,000,000 to 200:1 using low shear mixers (e.g., conical screw mixer, tumble mixer, ribbon mixer) or high shear mixers. Content uniformity is tested to ensure homogenous distribution of nintedanib or salt thereof and Pirfenidone or pyridine analog.

The invention also includes blending of micronized indolinone derivative compound, most preferably nintedanib as disclosed herein, with micronized PDE4 inhibitor by micronizing nintedanib or salt thereof. The mass median diameter of micronized nintedanib or salt thereof and PDE4 inhibitor in this embodiment could range from 0.5 to 10 microns, preferably from 1 to 5 microns and most preferably 2-3 microns. Micronized nintedanib or salt thereof and micronized PDE4 inhibitor can be produced by one or the combination of the following methods: titration, jet milling, ball milling, sieving or any other suitable method to reduce the particle size to the desired range. Micronized nintedanib or salt thereof and micronized PDE4 inhibitor are blended together in appropriate ratios ranging from 1:400,000 and 20,000:1 using low shear mixers (e.g., conical screw mixer, tumble mixer, ribbon mixer) or high shear mixers. Content uniformity is tested to ensure homogenous distribution of nintedanib or salt thereof and PDE4 inhibitor.

The invention also includes blending of micronized indolinone derivative compound, most preferably nintedanib as disclosed herein, with micronized prostacyclin analog. The mass median diameter in this embodiment could range from 0.5 to 10 microns, preferably from 1 to 5 microns and most preferably 2-3 microns. Micronized nintedanib or salt thereof and micronized prostacyclin analog can be produced by either or combinations of titration, jet milling, ball milling, sieving or any other suitable methods to obtain particle size in the desired range. Micronized nintedanib or salt thereof and micronized prostacyclin analog are blended together in appropriate ratios ranging from 1:100,000 and 20:1 using low shear mixers (e.g., conical screw mixer, tumble mixer, ribbon mixer) or high shear mixers. Content uniformity is tested to ensure homogenous distribution of nintedanib or salt thereof and prostacyclin analog.

### Carrier-Based Blending

Another dry powder manufacturing method for the indolinone derivative compound, most preferably nintedanib as disclosed herein, combination with a second active compound is by blending with a carrier. The carrier can include sugars such as, but not limited to, lactose, mannitol, sorbitol, erythritol, trehalose, cyclodextrins, dextrose, glucose monohydrate, maltitol, maltose, raffinose pentahydrate and xylitol. Carrier particles are used to improve drug particle flowability and provide a surface for the smaller active drug particles to coat, thus making it easier for the drug particles to disperse into primary particle for inhalation. It is also used as the bulking agent to improve dosing accuracy and minimizing the dose variability. The mass median particle size for a coarse carrier is on the order of 10-200 μm, more preferably 25-150 μm and most preferably 50-100 μm. The mass median diameter of the drug particles is on the order of 0.5-10 μm, more preferably 1-5 μm and most preferably 2-3 μm.

In one embodiment, the second active is Pirfenidone or pyridine analog. The ratio of nintedanib or salt thereof and Pirfenidone or pyridine analog can range from 1:2,000,000 to 200:1. The two active compounds can be either add directly to the coarse carrier, or form a pre-mix using low shear mixers (e.g., conical screw mixer, tumble mixer, ribbon mixer) and then added to the carrier at ratios 0.1% w/w total 99.9% w/w. The powder blend is mixed using low a low shear mixer (e.g., conical screw mixer, tumble mixer, ribbon mixer) or a high shear mixer. Content uniformity is tested to ensure homogenous distribution of nintedanib or salt thereof and Pirfenidone or pyridine analog.

In another embodiment, the second active is a PDE4 inhibitor. The ratio of nintedanib or salt thereof and PDE4 inhibitor can range from 1:400,000 to 20,000:1. The two active compounds can be either add directly to the coarse carrier, or form a pre-mix using low shear mixers (e.g., conical screw mixer, tumble mixer, ribbon mixer) and then added to the carrier at ratios 0.1% w/w total 99.9% w/w. The carrier-based powder blend is obtained by mixing the actives and carrier using low a low shear mixer (e.g., conical screw mixer, tumble mixer, ribbon mixer) or a high shear mixer. Content uniformity is tested to ensure the final powder blend has a homogenous distribution of nintedanib or salt thereof and Pirfenidone or pyridine analog.

In yet another embodiment, the second active is a prostacyclin analog. The ratio of nintedanib or salt thereof and prostacyclin analog can range from 1:400,000 to 20,000:1. The two active compounds can be either add directly to the coarse carrier, or form a pre-mix using low shear mixers (e.g., conical screw mixer, tumble mixer, ribbon mixer) and then added to the carrier at ratios 0.1% w/w total 99.9% w/w. The carrier-based powder blend is obtained by mixing the actives and carrier using low a low shear mixer (e.g., conical screw mixer, tumble mixer, ribbon mixer) or a high shear mixer. Content uniformity is tested to ensure the final powder blend has a homogenous distribution of nintedanib or salt thereof and pirfenidone or pyridine analog.

The invention also includes blending of the indolinone derivative compound, most preferably nintedanib as disclosed herein, with a PDE4 inhibitor by micronizing nintedanib or salt thereof and micronizing the PDE4 inhibitor to a certain size. The mass median diameter in this embodiment could range from 0.5 to 10 μm, preferably from 1 to 5 μm and most preferably 2-3 μm. Micronized nintedanib or salt thereof and micronized PDE4 inhibitor can be produced by jet milling or ball milling or sieving to obtain particle size in the desired range. Micronized nintedanib or salt thereof and micronized PDE4 inhibitor can be blended together in appropriate ratios ranging from 1:400,000 and 20,000:1 using low shear mixers (e.g., conical screw mixer, tumble mixer, ribbon mixer) or high shear mixers. Content uniformity is tested to ensure homogenous distribution of nintedanib or salt thereof and PDE4 inhibitor.

The invention also includes blending of the indolinone derivative compound, most preferably nintedanib as disclosed herein, with a prostacyclin analog by micronizing nintedanib or salt thereof and micronizing the prostacyclin analog to a certain size. The mass median diameter in this embodiment could range from 0.5 to 10 microns, preferably from 1 to 5 microns and most preferably 2-3 microns. Micronized nintedanib or salt thereof and micronized prostacyclin analog can be produced by jet milling or ball milling or sieving to obtain particle size in the desired range. Micronized nintedanib or salt thereof and micronized prostacyclin analog can be blended together in appropriate ratios ranging from 1:400,000 and 20,000:1 using low shear mixers (e.g., conical screw mixer, tumble mixer, ribbon mixer) or high shear mixers. Content uniformity is tested to ensure homogenous distribution of nintedanib or salt thereof and prostacyclin analog.

Suitable spray drying techniques are described in various literature references (e.g., Spray Drying Technology Review, R Wisniewski 2015; Spray Drying: An Overview, D Santos 2017). In summary, spray drying process involves continuous atomization of a liquid feed containing the drug either dissolved or emulsified or suspended in liquid into a hot gas such as heated air or nitrogen to evaporate the solvent from the atomized droplets. The liquid feed can be prepared in the form of solution, emulsion or suspension containing the components of the dry particles to be produced in a suitable solvent (e.g., aqueous solvent, organic solvent, aqueous-organic mixture or emulsion) and fed into an atomizer by means of a pump. A nozzle atomizer or a rotary atomizer may be used to convert the feed solution into aerosol droplets. The spray drying condition can vary, depending on the composition of the feed solution (or suspension or emulsion) and the feed rate, and can be determined by a person skill in the art. In general, depending on the flow direction of the atomized droplets and the heated air flow, the inlet temperature to the spray dryer is about 100° C. to about 400° C., and preferably is about 200° C. to about 300° C. The spray dryer outlet temperature will vary depending upon such factors as the feed temperature, the direction of the air and atomized droplet flow, and the properties of the materials being dried. Generally, the outlet temperature is about 50° C. to about 150° C., preferably about 90° C. to about 120° C., or about 95° C. to about 105° C. Optionally, the dry particles collected can further be fractionated by sieving or using a cyclone, and/or further separated according to density using techniques known to those of skill in the art.

To prepare the spray dry particles, generally, a solution, emulsion or suspension that contains the desired components of the dry powder (i.e., a feed stock) is prepared and spray dried under suitable conditions. Preferably, the dissolved or suspended solids concentration in the feed stock is at least about 1 g/L, at least about 2 g/L, at least about 5 g/L, at least about 10 g/L, at least about 15 g/L, at least about 20 g/L, at least about 30 g/L, at least about 40 g/L, at least about 50 g/L, at least about 60 g/L, at least about 70 g/L, at least about 80 g/L, at least about 90 g/L, or at least about 100 g/L. The feedstock can be a solution or suspension by dissolving or suspending suitable components (e.g., one or more active drugs, excipients, other active ingredients) in a suitable solvent. The solvent can be prepared from one or more liquids to form a liquid solution or an emulsion using a high shear homogenizer. The resulting solution, emulsion or suspension can be atomized preferably immediately after preparation into aerosol droplets, which in a hot stream of air or nitrogen, are dried to form fine respirable particles.

Atomization can be done in a number of ways in that the feedstock can be pumped into an atomizer nozzle, or array of nozzles, that produce fine droplets. Atomizers can be rotary, single fluid, two-fluid, or ultrasonic designs. The different designs have different advantages, applicability and disadvantages depending on the particular spray drying process required. The hot drying gas can be introduced in the same (concurrent) or opposite (counter-current) flow to the atomizer direction. The concurrent flow fastens the flow of the aerosol particles through the system into the particle separator, such as a cyclone, more quickly and therefore more efficiently. The counter-current flow method allows the aerosol particles a greater residence time in the chamber before going into the separator.

### Aerosol Dosing

The indolinone derivative compound, most preferably nintedanib as disclosed herein, can be administered at a therapeutically effective dosage, e.g., a dosage sufficient to provide treatment for the disease states previously described. For example, a daily aerosol dose of nintedanib in a nintedanib compound formulation to a 70 kg human.

The indolinone derivative compound, most preferably nintedanib as disclosed herein, can be administered at a therapeutically effective dosage, e.g., a dosage sufficient to provide treatment for the disease states previously described. In some embodiments, for example, a daily inhaled dry powder aerosol dose of nintedanib or indolinone in a nintedanib or indolinone compound formulation to a 70 kg human may be from about 0.001 mg to about 1.0 mg nintedanib per kg of body weigh per dose. The amount of active compound administered will, of course, be dependent on the subject and disease state being treated, the severity of the affliction, the manner and schedule of administration, the location of the disease (e.g., whether it is desired to effect intra-nasal or upper airway delivery, pharyngeal or laryngeal delivery, bronchial delivery, pulmonary delivery and/or pulmonary delivery with subsequent systemic or central nervous system absorption), and the judgment of the prescribing physician; for example, a likely dose range for aerosol administration of nintedanib in preferred embodiments, or in other embodiments of nintedanib or indolinone derivative compound would be about 0.1 mg to 10 mg per dose to about 0.1 mg to about 100 mg per day. Similarly, if pirfenidone or pyridone analog or PDE4 inhibitor or prostacyclin analog is included in the formulation, a daily aerosol dose to a 70 kg human remains from about 0.001 mg to about 1.0 mg nintedanib per kg of body weigh per dose.

### Dry Powder Inhaler (DPI)

Based upon allometric scaling of animal efficacy data and human modeling, it is observed that the human nintedanib or salt thereof dose may be as low as a range between about 0.04 mg and about 10 mg. If clinical observations support these low levels, a dry powder inhaled product may be a selected alternative to an aqueous nebulized product.

There are two major designs of dry powder inhalers. One design is the metering device in which a reservoir for the drug is placed within the device and the patient adds a dose of the drug into the inhalation chamber. The second is a factory-metered device in which each individual dose has been manufactured in a separate container. Both systems depend upon the formulation of drug into small particles of mass median diameters from about 1 to about 5 micron, and usually involve co-formulation with larger excipient particles (typically 100 micron diameter lactose particles, however particle size can be optimized). Drug powder is placed into the inhalation chamber (either by device metering or by breakage of a factory-metered dosage) and the inspiratory flow of the patient accelerates the powder out of the device and into the oral cavity. Non-laminar flow characteristics of the powder path cause the excipient-drug aggregates to disperse, and the mass of the large excipient particles causes their impaction at the back of the throat, while the smaller drug particles are deposited deep in the lungs.

In multi-dose reservoir systems, individual doses can be metered by volumetric measurement of powder into well-defined orifices in a disk (e.g., Turbuhaler®, AstraZeneca) or a cavity in a slide (e.g., Novolizer®, Viatris). The measuring compartments are filled from the powder bulk reservoirs mainly through the action of gravity. This requires the inhaler be kept in an upright position. In some special cases, forced metering is applied, for example by conducting compressed air through the powder bed in the bulk reservoir (e.g., Airmax™, Ivax Corporation). In general, multi-dose systems require certain properties of the powder formulation regarding flowability and homogeneity. A different metering concept is the Ratiopharm® Jethaler (Ratiopharm), which has a ring compact of the drug-excipient mixture, from which small amounts are grated with a scraper disk during inhalation. The concept is the same as that of the Ultrahaler® (Aventis).

Particle size of the nintedanib or salt thereof, or indolinone derivative or salt thereof may be optimized for aerosol administration for aerosol administration. If the particle size is larger than about 5 micron MMAD then the particles are deposited in upper airways. If the aerodynamic particle size of the aerosol is smaller than about 1 micron then it is delivered into the alveoli and may get transferred into the systemic blood circulation.

By non-limiting example, regardless if stand alone or co-formulated with pirfenidone or pyridone analog or PDE4 inhibitor or prostacyclin analog, the nintedanib or salt thereof, or indolinone derivative or salt thereof disclosed herein are prepared in dosages to disperse and deliver from about 0.01 mg to about 100 mg nintedanib or indolinone compound from a dry powder formulation.

By non-limiting example, a dry powder nintedanib or salt thereof, or indolinone derivative or salt thereof may be administered in the described respirable delivered dose in 10 or fewer actuations and/or inhalation breaths, or in 8 or fewer actuations and/or inhalation breaths, or in 6 or fewer actuations and/or inhalation breaths, or in 4 or fewer actuations and/or inhalation breaths, or in 2 or fewer actuations and/or inhalation breaths.

In some embodiments, a dry powder inhaler (DPI) is used to dispense the nintedanib or salt thereof, or indolinone derivative or salt thereof described herein. DPIs contain the drug substance in fine dry particle form. Typically, inhalation by a patient causes the dry particles to form an aerosol cloud that is drawn into the patient's lungs. The fine dry drug particles may be produced by any technique known in the art. Some well-known techniques include use of a jet mill or other comminution equipment, precipitation from saturated or super saturated solutions, spray drying, in situ micronization (Hovione), particle engineering (Pulmosphere™ Technosphere®, PRINT®) or supercritical fluid methods. Typical powder formulations include production of spherical pellets or adhesive mixtures. In adhesive mixtures, the drug particles are attached to larger carrier particles, such as lactose monohydrate of size about 50 to about 100 microns in diameter. The larger carrier particles decrease the adhesive forces on the carrier/drug agglomerates to improve drug dispersion. Turbulence and/or mechanical devices break the agglomerates into their constituent parts. The smaller drug particles are then drawn into the lungs while the larger carrier particles deposit in the mouth or throat. Some examples of adhesive mixtures are described in U.S. Pat. No. 5,478,578 and PCT Publication Nos. WO 95/11666, WO 87/05213, WO 96/23485, and WO 97/03649, all of which are incorporated by reference in their entirety. Additional excipients may also be included with the drug substance. Alternatively, porous particles may be used to deliver the drug without the need of the larger carrier particles. Such porous particles can be manufactured using the Pulmosphere™ or Technosphere® technologies produce particles that are large in size but small in density and in aerodynamic diameter. Additionally, making drug particles having a specific shape and size using the PRINT® technology can reduce the dispersion force and enable the drug particles to be delivered without the use of a carrier excipient.

There are three common types of DPIs, all of which may be used with the nintedanib or salt thereof, or indolinone derivative or salt thereof compounds described herein. In a single-dose DPI, a capsule containing one dose of dry drug substance/excipients is loaded into the inhaler. Upon activation, the capsule is breached, allowing the dry powder to be dispersed and inhaled using a dry powder inhaler. To dispense additional doses, the old capsule must be removed and an additional capsule loaded. Examples of single-dose DPIs are described in U.S. Pat. Nos. 3,807,400; 3,906,950; 3,991,761; and 4,013,075, all of which are hereby incorporated by reference in their entirety. In a multiple unit dose DPI, a package containing multiple single dose compartments is provided. For example, the package may comprise a blister pack, where each blister compartment contains one dose. Each dose can be dispensed upon breach of a blister compartment. Any of several arrangements of compartments in the package can be used. For example, rotary or strip arrangements are common. Examples of multiple unit does DPIs are described in EPO Patent Publication Nos. 0211595A2, 0455463A1, and 0467172A1, all of which are hereby incorporated by reference in their entirety. In a multi-dose DPI, a single reservoir of dry powder is used. Mechanisms are provided that measure out single dose amounts from the reservoir to be aerosolized and inhaled, such as described in U.S. Pat. Nos. 5,829,434; 5,437,270; 2,587,215; 5,113,855; 5,840,279; 4,688,218; 4,667,668; 5,033,463; and 4,805,811 and PCT Publication No. WO 92/09322, all of which are hereby incorporated by reference in their entirety.

In some embodiments, auxiliary energy in addition to or other than a patient's inhalation may be provided to facilitate operation of a DPI. For example, pressurized air may be provided to aid in powder de-agglomeration, such as described in U.S. Pat. Nos. 3,906,950; 5,113,855; 5,388,572; 6,029,662 and PCT Publication Nos. WO 93/12831, WO 90/07351, and WO 99/62495, all of which are hereby incorporated by reference in their entirety. Electrically driven impellers may also be provided, such as described in U.S. Pat. Nos. 3,948,264; 3,971,377; 4,147,166; 6,006,747 and PCT Publication No. WO 98/03217, all of which are hereby incorporated by reference in their entirety. Another mechanism is an electrically powered tapping piston, such as described in PCT Publication No. WO 90/13327, which is hereby incorporated by reference in its entirety. Other DPIs use a vibrator, such as described in U.S. Pat. Nos. 5,694,920 and 6,026,809, both of which are hereby incorporated by reference in their entirety. Finally, a scraper system may be employed, such as described in PCT Publication No. WO 93/24165, which is hereby incorporated by reference in its entirety.

Additional examples of DPIs for use herein are described in U.S. Pat. Nos. 4,811,731; 5,113,855; 5,840,279; 3,507,277; 3,669,113; 3,635,219; 3,991,761; 4,353,365; 4,889,144, 4,907,538; 5,829,434; 6,681,768; 6,561,186; 5,918,594; 6,003,512; 5,775,320; 5,740,794; and 6,626,173, all of which are hereby incorporated by reference in their entirety.

In some embodiments, a spacer or chamber may be used with any of the inhalers described herein to increase the amount of drug substance that gets absorbed by the patient, such as is described in U.S. Pat. Nos. 4,470,412; 4,790,305; 4,926,852; 5,012,803; 5,040,527; 5,024,467; 5,816,240; 5,027,806; and 6,026,807, all of which are hereby incorporated by reference in their entirety. For example, a spacer may delay the time from aerosol production to the time when the aerosol enters a patient's mouth. Such a delay may improve synchronization between the patient's inhalation and the aerosol production. A mask may also be incorporated for infants or other patients that have difficulty using the traditional mouthpiece, such as is described in U.S. Pat. Nos. 4,809,692; 4,832,015; 5,012,804; 5,427,089; 5,645,049; and 5,988,160, all of which are hereby incorporated by reference in their entirety.

Dry powder inhalers (DPIs), which involve deaggregation and aerosolization of dry powder particles, normally rely upon a burst of inspired air that is drawn through the unit to deliver a drug dosage. Such devices are described in, for example, U.S. Pat. No. 4,807,814, which is directed to a pneumatic powder ejector having a suction stage and an injection stage; SU 628930 (Abstract), describing a hand-held powder disperser having an axial air flow tube; Fox et al., Powder and Bulk Engineering, pages 33-36 (March 1988), describing a venturi adductor having an axial air inlet tube upstream of a venturi restriction; EP 347 779, describing a hand-held powder disperser having a collapsible expansion chamber, and U.S. Pat. No. 5,785,049, directed to dry powder delivery devices for drugs.

Commercial examples of capsule-based or blister pack-based dry powder inhalers that can be used with the nintedanib or salt thereof, or indolinone derivative or salt thereof formulations described herein include the Aerohaler, Aerolizer, Aspirair, Breezehaler, Diskhaler Forspiro, Exubera, Gyrohaler, Plastiape Monodose, Podhaler, Prohaler, Redihaler, Rotahaler, Turbohaler, Handihalerand Discus. Multi dose reservoir devices include E Flex, Jethaler, NEXThaler, Novolizer, PADD, Pulmojet, Spiromax, Swinghaler, Turbuhaler, and Ultrahaler. A commercial example of cassette-based dry powder inhaler is Spiros.

### Solid Lipid Particles

Preparation of nintedanib or a indolinone derivative compound or salt thereof compound solid lipid particles may involve dissolving the drug in a lipid melt (phospholipids such as phosphatidyl choline and phosphatidyl serine) maintained at least at the melting temperature of the lipid, followed by dispersion of the drug-containing melt in a hot aqueous surfactant solution (typically 1-5% w/v) maintained at least at the melting temperature of the lipid. The coarse dispersion will be homogenized for 1-10 min using a Microfluidizer® to obtain a nano emulsion. Cooling the nano emulsion to a temperature between 4-25° C. will re-solidify the lipid, leading to formation of solid lipid nanoparticles. Optimization of formulation parameters (type of lipid matrix, surfactant concentration and production parameters) will be performed so as to achieve a prolonged drug delivery. By non-limiting example, this approach may also be used to sequester and improve the water solubility of solid, AUC shape-enhancing formulations, such as low-solubility nintedanib or a indolinone derivative compound or salt thereof.

### Co-Precipitates

Co-precipitate nintedanib or a indolinone derivative compound or salt thereof compound formulations may be prepared by formation of co-precipitates with pharmacologically inert, polymeric materials. It has been demonstrated that the formation of molecular solid dispersions or co-precipitates to create an AUC shape-enhancing formulations with various water-soluble polymers can significantly slow their in vitro dissolution rates and/or in vivo absorption. In preparing powdered products, grinding is generally used for reducing particle size, since the dissolution rate is strongly affected by particle size. Moreover, a strong force (such as grinding) may increase the surface energy and cause distortion of the crystal lattice as well as reducing particle size. Co-grinding drug with hydroxypropyl methylcellulose, b-cyclodextrin, chitin and chitosan, crystalline cellulose, and gelatin, may enhance the dissolution properties such that AUC shape-enhancement is obtained for otherwise readily bioavailable nintedanib or a indolinone derivative compound or salt thereof compounds. By non-limiting example, this approach may also be used to sequester and improve the water solubility of solid, AUC shape-enhancing formulations, such as low-solubility nintedanib or a indolinone derivative compound or salt thereof compounds or salt forms for nanoparticle-based formulations.

### Dispersion-Enhancing Peptides

Compositions may include one or more di- or tripeptides containing two or more leucine residues. By further non-limiting example, U.S. Pat. No. 6,835,372 disclosing dispersion-enhancing peptides, is hereby incorporated by reference in its entirety. This patent describes the discovery that di-leucyl-containing dipeptides (e.g., dileucine) and tripeptides are superior in their ability to increase the dispersibility of powdered composition.

In another embodiment, highly dispersible particles including an amino acid are administered. Hydrophobic amino acids are preferred. Suitable amino acids include naturally occurring and non-naturally occurring hydrophobic amino acids. Some naturally occurring hydrophobic amino acids, including but not limited to, non-naturally occurring amino acids include, for example, beta-amino acids. Both D, L and racemic configurations of hydrophobic amino acids can be employed. Suitable hydrophobic amino acids can also include amino acid analogs. As used herein, an amino acid analog includes the D or L configuration of an amino acid having the following formula: —NH—CHR—CO—, wherein R is an aliphatic group, a substituted aliphatic group, a benzyl group, a substituted benzyl group, an aromatic group or a substituted aromatic group and wherein R does not correspond to the side chain of a naturally-occurring amino acid. As used herein, aliphatic groups include straight chained, branched or cyclic C1-C8 hydrocarbons which are completely saturated, which contain one or two heteroatoms such as nitrogen, oxygen or sulfur and/or which contain one or more units of desaturation. Aromatic groups include carbocyclic aromatic groups such as phenyl and naphthyl and heterocyclic aromatic groups such as imidazolyl, indolyl, thienyl, furanyl, pyridyl, pyranyl, oxazolyl, benzothienyl, benzofuranyl, quinolinyl, isoquinolinyl and acridintyl.

Suitable substituents on an aliphatic, aromatic or benzyl group include —OH, halogen (—Br, —Cl, —I and —F)—O(aliphatic, substituted aliphatic, benzyl, substituted benzyl, aryl or substituted aryl group), —CN, —NO2, —COOH, —NH2, —NH(aliphatic group, substituted aliphatic, benzyl, substituted benzyl, aryl or substituted aryl group), —N(aliphatic group, substituted aliphatic, benzyl, substituted benzyl, aryl or substituted aryl group)2, —COO(aliphatic group, substituted aliphatic, benzyl, substituted benzyl, aryl or substituted aryl group), —CONH2, —CONH(aliphatic, substituted aliphatic group, benzyl, substituted benzyl, aryl or substituted aryl group)), —SH, —S(aliphatic, substituted aliphatic, benzyl, substituted benzyl, aromatic or substituted aromatic group) and —NH—C(.dbd.NH)—NH2. A substituted benzylic or aromatic group can also have an aliphatic or substituted aliphatic group as a substituent. A substituted aliphatic group can also have a benzyl, substituted benzyl, aryl or substituted aryl group as a substituent. A substituted aliphatic, substituted aromatic or substituted benzyl group can have one or more substituents. Modifying an amino acid substituent can increase, for example, the lipophilicity or hydrophobicity of natural amino acids which are hydrophilic.

A number of the suitable amino acids, amino acids analogs and salts thereof can be obtained commercially. Others can be synthesized by methods known in the art.

Hydrophobicity is generally defined with respect to the partition of an amino acid between a nonpolar solvent and water. Hydrophobic amino acids are those acids which show a preference for the nonpolar solvent. Relative hydrophobicity of amino acids can be expressed on a hydrophobicity scale on which glycine has the value 0.5. On such a scale, amino acids which have a preference for water have values below 0.5 and those that have a preference for nonpolar solvents have a value above 0.5. As used herein, the term hydrophobic amino acid refers to an amino acid that, on the hydrophobicity scale, has a value greater or equal to 0.5, in other words, has a tendency to partition in the nonpolar acid which is at least equal to that of glycine.

Examples of amino acids which can be employed include, but are not limited to: glycine, proline, alanine, cysteine, methionine, valine, leucine, tyrosine, isoleucine, phenylalanine, tryptophan. Preferred hydrophobic amino acids include leucine, isoleucine, alanine, valine, phenylalanine and glycine. Combinations of hydrophobic amino acids can also be employed. Furthermore, combinations of hydrophobic and hydrophilic (preferentially partitioning in water) amino acids, where the overall combination is hydrophobic, can also be employed.

The amino acid can be present in the particles of the invention in an amount of at least 10 weight %. Preferably, the amino acid can be present in the particles in an amount ranging from about 20 to about 80 weight %. The salt of a hydrophobic amino acid can be present in the particles of the invention in an amount of at least 10 weight percent. Preferably, the amino acid salt is present in the particles in an amount ranging from about 20 to about 80 weight %. In preferred embodiments the particles have a tap density of less than about 0.4 g/cm3.

Methods of forming and delivering particles which include an amino acid are described in U.S. Pat. No. 6,586,008, entitled Use of Simple Amino Acids to Form Porous Particles During Spray Drying, the teachings of which are incorporated herein by reference in their entirety.

### Surface Modifiers

Nintedanib or a indolinone derivative compound or salt thereof compounds disclosed herein may be prepared in a pharmaceutical composition with suitable surface modifiers which may be selected from known organic and inorganic pharmaceutical excipients. Such excipients include low molecular weight oligomers, polymers, surfactants and natural products. Preferred surface modifiers include nonionic and ionic surfactants. Two or more surface modifiers can be used in combination.

Representative examples of surface modifiers include acetyl pyridinium chloride, gelatin, casein, lecithin (phosphatides), dextran, glycerol, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters (e.g., the commercially available Tweens™, such as e.g., Tween 20™, and Tween 80™, (ICI Specialty Chemicals)); polyethylene glycols (e.g., Carbowaxs 3350™, and 1450™, and Carbopol 934™, (Union Carbide)), dodecyl trimethyl ammonium bromide, polyoxyethylenestearates, colloidal silicon dioxide, phosphates, sodium dodecylsulfate, carboxymethylcellulose calcium, hydroxypropyl cellulose (HPC, HPC-SL, and HPC-L), hydroxypropyl methylcellulose (HPMC), carboxymethylcellulose sodium, methylcellulose, hydroxyethylcellulose, hydroxypropylcellulose, hydroxypropylmethyl-cellulose phthalate, noncrystalline cellulose, magnesium aluminum silicate, triethanolamine, polyvinyl alcohol (PVA), polyvinylpyrrolidone (PVP), 4-(1,1,3,3-tetaamethylbutyl)-phenol polymer with ethylene oxide and formaldehyde (also known as tyloxapol, superione, and triton), poloxamers (e.g., Pluronics F68™, and F108™, which are block copolymers of ethylene oxide and propylene oxide); poloxamines (e.g., Tetronic 908™, also known as Poloxamine 908™, which is a tetrafunctional block copolymer derived from sequential addition of propylene oxide and ethylene oxide to ethylenediamine (BASF Wyandotte Corporation, Parsippany, N.J.)); a charged phospholipid such as dimyristoyl phophatidyl glycerol, dioctylsulfosuccinate (DOSS); Tetronic 1508™; (T-1508) (BASF Wyandotte Corporation), dialkylesters of sodium sulfosuccinic acid (e.g., Aerosol OT™, which is a dioctyl ester of sodium sulfosuccinic acid (American Cyanamid)); Duponol P™, which is a sodium lauryl sulfate (DuPont); Tritons X-200™, which is an alkyl aryl polyether sulfonate (Rohm and Haas); Crodestas F-110™, which is a mixture of sucrose stearate and sucrose distearate (Croda Inc.); p-isononylphenoxypoly-(glycidol), also known as Olin-Log™, or Surfactant 10-G™, (Olin Chemicals, Stamford, Conn.); Crodestas SL-40™, (Croda, Inc.); and SA9OHCO, which is C18H37CH2(CON(CH3)-CH2(CHOH)4(CH2OH)2 (Eastman Kodak Co.); decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucarmide; n-octyl-β-D-glucopyranoside; octyl β-D-thioglucopyranoside; and the like.

Examples of surfactants for use in the solutions disclosed herein include, but are not limited to, ammonium laureth sulfate, cetamine oxide, cetrimonium chloride, cetyl alcohol, cetyl myristate, cetyl palmitate, cocamide DEA, cocamidopropyl betaine, cocamidopropylamine oxide, cocamide MEA, DEA lauryl sulfate, di-stearyl phthalic acid amide, dicetyl dimethyl ammonium chloride, dipalmitoylethyl hydroxethylmonium, disodium laureth sulfosuccinate, di(hydrogenated) tallow phthalic acid, glyceryl dilaurate, glyceryl distearate, glyceryl oleate, glyceryl stearate, isopropyl myristate nf, isopropyl palmitate nf, lauramide DEA, lauramide MEA, lauramide oxide, myristamine oxide, octyl isononanoate, octyl palmitate, octyldodecyl neopentanoate, olealkonium chloride, PEG-2 stearate, PEG-32 glyceryl caprylate/caprate, PEG-32 glyceryl stearate, PEG-4 and PEG-150 stearate & distearate, PEG-4 to PEG-150 laurate & dilaurate, PEG-4 to PEG-150 oleate & dioleate, PEG-7 glyceryl cocoate, PEG-8 beeswax, propylene glycol stearate, sodium C14-16 olefin sulfonate, sodium lauryl sulfoacetate, sodium lauryl sulphate, sodium trideceth sulfate, stearalkonium chloride, stearamide oxide, TEA-dodecylbenzene sulfonate, TEA lauryl sulfate

Most of these surface modifiers are known pharmaceutical excipients and are described in detail in the Handbook of Pharmaceutical Excipients, published jointly by the American Pharmaceutical Association and The Pharmaceutical Society of Great Britain (The Pharmaceutical Press, 1986), specifically incorporated by reference. The surface modifiers are commercially available and/or can be prepared by techniques known in the art. The relative amount of drug and surface modifier can vary widely and the optimal amount of the surface modifier can depend upon, for example, the particular drug and surface modifier selected, the critical micelle concentration of the surface modifier if it forms micelles, the hydrophilic-lipophilic-balance (HLB) of the surface modifier, the melting point of the surface modifier, the water solubility of the surface modifier and/or drug, the surface tension of water solutions of the surface modifier, etc.

In the present invention, the optimal ratio of drug to surface modifier is ˜0.1% to ˜99.9% nintedanib or a indolinone derivative compound or salt thereof compound, more preferably about 10% to about 90%.

### Microspheres

Microspheres can be used for pulmonary delivery of nintedanib or a indolinone derivative compound or salt thereof compounds by first adding an appropriate amount of drug compound to be solubilized in water. For example, an aqueous nintedanib or a indolinone derivative compound or salt thereof compound solution may be dispersed in methylene chloride containing a predetermined amount (0.1-1% w/v) of poly(DL-lactide-co-glycolide) (PLGA) by probe sonication for 1-3 min on an ice bath. Separately, a nintedanib or a indolinone derivative compound or salt thereof compound may be solubilized in methylene chloride containing PLGA (0.1-1% w/v). The resulting water-in-oil primary emulsion or the polymer/drug solution will be dispersed in an aqueous continuous phase consisting of 1-2% polyvinyl alcohol (previously cooled to 4° C.) by probe sonication for 3-5 min on an ice bath. The resulting emulsion will be stirred continuously for 2-4 hours at room temperature to evaporate methylene chloride. Microparticles thus formed will be separated from the continuous phase by centrifuging at 8000-10000 rpm for 5-10 min. Sedimented particles will be washed thrice with distilled water and freeze dried. Freeze-dried nintedanib or an indolinone derivative compound or salt thereof compound microparticles will be stored at −20° C.

By non-limiting example, a spray drying approach may be employed to prepare nintedanib or an indolinone derivative compound or salt thereof compound microspheres. An appropriate amount of nintedanib or an indolinone derivative compound or salt thereof compound will be solubilized in methylene chloride containing PLGA (0.1-1%). This solution will be spray dried to obtain the microspheres.

### Pharmacokinetics

Inhalation therapy of aerosolized nintedanib or an indolinone derivative compound enables direct deposition of the sustained-release or active substance in the respiratory tract (be that intra-nasal or pulmonary) for therapeutic action at that site of deposition or systemic absorption to regions immediately down stream of the vascular absorption site.

Similar to the intra-nasal and pulmonary applications described above, treatment or prevention of organs outside the respiratory tract requires absorption to the systemic vascular department for transport to these extra-respiratory sites. In the case of treating or preventing fibrotic or inflammatory diseases associated with the heart, liver and kidney, deposition of drug in the respiratory tract, more specifically the deep lung will enable direct access to these organs through the left atrium to either the carotid arteries or coronary arteries. This direct delivery will permit direct dosing of high concentration nintedanib or an indolinone derivative compound in the absence of unnecessary systemic exposure. Similarly, this route permits titration of the dose to a level for these indications.

Pharmacokinetics is concerned with the uptake, distribution, metabolism and excretion of a drug substance. A pharmacokinetic profile comprises one or more biological measurements designed to measure the absorption, distribution, metabolism and excretion of a drug substance. One way of visualizing a pharmacokinetic profile is by means of a blood plasma concentration curve, which is a graph depicting mean active ingredient blood plasma concentration on the Y-axis and time (usually in hours) on the X-axis. Some pharmacokinetic parameters that may be visualized by means of a blood plasma concentration curve include:
1) Cmax: The maximum plasma concentration in a patient;
2) AUC: area under the curve
3) TOE: time of exposure
4) T½: period of time it takes for the amount in a patient of drug to decrease by half
5) Tmax: The time to reach maximum plasma concentration in a patient

Pharmacokinetics (PK) is concerned with the time course of a therapeutic agent, such as nintedanib or a indolinone derivative compound concentration in the body. Pharmacodynamics (PD) is concerned with the relationship between pharmacokinetics and efficacy in vivo. PK/PD parameters correlate the therapeutic agent, such as exposure with efficacious activity. Accordingly, to predict the therapeutic efficacy of a therapeutic agent, such as with diverse mechanisms of action different PK/PD parameters may be used.

As used herein, the “peak period” of a pharmaceutical's in vivo concentration is defined as that time of the pharmaceutical dosing interval when the pharmaceutical concentration is not less than 50% of its maximum plasma or site-of-disease concentration. “Peak period” is used to describe an interval of nintedanib or an indolinone derivative compound dosing. When considering the treatment of lung diseases, a method or system described herein provides at least a two-fold enhancement in pharmacokinetic profile for treatment of the lung disease. The methods and systems described herein provide at least a two-fold enhancement in the lung tissue pharmacokinetic profile of nintedanib or indolinone or salt thereof compound as compared to oral administration.

The amount of nintedanib or indolinone or salt thereof compound that is administered to a human by inhalation may be calculated by measuring the amount of nintedanib or indolinone or salt thereof compound and associated metabolites that are found in the urine. About 80% of administered nintedanib is excreted in the urine. The calculation based on compound and metabolites in urine may be done through a 48 hour urine collection (following a single administration), whereby the total amount of nintedanib or indolinone or salt thereof compound delivered to the human is the sum of measured nintedanib and its metabolites. By non-limiting example, knowing that 80% of nintedanib is excreted, a 50 mg sum urinary measurement of nintedanib and its metabolites would translate to a delivered dose of about 63 mg (50 mg divided by 80%). If the inhaled aerosol fine-particle fraction (FPF) is 75%, one may assume that about 75% of the drug deposited in the lung (and about 25% was swallowed, and subsequently absorbed from the gut with 80% excreted in the urine). Integrating these two calculations, of a 63 mg delivered dose (as measured by urinary excretion), about 47 mg would be the amount of inhaled aerosol nintedanib delivered to the lung (the actual RDD; calculated as the product of 63 mg and a 75% FPF). This RDD can then be used in a variety of calculations, including lung tissue concentration.

The lung tissue Cmax and/or AUC of nintedanib or indolinone or salt thereof, that is obtained after administration of a single inhaled dose to the mammal is about the same or greater than the lung tissue Cmax and/or AUC of nintedanib or indolinone or salt thereof, that is obtained after a single dose of orally administered dose that is from about 80% to about 120% of the inhaled dose; and/or the plasma Cmax and/or AUC that is obtained after administration of a single inhaled dose to the mammal is less than the plasma Cmax and/or AUC of obtained after a single dose of orally administered nintedanib or indolinone or salt thereof, at a dose that is from about 80% to about 120% of the inhaled dose. The lung tissue Cmax that is obtained after administration of a single inhaled dose to the mammal is greater than the lung tissue obtained after a single dose of orally administered nintedanib or indolinone or salt thereof, at a dose that is from about 80% to about 120% of the inhaled dose. The lung tissue AUC of nintedanib or indolinone or salt thereof, that is obtained after administration of a single inhaled dose to the mammal is greater than the lung tissue AUC obtained after a single dose of orally administered nintedanib or indolinone or salt thereof, at a dose that is from about 80% to about 120% of the inhaled dose. The plasma Cmax of nintedanib or indolinone or salt thereof, that is obtained after administration of a single inhaled dose to the mammal is less than the plasma Cmax obtained after a single dose of orally administered nintedanib or indolinone or salt thereof, at a dose that is from about 80% to about 120% of the inhaled dose. The plasma AUC of nintedanib or indolinone or salt thereof, that is obtained after administration a single inhaled dose to the mammal is less than the plasma AUC obtained after a single dose of orally administered nintedanib or indolinone or salt thereof, compound at a dose that is from about 80% to about 120% of the inhaled dose.

In one aspect, described herein is a method of achieving a lung tissue Cmax of nintedanib or indolinone or salt thereof compound that is at least 1.5 times, at least 2 times, at least 3 times, at least 4 times, at least 5 times, at least 6 times, at least 1.5 times, at least 1.5 times, at least 1.5 times, at least 1.5 times, at least 7 times, at least 8 times, at least 9 times, at least 10 times, at least 20 times a Cmax of up to 200 mg of an orally administered dosage of nintedanib or indolinone or salt thereof, the method comprising dispersing a dry powder formulation comprising nintedanib or indolinone or salt thereof, and administering the dry powder formulation to a human. Described herein is a method of achieving a lung tissue Cmax of nintedanib or indolinone or salt thereof compound that is at least equivalent to or greater than a Cmax of up to 200 mg of an orally administered dosage of nintedanib or indolinone or salt thereof, the method comprising dispersing a dry powder formulation comprising nintedanib or indolinone or salt thereof, and administering the dry powder formulation to a human.

In one aspect, described herein is a method of achieving a lung tissue AUC₀₋₂₄ of nintedanib or indolinone or salt thereof, that is at least 1.5 times, at least 2 times, at least 3 times, at least 4 times, at least 5 times, at least 6 times, at least 1.5 times, at least 1.5 times, at least 1.5 times, at least 1.5 times, at least 7 times, at least 8 times, at least 9 times, at least 10 times, at least 1.5-20 times, at least 1.5-15 times, at least 1.5-10 times, at least 1.5-5 times, or at least 1.5-3 times AUC₀₋₂₄ of up to 200 mg of an orally administered dosage, the method comprising dispersing a dry powder formulation comprising nintedanib or indolinone or salt thereof compound and administering the dry powder formulation to a human. A method of achieving a lung tissue AUC₀₋₂₄ of nintedanib or indolinone or salt thereof compound that is at least equivalent to or greater than AUC₀₋₂₄ of up to 600 mg of an orally administered dosage of nintedanib or indolinone or salt thereof, the method comprising dispersing a dry powder formulation comprising nintedanib or indolinone or salt thereof and administering the dry powder formulation to a human.

The methods include a method of administering nintedanib or indolinone or salt thereof, to a human, comprising administering a dry powder formulation containing the nintedanib or indolinone or salt thereof, wherein the lung tissue Cmax achieved with the dry powder formulation is at least 1.5 times, at least 2 times, at least 3 times, at least 4 times, at least 5 times, at least 6 times, at least 1.5 times, at least 1.5 times, at least 1.5 times, at least 1.5 times, at least 7 times, at least 8 times, at least 9 times, at least 10 times, at least 20 times the lung tissue Cmax achieved with an orally administered nintedanib or indolinone or salt thereof, dosage that is from 80% to 120% of the dose amount of nintedanib that is administered by a DPI.

The methods include a method of administering nintedanib or indolinone or salt thereof, to a human, comprising administering a dry powder formulation containing the nintedanib or indolinone or salt thereof, wherein the lung tissue Cmax achieved with the dry powder formulation is at least equivalent to or greater than the lung tissue Cmax achieved with an orally administered nintedanib or indolinone or salt thereof, dosage that is from 80% to 120% of the dosage of nintedanib or indolinone or salt thereof, in the dry powder formulation of nintedanib or indolinone or salt thereof that is administered.

The methods include a method of administering nintedanib or indolinone or salt thereof, to a human, comprising administering a dry powder formulation containing the nintedanib or indolinone or salt thereof, wherein the plasma AUC₀₋₂₄ achieved with the dry powder formulation is less than the plasma AUC₀₋₂₄ achieved with an orally administered nintedanib or indolinone or salt thereof, dosage that is from 80% to 120% of the dosage of nintedanib or indolinone or salt thereof, in the dry powder formulation of nintedanib or indolinone or salt thereof, that is administered.

The methods include a method of administering nintedanib or indolinone or salt thereof comprising administering a dry powder formulation containing the nintedanib or indolinone or salt thereof, wherein the lung tissue AUC₀₋₂₄ achieved with the nebulized solution is at least 1.5 times, at least 2 times, at least 3 times, at least 4 times, at least 5 times, at least 6 times, at least 1.5 times, at least 1.5 times, at least 1.5 times, at least 1.5 times, at least 7 times, at least 8 times, at least 9 times, at least 10 times, at least 1.5-20 times, at least 1.5-15 times, at least 1.5-10 times, at least 1.5-5 times, or at least 1.5-3 times the lung tissue AUC₀₋₂₄ achieved with an orally administered nintedanib or indolinone or salt thereof compound dosage that is from 80% to 120% of the dosage of nintedanib or indolinone or salt thereof, in the dry powder formulation of nintedanib or indolinone or salt thereof. The methods include a method of administering nintedanib or indolinone or salt thereof, to a human, comprising administering a dry powder formulation containing the nintedanib or indolinone or salt thereof, wherein the lung tissue AUC₀₋₂₄ achieved with the dry powder formulation is at least 1.5 times the lung tissue AUC₀₋₂₄ achieved with an orally administered nintedanib or indolinone or salt thereof, dosage that is from 80% to 120% of the dosage of nintedanib or indolinone or salt thereof, in the dry powder formulation of nintedanib or indolinone or salt thereof compound.

The methods include a method of improving the pharmacokinetic profile obtained in a human following a single oral dose administration of nintedanib or indolinone or salt thereof. The single oral dose comprises up to about 200 mg of nintedanib or indolinone or salt thereof. The method of improving the pharmacokinetic profile further comprises a comparison of the pharmacokinetic parameters following inhalation administration to the same parameters obtained following oral administration and may require multiple measurements of a single patient over time comparing the pharmacokinetic parameters in a single patient varying by dosage, route of administration, form of active pharmaceutical ingredient and other parameters as described herein. A prolonged improvement in pharmacokinetic profile is obtained by repeated and frequent administrations of the dry powder formulation of nintedanib or indolinone or salt thereof, as described herein by inhalation. Repeated administration of nintedanib or indolinone or salt thereof, by inhalation provides more frequent direct lung exposure benefiting the human through repeat high Cmax levels. The inhaled nintedanib or indolinone or salt thereof, doses are administered once a day, twice a day, three times a day, four times a day, every other day, twice a week, three times a week, four times a week, five times a week, six times a week, seven times a week, or any combination thereof.

Small intratracheal aerosol doses deliver a rapidly-eliminated high lung Cmax and low AUC. Human, animal and in vitro studies all indicate that nintedanib efficacy is dose responsive (i.e. larger doses correlate with improved efficacy) and suggest Cmax is a key driver in nintedanib efficacy. While lung Cmax appears important for efficacy, more regular nintedanib exposure is important to enhance this effect. In the context of treating lung diseases in a human, more frequent direct-lung administration of nintedanib or indolinone or salt thereof compound may provide benefit through both repeat high Cmax dosing and providing more regular exposure of the active therapeutic agent.

Methods of treatment include a method for the treatment of lung disease in a mammal comprising administering directly to the lungs of the mammal in need thereof nintedanib or salt thereof, or a indolinone derivative compound or salt thereof, on a continuous dosing schedule, wherein the observed lung tissue Cmax of a dose of nintedanib or indolinone derivative or salt thereof greater than 10, 100, 1000 or 10,000 ng/mL lung epithelial lining fluid. When co-formulated or administered in combination with Pirfenidone, the resulting blood Pirfenidone Cmax is less than 10 μg/mL, less than 5 μg/mL, less than 2.5 μg/mL. When co-formulated and administered in combination with a PDE4 inhibitor, the resulting blood PDE4 inhibitor Cmax is less than 10 μg/mL, less than 5 μg/mL, less than 2.5 μg/mL, less than 1.0 μg/mL, less than 0.5 μg/mL, less than 0.1 μg/mL. When co-formulated and administered in combination with a prostacyclin analog, the resulting blood prostacyclin analog Cmax is less than 10 ng/mL, less than 5 ng/mL, less than 2.5 ng/mL, less than 1.0 ng/mL, less than 0.5 ng/mL, less than 0.1 ng/mL.

### Dry Powder Inhalers

Dry powder inhalers for use to disperse nintedanib dry powder alone, with Pirfenidone or with PDE4 inhibitor or prostacyclin, or combinations thereof, can be simple dry powder inhaler devices known to those who are skilled in the art such as the high resistance devices (e.g., Easyhaler, Handihaler, Spinhaler, Rotahaler, Twisthaler), the medium resistance devices (e.g., Turbuhaler, Accuhaler, Diskus, Ellipta, Novolizer, and Genuair) and the low resistance devices (Aerolizer Breezhaler). The Plastiape RS01 devices with low, medium and high flow resistance are also suitable for dispersing the nintedanib dry powder formulation or combination formulation of nintedanib (or indolinone analog) with Pirfenidone (or pyridine analog) or with PDE4 inhibitor or prostacyclin. Additionally, these dry powder inhalation devices can be attached to an accessory that measures the flow rate and using artificial intelligence, provide feedback to the patient to ensure the patient generates adequate airflow rate during dosing to ensure proper aerosol dispersion. Additionally, active breath actuated dry powder devices such as the Spiros, Exubra that provides external power to disperse the dry powder formulation.

### Methods of Dosing and Treatment Regimens

The term “continuous dosing schedule” refers to the administration of a particular therapeutic agent at regular intervals. Continuous dosing schedule refers to the administration of a particular therapeutic agent at regular intervals without any drug holidays from the particular therapeutic agent. A continuous dosing schedule refers to the administration of a particular therapeutic agent in alternating cycles of drug administration followed by a drug holiday (e.g. wash out period) from the particular therapeutic agent. For example, in some embodiments the therapeutic agent is administered once a day, twice a day, three times a day, once a week, twice a week, three times a week, four times a week, five times a week, six times a week, seven times a week, every other day, every third day, every fourth day, daily for a week followed by a week of no administration of the therapeutic agent, daily for a two weeks followed by one or two weeks of no administration of the therapeutic agent, daily for three weeks followed by one, two or three weeks of no administration of the therapeutic agent, daily for four weeks followed by one, two, three or four weeks of no administration of the therapeutic agent, weekly administration of the therapeutic agent followed by a week of no administration of the therapeutic agent, or biweekly administration of the therapeutic agent followed by two weeks of no administration of the therapeutic agent.

The amount of nintedanib or a indolinone derivative compound is administered once-a-day. In some other embodiments, the amount of nintedanib or an indolinone derivative compound is administered twice-a-day. In some other embodiments, the amount of nintedanib or an indolinone derivative compound is administered three times a day.

Where improvement in the status of the disease or condition in the human is not observed, the daily dose of nintedanib or a indolinone derivative compound is increased for example, a once-a-day dosing schedule is changed to a twice-a-day dosing schedule. A three times a day dosing schedule is employed to increase the amount of nintedanib or a indolinone derivative compound that is administered. Frequency of administration by inhalation is increased in order to provide repeat high Cmax levels on a more regular basis. The frequency of administration by inhalation is increased in order to provide maintained or more regular exposure to Nintedanib. The frequency of administration by inhalation is increased in order to provide repeat high Cmax levels on a more regular basis and provide maintained or more regular exposure to nintedanib.

The amount of repeat high Cmax dosing providing more regular exposure of the active therapeutic agent that is given to the human varies depending upon factors such as, but not limited to, condition and severity of the disease or condition, and the identity (e.g., weight) of the human, and the particular additional therapeutic agents that are administered (if applicable).

### EXAMPLES

### Example 1. PDGF-Induced Fibroblast Proliferation

The impact of nintedanib on inhibiting PDGF-induced fibroblast proliferation was determined in primary human fibroblasts. Briefly, fibroblasts were seeded at 2,500 cells/well in 96-well flat clear bottom Falcon plates in 10% FBS F12/DMEM Media with 1% Pen/Strep. These cells were left in a 37 degree incubator (5% CO₂) for 24 hours to allow the cells to adhere to the plate. The media was then removed, washed with PBS and replaced the media with 0.5% FBS F12/DMEM Media with 1% Pen/Strep for another 24 hours. To characterize the impact exposure duration of each drug on inhibiting proliferation, cells were pretreated with or without drug (0.5 to 50 nM) for 30 minutes, washed and either replaced with 0.5% FBS F12/DMEM media with 1% Pen/Strep+/−20 ng/mL PDGF-BB (short-duration drug exposure mimicking pulmonary inhalation pharmacokinetics) or 0.5% FBS F12/DMEM media with 1% Pen/Strep+/−20 ng/mL PDGF-BB and the initial drug concentration (long duration drug exposure mimicking oral pharmacokinetics). After 72 hours of viable cells was assessed using the MTS assay. Drug concentrations tested were not cytotoxic (data not shown).

TABLE 1
Impact of nintedanib and exposure duration
on PDGF-induced fibroblast differentiation.

| | Nintedanib Exposure | | | |
|---|---|---|---|---|
| Nintedanib | Short Duration | | Long Duration | |
| nM | Proliferation* | SEM | Proliferation* | SEM |
| 0 | 0.160 | 0.080 | 0.160 | 0.065 |
| 0.5 | 0.115 | 0.070 | 0.003 | 0.095 |
| 5.0 | 0.011 | 0.185 | −0.359 | 0.120 |
| 50.0 | −0.175 | 0.047 | −0.642 | 0.068 |

*Relative proliferation

Results from Table 1 show that nintedanib is dose-responsive in inhibiting PDGF-induced fibroblast proliferation. The data also show that only short-term nintedanib exposure (supportive of inhalation pulmonary pharmacokinetics) is required for this activity with a fifty-percent inhibitory concentration (IC50) of about 3 nM (about 1.6 ng/mL).

### Example 2. Formulations

TABLE 2
Exemplary Nintedanib Dry Powder Formulations

| | Nintedanib base, | | | | |
|---|---|---|---|---|---|
| | Nintedanib Salt, | | | | |
| | Nintedanib HCl | | | | |
| Formu- | or Nintedanib | Lactose | Trehalose | Leucine | Saccharin |
| lation | HBr (%)^{a} | (%) | (%) | (%) | (%) |
| 1 | 90 | 10 | 0 | 0 | 0 |
| 2 | 50 | 50 | 0 | 0 | 0 |
| 3 | 20 | 80 | 0 | 0 | 0 |
| 4 | 15 | 85 | 0 | 0 | 0 |
| 5 | 10 | 90 | 0 | 0 | 0 |
| 6 | 5 | 95 | 0 | 0 | 0 |
| 7 | 90 | 0 | 10 | 0 | 0 |
| 8 | 50 | 0 | 50 | 0 | 0 |
| 9 | 20 | 0 | 80 | 0 | 0 |
| 10 | 15 | 0 | 85 | 0 | 0 |
| 11 | 10 | 0 | 90 | 0 | 0 |
| 12 | 5 | 0 | 95 | 0 | 0 |
| 13 | 90 | 5 | 0 | 5 | 0 |
| 14 | 50 | 45 | 0 | 5 | 0 |
| 15 | 20 | 75 | 0 | 5 | 0 |
| 16 | 15 | 80 | 0 | 5 | 0 |
| 17 | 10 | 85 | 0 | 5 | 0 |
| 18 | 5 | 90 | 0 | 5 | 0 |
| 19 | 90 | 0 | 0 | 10 | 0 |
| 20 | 50 | 40 | 0 | 10 | 0 |
| 21 | 20 | 70 | 0 | 10 | 0 |
| 22 | 15 | 75 | 0 | 10 | 0 |
| 23 | 10 | 80 | 0 | 10 | 0 |
| 24 | 5 | 85 | 0 | 10 | 0 |
| 25 | 90 | 0 | 5 | 5 | 0 |
| 26 | 50 | 0 | 45 | 1 | 0 |
| 27 | 20 | 0 | 75 | 1 | 0 |
| 28 | 15 | 0 | 80 | 5 | 0 |
| 29 | 10 | 0 | 85 | 1 | 0 |
| 30 | 5 | 0 | 90 | 5 | 0 |
| 31 | 90 | 0 | 0 | 10 | 0 |
| 32 | 50 | 0 | 40 | 10 | 0 |
| 33 | 20 | 0 | 70 | 10 | 0 |
| 34 | 15 | 0 | 75 | 10 | 0 |
| 35 | 10 | 0 | 80 | 10 | 0 |
| 36 | 5 | 0 | 85 | 10 | 0 |
| 37 | 90 | 0 | 0 | 0 | 0 |
| 38 | 90 | 10 | 0 | 0 | <1 |
| 39 | 50 | 50 | 0 | 0 | <1 |
| 40 | 20 | 80 | 0 | 0 | <1 |
| 41 | 15 | 85 | 0 | 0 | <1 |
| 42 | 10 | 90 | 0 | 0 | <1 |
| 43 | 5 | 95 | 0 | 0 | <1 |
| 44 | 90 | 0 | 10 | 0 | <1 |
| 45 | 50 | 0 | 50 | 0 | <1 |
| 46 | 20 | 0 | 80 | 0 | <1 |
| 47 | 15 | 0 | 85 | 0 | <1 |
| 48 | 10 | 0 | 90 | 0 | <1 |
| 49 | 5 | 0 | 95 | 0 | <1 |
| 50 | 90 | 5 | 0 | 5 | <1 |
| 51 | 50 | 45 | 0 | 5 | <1 |
| 52 | 20 | 75 | 0 | 5 | <1 |
| 53 | 15 | 80 | 0 | 5 | <1 |
| 54 | 10 | 85 | 0 | 5 | <1 |
| 55 | 5 | 90 | 0 | 5 | <1 |
| 56 | 90 | 0 | 0 | 10 | <1 |
| 57 | 50 | 40 | 0 | 10 | <1 |
| 58 | 20 | 70 | 0 | 10 | <1 |
| 59 | 15 | 75 | 0 | 10 | <1 |
| 60 | 10 | 80 | 0 | 10 | <1 |
| 61 | 5 | 85 | 0 | 10 | <1 |
| 62 | 90 | 0 | 5 | 5 | <1 |
| 63 | 50 | 0 | 45 | 5 | <1 |
| 64 | 20 | 0 | 75 | 5 | <1 |
| 65 | 15 | 0 | 80 | 5 | 1 |
| 66 | 10 | 0 | 85 | 5 | 1 |
| 67 | 5 | 0 | 90 | 5 | <1 |
| 68 | 90 | 0 | 0 | 10 | <1 |
| 69 | 50 | 0 | 40 | 10 | <1 |
| 70 | 20 | 0 | 70 | 10 | <1 |
| 71 | 15 | 0 | 75 | 10 | <1 |
| 72 | 10 | 0 | 80 | 10 | <1 |
| 73 | 5 | 0 | 85 | 10 | <1 |
| 74 | 90 | 0 | 0 | 0 | <1 |
| 75 | 100 | 0 | 0 | 0 | 0 |
| 76 | 100 | 0 | 0 | 0 | <1 |

^{a}Nintedanib salt is any salt form described herein

### Example 3. Nintedanib Dry Powder Formulations

Five nintedanib hydrobromide formulations were manufactured by spray-drying at a 25 g scale; the process parameters for all five spray-dried blends were kept constant. Each formulation was filled into size-3 HPMC capsules and set down on stability, stored in containers and sealed in pouches, for three months at 25° C./60% RH and 40° C./75% RH. Sufficient samples for two additional time points were set down at each condition. One micronized nintedanib hydrobromide mixture was manufactured with pharmaceutical grade lactose monohydrate containing 1000 fines at a 2.5 g scale. The formulation was filled into size-3 HPMC capsules and set down on stability, stored in containers and sealed in pouches, for one month at 25° C./60% RH and 40° C./75% RH. Sufficient samples for two additional time points were set down at each condition. Physiochemical properties and aerosol performance were assessed for each formulation. HPLC assay parameters for sample analysis are detailed in Table 3.

TABLE 3
HPLC Assay Method Parameters

| Column | Waters Bridgehead C18 RP18 150 × | | |
|---|---|---|---|
| | 4.6 mm, 3.5 μm) | | |
| Wavelength | 390 nm (Including 3D scan) | | |
| Column Temperature | 45° C. | | |
| Tray Temperature | Ambient (20° C.) | | |
| Injection Volume | 5 μL | | |
| Flow Rate | 2.0 mL/min | | |
| Run Time | 12 minutes | | |
| Mobile Phase A | 10 mM ammonium acetate adjusted to | | |
| | pH 4.0 with acetic acid | | |
| Mobile Phase B | Acetonitrile | | |
| Diluent | 40/60 v/v acetonitrile/water | | |
| | Time (min) | A (%) | B (%) |
| Gradient Conditions | 0 | 85 | 15 |
| | 1.25 | 80 | 20 |
| | 4.5 | 45 | 55 |
| | 8 | 30 | 70 |
| | 9 | 85 | 15 |
| | 10.5 | 85 | 15 |

Three assay determinations were made for each assay and the mean value reported. Moisture content was measured by Karl Fischer coulometry using a Metrohm 851 Tritrando coulometer and sample oven. 50 mg of each sample was heated to 80° C. Lactose monohydrate was used as a bracketing standard. Single measurements were made for each sample. The emitted dose was measured using Dosage Unit Sampling Apparatus (DUSA) at 100 L/min. The samples were recovered using 50 mL of diluent. Ten determinations were performed and mean value reported

The particle size distribution of the emitted dose was measured using a Next Generation Impactor (NGI) at 100 L/min. Three determinations using one capsule each were performed and the mean values of fine particle dose and fine particle fraction (less than 5 microns) were calculated. All aerosol performance assessments were performed using Plastiape RS01 Monodose low resistance inhalation devices.

Particle size distribution was measured using a Malvern Mastersizer 3000. The sample for analysis (10 mg) was suspended in 0.1% lecithin in isooctane (10 mL) and sonicated to disperse the particles prior to measurement. A single sample was prepared for each measurement.

The melting behavior of the formulations was measured using a TA Instrument Discovery DSC. The sample (2-3 mg) was placed in a sample pan with a pierced lid and heated from 25° C. to 270-300° C. at 10° C./min.

The effect of moisture sorption on the formulations was measured using a Surface Measurement Systems DVS Advantage. The sample for analysis (10-20 mg) was dried at 0% RH and then the RH changed in 10% steps to a maximum of 90% RH and then returning to 0% RH. The steps changes were triggered by a change in mass with time limit: dm/dt=0.01%/min.

Scanning electron microscopy and x-ray powder diffraction were performed at the University of Bath.

The following criteria were targeted for spray dried Nintedanib formulations:
PSD D90<5 mcm
GSD <1.8
Fine Particle Dose (FPD)≤2 mg

For the micronized drug substance:
PSD D90≤5 mcm
For the capsule filling, the weight range was 28.5-31.5 mg

Spray drying was performed (Table 4) and batch yields recorded (Table 5).

TABLE 4
Spray Drying Conditions and Parameters

| Spray Drying Condition/Parameter | Value |
|---|---|
| Solids Content (% w/v) | 4 |
| Feedstock feed rate (ml/min) | 2 |
| Outlet temperature (° C.) | 75 |
| Atomization gas pressure (bar) | 2.5 |
| Atomization gas flow (L/min) | 17 |
| Drying gas pressure (bar) | 2.5 |
| Drying gas flow (kg/hr) | 17 |

TABLE 5
Yields (Spray Dried Batches)

| Composition | Yield |
|---|---|
| Trehalose:leucine:NHBr 80:10:10 wt % | 24.1 g (96%) |
| Lactose:leucine:NHBr 80:10:10 wt % | 23.9 g (96%) |
| Trehalose:NHBr 90:10 wt % | 7.1 g (28%) |
| Lactose:NHBr 90:10 wt % | 11.0 g (44%) |
| Lactose:leucine:NHBr 70:20:10 wt % | 23.9 g (96%) |

Capsule Filling (Spray Dried Powder). HPMC size 3 capsules were filled using a 3Pi fill to weight machine at 20% RH. Weight limits of 28.5 to 31.5 mg were used (Table 7).

TABLE 7
Capsule Fill Weights and Number Filled

Fill weight (mg)

| Formulation | Mean | Min | Max |
|---|---|---|---|
| Trehalose:leucine:NHBr 80:10:10 wt % | 30.31 | 28.61 | 31.50 |
| Lactose:leucine:NHBr 80:10:10 wt % | 30.76 | 28.60 | 31.50 |
| Trehalose:NHBr 90:10 wt % | 29.92 | 28.50 | 31.50 |
| Lactose:NHBr 90:10 wt % | 30.10 | 29.17 | 31.16 |
| Lactose:leucine:NHBr 70:20:10 wt % | 30.40 | 28.67 | 31.47 |

Capsules were stored in plastic securitainer pots, 10 capsules per pot, and each pot was sealed in individual foil overwraps before being placed in the stability chambers.

### API Micronisation

Nintedanib hydrobromide was micronised using a Hosokawa 50AS jet mill. Two batches were prepared and particle size distribution measured using a Mastersizer 3000 (Table 8).

TABLE 8
Micronisation Parameters and PSD of Micronised API

| Parameter/Result | Batch 1 | Batch 2 |
|---|---|---|
| Feed rate g/min | ~1 | ~2 |
| Feed pressure bar | 5 | 5 |
| Grinding pressure bar | 3 | 3 |
| Yield (g/%) | 3.3/55.6 | 3.9/65.7 |
| PSD D10 μm | 0.04 | 0.60 |
| PSD D50 μm | 1.03 | 1.51 |
| PSD D90 μm | 2.66 | 2.87 |

### Manufacture of Lactose Blend

The micronised API was hand-mixed using a spatula with lactose monohydrate, 10% fines (GRN4948 to make 2.5 g of nintedanib HBr:lactose monohydrate 10:90 wt %. 40 capsules were hand filled with 30.0+/−0.5 mg of formulation at laboratory conditions (21° C., 50% RH).

### Assessment of Micronised Drug Substance

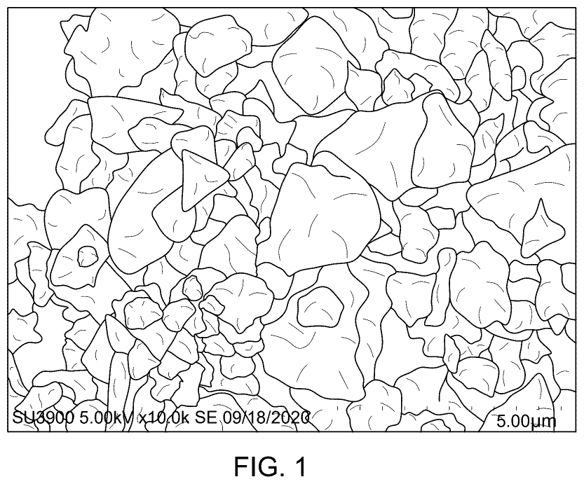

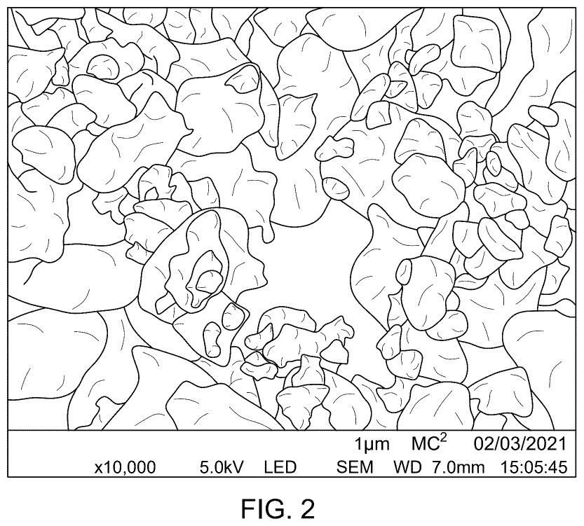

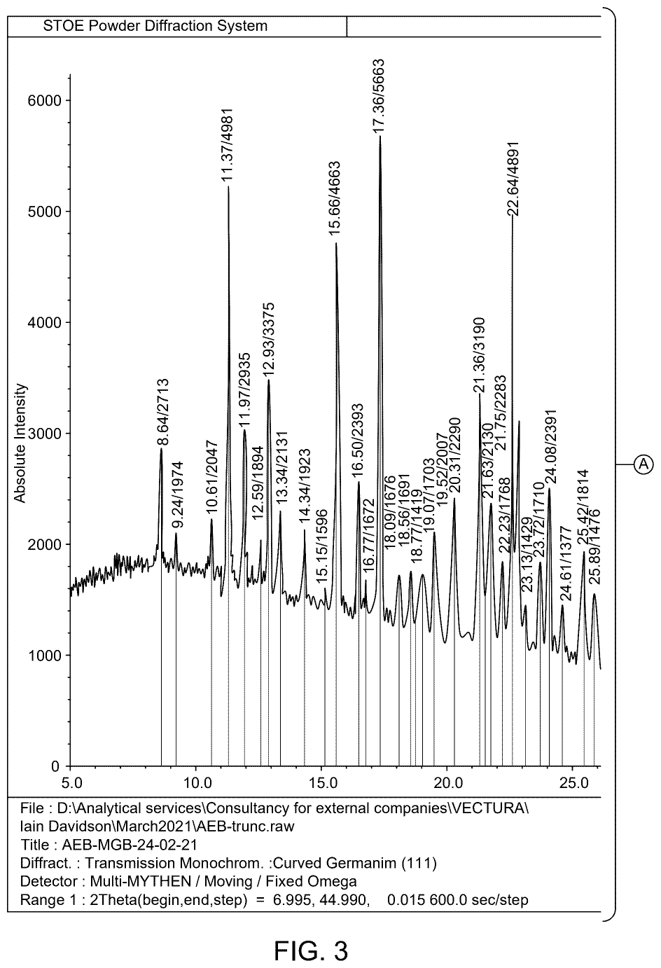

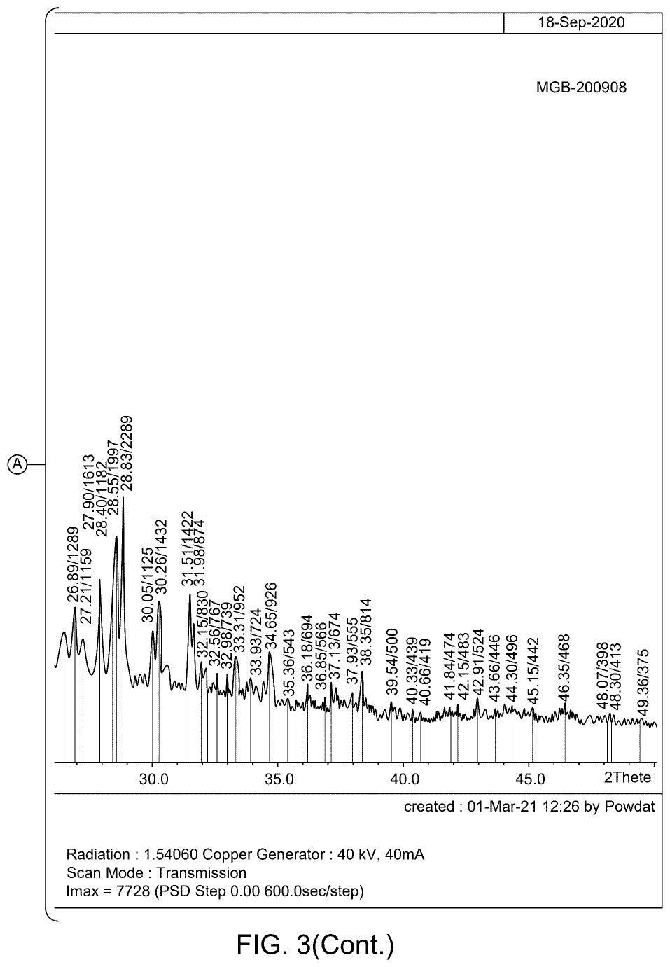

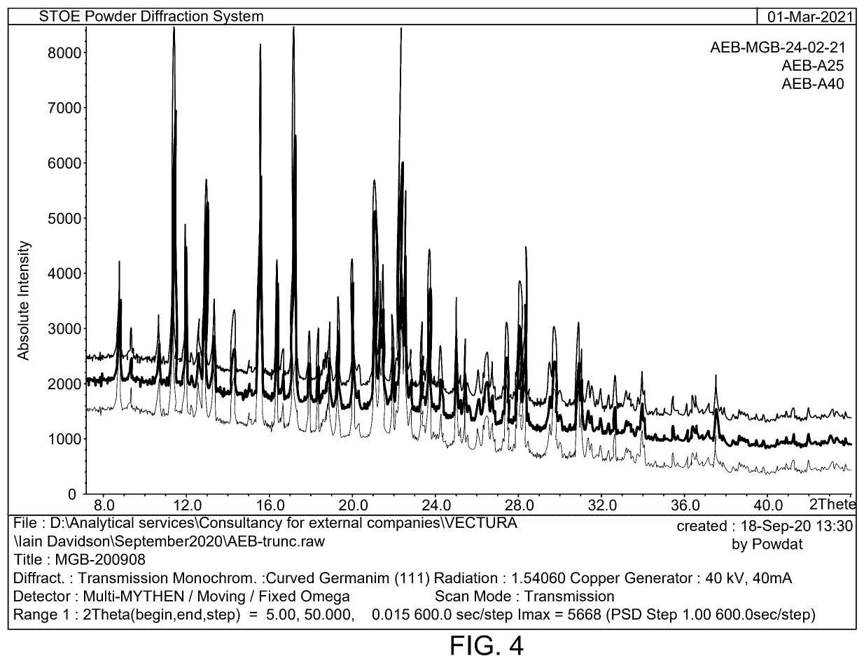

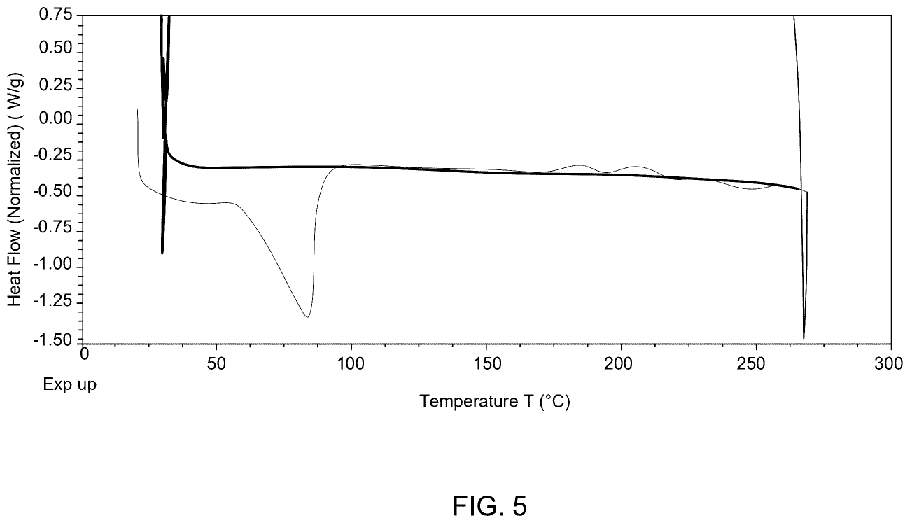

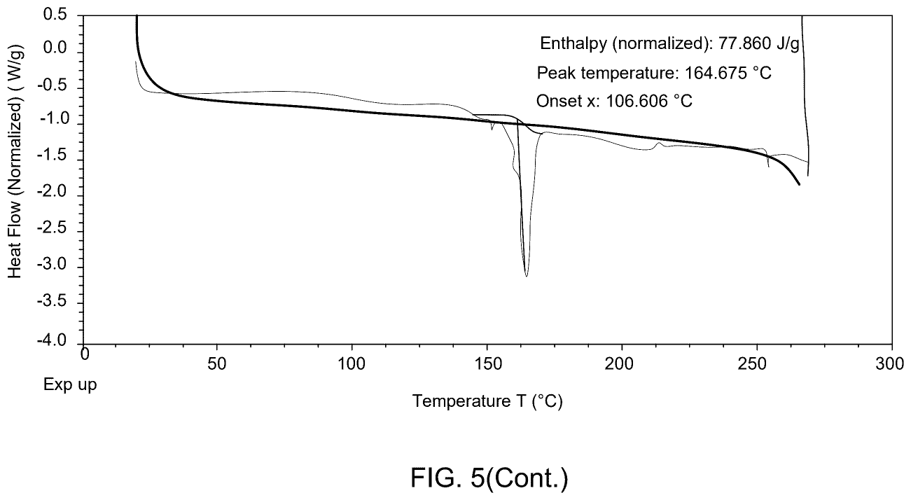

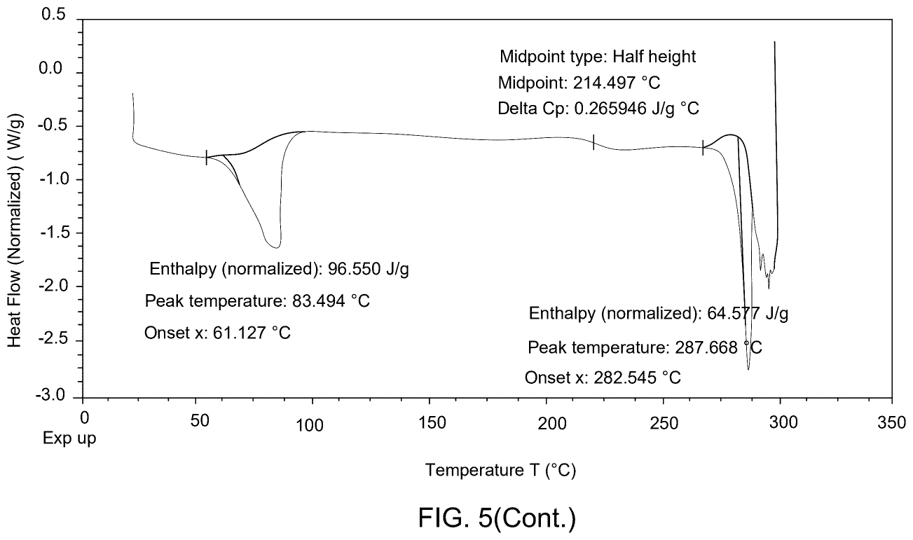

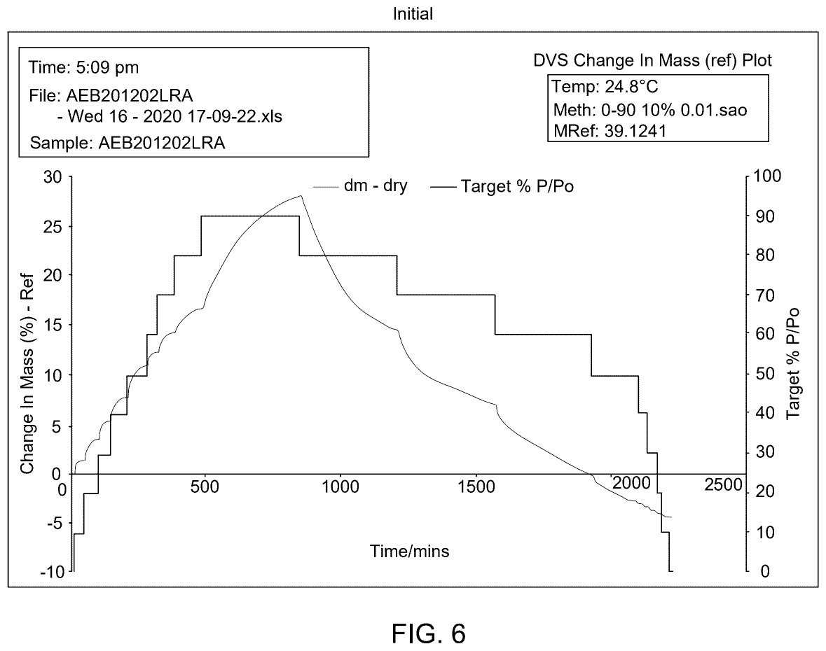

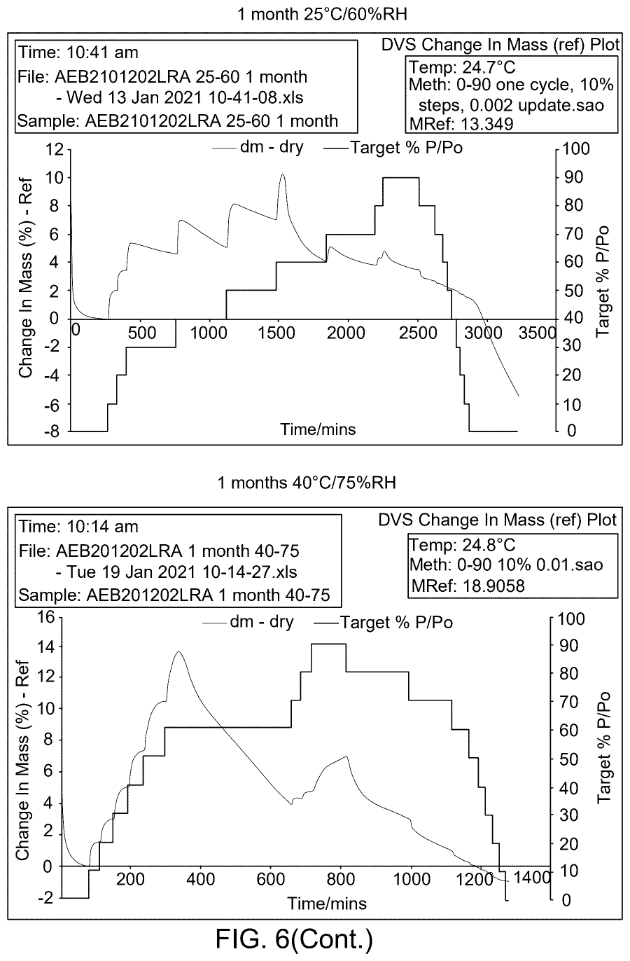

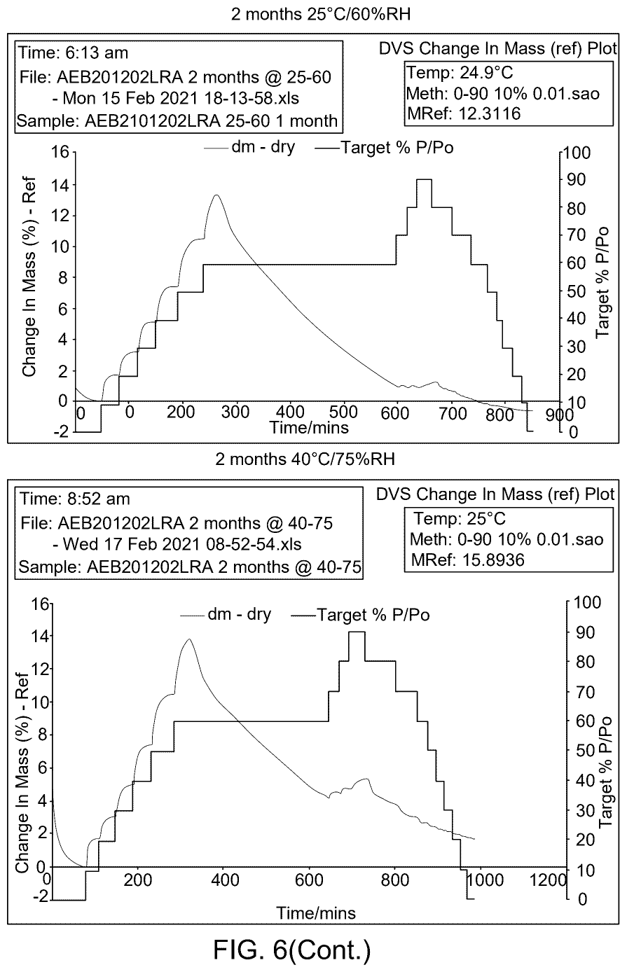

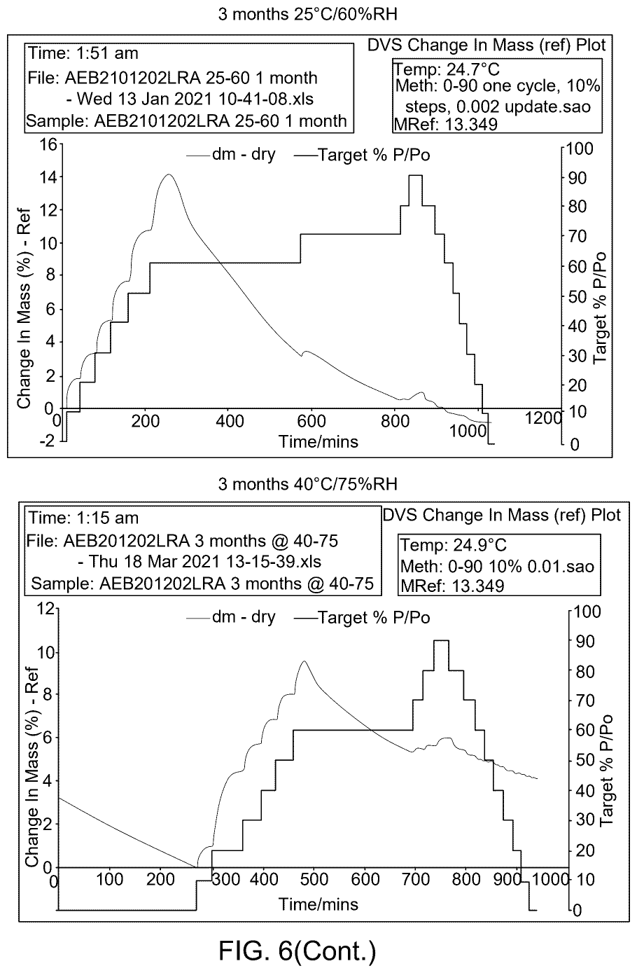

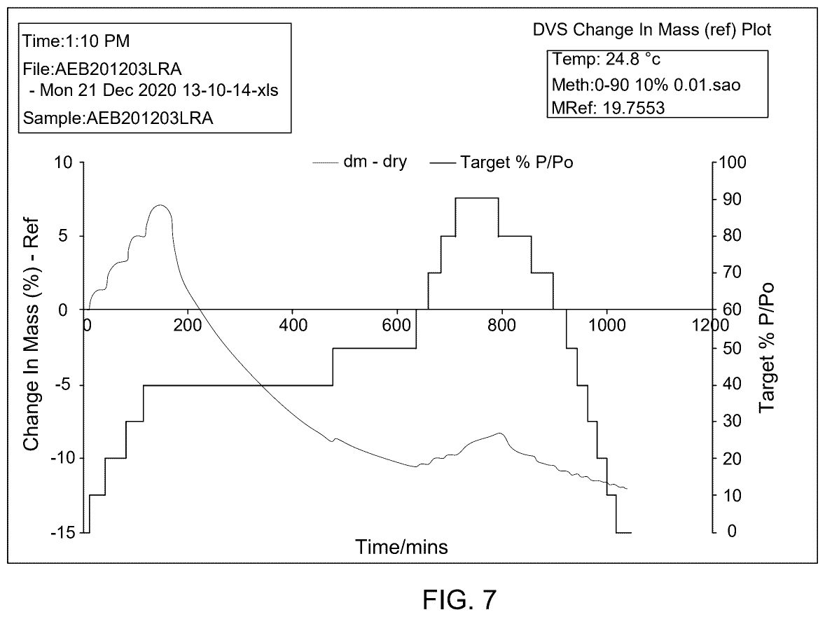

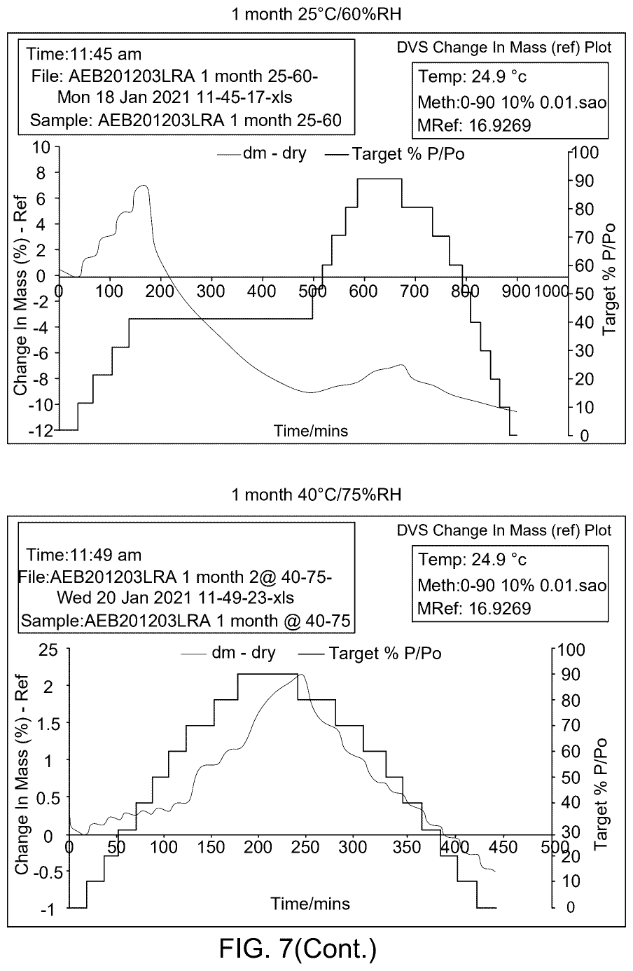

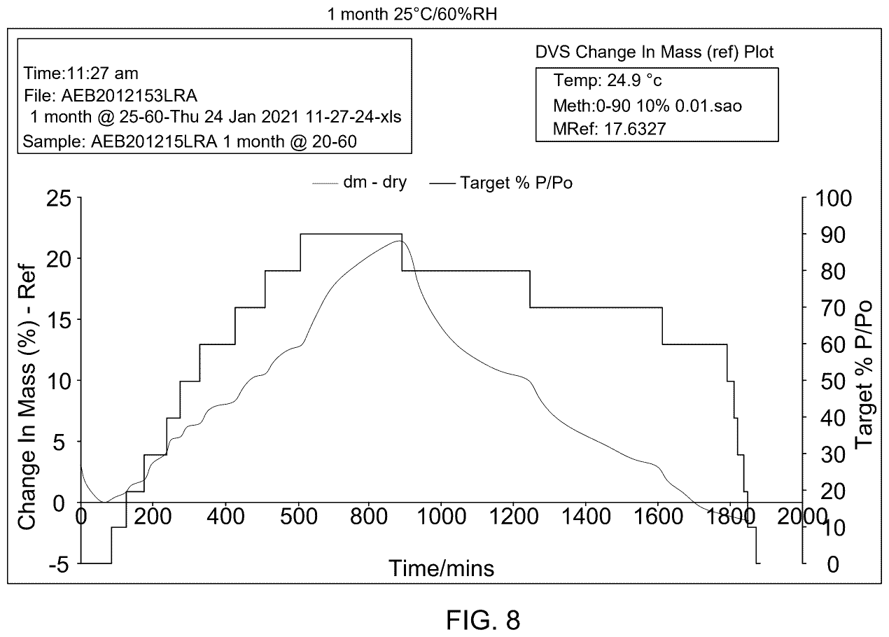

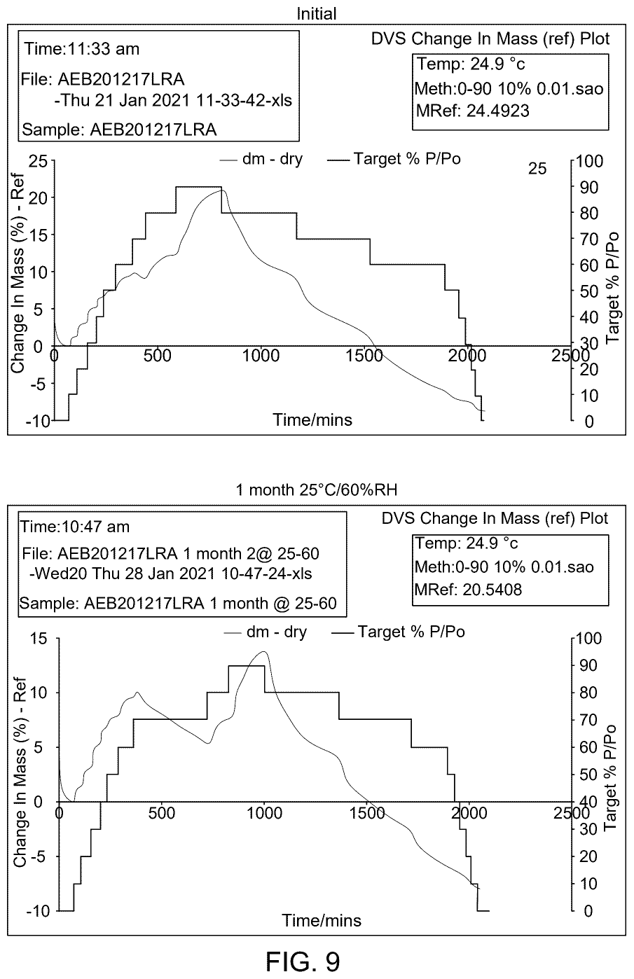

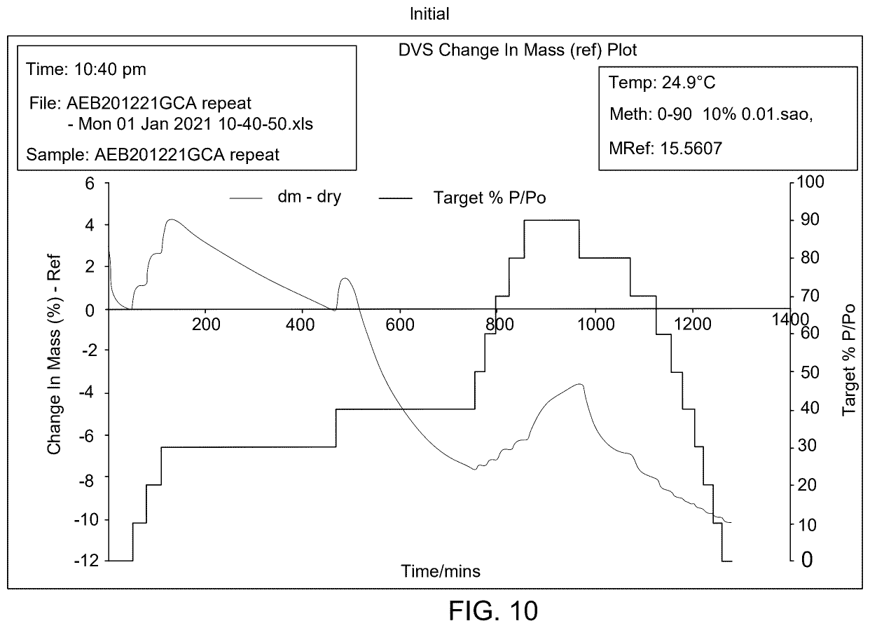

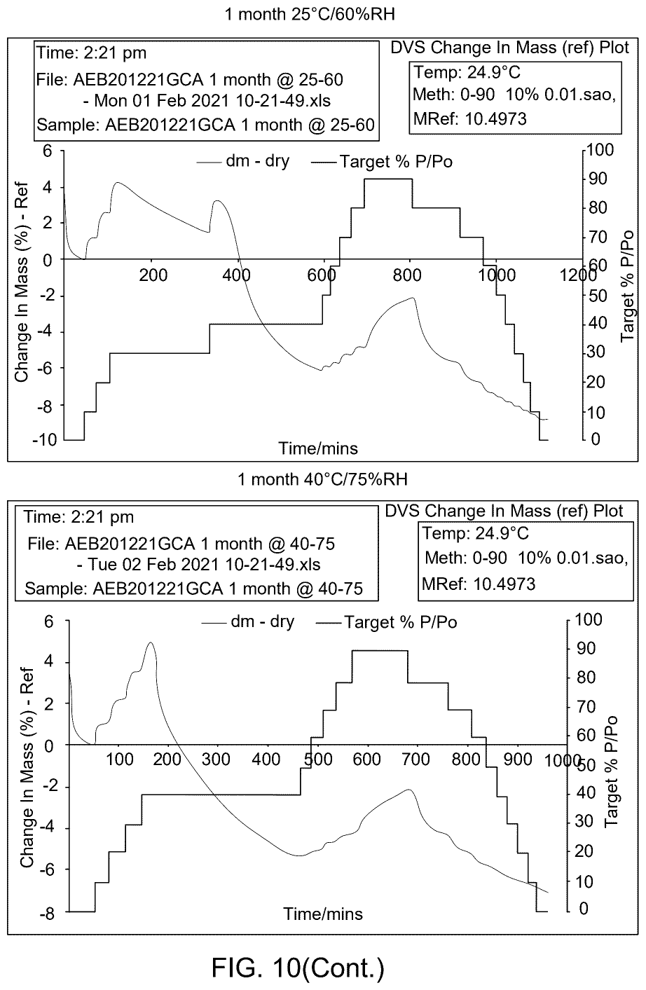

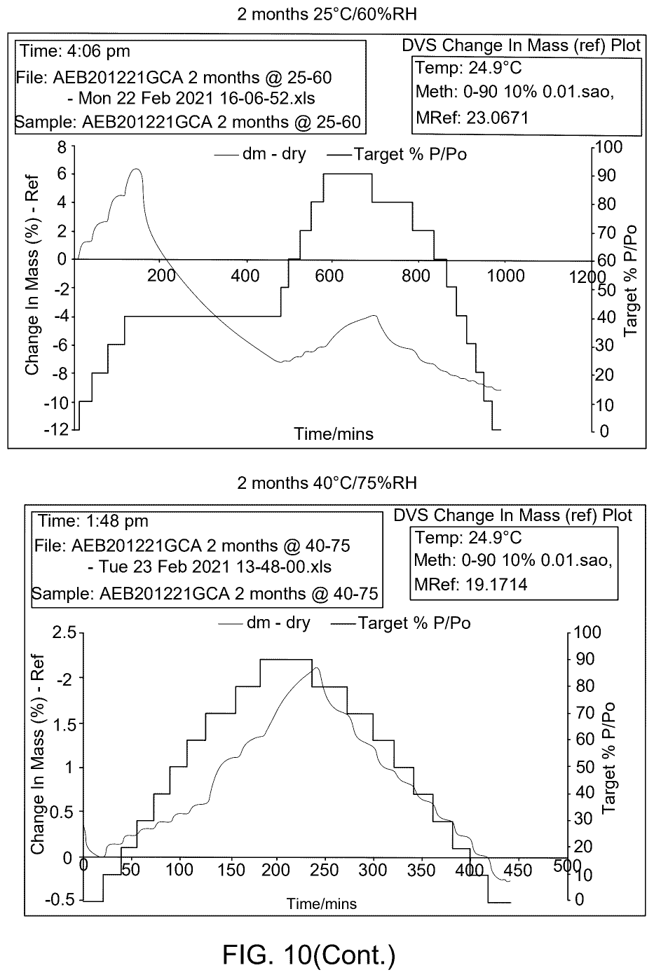

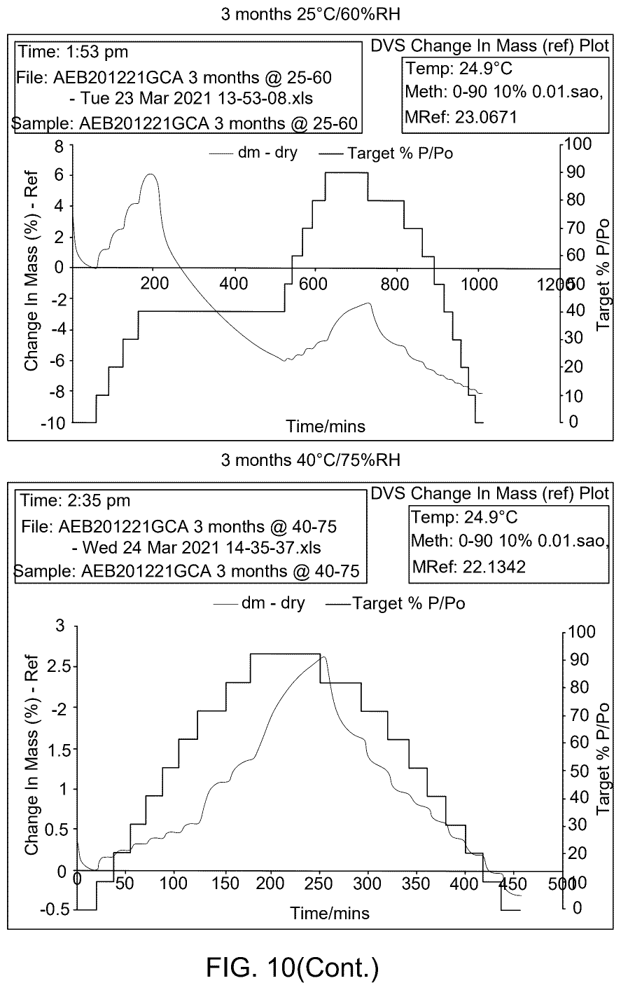

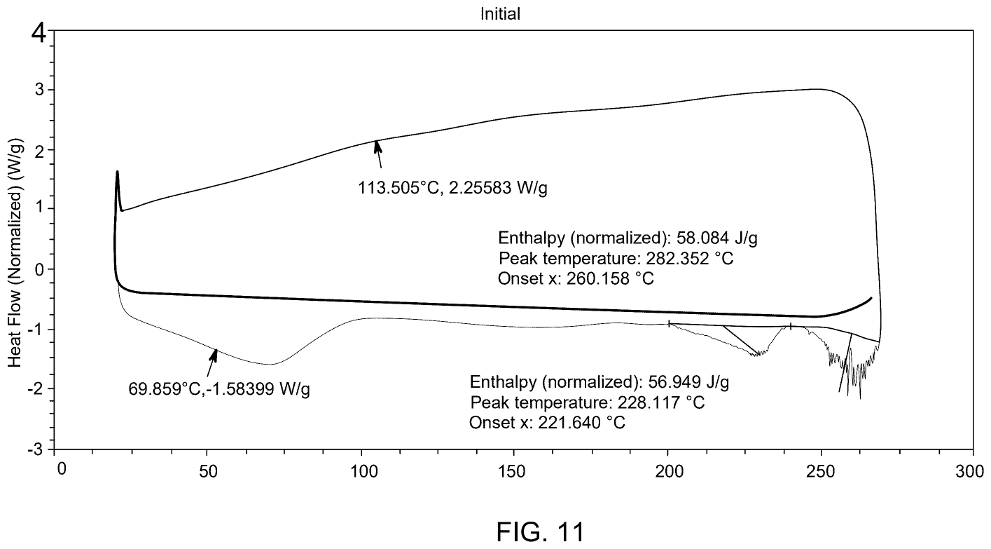

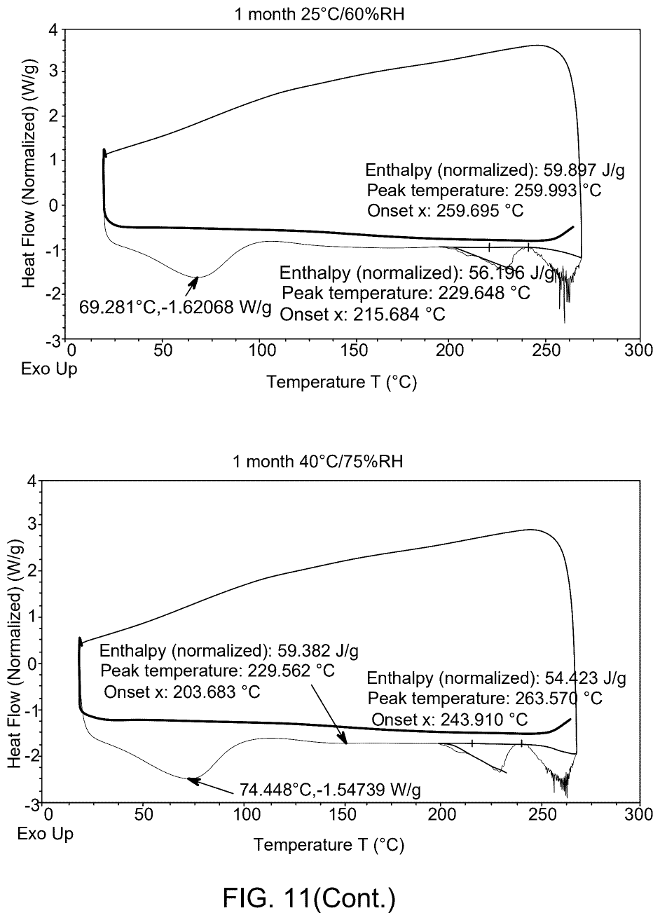

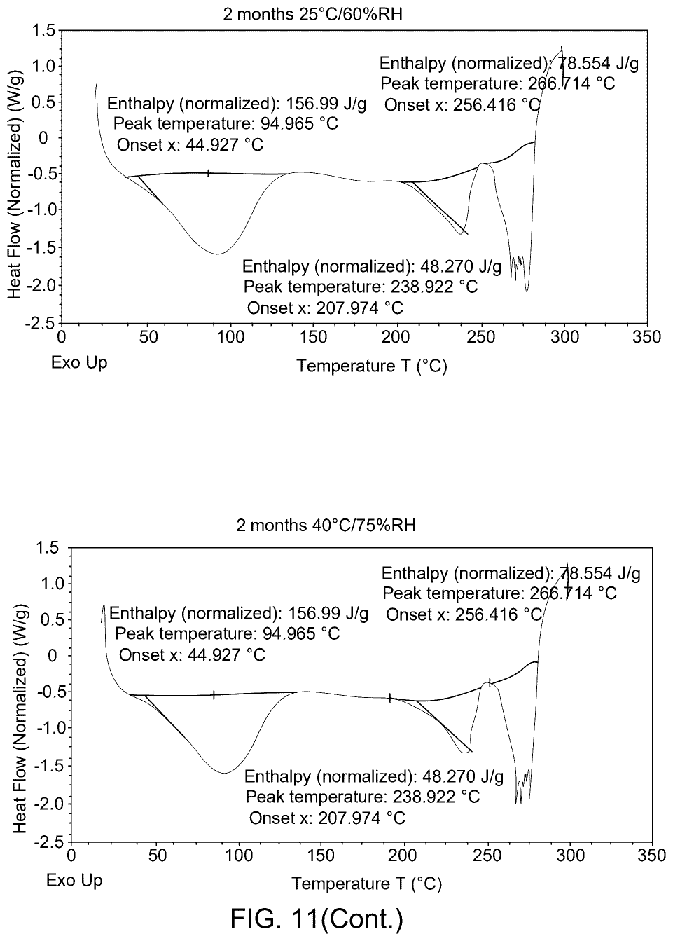

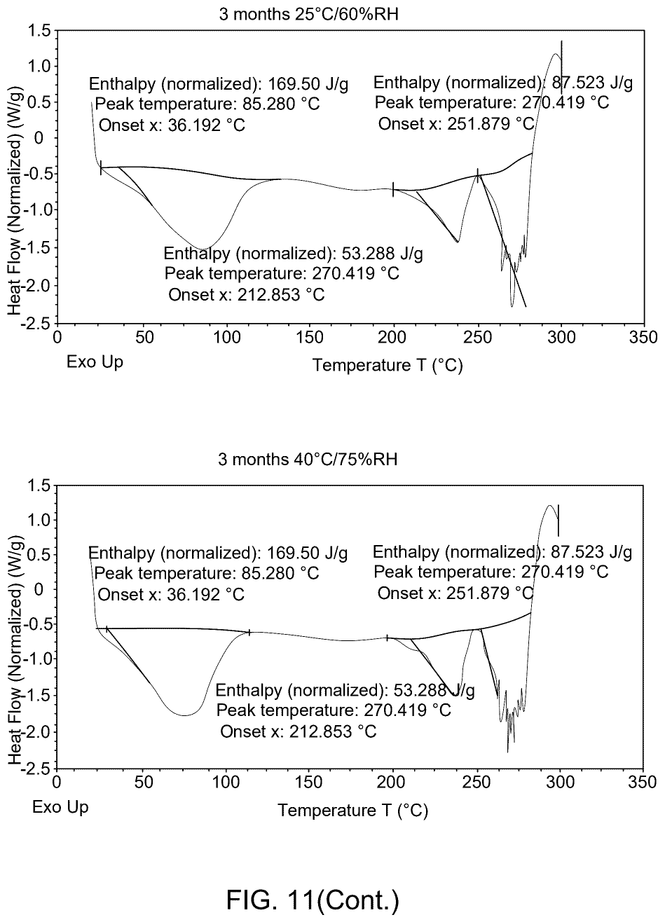

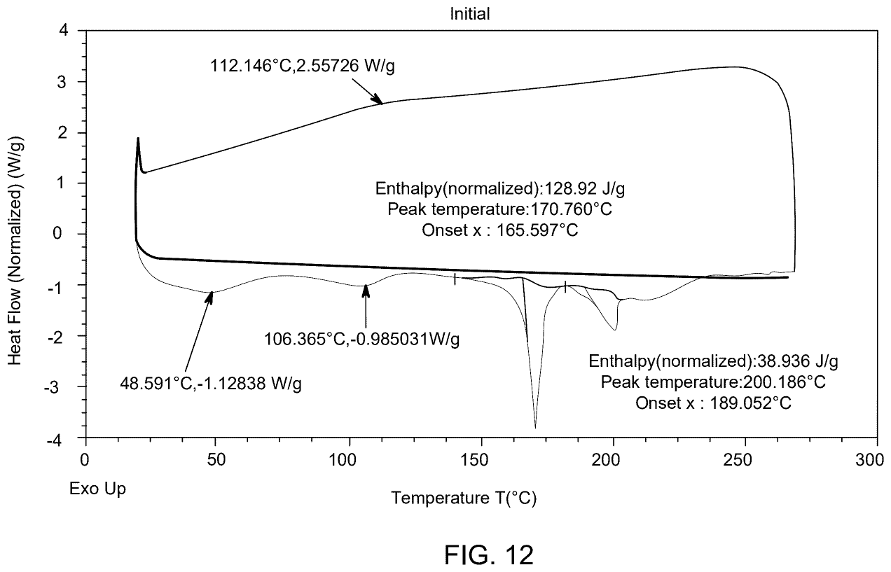

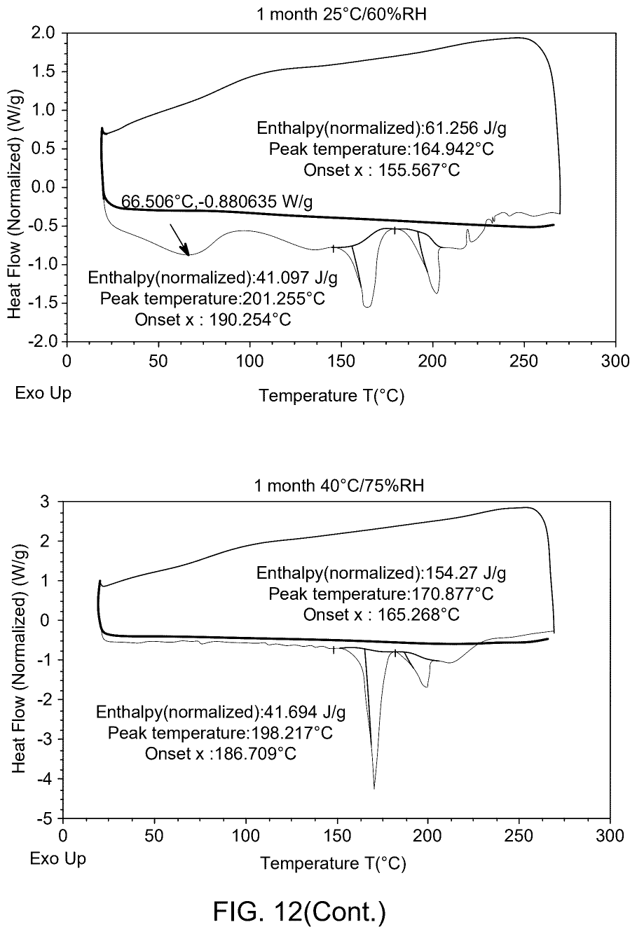

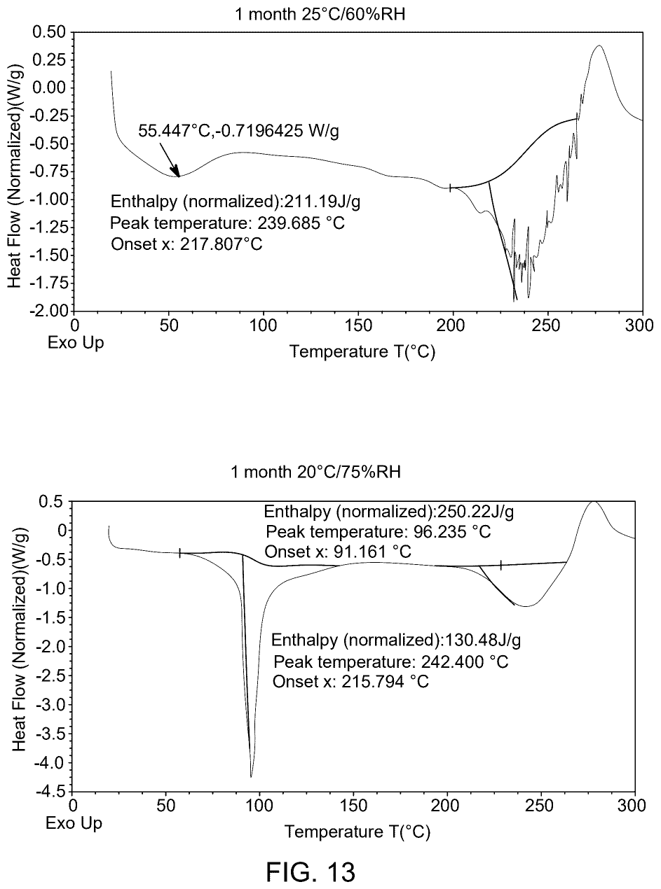

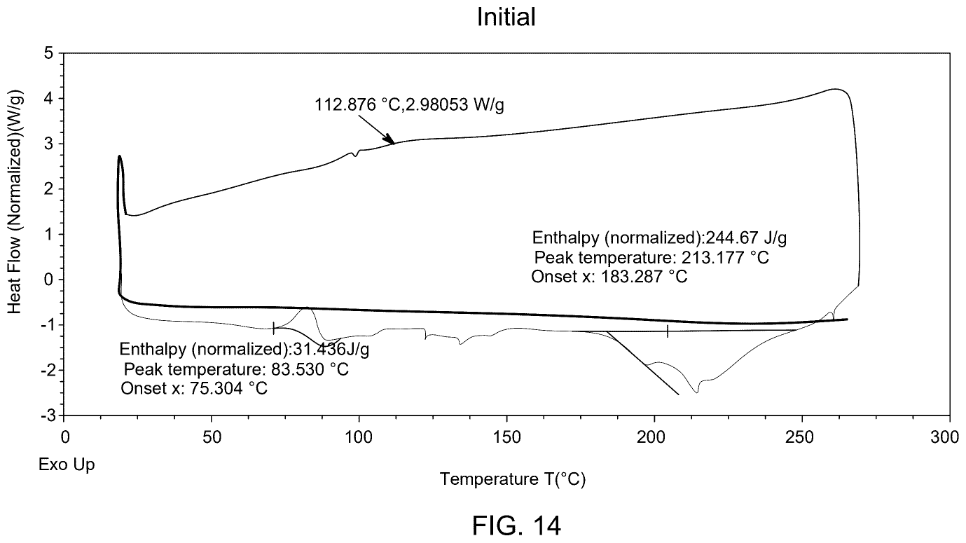

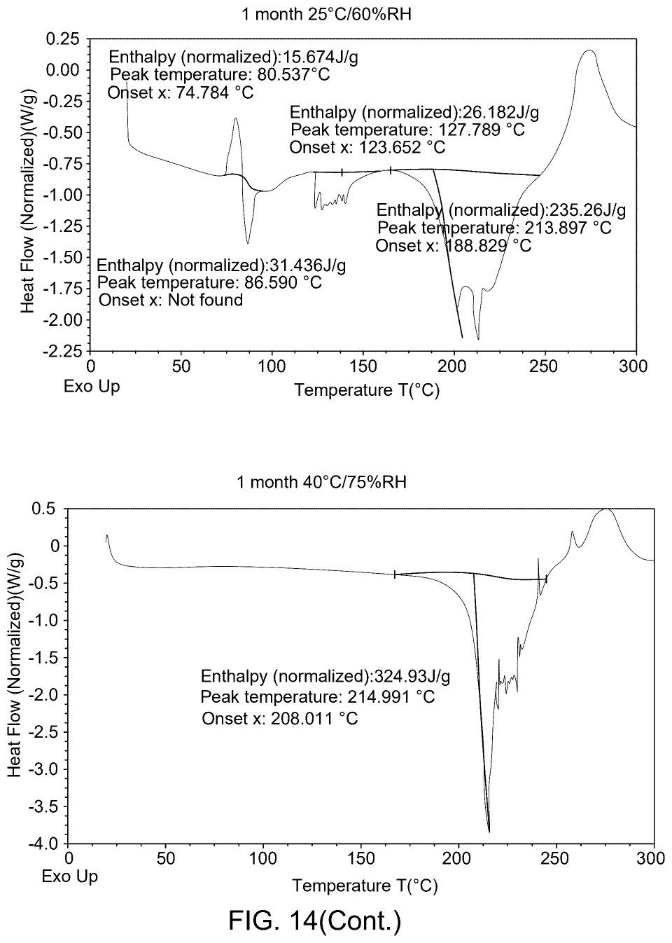

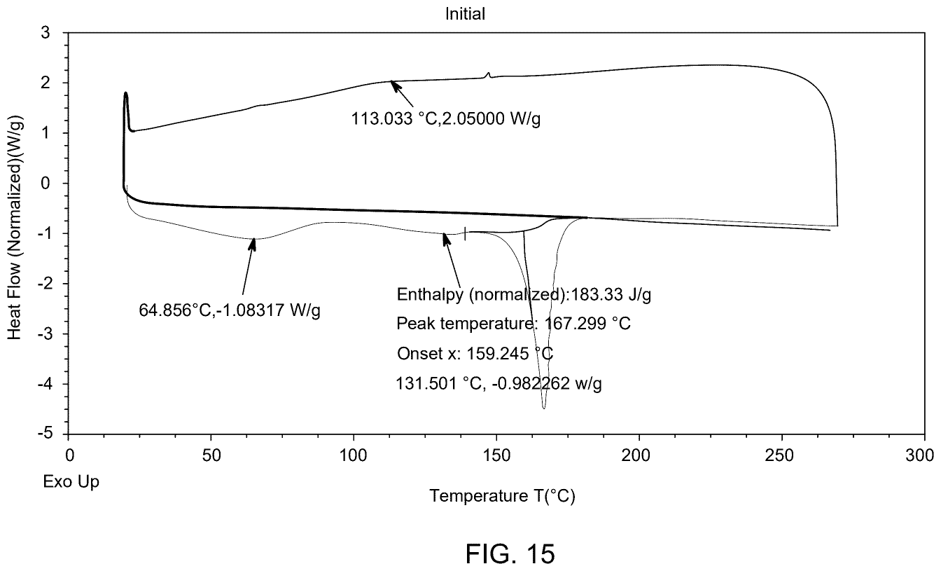

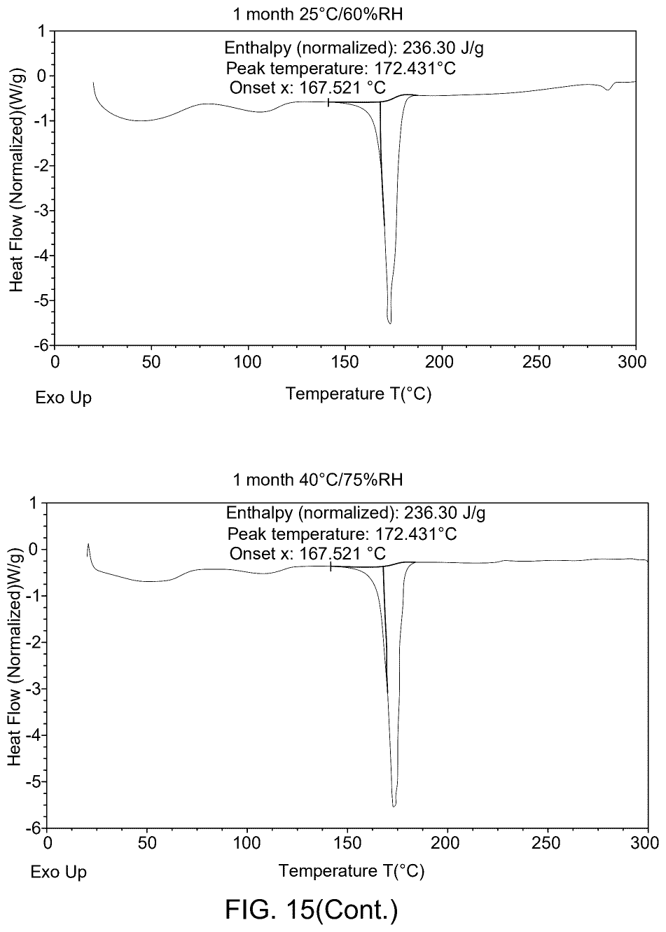

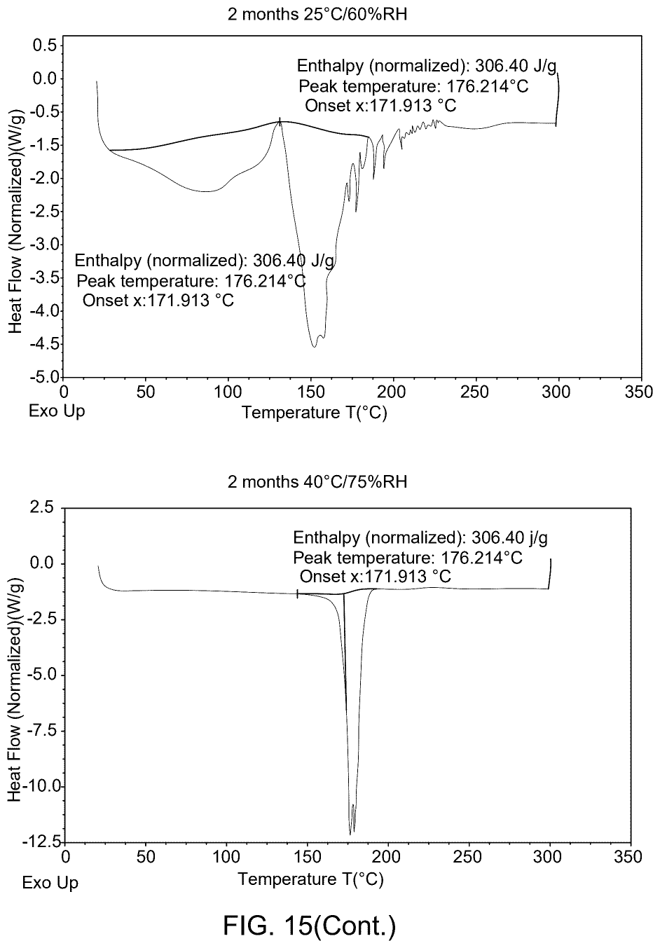

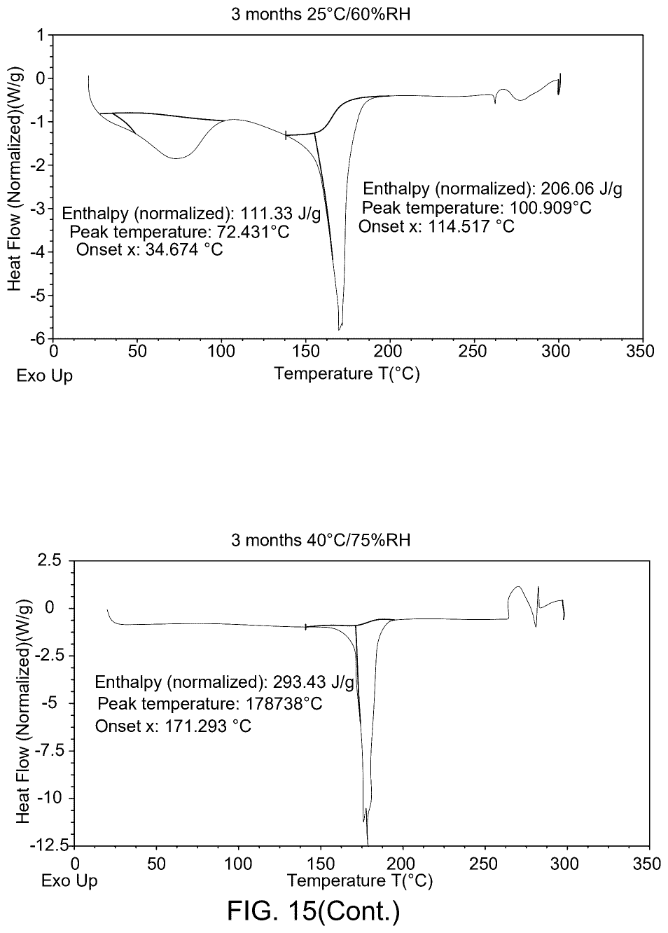

Micronised nintedanib HBr was stored in glass snap cap vials sealed in aluminum pouches at 25° C./60% RH and 40° C./75% RH for one month after which the PSD and moisture content were measured (Table 9). SEM images were recorded (FIGS. 1 and 2) and the polymorphic stability was assessed by XRPD (FIGS. 3 and 4) and DSC (FIG. 6). The characterization was then repeated after a further four months at laboratory conditions.

TABLE 9
Micronised Nintedanib HBr PSD & Moisture Analysis

| | | | | Water |
|---|---|---|---|---|
| Sample | D10 (mcm) | D50 (mcm) | D90 (mcm) | Content (%) |
| T0 | 0.5 | 1.5 | 2.8 | 5.3 |
| 1 month 25/60 | 0.6 | 1.6 | 3.0 | 5.3 |
| 1 month 40/75 | 0.6 | 1.5 | 2.6 | 5.2 |
| +4 months | 0.6 | 1.5 | 2.9 | 4.7 |

All of the results show that micronised nintedanib HBr did not change during the study. The DSC trace after one month at both 25° C./60% RH and 40° C./75% RH showed a higher, sharper melting point than at initial, but this was not seen after a further four months. XRPD showed no changes in structure for any of the samples. The change in the DSC trace at 1 month may have been due to changes in the hydration state of the sample.

It is thought that there may be an incompatibility between the drug substance and glassware; this was not assessed in this study. All future samples will be stored in plastic containers.

### Formulation Assessment—Assay

TABLE 10
Mean Assay of Nintedanib (n = 3)

| | Batch | | | | | | | | |
|---|---|---|---|---|---|---|---|---|---|
| | Mean Assay (% w/w) | | | | | | | | |
| Time point (months) | Initial * | 1 | | 2 | | 3 | 5 | | 6 |
| Condition | — | 25/60 | 40/75 | 25/60 | 40/75 | 25/60 | 40/75 | — | — |
| (° C./% RH) | | | | | | | | | |
| Trehalose:leucine: | 7.6 | 7.4 | 7.6 | 7.7 | 7.8 | 7.8 | 7.6 | — | — |
| NHBr 80:10:10 wt % | | | | | | | | | |
| Lactose:leucine: | 7.6 | 7.5 | 8.5 | — | — | — | — | — | — |
| NHBr 80:10:10 wt % | | | | | | | | | |
| Trehalose:NHBr | 7.0 | 6.8 | 6.9 | — | — | — | — | — | — |
| 90:10 wt % | | | | | | | | | |
| Lactose:NHBr | 7.3 | 7.1 | 8.4 | — | — | — | — | — | — |
| 90:10 wt % | | | | | | | | | |
| Lactose:leucine: | 8.1 | 8.01 | 9.2 | 8.2 | 9.1 | 8.4 | 9.4 | — | — |
| NHBr 70:20:10 wt % | | | | | | | | | |
| Lactose:Micronized | — | — | — | — | — | — | — | 7.9 | 8.1 |
| NHBr 90:10% | | | | | | | | | |

* single determinations only performed at initial

The nintedanib content at initial for all formulations is lower than the 8.7% maximum expected theoretical value. The theoretical value is derived from converting the hydrobromide salt to the free base which requires a moiety correction factor of 0.87 therefore giving a maximum theoretical value of 8.7% in a 100% w/w formulation.

Formulation Assessment—Moisture Content is shown in Table 11.

TABLE 11
Moisture Content (n = 1)

| | Batch | | | | | | | | |
|---|---|---|---|---|---|---|---|---|---|
| | Moisture Content (%) | | | | | | | | |
| Time point (months) | Initial | 1 | 2 | 3 | 5 | | 6 | | |
| Condition | — | 25/60 | 40/75 | 25/60 | 40/75 | 25/60 | 40/75 | — | — |
| (° C./% RH) | | | | | | | | | |
| Trehalose:leucine: | 3.7 | 2.7 | 2.2 | 3.6 | 3.6 | 3.6 | 3.7 | — | — |
| NHBr 80:10:10 | | | | | | | | | |
| wt % | | | | | | | | | |
| Lactose:leucine: | 2.9 | 3.0 | 0.3 | — | — | — | — | — | — |
| NHBr 80:10:10 | | | | | | | | | |
| wt % | | | | | | | | | |
| Trehalose:NHBr | 3.6 | 3.6 | 1.9 | — | — | — | — | — | — |
| 90:10 wt % | | | | | | | | | |
| Lactose:NHBr | 3.1 | 3.6 | 0.2 | — | — | — | — | — | — |
| 90:10 wt % | | | | | | | | | |
| Lactose:leucine: | 3.0 | 2.1 | 0.3 | 1.5 | 0.2 | 1.8 | 0.2 | — | — |
| NHBr 70:20:10 | | | | | | | | | |
| wt % | | | | | | | | | |
| Lactose:Micronized | — | 5.3 | 5.2 | — | — | — | — | 0.6 | 0.6 |
| NHBr 90:10% | | | | | | | | | |

### Formulation Assessment—Emitted Dose

TABLE 12
Mean Emitted Dose (n = 10)

| | Batch | | | | | | | | |
|---|---|---|---|---|---|---|---|---|---|
| | Emitted dose (mg) (%) | | | | | | | | |
| Time point (months) | Initial | 1 | | 2 | | 3 | | 5 | 6 |
| Condition | — | 25/60 | 40/75 | 25/60 | 40/75 | 25/60 | 40/75 | — | — |
| (° C./% RH) | | | | | | | | | |
| Trehalose:leucine: | 2.14 | 2.09 | 2.14 | 2.13 | 2.01 | 2.15 | 2.17 | — | — |
| NHBr 80:10:10 | (93%) | (91%) | (93%) | (92%) | (87%) | (93%) | (94%) | | |
| wt % | | | | | | | | | |
| Lactose:leucine: | 2.21 | 2.15 | 1.97 | — | — | — | — | — | — |
| NHBr 80:10:10 | (95%) | (92%) | (84%) | | | | | | |
| wt % | | | | | | | | | |
| Trehalose:NHBr | 2.09 | 1.67 | 1.08 | — | — | — | — | — | — |
| 90:10 wt % | (100%) | (80%) | (52%) | | | | | | |
| Lactose:NHBr | 1.94 | 1.78 | 0.25 | — | — | — | — | — | — |
| 90:10 wt % | (88%) | (81%) | (11%) | | | | | | |
| Lactose:leucine: | 2.31 | 2.25 | 2.25 | 1.54 | 1.95 | 2.24 | 2.29 | — | — |
| NHBr 70:20:10 | (94%) | (91%) | (91%) | (63%) | (79%) | (91%) | (93%) | | |
| wt % | | | | | | | | | |
| Lactose:Micronized | 2.20 | — | — | — | — | — | N/A | 2.17 | 2.12 |
| NHBr 90:10% | (93%) | | | | | | | (91%) | (89%) |

*Emitted Dose result as generated by NGI testing

Emitted dose data at initial demonstrated that all formulations were evacuated from the capsule with at least 85% recovery for all formulations. After storage, two of the spray dried batches had poor emitted dose and this, combined with other data, resulted in the study of these being discontinued. For the two spray dried formulations that were assessed after 3 months storage (at both storage conditions), and for the micronised formulation after 5 and 6 months storage, typical emitted dose data were observed.

### Formulation Assessment—Fine Particle Dose and Fine Particle Fraction (Table 13).

TABLE 13
Mean Fine Particle Dose (n = 3)

| | Batch | | | | | | | | |
|---|---|---|---|---|---|---|---|---|---|
| | Fine particle dose (<5 mcm) (mg) | | | | | | | | |
| | (Fine particle fraction) | | | | | | | | |
| Time point (months) | Initial | 1 | | 2 | | 3 | | 5 | 6 |
| Condition | — | 25/60 | 40/75 | 25/60 | 40/75 | 25/60 | 40/75 | — | — |
| (° C./% RH) | | | | | | | | | |
| Trehalose:leucine: | 0.68 | 0.58 | 0.45 | 0.52 | 0.57 | 0.52 | 0.47 | — | — |
| NHBr 80:10:10 | (32%) | (27%) | (22%) | (25%) | (28%) | (25%) | (23%) | | |
| wt % | | | | | | | | | |
| Lactose:leucine: | 0.73 | 0.19 | 0.16 | — | — | — | — | — | — |
| NHBr 80:10:10 | (36%) | (9%) | (8%) | | | | | | |
| wt % | | | | | | | | | |
| Trehalose:NHBr | 0.64 | 0.15 | 0.003 | — | — | — | — | — | — |
| 90:10 wt % | (32%) | (8%) | (0.2%) | | | | | | |
| Lactose:NHBr | 0.47 | 0.52 | 0.003 | — | — | — | — | — | — |
| 90:10 wt % | (23%) | (25%) | (0.3%) | | | | | | |
| Lactose:leucine: | 0.56 | 0.80 | 0.55 | 0.76 | 0.52 | 0.94 | 0.59 | — | — |
| NHBr 70:20:10 | (27%) | (40%) | (25%) | (41%) | (24%) | (47%) | (28%) | | |
| wt % | | | | | | | | | |
| Lactose:Micronized | 1.32 | — | — | — | — | — | — | 1.30 | 1.30 |
| NHBr 90:10% | (60%) | | | | | | | (60%) | (61%) |

Initial data shows the spray dried formulations had FPF in the range of 23-36%. The micronized drug/lactose formulation had a higher performance and had an FPF of 600%. Further analysis after 1 month storage at 25° C./60% RH and 40° C./75% RH indicated significant change in aerosol performance for three of the spray dried formulations resulting in these being discontinued from further analysis. Of the two spray dried formulations that were assessed after 2 and 3 months storage at 25° C./60% RH and 40° C./75% RH, Trehalose:leucine:NHBr 80:10:10 wt % demonstrated consistent performance (22-28% FPF after storage at 25° C./60% RH and 40° C./75% RH compared to 3200 FPF at initial), with no demonstrable trend on storage at either condition. Lactose:leucine:NHBr 70:20:10 wt %⁰ had variable performance, with a noticeable difference in FPF at the different storage conditions. Notably, the FPF appeared to be greater for the when stored at 25° C./60% RH.

The micronised drug/lactose batch after 5 and 6 months storage at 25° C./60% RH and 40° C./75% RH demonstrated consistent physical performance compared to initial, with the FPF remaining around 6000.

Physical Properties—Stability of Particle Size Distribution by Laser Diffraction is shown in Table 14.

TABLE 14
Particle size by laser diffraction

| | Batch | | | | | | | | | | | | | | |
|---|---|---|---|---|---|---|---|---|---|---|---|---|---|---|---|
| | Trehalose:leucine: | | | Lactose:leucine: | | | Lactose:leucine: | | | | | | | | |
| | NHBr 80:10:10 | | | NHBr 80:10:10 | | | NHBr 70:20:10 | | | Trehalose:NHBr | | | Lactose:NHBr | | |
| | wt % | | | wt % | | | wt % | | | 90:10 wt % | | | 90:10 wt % | | |
| Mcm | D 10 | D 50 | D 90 | D 10 | D 50 | D 90 | D 10 | D 50 | D 90 | D 10 | D 50 | D 90 | D 10 | D 50 | D 90 |
| Initial | 1.0 | 2.2 | 4.6 | 1.0 | 2.3 | 4.9 | 1.1 | 2.7 | 5.6 | — | — | — | 1.1 | 3.3 | 9.7 |
| 1 mth | 1.7 | 3.1 | 5.5 | 2.9 | 5.6 | 10.4 | 1.8 | 3.2 | 5.7 | 3.5 | 8.2 | 16.4 | 2.0 | 4.5 | 10.7 |
| 25/60 | | | | | | | | | | | | | | | |
| 1 mth | 1.7 | 3.1 | 5.6 | 2.8 | 5.8 | 10.9 | 1.9 | 3.2 | 5.6 | — | — | — | — | — | — |
| 40/75 | | | | | | | | | | | | | | | |
| 2 mth | 1.7 | 3.2 | 5.8 | — | — | — | 1.8 | 3.1 | 5.5 | — | — | — | — | — | — |
| 25/60 | | | | | | | | | | | | | | | |
| 2 mth | 1.8 | 3.3 | 6.2 | — | — | — | 1.9 | 3.4 | 5.9 | — | — | — | — | — | — |
| 40/75 | | | | | | | | | | | | | | | |
| 3 mth | 1.1 | 2.5 | 5.5 | — | — | — | 1.1 | 2.6 | 5.4 | — | — | — | — | — | — |
| 25/60 | | | | | | | | | | | | | | | |
| 3 mth | 1.1 | 2.7 | 6.1 | — | — | — | 1.1 | 3.2 | 6.1 | — | — | — | — | — | — |
| 40/75 | | | | | | | | | | | | | | | |

Batches Trehalose:NHBr 90:10 wt % and Lactose:NHBr 90:10 wt % showed a change in particle size after 1 month storage at 25° C./60% RH due to agglomeration of the particles and at 40° C./75% RH the samples were fused so that no measurement was possible. After two months storage at 25° C./60% RH and 40° C./75% RH, samples at both conditions had completely agglomerated. These batches did not contain leucine in the matrix which is known to form a hydrophobic surface layer.

Batch Lactose:leucine:NHBr 80:10:10 wt % showed an increase in size at both storage conditions after 1 month. After two months the samples at both conditions had agglomerated so that no measurement was possible. This batch contains lactose and so is more susceptible to moisture driven changes than the equivalent trehalose batch, Trehalose:leucine:NHBr 80:10:10 wt %.

Batches Trehalose:leucine:NHBr 80:10:10 wt % containing trehalose and 10% leucine, and Lactose:leucine:NHBr 70:20:10 wt % containing lactose and 20% leucine, showed a similar small increase in particle size during the course of the study and showed no signs of significant agglomeration.

DVS results are shown in FIG. 6.

Initially the sample was amorphous, taking up 25% by weight of water with no sign of crystallization. At all subsequent timepoints at both storage conditions, the sample was amorphous but recrystallized above 60% RH, shown by the loss in mass. At 40° C./75% RH after 2 and 3 months storage the samples showed the increase in final mass expected as amorphous trehalose forms the crystalline trehalose dihydrate.

The sample was initially amorphous and crystallized above 30% RH, leading to a large loss in mass. Identical behavior was seen after 1 month storage at 25° C./60% RH but at 40° C./75% RH the sample had crystallized. No further testing was done for this formulation.

The initial sample had agglomerated prior to analysis and so no testing was done. At 1 month the sample stored at 25° C./60% RH was entirely amorphous, 20% water adsorbed, and showed no recrystallisation. However, the sample stored at 40° C./75% RH had completely agglomerated and so no testing could be done. No further testing was done for this formulation.

Initially the formulation was amorphous, 20% water absorbed, and did not recrystallize. After 1 month storage at 25° C./60% RH, the sample remained amorphous but crystallized above 60% RH and showed a large mass loss. The sample stored at 40° C./75% RH had completely agglomerated and so no testing could be done. No further testing was done for this formulation.

Initially the sample was amorphous but crystallized above 30% RH with a large mass loss. Similar behavior was seen after 1 month storage at both conditions and after 2 and 3 months at 25° C./60% RH. After 2 and 3 months at 40° C./75% RH, the sample was entirely crystalline, only absorbing 2.5% of water and showed no sign of recrystallisation.

### DSC

The DSC thermogram shows multiple transitions with no evidence of crystalline material. The thermogram is the same for all time points at each conditions. Heating and cooling cycles have been provided for initial and at one month. For 2 and 3 months heating cycles only are shown.

Initially the thermogram shows two broad events and melts at 170° C. and 210° C. A similar pattern was seen for the sample stored at 25° C./60% RH for 1 month but the sample stored for 1 month at 40° C./75% RH showed only the two melts, indicating that this sample had crystallized. No further testing was done for this formulation.

The initial sample was not analyzed as it had absorbed moisture and crystallized prior to testing After 1 month at 25° C./60% RH the sample gave broad transitions typical of an amorphous structure. The samples stored at 40° C./75% RH had crystallized, showing a sharp melt possibly due to trehalose dihydrate. No further testing was done for this formulation.

The thermograms for the initial sample and that stored at 25° C./60% RH for 1 month showed complex, broad transitions. The samples stored at 40° C./75% RH had sharp melt at 215° C., possible due to crystalline anhydrous lactose. No further testing was done for this formulation.

Initially this formulation showed broad moisture loss peaks and a sharp melt around 170-175° C. Similar thermograms are seen at 25° C./60% RH at each time point and at 40° C./75% RH after 1 month. After 2 and 3 months at 40° C./75% RH, the thermograms show only the sharp melt around 175° C., indicating that these samples had crystallized.

Initial assessment of the five spray dried formulations and a micronised drug substance/lactose formulation indicated that the spray dried batches had an FPF of 20-30% which is lower than ideal for a DPI formulation. In contrast, the micronised formulation had an FPF of 60% which is more typical for a DPI product.

Following assessment after 1 month storage it was determined that three out of the five spray dried batches (Lactose:L-leucine:Nintedanib Hydrobromide 80:10:10 wt %, Trehalose:Nintedanib Hydrobromide, 90:10 wt % and Lactose:Nintedanib Hydrobromide, 90:10 wt %) contained significant amounts of agglomerated particles and therefore a reduced aerosol performance was observed (max. FPF <25% at 25/60, less at accelerated stability condition). No further analysis was carried out on these batches.

In comparison, the micronised drug substance/lactose formulation, which was assessed after 5 and 6 months storage (in ambient conditions) demonstrated a similar performance to the initial time point.

### Example 4: Inhaled (Liquid Nebulized) Nintedanib, Inhaled (Liquid Nebulized) Nintedanib Plus Pirfenidone Fixed Combination, and Single-Dose Oral (Gavage) Nintedanib Pharmacokinetics in Sheep

Merino cross-bred female sheep (ewes), 6-12 months of age and live-weight of 30-40 kg, were used in this study; oral dosing optimization was performed on n=4 animals, while a separate group of n=10 sheep were selected for the Main (drug dosing) study. All animals had received standard vaccinations and were treated orally with anthelminthic (in line with standard management practices) prior to arrival at the animal facility to eliminate any worm parasites before experimentation. Experimental animals were rotated between indoor small group pen housing and metabolism cages under controlled ambient conditions (20-22° C.) and maintained on a 12 h light/dark throughout the experimental period. Sheep in the Main study were randomly allocated to one of three groups (Group 1 or Group 2, n=5/group).

### Oral Dosing Optimization

A procedure for closure of the reticular groove in sheep was optimized to enable oral drug delivery directly into the abomasum or true stomach. This procedure involved oral administration of a solution of 10% copper sulphate (w/v, 20 mL) to the back of the throat using a drenching gun. This was followed by the oral delivery (via an esophageal feeding tube) of 300 mL of a glucose solution (delivering 75 g glucose), and subsequent monitoring of blood glucose levels to confirm direct delivery into the abomasum following closure of the reticular groove. Blood samples collected at pre- (0 min), and at 15 min, 30 min and 45 min post-glucose administration were immediately tested using a standard glucose test strip and digital blood glucose analyzer/reader.

Main Study: Nebulized Vs. Oral Delivered Pharmacokinetics

Nintedanib salt formulations and nintedanib in fixed combination with pirfenidone for inhalation and oral dosing are listed in Table 15. All formulations used in this study were stored at room temperature (RT) and protected from light prior to use; drugs were prepared on day of use.

TABLE 15
Drug preparations and Treatment allocation

| Treatment (Route) | Dose Solution (mg/mL) |
|---|---|
| Treatment 1 & 1B (Inhalation) | Nintedanib (0.25) |
| Treatment 2 (Inhalation) | Nintedanib (0.25) + Pirfenidone (12.5) |
| Treatment 3 (Oral) | Nintedanib (3.0) |

Sheep arrived into the facility as one group and were acclimatized over 2-3 weeks (group housing to indoor cage/pens) prior to experimentation. Sheep allocated to Group 1 (n=5; sheep ID #11, 12, 13, 14, 16) or Group 2 (n=5; sheep ID #2, 17, 18, 20, 21) received Treatments 1, 2 and 3. Group 1 sheep also received Treatment 1b, an addition/modification to the original study protocol. Treatments were administered over Weeks 3-9 of the study; each experimental Treatment was performed over a one week period in either Group 1 or Group 2 sheep (e.g., one group received Treatment while the other group was rested).

Group 1 and Group 2 sheep were allocated separate sampling schedules for peripheral blood and bronchoalveolar lavage fluid (BALF) collections (Table 16).

For all drug dosing procedures, sheep were removed from their metabolism cage and positioned in a specialized restraining harness to restrict movement of the head and neck and to facilitate drug administration and BALF sample collections.

### Nebulized Inhaled Dosing

For nebulized aerosol drug administration to the lungs (inhalation dosing), a lubricated cuffed endotracheal (ET) tube (Portex, 7.0-8.0 mm internal diameter) was inserted via the nasal passage (guided by a fibre-optic endoscope) into the trachea. Nintedanib (Treatment 1 and 1b) and nintedanib in fixed combination with Pirfenidone (Treatment 2) formulations were aerosolized via an eFlow Inline nebulizer (PARI Pharma GmbH) placed in line with a dual phase control ventilator/respirator (Harvard Apparatus, MA, USA), providing a closed respiratory loop.

Respiration was set to 20 breaths/min, 50% inspiration and a tidal volume of 350 mL. Filters (Hudson RCl, NC, USA) were placed in the expiratory line to collect any expired drug dose. Following drug administration all lines, filters and nebulizer components were rinsed with 50 mL sterile saline to collect any remaining or expired dose. Washout sample aliquots (500 μl) were frozen on dry ice in 1.5 mL Eppendorf tubes, then stored at −80° C. prior to shipment for drug content analyses.

The precise start and finish times for the dose nebulization, and the final duration of the inhaled dose were accurately recorded.

### Oral Dosing

Oral drug administration (Treatment 3) followed the procedure as detailed below (oral dosing optimization). A feeding tube (7 mm internal diameter) was inserted via the nasal passage (guided by a fibre-optic endoscope) into the upper region of the esophagus. CuSO4 was administered to the back of the throat using a drenching gun, followed 40 seconds later by delivery of 25 mL of the nintedanib solution (3 mg/mL) through the feeding tube. Oral nintedanib dosing was immediately followed by oral delivery of 300 mL of glucose solution (‘chaser’) through the same feeding tube (including a thorough rinse of the 50 mL Falcon™ tube containing the nintedanib preparation) over a period of 20-25 seconds.

### Peripheral Blood Collections

Blood samples collected prior to and following drug administration (Table 16) were placed into K3EDTA-coated tubes. Cell-free plasma sample aliquots (500 mcl) were frozen on dry ice in 1.5 mL Eppendorf tubes, then stored at −80° C. prior to shipment for drug content analyses.

### Bronchoalveolar Lavage Fluid (BALF) Collections

BALF samples were collected prior to and following drug administration (Table 2). Sampling was carried out by intra-lung infusion of 25 ml of sterile saline via a catheter through the biopsy port of the bronchoscope, followed by immediate retrieval of the BALF sample into the collection syringe.

BALF was collected from separate lung segments/lobes to avoid contamination between sampling time-points: the right apical lobe (RA) was used for all pre-dose sampling and post-dose samples were collected from right middle (RM); right caudal (RC); left caudal (LC); left middle (LM); and left apical (LA) lung lobes. BALF sampling details (volumes, exact time of collection) were recorded and samples immediately frozen on dry ice, then stored at −80° C. prior to shipment for drug content analyses.

TABLE 16
Blood and BALF sampling schedule for Group 1 and Group 2 sheep
Blood collection¹ time-points (minutes post-dose)

| | | | | | | | | | | | | | | | |
|---|---|---|---|---|---|---|---|---|---|---|---|---|---|---|---|
| Treatment | pre- | 0 | 2 | 10 | 30 | 45 | 60 | 120 | 240 | 360 | 480 | 600 | 720 | 1440 | 2880 |

| | |
|---|---|
| 1 | Grp 1 |
| | Grp 2 |
| 1b | Grp 1 |
| | Grp 2 |
| 2 | Grp 1 |
| | Grp 2 |

BALF collection time-points (minutes post-dose) and lung lobes²

| Treatment | | pre- | 0 | 2 | 10 | 30 | 45 | 60 | 120 | 240 | 360 | 480 | 600 | 720 | 1440 | 2880 |
|---|---|---|---|---|---|---|---|---|---|---|---|---|---|---|---|---|
| 1 | Grp 1 | RA | | | LM | RC | | LC | | RM | | | | LA | | |
| | Grp 2 | RA | | | | | LM | | RC | | | LC | | RM | LA | |
| 1b | Grp 1 | RA | | RM | LM | RC | | LC | | | | | | | | |
| | Grp 2 | | | | | | | | | | | | | | | |
| 2 | Group 1 | RA | | | | LM | | | RC | | LC | | RM | | LA | |
| | Group 2 | RA | | | | | | LM | | RC | | LC | | RM | | LA |

¹Blood and BALF collection times highlighted;
²Lung lobes sampled: RA = right apical; LM = left middle; RC = right caudal; LC = left caudal; RM = right middle; LA = left apical.

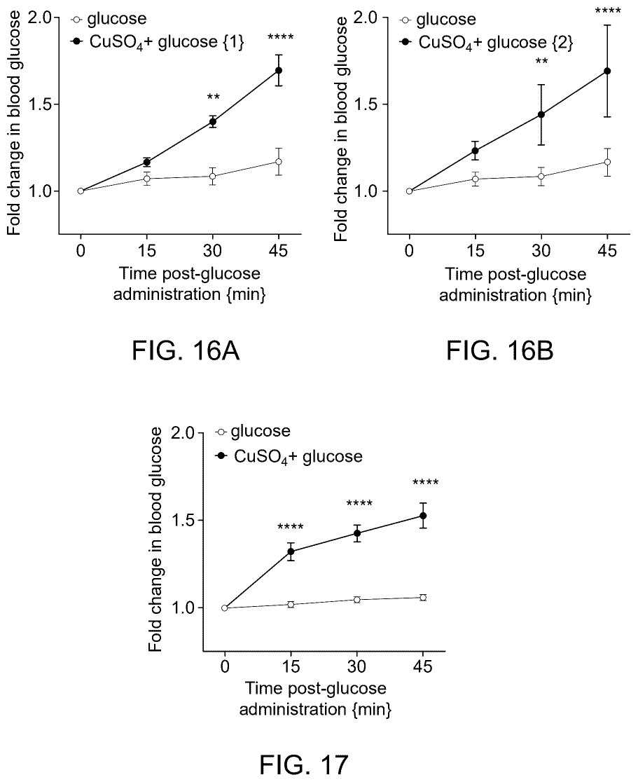

Results for the oral dosing optimization experiment are summarized in FIG. 4. In all four sheep, elevated blood glucose levels were detected at the 15-, 30- and 45-min time-points following separate repeat CuSO4 administrations (FIGS. 16A and 16B). In contrast, a sustained elevation in blood glucose was not observed in the absence of CuSO4 administration (glucose alone). The elevated blood glucose levels observed here confirm effective closure of the reticular groove following CuSO4.

Ten (10) sheep were allocated to the Main Study. Sheep details, live-weights and dosing data (times, volumes, dose delivered) for all Treatments are shown in Table 16.

Inhaled drug dosing (Treatments 1, 1b and 2) was carried out using a ventilator and PARI nebulizer device within a closed respiratory loop while sheep were suspended in a customized harness. This procedure was well tolerated by all sheep. The inhalation rate of 20 breaths/min allowed the full pulmonary dose to be delivered within 20 min (range 11.8-19.3 min). Average dosing times were similar across Treatments (Table 16): 15.5±2.2 min (mean±SD) for inhaled nintedanib (Treatments 1, 1b). The inhalation rate of 20 breaths/min allowed the full pulmonary dose to be delivered within 20 min (range 12.3-18.2 min). Average dosing times were similar across Treatments (Table 16): 15.4±2.2 min (mean±SD) for inhaled nintedanib in fixed combination with pirfenidone (Treatments 2).

Following on from the oral optimization study, the administration of CuSO4 followed by glucose was first tested in the week prior to nintedanib dosing to confirm effective closure of the reticular groove in each test sheep. The oral dosing of nintedanib (Treatment 2), together with the glucose ‘chaser’, was given without any adverse responses observed. The nintedanib dose delivered in all sheep was 75 mg (25 mL of 3 mg/mL preparation), which equated to 2.07±0.03 mg/kg sheep live-weight (Table 17).

TABLE 17
Sheep drug dosing details

| | | Drug dosing details: time/volume/dose delivered | | | | | | | | | | |
|---|---|---|---|---|---|---|---|---|---|---|---|---|
| | | | | | | | | Treatment 2 | | | | |
| | | Treatment 1 | | | Treatment 1b | | | nintedanib^{a} + | | | Treatment 3 | |
| | | Inhaled nintedanib | | | Inhaled nintedanib | | | pirfenidone^{b} | | | Oral nintedanib | |
| | | | dose | total | | dose | total | | dose | total | dose | |
| Sheep | Weight | time | vol | dose | time | vol | dose | time | vol | dose | vol | dose^{c} |
| ID | (kg) | min | ml | mg | min | ml | mg | min | ml | mg | ml | mg/kg |
| 11 | 35.4 | 17.28 | 7.83 | 1.96 | 16.58 | 7.30 | 1.83 | 17.17 | 7.63 | 1.91^{a} | 25.0 | 2.12 |
| | | | | | | | | | | 95.4^{b} | | |
| 12 | 36.2 | 14.00 | 7.51 | 1.88 | 16.13 | 7.19 | 1.87 | 14.25 | 7.73 | 1.97^{a} | 25.0 | 2.07 |
| | | | | | | | | | | 96.6^{b} | | |
| 13 | 36.8 | 15.50 | 7.71 | 1.93 | 15.47 | 7.83 | 1.9 | 14.15 | 7.85 | 1.96^{a} | 25.0 | 2.04 |
| | | | | | | | | | | 98.1^{b} | | |
| 14 | 35.0 | 13.25 | 7.37 | 1.84 | 11.83 | 7.33 | 1.83 | 12.25 | 7.58 | 1.90^{a} | 25.0 | 2.14 |
| | | | | | | | | | | 94.8^{b} | | |
| 16 | 40.8 | 15.08 | 7.62 | 1.91 | 17.85 | 7.75 | 1.94 | 16.75 | 7.71 | 1.93^{a} | 25.0 | 1.84 |
| | | | | | | | | | | 96.4^{b} | | |
| 2 | 34.1 | 19.28 | 7.48 | 1.87 | — | — | — | 12.88 | 7.69 | 1.92^{a} | 25.0 | 2.20 |
| | | | | | | | | | | 96.1^{b} | | |
| 17 | 34.5 | 16.27 | 7.41 | 1.85 | — | — | — | 16.88 | 7.67 | 1.91^{a} | 25.0 | 2.17 |
| | | | | | | | | | | 95.9^{b} | | |
| 18 | 40.3 | 12.65 | 7.21 | 1.80 | — | — | — | 17.53 | 7.56 | 1.89^{a} | 25.0 | 1.86 |
| | | | | | | | | | | 94.5^{b} | | |
| 20 | 35.6 | 18.13 | 7.24 | 1.81 | — | — | — | 18.22 | 7.80 | 1.95^{a} | 25.0 | 2.11 |
| | | | | | | | | | | 97.5^{b} | | |
| 21 | 34.5 | 13.68 | 7.09 | 1.77 | — | — | — | 13.70 | 7.66 | 1.92^{a} | 25.0 | 2.17 |
| | | | | | | | | | | 95.8^{b} | | |
| Mean | 36.3 | 15.51 | 7.45 | 1.86 | 15.57 | 7.48 | 1.87 | 15.38 | 7.69 | 1.93^{a} | — | 2.07 |
| | | | | | | | | | | 96.1^{b} | | |
| SD¹ | 2.38 | 2.21 | 0.23 | 0.06 | 2.27 | 0.29 | 0.05 | 2.15 | 0.09 | 0.03^{a} | — | 0.13 |
| | | | | | | | | | | 1.11^{b} | | |
| SEM² | 0.75 | 0.70 | 0.07 | 0.02 | 1.01 | 0.13 | 0.02 | 0.68 | 0.3 | 0.01^{a} | — | 0.04 |
| | | | | | | | | | | 0.35^{b} | | |

^{a}Treatment 2, nintedanib component;
^{b}Treatment 2, pirfenidone component;
^{c}Treatment 3 oral dose (25 mL × 3 mg/mL nintedanib) shown as mg/kg sheep live-weight
¹SD = standard deviation;
²SEM = standard error of the mean

There was a sustained and significant increase in blood glucose levels seen after oral nintedanib dosing in all sheep, confirming direct passage of the orally administered drug into the true stomach (FIG. 17).

### Blood and BALF Collections

Blood collections were taken at six (6) time-points for each of Treatments 1, 1b and 2, as shown in Table 2. BALF samples were collected across different time-points for each of Treatments 1, 1b, 2 and 3 (Table 15); samples were taken from separate lung lobes (FIG. 3) to avoid sample dilution between time-points. Results are shown in Table 19.

### Pharmacokinetic Results

Comparative nebulized inhaled and oral delivered nintedanib are shown in Table 18 below.

TABLE 18
Sheep pharmacokinetics-nebulized inhaled vs. oral nintedanib

| | | | Inhaled |
|---|---|---|---|
| | Oral | Inhaled Nebulized | vs. Oral |
| | Nintedanib | Nintedanib | (Fold) |
| Formulation | 3 mg/mL in water | Liquid | — |
| Nintedanib Dose: | (75 mg; 2.14 mg/kg) | (2 mg; 0.06 mg/kg) | (−35.7) |
| ELF^{a}: | | | |
| Cmax (ng/mL) | 74.0 | 7375.0 | (+99.7) |
| AUC (mg hr/L) | 1.2 | 5.4 | (+4.5) |
| T1/2 (hr) | 10 | 0.25(α) | — |
| Tmax (hr) | 10 | IPD^{b} | — |
| Plasma: | | | |
| Cmax (ng/mL) | 3.6 | 5.9 | (+1.6) |
| AUC (mg hr/L) | 0.058 | 0.007 | (−8.3) |
| T1/2 (hr) | 10 | 0.25(α) | — |
| Tmax (hr) | 9 | IPD^{b} | — |

^{a}Lung epithelial lining fluid;
^{b}Immediate post dose

Results from Table 17 indicate inhalation of ˜36-fold less nebulized nintedanib delivers ˜100-fold greater epithelial lining fluid (ELF) Cmax and ˜5-fold greater AUC than oral. Comparatively, inhalation of a nebulized nintedanib solution results in an ˜8-fold lower plasma AUC, with an ˜2-fold greater plasma Cmax.

Comparative nebulized inhaled nintedanib monotherapy and nebulized inhaled nintedanib delivered in fixed combination with Pirfenidone are shown in Table 19.

TABLE 19
Sheep pharmacokinetics-nebulized inhaled vs. oral
nintedanib and fixed combination comparison

| | | Inhaled | |
|---|---|---|---|
| | Inhaled | Nebulized | Monotherapy |
| | Nebulized | Nintedanib | vs. Fixed |
| | Nintedanib | (Fixed Combi- | Combination |
| | (Mono- | nation with | Nintedanib |
| | therapy) | Pirfenidone) | (%) |
| Formulation | Liquid | Liquid | — |
| Nintedanib Dose: | (2 mg; | (2 mg; | |
| | 0.06 mg/kg) | 0.06 mg/kg) | |
| ELF^{a}: | | | |
| Cmax (ng/ml) | 7882.5 | 3975.6 | 50 |
| AUC (mg hr/L) | 7.3 | 6.4 | 88 |
| Plasma: | | | |
| Cmax (ng/ml) | 3.9 | 5.7 | 146 |
| AUC (mg hr/L) | 0.004 | 0.005 | 125 |

^{a}Lung epithelial lining fluid;
^{b}Immediate post dose

Nintedanib levels following nebulized inhaled administration of nintedanib in fixed combination with Pirfenidone show a lower ELF Cmax (50% lower) and lower ELF AUC (12% lower). Conversely, plasma Cmax and AUC were increased (46% and 25% higher, respectfully). These results suggest a possible drug-drug interaction whereby in the presence of fixed combination Pirfenidone, nintedanib may be eliminated to the plasma more quickly (lower ELF Cmax and AUC, higher plasma Cmax and AUC). In support of this hypothesis, formulation results indicate Pirfenidone stabilize nintedanib in solution (Example 7). As a means to optimize these fixed combination interactions and reduce drug-drug interactions in vivo, both the amount of nintedanib and Pirfenidone may be optimized (Example 7).

### Example 5: Comparative Inhaled (Liquid Nebulized) Nintedanib and Dry Powder (DPI) Nintedanib Pharmacokinetics in Sheep

### DPI Bridging Study

In a similar manner as employed in Example 4, nintedanib HBr aqueous (nebulized aqueous formulation) and various dry powder nintedanib formulations were compared. Formulations tested in this study were stored at room temperature (RT), protected from light and prepared on day of use (Table 20).

TABLE 20
Drug preparations and Treatment allocation

| | Test | Identification/ | Target Dose for |
|---|---|---|---|
| Treatment # | article | Batch number | delivery (mg) |
| Treatment 1 | Nintedanib | AP02 | 8 mL admixture |
| (Nebulized dose) | HBr | | (2 mg Nintedanib) |
| Treatment 2 | Nintedanib | Trehalose: | 24 mg powder |
| (DPI Form #1) | | leucine:NHBr | (2 mg Nintedanib) |
| | HBr | 80:10:10 wt % | |
| Treatment 3 | Nintedanib | Lactose: | 24 mg powder |
| (DPI Form #2) | | leucine:NHBr | (2 mg Nintedanib) |
| | HBr | 70:20:10 wt % | |
| Treatment 4 | Nintedanib | Lactose:Micronized | 24 mg powder |
| (DPI Form #3) | HBr | NHBr 90:10% | (2 mg Nintedanib) |

Sheep arrived in the facility as one group and were acclimatized over 5-10 days (group housing within indoor cage/pens) prior to experimentation. Sheep received Treatments 1-4 over Weeks 3-4 of the study. Experimental Treatments in each sheep were followed by a 72-96 h washout period prior to the next scheduled Treatment.

For all drug dosing procedures, sheep were removed from their metabolism cage and positioned in a specialized restraining harness to restrict movement of the head and neck and to facilitate drug administration, lung function measures and BALF sample collections.

### DPI and Nebulized Inhaled Dosing

For all dosing procedures, a lubricated cuffed endotracheal (ET) tube (Portex, 7.0-8.0 mm internal diameter) was inserted via the nasal passage (guided by a fibre-optic endoscope) into the trachea. The nebulizer (for Treatment 1) and Penn Century™ insufflator device (Treatments 2-4) were placed within the respiratory line. Prior to all inhaled dosing procedures (Treatments 1-4), ventilator respiration was set to 20 breaths per minute (BPM), 50% inspiration and a tidal volume of 350 mL.

The liquid nintedanib formulation (Treatment 1) was nebulized using an eFlow® inline nebulizer (PARI Pharma GmbH) placed in line with a dual phase control ventilator/respirator (Harvard Apparatus, MA, USA), providing a closed respiratory loop.

The micronized/spray-dried nintedanib formulations (Treatments 2-4) were pre-weighed into the Penn Century™ insufflator device, which was then connected to a regulated pressurized O2 gas source. Just prior to the DPI dose delivery, ventilation was decreased to 10 BPM, and the powder dose was emitted in parallel with 2-3 actuations (over ˜20-30 seconds). At the completion of DPI dosing the ventilation rate was returned to 20 BPM.

For all dosing Treatments, filter chambers (Hudson RCl, NC, USA) were placed in the expiratory line to collect any expired drug dose. Following drug administration, all lines, filters and nebulizer (for Treatment 1) components were rinsed with 50 mL sterile MilliQ water to collect any remaining or expired dose. Washout sample aliquots (500 mcl taken from the 50 mL washout volume) were frozen on dry ice (in Eppendorf tubes), then stored at −80° C. prior to shipment for drug content analyses.

The precise start and finish times for the dose nebulization, and the final duration of the inhaled dose were accurately recorded (see Table 21).

### Peripheral Blood Collections

Blood samples (3 mL) were collected into K3EDTA-coated tubes from all sheep prior to and following each drug administration: pre-dose (t0), and at t2 min, t5 min, t10 min, t15 min, t30 min, t60 min, t120 min, t240 min, t480 min, t720 min post-dose completion. Cell-free plasma sample aliquots (500 mcl) were frozen on dry ice in 1.5 mL Eppendorf tubes, then stored at −80° C. prior to shipment for drug content analyses.

### Bronchoalveolar Lavage Fluid (BALF) Collections

BALF samples were collected prior to and following drug administration from 3 animals. Sampling was carried out by intra-lung infusion of 25 ml of sterile saline via a catheter through the biopsy port of the bronchoscope, followed by immediate retrieval of the BALF sample into the collection syringe.

BALF was collected from separate lung segments/lobes to avoid contamination between sampling time-points. The right apical (RA) lobe was used for all pre-dose (t0) sampling and post-dose samples were collected from left caudal (LC) lobe at t2 min and the right caudal (RC) lobe at t10 min.

BALF sampling details (volumes, exact time of collection following dosing) were recorded and samples immediately frozen on dry ice, then stored at −80° C. prior to shipment for drug content analyses.

### Lung Function Assessments

Lung function measures were recorded in awake, consciously breathing sheep according to established protocols. Lung measures were assessed during quiet breathing, from 5 min prior to and for up to 10 min following each drug administration. Lung parameters (dynamic compliance, transpulmonary pressure, lung volume, breathing and flow rate) were derived from averaged measures of five epochs of five breaths, and data analyzed using LabChart™ software.

### Dpi Bridging Study Dosing Data

Six (n=6) sheep were allocated to this Study. Sheep details, live-weights and dosing data (time, volume/dose delivered) for Treatments 1-4 are shown in Table 21.

Inhaled nebulized drug dosing (Treatment 1) was carried out using a ventilator and PARI eFlow inline device within a closed respiratory loop while sheep were suspended in a customized harness. This procedure was well tolerated by all sheep. For Treatment 1, the inhalation rate of 20 BPM allowed the full pulmonary dose to be delivered within 20 min (range: 17.98-20.00 min; mean±SEM: 18.73±0.23 min).

For delivery of the dry powder inhaled (DPI) formulations, ventilation was reduced to 10 BPM, with 24-30 mg of powder delivered over 2-3 actuations via the Penn Century device. Total dose of micronized powder delivered for Treatments 2-4 ranged from 14-26 mg, with the most efficient delivery observed in DPI doses #2 and #3 (95% effective dose emission/delivered). DPI dose #1 formulation showed signs of aggregation/compaction within the Penn Century device after dosing, with a variable 46-73% powder dose emission.

TABLE 21
Sheep drug dosing details

| | | Drug dosing details | | | | | | | | |
|---|---|---|---|---|---|---|---|---|---|---|
| | | Treatment 1 | | | Treatment 2 | | Treatment 3 | | Treatment 4 | |
| | | Nebulized nintedanib | | | DPI Form #1 | | DPI Form #2 | | DPI Form #3 | |
| | | dose | total | NTB | total | NTB | total | NTB | total | NTB |
| Sheep | Weight | time | dose¹ | dose | dose² | dose | dose³ | dose | dose⁴ | dose |
| ID | (kg) | min | ml | mg | mg | mg | mg | mg | mg | mg |
| 86 | 30.9 | 20.00 | 7.86 | 1.965 | 14 | 1.166 | 26 | 2.167 | 25 | 2.083 |
| 87 | 37.5 | 19.07 | 7.99 | 1.998 | 20 | 1.667 | 23 | 1.917 | 26 | 2.167 |
| 88 | 30.7 | 18.13 | 7.91 | 1.978 | 22 | 1.833 | 24 | 2.000 | 25 | 2.083 |
| 90 | 34.3 | 17.98 | 7.78 | 1.945 | 21 | 1.750 | 24 | 2.000 | 24 | 2.000 |
| 91 | 35.6 | 18.75 | 7.99 | 1.998 | 20 | 1.667 | 22 | 1.833 | 24 | 2.000 |
| 92 | 38.6 | 18.45 | 7.88 | 1.970 | 18 | 1.500 | 24 | 2.000 | 25 | 2.083 |
| Mean | 34.6 | 18.73 | 7.90 | 1.975 | 19.2 | 1.597 | 23.8 | 1.986 | 24.8 | 2.069 |
| SEM⁵ | 1.0 | 0.23 | 0.04 | 0.006 | 1.3 | 0.107 | 0.4 | 0.035 | 0.2 | 0.028 |

¹Treatment 1, 8 mL loaded into eFlow (PARI) inline nebulizer;
²Treatment 2: 30 mg powder loaded;
³Treatment 3: 24-27 mg powder loaded;
⁴Treatment 4: 24-26 mg powder loaded;
⁵SEM:standard error of the mean

### Blood and BALF Collections

Blood samples were collected from all animals at eleven (11) time-points for each of Treatments 1-4. For Treatments 1-4, BALF samples were collected from three (3) animals at three (3) time-points from separate lung lobes. Mean BALF collection times for the ‘2 min’ and ‘10 min’ time-points were 3.18±0.35 min and 13.63±0.82 min respectively (mean±SEM), with no significant differences across Treatments 1-4. BALF volumes did not differ significantly between time-points (0, 2, 10 min) or across Treatments 1-4 (range: 5.5-14.0 mL; mean±SEM: 8.53±0.31 mL).

### Lung Function Analyses

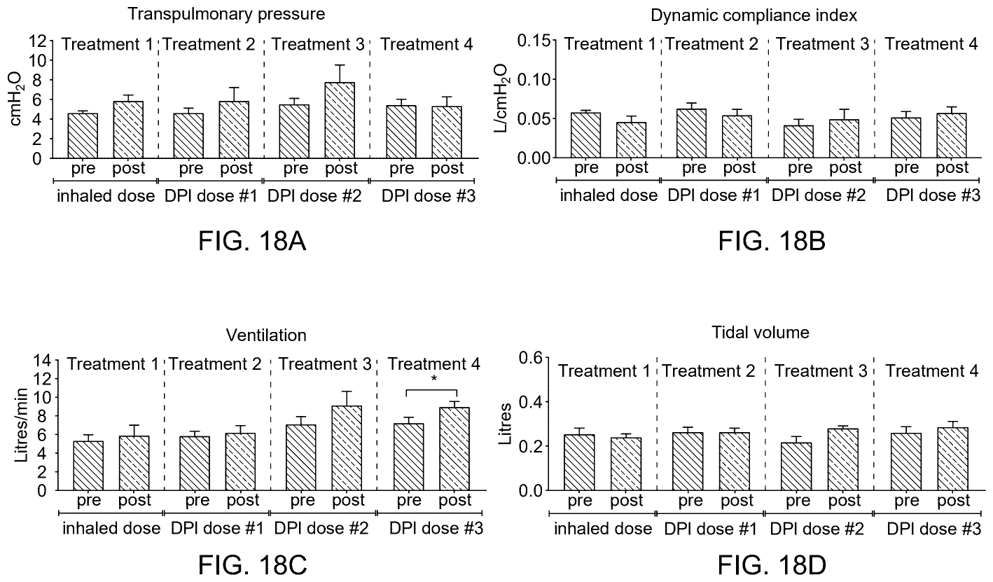

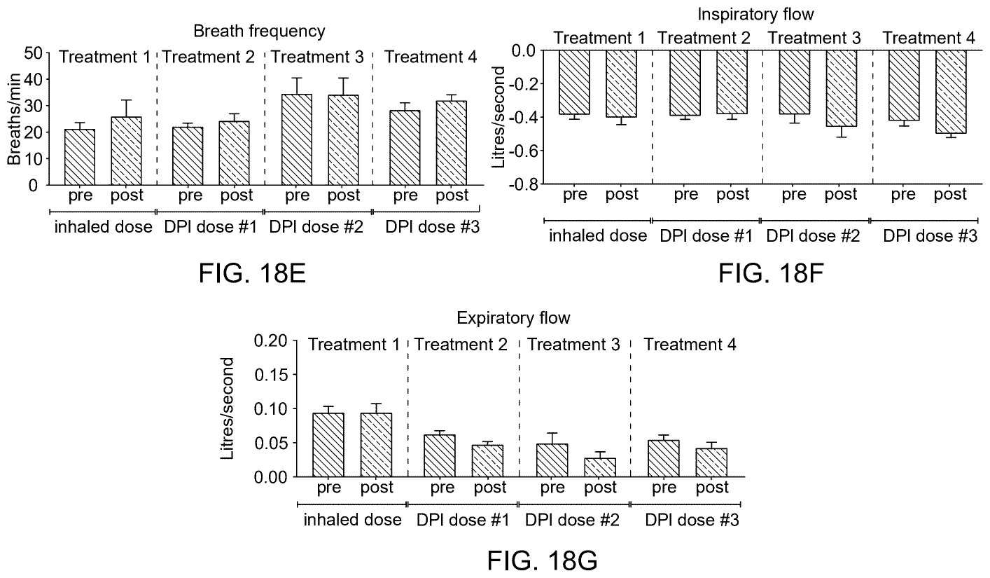

Lung function was assessed before (pre-) and after (post-) Treatments 1-4 in all sheep. Mean values for each lung function parameter assessed is summarized in FIG. 18. No significant changes (pre- vs post-) were seen in the mean transpulmonary pressures (airway resistance) or dynamic compliance in sheep following each of Treatments 1-4. During Treatment 4 there was a small but significant increase in the ventilation rate (FIG. 18C) (mean±SEM increase from 7.23±0.63 L/min to 8.96±0.64 L/min), with no effect on other lung function parameters. This response likely reflects a mild transient response to Treatment 4 dosing (90% lactose:10% nintedanib—micronized).

### Pharmacokinetic Results

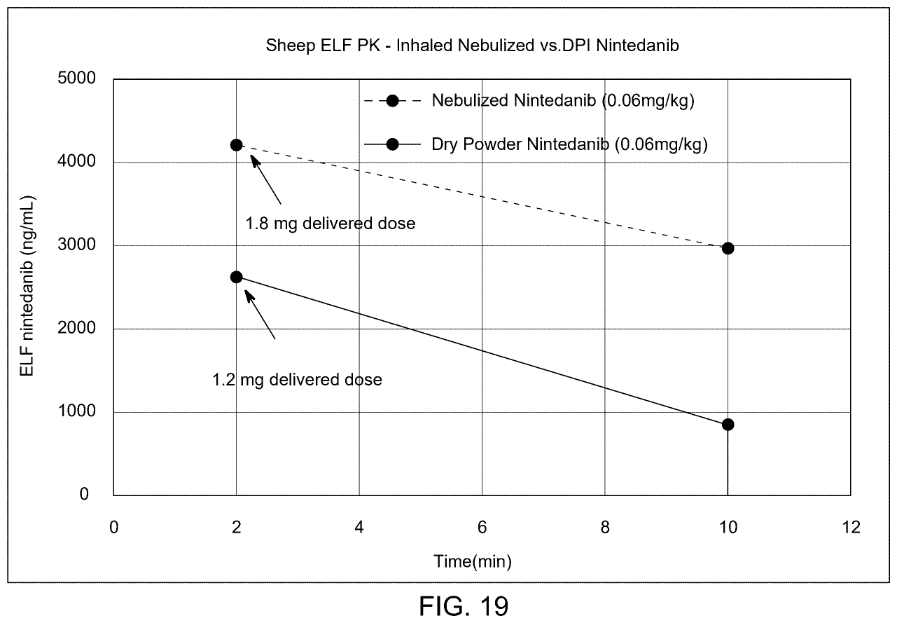

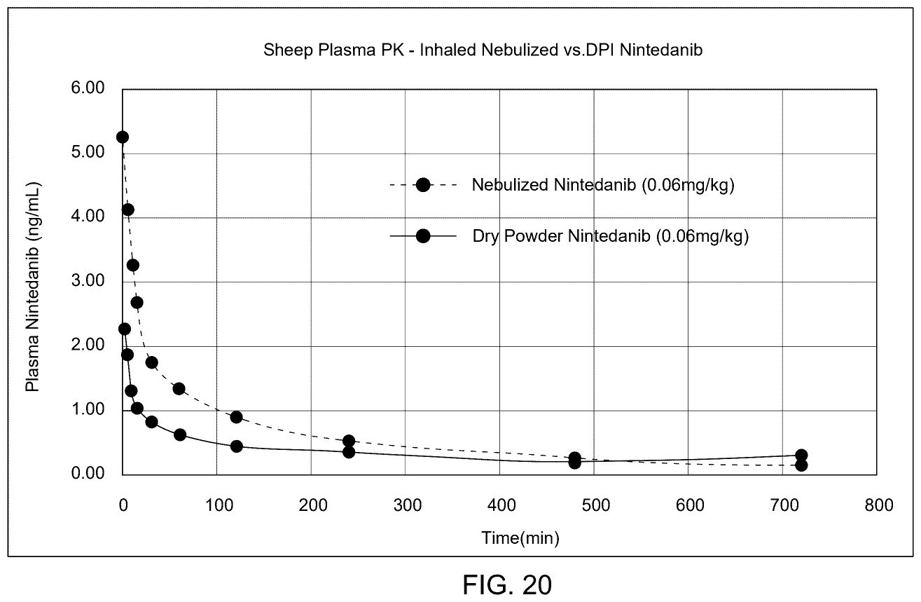

Comparative inhaled dry powder (lactose monohydrate:micronized nintedanib (90:10 wt %)) and aqueous nebulized inhaled delivered nintedanib are shown in FIGS. 19 and 20.

FIG. 19 indicates that both inhalation-delivered aqueous nebulized and dry powder nintedanib eliminate from the lung at a similar rate. This data indicates the simple micronized nintedanib dry powder formulation (90% lactose:10% nintedanib) readily dissolves in the lung and provides a substantial pulmonary bioavailable delivered dose. Importantly, delivered dose calculations (measuring nintedanib remaining in the ventilator tubing after administration was complete and subtracting from the device loaded dose) show that nebulized formulation delivered 1.8 mg nintedanib, while this dry powder formulation delivered 1.2 mg, delivering a dry powder ELF Cmax and AUC about 61% and 44% of the nebulized formulation, respectively. Adjusting the dry powder pharmacokinetic data for fine particle fraction and delivered dose resulted in these curves overlapping, supporting: 1. the two formulations are indeed similarly bioavailable; and 2. additional particle engineering and device optimization improves dry powder delivery.

Similar as suggested in FIG. 19, the parallel curves (same plasma Tmax and same plasma elimination rate) represented in FIG. 20 further supports the equivalent pulmonary bioavailability and elimination between nebulized and dry powder nintedanib.

In support of these results, in vitro dissolution studies were performed. Briefly, the rate of dissolution of nintedanib for each formulation was determined in a simulated lung fluid, using a USP dissolution apparatus.

Simulated lung fluid 4 (SLF 4) was selected to perform the dissolution experiments. However, this buffer contains citrate and high levels of chloride, both of which are known to be incompatible with nintedanib hydrobromide. Therefore, the mixture was adjusted to replace citrate with phosphate and reduce the total chloride concentration to less than 67 mmol. Calcium carbonate was omitted as this is known to cause the pH to drift. In addition, DPPC was not added to the mixture. A study was performed to investigate the effects of chloride concentration and pH, resulting in the selection of 48 mmol total chloride concentration and pH 6.0 to perform the experiments. The SLF was prepared as shown in Table 22.

All of the components except the sodium chloride were added to 4.5 L of deionized water in the order shown. Sodium chloride was measured into a separate beaker and allowed to dissolve in an additional 100 ml of deionized water with stirring. The pH of the components in the 4.5 L of water was adjusted to pH 6.0 with M NaH and the sodium chloride solution was then added. The resulting solution was transferred to a 5 L volumetric and made up to volume with deionized water.

TABLE 22
Components of Simulated Lung Fluid (SLF)

| | | Concen- | Concen- | Amount |
|---|---|---|---|---|
| | Mol. Wt. | tration | tration | in 5 L |
| Component | (g/mol) | (g/L) | (mmol/L) | (g) |
| Magnesium chloride | 203.3 | 0.2033 | 1.000 | 1.017 |
| hexahydrate | | | | |
| Sodium chloride | 58.4 | 2.3376 | 40.0 | 11.688 |
| Potassium chloride | 74.6 | 0.2982 | 3.997 | 1.491 |
| Sodium sulphate | 142.0 | 0.0710 | 0.500 | 0.355 |
| Calcium chloride dihydrate | 147 | 0.3676 | 2.501 | 1.838 |
| Sodium acetate trihydrate | 136.1 | 0.9526 | 6.999 | 4.763 |
| Sodium phosphate | 156.0 | 0.5221 | 3.347 | 2.611 |
| monobasic dihydrate | | | | |

Each vessel was filled with 500 mL of the SLF at 37° C. and 250 mg of the formulation was added with stirring at 125 rpm. Samples were taken using a 13 mm water wettable PTFE syringe filter—2 mL with 0.5 mL discard—at 0.5, 1, 2, 4, 6, 8, 10, 15, 30 and 60 minutes. Samples were diluted 1:4 with sample diluent and analyzed in duplicate using the PLC assay method.

Dissolution results indicate the micronized nintedanib dry powder formulation studied in FIGS. 19 and 20 (90% lactose: 10% micronized nintedanib) is fully dissolved in 6 minutes (Table 23; Formulation 6). Conversely, a spray dried lactose:10% nintedanib formulation of otherwise the same content (Table 23; Formulation 4) was only 62% dissolved at 10 min.

TABLE 23
Contents of Nintedanib Dry Powder Formulations

| | Dissolu- | % Dis- | Time |
|---|---|---|---|
| | tion | solved | to 100% |
| | Formula- | at 10 | Dissolved |
| Composition | tion # | min | (min) |
| Spray Dried trehalose dihydrate:L- | 1 | 100 | 6 |
| leucine:Nintedanib (80:10:10 wt %) | | | |
| Spray Dried lactose:L-leucine: | 2 | 100 | 6 |
| Nintedanib (80:10:10 wt %) | | | |
| Spray Dried trehalose:Nintedanib | 3 | 100 | 8 |
| (90:10 wt %) | | | |
| Spray Dried lactose:Nintedanib | 4 | 62 | >16 |
| (90:10 wt %) | | | |
| Spray Dried lactose:L-leucine: | 5 | 100 | 10 |
| Nintedanib (70:20:10 wt %) | | | |
| Lactose monohydrate:Micronized | 6 | 100 | 6 |
| Nintedanib (90:10 wt %) | | | |

In further support of the sheep pharmacokinetic results, Table 12 indicates that while Table 23 Formulation 6 (lactose monohydrate:micronized nintedanib (90:10 wt %)) exhibited a similar emitted device dose (93%) as the other spray dried formulations (Table 22, Formulations 1-5), fine particle fraction was much greater (Table 13; 60% vs. a range of 23% to 36% for these spray dried formulations). Moreover, this in vitro measured fine particle fraction delivered a 1.32 mg fine particle dose (from a 2.2 mg device loaded nintedanib dose) and these characteristics were maintained for at least 6 months (Table 13). Combining this information, data from Tables 12, 13 and 23 indicate Formulation 6 (lactose monohydrate:micronized nintedanib (90:10 wt %)) exhibits an acceptable fine particle fraction, is stable in these characteristics for at least 6 months, and is efficiently dissolved. Moreover, the fine particle dose (1.32 mg) is equivalent to the 1.2 mg delivered dose measured in the sheep study (FIGS. 19 and 20). It has been shown previously that a fully-soluble aqueous nintedanib nebulized solution is effective in animal models (Surber et al., 2020; Epstein-Shochet et al., 2020). Therefore, it is critical for a dry powder nintedanib formulation to exhibit rapid dissolution to create bioavailable drug in order to maintain the pharmacokinetic/pharmacodynamic relationship with the aqueous nebulized product and equivalent activity. Given this lactose monohydrate:micronized nintedanib (90:10 wt %) formulation dissolution rate and bioavailability are equivalent to that delivered by inhalation of an aqueous solution (FIGS. 19 and 20), maintaining a similar dissolution threshold is critical. From data in Table 23, it is evident that a successful dry powder nintedanib formulation must have a dissolution rate dissolving more than 60% of powder nintedanib in the first 10 minutes, more preferably providing full dissolution in the first 10 minutes. Further, while the fine particle fraction, fine particle dose and actual sheep delivered dose reflect consistency, and dry powder nintedanib modeled performance in humans compared to the approved oral therapy (Table 25) maintains a substantial pharmacokinetic advantage, it is evident that the delivered dose following inhalation of the same device loaded dose of an aqueous nintedanib nebulized formulation is more efficient (FIG. 19). More specifically, compared to the nebulized formulation, the dry powder delivered dose was less efficient and adjusting the dry powder pharmacokinetic data for fine particle fraction and delivered dose resulted in these curves overlapping, supporting that while the two formulations are indeed similarly bioavailable, additional particle engineering and device optimization will further improve dry powder delivery.

To characterize how dry powder nintedanib may perform in humans, a clinical study was first conducted comparing inhaled nebulized and oral nintedanib in healthy volunteers. In this study, subjects were dosed either 2 mg aqueous nebulized nintedanib using the PARI eFlow electronic nebulizer or 150 mg oral pirfenidone (commercial source). Safety results from this study indicated inhaled nebulized nintedanib was well-tolerated with only minimum, Grade 1 side effects such as mild reversible cough observed. Human pharmacokinetic results are presented in Table 24.

TABLE 24
Human ELF and plasma pharmacokinetics-
inhaled nebulized vs. oral nintedanib

| | Oral | Inhaled Neb | |
|---|---|---|---|
| | Tablet | Liquid | Inhaled vs. Oral |
| Formulation | (150 mg) | (2 mg) | (Fold) |
| ELF: | | | |
| Cmax (ng/ml) | 72.7 | 4656.6 | (+64.1) |
| AUC (mg hr/L) | 1.2 | 3.4 | (+2.8) |
| Plasma: | | | |
| Cmax (ng/ml) | 13.5 | 2.8 | (−4.8) |
| AUC (mg hr/L) | 0.163 | 0.004 | (−42.6) |
| Tmax (hr) | 7.8 | 0.2 | |

Table 24 indicates that 2 mg inhaled aqueous nebulized nintedanib delivered ELF Cmax and AUC levels ˜64-fold and ˜3-fold higher, respectively than 150 mg oral nintedanib. Moreover, 2 mg inhaled aqueous nintedanib results in plasma Cmax and AUC levels ˜5-fold and 43-fold, respectively lower than oral. Combined, these results support the hypothesis that inhaled nintedanib will deliver oral-comparable to superior lung-delivered nintedanib with fewer side effects than that observed with the oral product.

To understand how dry powder nintedanib will perform in humans, comparable aqueous nebulized and dry powder inhaled sheep data was extrapolated to the above human observations. Briefly, sheep measured plasma and ELF nintedanib levels resulting from aqueous nebulized inhalation were bridged to the human data in Table 24. Next the collected sheep dry powder nintedanib pharmacokinetics data relationship with collected sheep aqueous nebulized nintedanib data was directly compared to the measured human oral results. This extrapolated comparison is presented in Table 25.

TABLE 25
Human ELF and plasma pharmacokinetics-inhaled
dry powder nintedanib vs. oral nintedanib

| | | Inhaled DPI | |
|---|---|---|---|
| | | 90% lactose: | |
| | Oral | 10% nintedanib | Inhaled |
| | Commercial | (micronized) | vs. Oral |
| Formulation | (150 mg) | (2 mg) | (Fold) |
| ELF: | | | |
| Cmax (ng/ml) | 72.7 | 2887.1 | (+39.7) |
| AUC (mg hr/L) | 1.2 | 1.7 | (+1.4) |
| Plasma: | | | |
| Cmax (ng/ml) | 13.5 | 1.7 | (−7.9) |
| AUC (mg hr/L) | 0.163 | 0.002 | (−65.7) |

Similar to that observed in Table 23, results in Table 24 indicate inhaled nintedanib delivery results in superior lung levels with lower systemic exposure. Specifically, 2 mg dry powder nintedanib will deliver lung ELF Cmax and AUC levels ˜40-fold and ˜1.4-fold higher, respectively than 150 mg oral nintedanib. Moreover, 2 mg dry powder nintedanib results in plasma Cmax and AUC levels ˜8-fold and 66-fold, respectively lower than oral. Combined, these results support the hypothesis that inhaled dry powder nintedanib will also deliver oral-comparable to superior lung-delivered nintedanib with fewer side effects than that observed with the oral product.

### Example 6. Lactose Carrier-Nintedanib Blend Formulation Optimization

A formulation optimization study was conducted to evaluate the effect of force control agents, nintedanib particle size, lactose carrier size and device resistance on the aerodynamic properties of nintedanib HBr dry powder formulations.

Nintedanib was first micronized to the target size, then dry powder formulations were formulated by three dimensional gravitational blending with lactose and, where applicable, force control agent. The prepared formulations were filled in capsules and tested for aerosol dispersion characteristics using the RS01® dry powder inhaler of different resistances (Plastiape, Italy). Selected formulations were placed on stability at 25 C and 40 C and tested at regular intervals. The following summarizes the work completed to date.

Micronization. Nintedanib HBr was milled to a target D50 of 1.5 microns and D90≤5 microns. Micronization was performed using a Fluid Energy jet mill in two passes to obtain a coarse and fine materials. The milling parameters are summarized below.

TABLE 26
Milling Parameters

| | Feed Rate | Grinding | Feed |
|---|---|---|---|
| Pass # | (g/minute) | Pressure (bar) | Pressure (bar) |
| 1 | ~2 | 7 | 5.5 |
| 2 | ~1 | 5 | 3 |

Particle size distribution of the micronized nintedanib HBr was measured using a Malvern Zetasizer Nano-ZS particle size analyzer (Malvern, PA) by suspending the powder in isooctane solution containing 0.1% soy lecithin and sonicated to disperse prior to measurement. The measured values are shown in the Table 27.

TABLE 27
Particle size distribution

| Milled Nintedanib | D10 (μm) | D50 (μm) | D90 (μm) |
|---|---|---|---|
| Coarse | 1.6 | 2.1 | 2.8 |
| Fine | 1.0 | 1.3 | 1.7 |

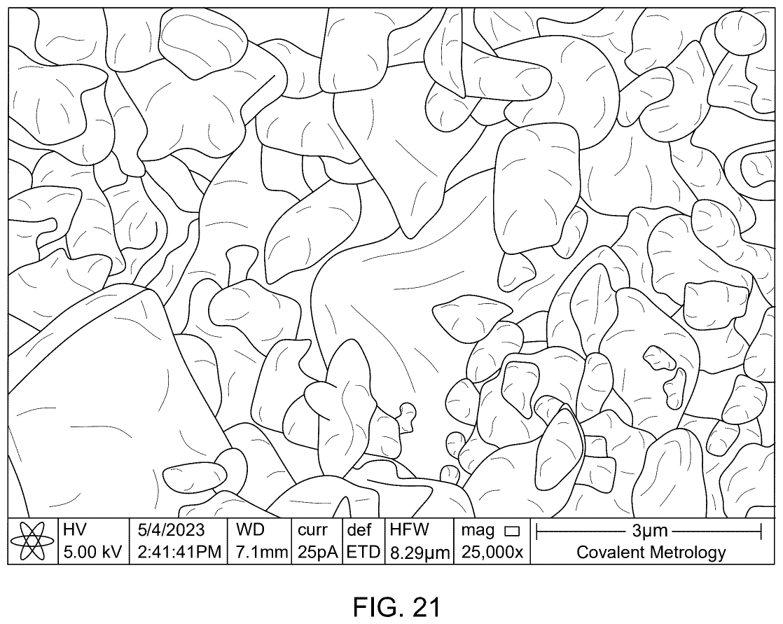

Scanning electron microscopy (SEM) and x-ray powder diffraction was performed on the fine nintedanib HBr. SEM images revealed micronized nintedanib HBr has tomahawk shape with particle size typically less than 3 um (FIG. 21).

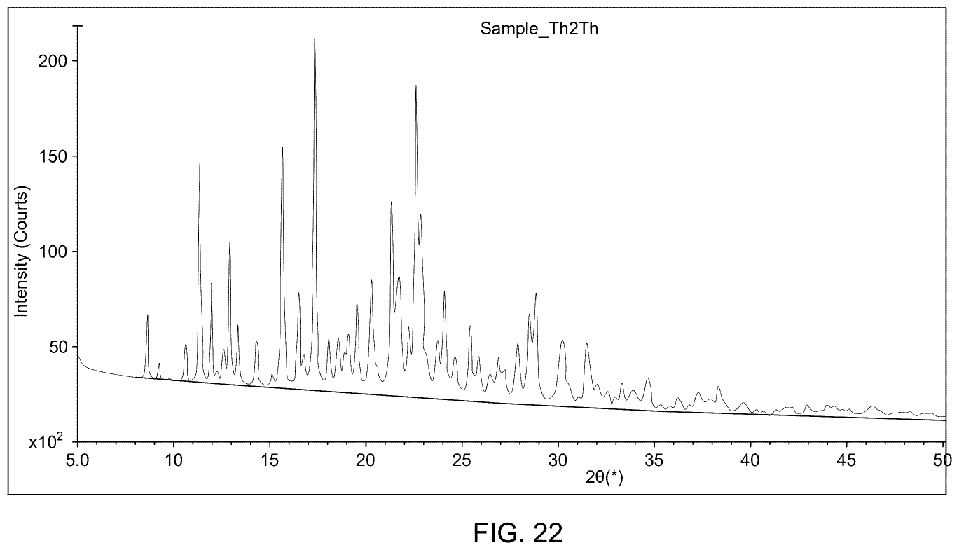

The polymorphic form of micronized nintedanib HBr was determined by X-ray powder diffraction and is comparable to that of the micronized nintedanib HBr generated during the nintedanib powder feasibility study (FIGS. 3 and 4), indicating that nintedanib HBr retains its polymorphic form post micronization (FIG. 22).

Formulation Blending. Micronized nintedanib HBr was blended with a lactose carrier (Lactohale LH200, Respitose ML003) and with force control agents (L-leucine, magnesium stearate [MgSt], Lactohale LH300) at various combinations show in Table 28. The force control agent, when applicable, was added to the lactose carrier, Lactohale 200, in layers in a 50 mL metal vessel. The excipients were mixed in a Turbula tumble blender for 15 minutes at 48 rpm. Micronized nintedanib HBr was added to the resulting powder in layers and mixed in the Turbula blender for 30 minutes at 48 rpm.

TABLE 28
Micronized nintedanib blends

| | Nintedanib or salt | | |
|---|---|---|---|
| | thereof, including | Lactose | Force control |
| Formulation | hydrobromide | Carrier | agent |
| 101-04-45-1 | 10% fine | 90% LH200^{a} | N/A |
| 101-04-45-2 | 10% coarse | 90% LH200^{a} | N/A |
| 101-04-45-3 | 10% fine | 85% LH200^{a} | 5% LH300^{c} |
| 101-04-45-4 | 10% fine | 87.5% LH200^{a} | 2.5% Magnesium |
| | | | stearate |
| 101-04-45-5 | 10% fine | 87.5% LH200^{a} | 2.5% Leucine |
| 101-04-45-6 | 10% fine | 90% ML003^{b} | N/A |

^{a}Lactohale LH200 from DFE Pharma with D10: 5-15 μm, D50: 50-100 μm, D90: 120-160 μm
^{b}Respitose ML003 from DFE Pharma with D10: 1-6 μm, D50: 20-50 μm, D90: 65-140 μm
^{c}Lactohale LH300 from DFE Pharma with D50 ≤ 5 μm, D90 ≤ 10 μm

Once complete the powder formulation is tested for content uniformity (% RSD ≤15%) by obtaining samples from various locations in the blend and the nintedanib content is analyzed by UV-vis spectrophotometer at 390 nm wavelength. The content uniformity results are shown in Table 29.

TABLE 29
Dry powder formulation content uniformity

| | | Nintedanib | |
|---|---|---|---|
| | | HBr | |
| Formulation | Composition | Contents | RSD |
| 101-04-45-1 | 10% NHBr in LH200 lactose | 9.74% | 9.4% |
| 101-04-45-2 | 10% coarse NHbr in LH200 | 9.82% | 10.3% |
| | lactose | | |
| 101-04-45-3 | 10% NHBr, 5% LH300 in | 9.78% | 7.2% |
| | LH200 lactose | | |
| 101-04-45-4 | 10% NHBr, 2.5% MgSt in | 9.55% | 8.2% |
| | LH200 lactose | | |
| 101-04-45-5 | 10% NHbr, 2.5% leucine in | 9.87% | 8.6% |
| | LH200 lactose | | |
| 101-04-45-6 | 10% NHBr in ML003 lactose | 9.89% | 8.6% |

In vitro aerosol performances (fine particle fraction and MMAD) of the above formulations are tested by Next-Generation Impactor (NGI). Approximately 24 mg of the powder is filled into gelatin capsules and dispersed using a low resistance Plastiape RS01 DPI device at a flow rate of 100 L/min over 2.4 s to inspire 4 L air, a volume generally considered as the normal forced inhalation capacity of an average-sized male. The amount of nintedanib HBr on each stage is recovered and analyzed by HPLC. One actuation was collected for each NGL. The emitted dose (amount of nintedanib emitted from the device), fine particle dose and fine particle fraction (amount and fraction of nintedanib by mass in aerosol particles ≤5 μm, respectively), and mass median aerodynamic diameter (MMAD) are shown in Table 30. Formulations 101-04-45-1 through 101-04-45-3 and 101-04-45-5 have high emitted dose, fine particle dose, find particle fraction compared to formulations 101-04-45-4 and 101-04-45-6. The aerosol performance attributes of the former formulations are comparable.

TABLE 30
Aerosol Performance of Nintedanib Dry Powder Formulation

| | Emitted | Fine Particle | | Mass Median |
|---|---|---|---|---|
| Formulation | Dose | Dose | Fine Particle | Aerodynamic |
| No. | (μg) | (μg) | Fraction (%) | Diameter (μm) |
| 101-04-45-1 | 1767 | 1239 | 70.1 | 2.39 |
| 101-04-45-2 | 1835 | 1215 | 66.2 | 2.49 |
| 101-04-45-3 | 1817 | 1260 | 69.3 | 2.40 |
| 101-04-45-4 | 1787 | 1020 | 57.1 | 3.00 |
| 101-04-45-5 | 1814 | 1305 | 72.0 | 2.36 |
| 101-04-45-6 | 1848 | 1158 | 62.7 | 2.44 |

Formulations 101-04-45-1 through 101-04-45-3 and 101-04-45-5 were further evaluated for aerosol performance. The test samples were placed in sealed glass vials and stored at 25° C./60% RH and 40° C./75% RH and were tested after 1 month of storage (only the 25° C./60% RH condition was tested for formulation 101-04-45-05). Aerosol performance summary are presented in Table 31. Except for formulation 101-04-45-5, no substantial changes in the aerosol performance were observed in the other three formulations. Formulation 101-04-45-5 experienced a significant decrease in the fine particle dose and fine particle fraction (>5%) after one month of storage at 25° C./60% RH.

TABLE 31
Stability of Nintedanib Dry Powder Formulation at 1-month

| | % Change from Initial | | | | | | | |
|---|---|---|---|---|---|---|---|---|
| | | | | | Fine Particle | | | |
| Formulation | Emitted Dose | | Fine Particle Dose | | Fraction | | MMAD | |
| No. | 25° C. | 40° C. | 25° C. | 40° C. | 25° C. | 40° C. | 25° C. | 40° C. |
| 101-04-45-1 | 100.5 | 101.4 | 101.2 | 99.8 | 100.7 | 98.5 | 105.9 | 103.3 |
| 101-04-45-2 | 99.5 | 99.9 | 100.4 | 101.2 | 100.9 | 101.4 | 104.4 | 103.2 |
| 101-04-45-3 | 101.2 | 95.0 | 103.3 | 101.1 | 102.2 | 106.5 | 105.0 | 99.2 |
| 101-04-45-5 | 97.0 | NT | 92.0 | NT | 94.9 | NT | 102.1 | NT |

In addition, formulations 101-04-45-1 thorough 101-04-45-3 and 101-04-45-5 were tested in devices of different flow resistances. Testing of aerosol performance of these formulations in different flow resistance devices is important because many elderly patient with lung disease cannot inspire air at high flow rate required for low resistance device, therefore, testing medium and high resistance devices which require lower inhalation flow rates are important. Testing was performed using monodose RS01 devices (Berry Global, Italy) with low resistance, medium resistance and high resistance with the NGI operating at 100 LPM, 85 LPM and 60 RPM, respectively, to generate a pressure drop of approximately 4 kPa (as specified by USP<601>).

Test samples from the 1-month, 25° C./60% RH stability pull was used for testing. One 24 mg fill capsule was inserted into the inhalation device and subsequently actuated into the NGI at a corresponding flow rate listed in the paragraph above. Nintedanib was collected and analyzed by HPLC method. Aerosol performance characteristics were determined using the Inhalytix software (Copely, UK). The emitted dose mass, emitted dose fraction (relative to the nominal dose), fine particle dose and fine particle fraction and aerosol mass median aerodynamic diameter of each device resistance type are shown in Tables 32 and 33. The emitted dose and emitted dose fraction of formulations 101-04-45-3 and 101-04-45-5 which contain force control agents fine lactose and leucine, in the respective order, were found to be not affected by device resistance. Formulations 101-04-45-1 and 101-04-45-2 which do not contain force control agents, on the other hand, were found to be lower compared to those of low and medium resistance devices.

TABLE 32
Effect of Device Resistance on the Emitted Dose and Emitted
Dose Fraction of Nintedanib Dry Powder Formulations

| | Emitted Dose, μg | | | Emitted Dose, % of Nominal | | |
|---|---|---|---|---|---|---|
| Formulation | Low | Medium | High | Low | Medium | High |
| No. | Resistance | Resistance | Resistance | Resistance | Resistance | Resistance |
| 101-04-45-1 | 1776 | 1742 | 1591 | 88.8 | 87.1 | 79.5 |
| 101-04-45-2 | 1826 | 1821 | 1740 | 91.3 | 91.1 | 87.0 |
| 101-04-45-3 | 1839 | 1811 | 1802 | 91.9 | 90.6 | 90.1 |
| 101-04-45-5 | 1759 | 1762 | 1795 | 87.9 | 88.1 | 89.8 |

The fine particle dose and fine particle fractions of all formulations tested with the medium resistance devices showed a slight increase over those of low resistance devices while those of the high resistance devices were substantially lower compared to those of the low resistance and medium resistance devices. Of the four formulations tested, formulations 101-04-45-1 and 101-04-45-3 exhibited consistently high fine particle dose and fine particle fraction. Formulation 101-04-45-5, on the other hand, consistently exhibited lower fine particle dose and fine particle fraction in medium resistance and high resistance devices. The MMAD was found not to be affected by device resistance for all four formulations tested.

TABLE 33
Effect of Device Resistance on the Fine Particle Dose and Fine
Particle Fraction of Nintedanib Dry Powder Formulations

| | | | | | | | Mass Median Aerodynamic | | |
|---|---|---|---|---|---|---|---|---|---|
| Formulation | Fine Particle Dose, μg | | | Fine Particle Fraction, % | | | Diameter, μm | | |
| No. | Low | Medium | High | Low | Medium | High | Low | Medium | High |
| 101-04-45-1 | 1254 | 1273 | 1045 | 70.6 | 73.1 | 65.7 | 2.53 | 2.47 | 2.36 |
| 101-04-45-2 | 1220 | 1301 | 1186 | 66.8 | 71.5 | 68.2 | 2.60 | 2.32 | 2.61 |
| 101-04-45-3 | 1302 | 1322 | 1167 | 70.8 | 73.0 | 64.8 | 2.52 | 2.37 | 2.71 |
| 101-04-45-5 | 1201 | 1215 | 1016 | 68.3 | 69.0 | 56.6 | 2.41 | 2.53 | 2.65 |

In summary, formulation 101-04-45-03 formulated with 5% fine lactose as the force control agent exhibited good aerosol performance, is stable through 1 month of storage at 25° C./60% RH and 40° C./75% RH conditions, and is minimally affected by device resistance. Formulation 101-04-45-3 is the preferred formulation for further development based on its overall aerosol performance characteristics. By nonlimiting example, a preferred embodiment contains micronized nintedanib or salt thereof, including the hydrobromide salt in solid particles with a particle size distribution defined as having a D10 between about 0.1 μm and about 1 μm, a D50 between about 1 μm and about 2.5 μm, and a D90 between about 1.5 μm and about 5 μm at a formulation content between about 1% and about 20% on a weight by weight basis. The preferred embodiment may further contain lactose with a particle size distribution defined as having a D10 between about 5 μm to about 15 μm, a D50 between about 50 μm to about 100 μm, and a D90 between about 120 μm to about 160 μm at a formulation content between about 60% and about 99% on a weight by weight basis. The preferred embodiment may further contains lactose fines with a particle size distribution defined as having a D50 less than about 5 μm and a D90 less than about 10 μm at a formulation content between more than 0% and about 20% on a weight by weight basis. Moreover, the preferred formulation described herein enables a high emitted dose from medium and high-resistance dry powder inhalation devices. For clarity, medium and high resistance devices are designed to require lower inhalation flow rates to actuate and disperse dry powder formulation dosages and are more well-suited for a human with pulmonary disease and reduced lung function whose inhalation flow rates may otherwise be insufficient to efficiently actuate and disperse the dry powder dose for inhalation administration from a low resistance device.

### Example 7. Liquid Nintedanib and Pirfenidone Fixed Dose Combination Formulations

Data from Example 5 indicate nebulized inhaled administration of nintedanib in fixed combination with pirfenidone delivers a lower ELF levels, and increased plasma levels. This observation suggests a possible drug-drug interaction with pirfenidone whereby in the presence of fixed combination pirfenidone, nintedanib may be eliminated to the plasma more quickly. In support of this hypothesis, formulation results indicate pirfenidone stabilize nintedanib in solution. As a means to optimize these fixed combination interactions and reduce drug-drug interactions in vivo, both the amount of nintedanib and pirfenidone may be optimized. As an initial means to characterize the pirfenidone-nintedanib interaction, the physical stability of nintedanib in the presence of pirfenidone at different concentrations were investigated. It is previously known that nintedanib (of various salt forms including esylate, hydrobromide and others) is physically unstable in solutions containing 30 mM sodium chloride or higher. It is also previously known that pirfenidone, when added to such nintedanib solution containing sodium chloride can physically stabilize nintedanib. As a surrogate to understand the in vivo concentration limits and or ratio of the undesired in vivo drug-drug interaction, the chemical-chemical interaction between pirfenidone and nintedanib was explored. In this study the stability of 0.25 mg/mL nintedanib solution in a 67 mM sodium chloride at 0, 2.5, 5, 7.5, 10 and 12.5 mg/mL pirfenidone was assessed. By design, the limit of stability was inferred as the limit of the chemical-chemical or drug-drug interaction in vivo. First a 67 mM sodium chloride solution was prepared by dissolving 0.8 g sodium chloride in water and dilute to 200 mL. A 12.5 mg/mL solution was prepared by dissolving 1.25 g pirfenidone in saline solution prepared above to 100 mL. An appropriate amount of nintedanib HBr was added to the 12.5 mg/mL solution and combine with 67 mM sodium chloride solution to produce a series of formulations shown in Table 34. The solutions were heated to 40 C while mixing for 30 minutes, which upon completion the nintedanib in all formulations was completely dissolved.

TABLE 34
Preparation of test solutions for
pirfenidone-nintedanib interaction study

| | | Volume of | | |
|---|---|---|---|---|
| | | 12.5 mg/mL | | |
| | | Pirfenidone, | | |
| | | 67 mM | Volume of | |
| | Nintedanib | sodium | 67 mM | |
| Test | HBR | chloride | sodium | |
| solution | (expressed | solution | chloride | |
| Number | as base) | added | added | Test solution |
| 1 | 5 mg | N/A | 20 mL | 0.25 mg nintedanib, |
| | | | | 0 mg/mL pirfenidone |
| | | | | 67 mM NaCl |
| 2 | 5 mg | 4 mL | 15 mL | 0.25 mg/mL nintedanib |
| | | | | 2.5 mg/mL pirfenidone |
| | | | | 67 mM NaCl |
| 3 | 5 mg | 8 mL | 12 mL | 0.25 mg/mL nintedanib |
| | | | | 5 mg/mL pirfenidone |
| | | | | 67 mM NaCl |
| 4 | 5 mg | 12 mL | 8 mL | 0.25 mg/mL nintedanib |
| | | | | 7.5 mg/mL pirfenidone |
| | | | | 67 mM NaCl |
| 5 | 5 mg | 16 mL | 4 mL | 0.25 mg/mL nintedanib |
| | | | | 10 mg/mL pirfenidone |
| | | | | 67 mM NaCl |
| 6 | 5 mg | 20 mL | N/A | 0.25 mg/mL nintedanib |
| | | | | 12.5 mg/mL pirfenidone |
| | | | | 67 mM NaCl |

The pirfenidone-nintedanib interaction results are summarized in Table 35.

TABLE 35
Preparation of test samples for pirfenidone-
nintedanib interaction study

| Test Solution | Composition | Results |
|---|---|---|
| 1 | 0.25 mg nintedanib, | Precipitates formed |
| | 0 mg/mL pirfenidone | after 2 hours |
| | 67 mM NaCl | |
| 2 | 0.25 mg/mL nintedanib | Precipitates formed |
| | 2.5 mg/mL pirfenidone | after 2 hours |
| | 67 mM NaCl | |
| 3 | 0.25 mg/mL nintedanib | Precipitates formed |
| | 5 mg/mL pirfenidone | after 10 hours |
| | 67 nM NaCl | |
| 4 | 0.25 mg/mL nintedanib | No precipitations |
| | 7.5 mg/mL pirfenidone | through 2 weeks |
| | 67 mM NaCl | |
| 5 | 0.25 mg/mL nintedanib | No precipitations |
| | 10 mg/mL pirfenidone | through 2 weeks |
| | 67 mM NaCl | |
| 6 | 0.25 mg/mL nintedanib | No precipitations |
| | 12.5 mg/mL pirfenidone | through 2 weeks |
| | 67 mM NaCl | |

Results indicate that, on a weight by weight basis, a ratio greater than about 30 parts pirfenidone to 1 part nintedanib (greater than about 7.5 mg/mL pirfenidone per 0.25 mg/mL nintedanib) stabilizes nintedanib in the presence of about 67 mM sodium chloride and, as defined herein, is the limit of the chemical-chemical interaction under this condition. From these data, it is inferred that less than 30 parts pirfenidone per part nintedanib on a weight by weight basis would diminish stabilization and thus reduce the chemical-chemical interaction in under these conditions. To avoid pharmacokinetic alterations that exist when pirfenidone and nintedanib are administered in fixed combination or otherwise co-administered, fewer than 30 parts pirfenidone to 1 part nintedanib should be considered when formulated together or administered in vivo. However, under physiologic conditions, the chloride content is closer to 150 mM, thus this ratio may be extended to not exceed 67 parts pirfenidone per part nintedanib on a weight by weight basis to avoid pharmacokinetic alterations that exist when pirfenidone and nintedanib are administered in fixed combination or otherwise co-administered.

As a means to optimize the invention, the ratio of co-formulated combination nintedanib and pirfenidone is optimized to circumvent a co-formulation chemical interaction and possible in vivo physiologic effect that increases the rate that inhalation delivered nintedanib is eliminated from the lung to the plasma compared to that of nintedanib delivered without co-formulated pirfenidone.

By non-limiting example, 2:100 nintedanib:pirfenidone ratio on a weight by weight basis reduces the pulmonary and increases the plasma nintedanib Cmax about 30-50%. To maximize the pulmonary residence time of inhaled nintedanib in co-formulated combination with pirfenidone it is desired to reduce this pharmacokinetic effect. By non-limiting example, this undesired pharmacokinetic effect is minimized by reducing the pirfenidone content to less than or equal to about 100 mg per dose with a nintedanib:pirfenidone content ratio to between about 1:1 to about 1:67. By another non-limiting example, this undesired pharmacokinetic effect is minimized by reducing the pirfenidone dose to less than or equal to about 100 mg, while maintaining a 1:20 to 1:67 nintedanib:pirfenidone content ratio on a weight by weight basis. By another non-limiting example, this undesired pharmacokinetic effect is minimized by increasing the nintedanib co-formulation content such that the resulting nintedanib:pirfenidone content ratio on a weight by weight basis is less than 1:67.

Methods of the invention include optimizing the co-formulated combination nintedanib or indolinone compound and pirfenidone or pyridone analog ratio to improve therapeutic benefit, including efficacy, safety, tolerability and compliance. By non-limiting example, 100 mg pirfenidone exists at the upper range of pirfenidone tolerability as a nebulized, stand-alone solution and is near the upper threshold of that possible for a compliant and well-tolerated dry powder product. By non-limiting example, it is predicted the efficacy of this nintedanib or indolinone compound and pirfenidone or pyridone analog co-formulated dry powder product will be greater than either active ingredient alone. To maximize the safety/tolerability/compliance-to-efficacy relationship, reducing the amount of overall administered dry powder increases compliance and increases both safety and tolerability of the combination product. Taking advantage of the added effect of co-formulating with nintedanib or indolinone compound, the amount of pirfenidone or pyridone analog in the co-formulated dry powder product may be reduced, while maintaining the overall added benefit of administering both nintedanib or indolinone and pirfenidone or pyridone analog to a patient. By non-limiting example, this desired outcome is created by reducing the pirfenidone dose to less than or equal to about 100 mg, while maintaining a 1:20 to 1:67 nintedanib:pirfenidone content ratio on a weight by weight basis. Optimized nintedanib/pirfenidone combination ratios and formulations are shown in Table 36.

TABLE 36
Optimized nintedanib/pirfenidone combination ratios and formulations

| | Formulation | | | | | | |
|---|---|---|---|---|---|---|---|
| | (nintedanib/ | | | | Propylene | Sodium | Sodium |
| Formulation | pirfenidone | Nintedanib | Pirfenidone | Glycine | Glycol | chloride | saccharin |
| Number | ratio) | (mg/mL) ^{a} | (mg/mL) | (mM) | (%) | (mM) | (mM) |
| 1 | 4:1 | 1 | 0.25 | 0 | 0 | 0 | 0 |
| 2 | 3:1 | 1 | 0.33 | 0 | 0 | 0 | 0 |
| 3 | 2:1 | 1 | 0.5 | 0 | 0 | 0 | 0 |
| 4 | 1:1 | 1 | 1 | 0 | 0 | 0 | 0 |
| 5 | 1:2.5 | 1 | 2.5 | 0 | 0 | 0 | 0 |
| 6 | 1:5 | 1 | 5 | 0 | 0 | 0 | 0 |
| 7 | 1:10 | 1 | 10 | 0 | 0 | 0 | 0 |
| 8 | 1:20 | 1 | 20 | 0 | 0 | 0 | 0 |
| 9 | 1:25 | 0.5 | 12.5 | 0 | 0 | 0 | 0 |
| 10 | 1:50 | 0.25 | 12.5 | 0 | 0 | 0 | 0 |
| 11 | 1:67 | 0.25 | 16.75 | 0 | 0 | 0 | 0 |
| 12 | 1:75 | 0.2 | 15 | 0 | 0 | 0 | 0 |
| 13 | 1:100 | 0.2 | 20 | 0 | 0 | 0 | 0 |
| 14 | 1:200 | 0.1 | 20 | 0 | 0 | 0 | 0 |
| 15 | 1:250 | 0.08 | 20 | 0 | 0 | 0 | 0 |
| 16 | 1:300 | 0.067 | 20 | 0 | 0 | 0 | 0 |
| 17 | 1:400 | 0.05 | 20 | 0 | 0 | 0 | 0 |
| 18 | 1:500 | 0.04 | 20 | 0 | 0 | 0 | 0 |
| 19 | 1:600 | 0.033 | 20 | 0 | 0 | 0 | 0 |
| 20 | 1:700 | 0.029 | 20 | 0 | 0 | 0 | 0 |
| 21 | 1:800 | 0.025 | 20 | 0 | 0 | 0 | 0 |
| 22 | 1:900 | 0.022 | 20 | 0 | 0 | 0 | 0 |
| 23 | 1:1000 | 0.02 | 20 | 0 | 0 | 0 | 0 |
| 24 | 4:1 | 1 | 0.25 | 10 | 0 | 0 | 0 |
| 25 | 3:1 | 1 | 0.33 | 10 | 0 | 0 | 0 |
| 26 | 2:1 | 1 | 0.5 | 10 | 0 | 0 | 0 |
| 27 | 1:1 | 1 | 1 | 10 | 0 | 0 | 0 |
| 28 | 1:2.5 | 1 | 2.5 | 10 | 0 | 0 | 0 |
| 29 | 1:5 | 1 | 5 | 10 | 0 | 0 | 0 |
| 30 | 1:10 | 1 | 10 | 10 | 0 | 0 | 0 |
| 31 | 1:20 | 1 | 20 | 10 | 0 | 0 | 0 |
| 32 | 1:25 | 0.5 | 12.5 | 10 | 0 | 0 | 0 |
| 33 | 1:50 | 0.25 | 12.5 | 10 | 0 | 0 | 0 |
| 34 | 1:67 | 0.25 | 16.75 | 10 | 0 | 0 | 0 |
| 35 | 1:75 | 0.2 | 15 | 10 | 0 | 0 | 0 |
| 36 | 1:100 | 0.2 | 20 | 10 | 0 | 0 | 0 |
| 37 | 1:200 | 0.1 | 20 | 10 | 0 | 0 | 0 |
| 38 | 1:250 | 0.08 | 20 | 10 | 0 | 0 | 0 |
| 39 | 1:300 | 0.067 | 20 | 10 | 0 | 0 | 0 |
| 40 | 1:400 | 0.05 | 20 | 10 | 0 | 0 | 0 |
| 41 | 1:500 | 0.04 | 20 | 10 | 0 | 0 | 0 |
| 42 | 1:600 | 0.033 | 20 | 10 | 0 | 0 | 0 |
| 43 | 1:700 | 0.029 | 20 | 10 | 0 | 0 | 0 |
| 44 | 1:800 | 0.025 | 20 | 10 | 0 | 0 | 0 |
| 45 | 1:900 | 0.022 | 20 | 10 | 0 | 0 | 0 |
| 46 | 1:1000 | 0.02 | 20 | 10 | 0 | 0 | 0 |
| 47 | 4:1 | 1 | 0.25 | 100 | 0 | 0 | 0 |
| 48 | 3:1 | 1 | 0.33 | 100 | 0 | 0 | 0 |
| 49 | 2:1 | 1 | 0.5 | 100 | 0 | 0 | 0 |
| 50 | 1:1 | 1 | 1 | 100 | 0 | 0 | 0 |
| 51 | 1:2.5 | 1 | 2.5 | 100 | 0 | 0 | 0 |
| 52 | 1:5 | 1 | 5 | 100 | 0 | 0 | 0 |
| 53 | 1:10 | 1 | 10 | 100 | 0 | 0 | 0 |
| 54 | 1:20 | 1 | 20 | 100 | 0 | 0 | 0 |
| 55 | 1:25 | 0.5 | 12.5 | 100 | 0 | 0 | 0 |
| 56 | 1:50 | 0.25 | 12.5 | 100 | 0 | 0 | 0 |
| 57 | 1:67 | 0.25 | 16.75 | 100 | 0 | 0 | 0 |
| 58 | 1:75 | 0.2 | 15 | 100 | 0 | 0 | 0 |
| 59 | 1:100 | 0.2 | 20 | 100 | 0 | 0 | 0 |
| 60 | 1:200 | 0.1 | 20 | 100 | 0 | 0 | 0 |
| 61 | 1:250 | 0.08 | 20 | 100 | 0 | 0 | 0 |
| 62 | 1:300 | 0.067 | 20 | 100 | 0 | 0 | 0 |
| 63 | 1:400 | 0.05 | 20 | 100 | 0 | 0 | 0 |
| 64 | 1:500 | 0.04 | 20 | 100 | 0 | 0 | 0 |
| 65 | 1:600 | 0.033 | 20 | 100 | 0 | 0 | 0 |
| 66 | 1:700 | 0.029 | 20 | 100 | 0 | 0 | 0 |
| 67 | 1:800 | 0.025 | 20 | 100 | 0 | 0 | 0 |
| 68 | 1:900 | 0.022 | 20 | 100 | 0 | 0 | 0 |
| 69 | 1:1000 | 0.02 | 20 | 100 | 0 | 0 | 0 |
| 70 | 4:1 | 1 | 0.25 | 10 | 1 | 0 | 0 |
| 71 | 3:1 | 1 | 0.33 | 10 | 1 | 0 | 0 |
| 72 | 2:1 | 1 | 0.5 | 10 | 1 | 0 | 0 |
| 73 | 1:1 | 1 | 1 | 10 | 1 | 0 | 0 |
| 74 | 1:2.5 | 1 | 2.5 | 10 | 1 | 0 | 0 |
| 75 | 1:5 | 1 | 5 | 10 | 1 | 0 | 0 |
| 76 | 1:10 | 1 | 10 | 10 | 1 | 0 | 0 |
| 77 | 1:20 | 1 | 20 | 10 | 1 | 0 | 0 |
| 78 | 1:25 | 0.5 | 12.5 | 10 | 1 | 0 | 0 |
| 79 | 1:50 | 0.25 | 12.5 | 10 | 1 | 0 | 0 |
| 80 | 1:67 | 0.25 | 16.75 | 10 | 1 | 0 | 0 |
| 81 | 1:75 | 0.2 | 15 | 10 | 1 | 0 | 0 |
| 82 | 1:100 | 0.2 | 20 | 10 | 1 | 0 | 0 |
| 83 | 1:200 | 0.1 | 20 | 10 | 1 | 0 | 0 |
| 84 | 1:250 | 0.08 | 20 | 10 | 1 | 0 | 0 |
| 85 | 1:300 | 0.067 | 20 | 10 | 1 | 0 | 0 |
| 86 | 1:400 | 0.05 | 20 | 10 | 1 | 0 | 0 |
| 87 | 1:500 | 0.04 | 20 | 10 | 1 | 0 | 0 |
| 88 | 1:600 | 0.033 | 20 | 10 | 1 | 0 | 0 |
| 89 | 1:700 | 0.029 | 20 | 10 | 1 | 0 | 0 |
| 90 | 1:800 | 0.025 | 20 | 10 | 1 | 0 | 0 |
| 91 | 1:900 | 0.022 | 20 | 10 | 1 | 0 | 0 |
| 92 | 1:1000 | 0.02 | 20 | 10 | 1 | 0 | 0 |
| 93 | 4:1 | 1 | 0.25 | 10 | 10 | 0 | 0 |
| 94 | 3:1 | 1 | 0.33 | 10 | 10 | 0 | 0 |
| 95 | 2:1 | 1 | 0.5 | 10 | 10 | 0 | 0 |
| 96 | 1:1 | 1 | 1 | 10 | 10 | 0 | 0 |
| 97 | 1:2.5 | 1 | 2.5 | 10 | 10 | 0 | 0 |
| 98 | 1:5 | 1 | 5 | 10 | 10 | 0 | 0 |
| 99 | 1:10 | 1 | 10 | 10 | 10 | 0 | 0 |
| 100 | 1:20 | 1 | 20 | 10 | 10 | 0 | 0 |
| 101 | 1:25 | 0.5 | 12.5 | 10 | 10 | 0 | 0 |
| 102 | 1:50 | 0.25 | 12.5 | 10 | 10 | 0 | 0 |
| 103 | 1:67 | 0.25 | 16.75 | 10 | 10 | 0 | 0 |
| 104 | 1:75 | 0.2 | 15 | 10 | 10 | 0 | 0 |
| 105 | 1:100 | 0.2 | 20 | 10 | 10 | 0 | 0 |
| 106 | 1:200 | 0.1 | 20 | 10 | 10 | 0 | 0 |
| 107 | 1:250 | 0.08 | 20 | 10 | 10 | 0 | 0 |
| 108 | 1:300 | 0.067 | 20 | 10 | 10 | 0 | 0 |
| 109 | 1:400 | 0.05 | 20 | 10 | 10 | 0 | 0 |
| 110 | 1:500 | 0.04 | 20 | 10 | 10 | 0 | 0 |
| 111 | 1:600 | 0.033 | 20 | 10 | 10 | 0 | 0 |
| 112 | 1:700 | 0.029 | 20 | 10 | 10 | 0 | 0 |
| 113 | 1:800 | 0.025 | 20 | 10 | 10 | 0 | 0 |
| 114 | 1:900 | 0.022 | 20 | 10 | 10 | 0 | 0 |
| 115 | 1:1000 | 0.02 | 20 | 10 | 10 | 0 | 0 |
| 116 | 4:1 | 1 | 0.25 | 10 | 1 | 30 | 0 |
| 117 | 3:1 | 1 | 0.33 | 10 | 1 | 30 | 0 |
| 118 | 2:1 | 1 | 0.5 | 10 | 1 | 30 | 0 |
| 119 | 1:1 | 1 | 1 | 10 | 1 | 30 | 0 |
| 120 | 1:2.5 | 1 | 2.5 | 10 | 1 | 30 | 0 |
| 121 | 1:5 | 1 | 5 | 10 | 1 | 30 | 0 |
| 122 | 1:10 | 1 | 10 | 10 | 1 | 30 | 0 |
| 123 | 1:20 | 1 | 20 | 10 | 1 | 30 | 0 |
| 124 | 1:25 | 0.5 | 12.5 | 10 | 1 | 30 | 0 |
| 125 | 1:50 | 0.25 | 12.5 | 10 | 1 | 30 | 0 |
| 126 | 1:67 | 0.25 | 16.75 | 10 | 1 | 30 | 0 |
| 127 | 1:75 | 0.2 | 15 | 10 | 1 | 30 | 0 |
| 128 | 1:100 | 0.2 | 20 | 10 | 1 | 30 | 0 |
| 129 | 1:200 | 0.1 | 20 | 10 | 1 | 30 | 0 |
| 130 | 1:250 | 0.08 | 20 | 10 | 1 | 30 | 0 |
| 131 | 1:300 | 0.067 | 20 | 10 | 1 | 30 | 0 |
| 132 | 1:400 | 0.05 | 20 | 10 | 1 | 30 | 0 |
| 133 | 1:500 | 0.04 | 20 | 10 | 1 | 30 | 0 |
| 134 | 1:600 | 0.033 | 20 | 10 | 1 | 30 | 0 |
| 135 | 1:700 | 0.029 | 20 | 10 | 1 | 30 | 0 |
| 136 | 1:800 | 0.025 | 20 | 10 | 1 | 30 | 0 |
| 137 | 1:900 | 0.022 | 20 | 10 | 1 | 30 | 0 |
| 138 | 1:1000 | 0.02 | 20 | 10 | 1 | 30 | 0 |
| 139 | 4:1 | 1 | 0.25 | 10 | 1 | 100 | 0 |
| 140 | 3:1 | 1 | 0.33 | 10 | 1 | 100 | 0 |
| 141 | 2:1 | 1 | 0.5 | 10 | 1 | 100 | 0 |
| 142 | 1:1 | 1 | 1 | 10 | 1 | 100 | 0 |
| 143 | 1:2.5 | 1 | 2.5 | 10 | 1 | 100 | 0 |
| 144 | 1:5 | 1 | 5 | 10 | 1 | 100 | 0 |
| 145 | 1:10 | 1 | 10 | 10 | 1 | 100 | 0 |
| 146 | 1:20 | 1 | 20 | 10 | 1 | 100 | 0 |
| 147 | 1:25 | 0.5 | 12.5 | 10 | 1 | 100 | 0 |
| 148 | 1:50 | 0.25 | 12.5 | 10 | 1 | 100 | 0 |
| 149 | 1:67 | 0.25 | 16.75 | 10 | 1 | 100 | 0 |
| 150 | 1:75 | 0.2 | 15 | 10 | 1 | 100 | 0 |
| 151 | 1:100 | 0.2 | 20 | 10 | 1 | 100 | 0 |
| 152 | 1:200 | 0.1 | 20 | 10 | 1 | 100 | 0 |
| 153 | 1:250 | 0.08 | 20 | 10 | 1 | 100 | 0 |
| 154 | 1:300 | 0.067 | 20 | 10 | 1 | 100 | 0 |
| 155 | 1:400 | 0.05 | 20 | 10 | 1 | 100 | 0 |
| 156 | 1:500 | 0.04 | 20 | 10 | 1 | 100 | 0 |
| 157 | 1:600 | 0.033 | 20 | 10 | 1 | 100 | 0 |
| 158 | 1:700 | 0.029 | 20 | 10 | 1 | 100 | 0 |
| 159 | 1:800 | 0.025 | 20 | 10 | 1 | 100 | 0 |
| 160 | 1:900 | 0.022 | 20 | 10 | 1 | 100 | 0 |
| 161 | 1:1000 | 0.02 | 20 | 10 | 1 | 100 | 0 |
| 162 | 4:1 | 1 | 0.25 | 10 | 1 | 300 | 0 |
| 163 | 3:1 | 1 | 0.33 | 10 | 1 | 300 | 0 |
| 164 | 2:1 | 1 | 0.5 | 10 | 1 | 300 | 0 |
| 165 | 1:1 | 1 | 1 | 10 | 1 | 300 | 0 |
| 166 | 1:2.5 | 1 | 2.5 | 10 | 1 | 300 | 0 |
| 167 | 1:5 | 1 | 5 | 10 | 1 | 300 | 0 |
| 168 | 1:10 | 1 | 10 | 10 | 1 | 300 | 0 |
| 169 | 1:20 | 1 | 20 | 10 | 1 | 300 | 0 |
| 170 | 1:25 | 0.5 | 12.5 | 10 | 1 | 300 | 0 |
| 171 | 1:50 | 0.25 | 12.5 | 10 | 1 | 300 | 0 |
| 172 | 1:67 | 0.25 | 16.75 | 10 | 1 | 300 | 0 |
| 173 | 1:75 | 0.2 | 15 | 10 | 1 | 300 | 0 |
| 174 | 1:100 | 0.2 | 20 | 10 | 1 | 300 | 0 |
| 175 | 1:200 | 0.1 | 20 | 10 | 1 | 300 | 0 |
| 176 | 1:250 | 0.08 | 20 | 10 | 1 | 300 | 0 |
| 177 | 1:300 | 0.067 | 20 | 10 | 1 | 300 | 0 |
| 178 | 1:400 | 0.05 | 20 | 10 | 1 | 300 | 0 |
| 179 | 1:500 | 0.04 | 20 | 10 | 1 | 300 | 0 |
| 180 | 1:600 | 0.033 | 20 | 10 | 1 | 300 | 0 |
| 181 | 1:700 | 0.029 | 20 | 10 | 1 | 300 | 0 |
| 182 | 1:800 | 0.025 | 20 | 10 | 1 | 300 | 0 |
| 183 | 1:900 | 0.022 | 20 | 10 | 1 | 300 | 0 |
| 184 | 1:1000 | 0.02 | 20 | 10 | 1 | 300 | 0 |
| 185 | 4:1 | 1 | 0.25 | 10 | 1 | 100 | 0.5 |
| 186 | 3:1 | 1 | 0.33 | 10 | 1 | 100 | 0.5 |
| 187 | 2:1 | 1 | 0.5 | 10 | 1 | 100 | 0.5 |
| 188 | 1:1 | 1 | 1 | 10 | 1 | 100 | 0.5 |
| 189 | 1:2.5 | 1 | 2.5 | 10 | 1 | 100 | 0.5 |
| 190 | 1:5 | 1 | 5 | 10 | 1 | 100 | 0.5 |
| 191 | 1:10 | 1 | 10 | 10 | 1 | 100 | 0.5 |
| 192 | 1:20 | 1 | 20 | 10 | 1 | 100 | 0.5 |
| 193 | 1:25 | 0.5 | 12.5 | 10 | 1 | 100 | 0.5 |
| 194 | 1:50 | 0.25 | 12.5 | 10 | 1 | 100 | 0.5 |
| 195 | 1:67 | 0.25 | 16.75 | 10 | 1 | 100 | 0.5 |
| 196 | 1:75 | 0.2 | 15 | 10 | 1 | 100 | 0.5 |
| 197 | 1:100 | 0.2 | 20 | 10 | 1 | 100 | 0.5 |
| 198 | 1:200 | 0.1 | 20 | 10 | 1 | 100 | 0.5 |
| 199 | 1:250 | 0.08 | 20 | 10 | 1 | 100 | 0.5 |
| 200 | 1:300 | 0.067 | 20 | 10 | 1 | 100 | 0.5 |
| 201 | 1:400 | 0.05 | 20 | 10 | 1 | 100 | 0.5 |
| 202 | 1:500 | 0.04 | 20 | 10 | 1 | 100 | 0.5 |
| 203 | 1:600 | 0.033 | 20 | 10 | 1 | 100 | 0.5 |
| 204 | 1:700 | 0.029 | 20 | 10 | 1 | 100 | 0.5 |
| 205 | 1:800 | 0.025 | 20 | 10 | 1 | 100 | 0.5 |
| 206 | 1:900 | 0.022 | 20 | 10 | 1 | 100 | 0.5 |
| 207 | 1:1000 | 0.02 | 20 | 10 | 1 | 100 | 0.5 |
| 208 | 4:1 | 1 | 0.25 | 10 | 1 | 100 | 1 |
| 209 | 3:1 | 1 | 0.33 | 10 | 1 | 100 | 1 |
| 210 | 2:1 | 1 | 0.5 | 10 | 1 | 100 | 1 |
| 211 | 1:1 | 1 | 1 | 10 | 1 | 100 | 1 |
| 212 | 1:2.5 | 1 | 2.5 | 10 | 1 | 100 | 1 |
| 213 | 1:5 | 1 | 5 | 10 | 1 | 100 | 1 |
| 214 | 1:10 | 1 | 10 | 10 | 1 | 100 | 1 |
| 215 | 1:20 | 1 | 20 | 10 | 1 | 100 | 1 |
| 216 | 1:25 | 0.5 | 12.5 | 10 | 1 | 100 | 1 |
| 217 | 1:50 | 0.25 | 12.5 | 10 | 1 | 100 | 1 |
| 218 | 1:67 | 0.25 | 16.75 | 10 | 1 | 100 | 1 |
| 219 | 1:75 | 0.2 | 15 | 10 | 1 | 100 | 1 |
| 220 | 1:100 | 0.2 | 20 | 10 | 1 | 100 | 1 |
| 221 | 1:200 | 0.1 | 20 | 10 | 1 | 100 | 1 |
| 222 | 1:250 | 0.08 | 20 | 10 | 1 | 100 | 1 |
| 223 | 1:300 | 0.067 | 20 | 10 | 1 | 100 | 1 |
| 224 | 1:400 | 0.05 | 20 | 10 | 1 | 100 | 1 |
| 225 | 1:500 | 0.04 | 20 | 10 | 1 | 100 | 1 |
| 226 | 1:600 | 0.033 | 20 | 10 | 1 | 100 | 1 |
| 227 | 1:700 | 0.029 | 20 | 10 | 1 | 100 | 1 |
| 228 | 1:800 | 0.025 | 20 | 10 | 1 | 100 | 1 |
| 229 | 1:900 | 0.022 | 20 | 10 | 1 | 100 | 1 |
| 230 | 1:1000 | 0.02 | 20 | 10 | 1 | 100 | 1 |

^{a} Nintedanib amounts in nintedanib base or nintedanib base within a nintedanib salt thereof

### Example 9. High Drug Loading Fixed Dose Combination of Nintedanib HBr and Pirfenidone with Controlled Particle Morphology

High drug loading fix-dosed combination formulation of nintedanib HBr, base or other salt thereof pirfenidone can be formulated by dissolving nintedanib and pirfenidone in hot water (50° C.) with shelf forming agents (L-leucine, trileucine, sodium stearate, magnesium stearate) and glass formers as stabilizing agents (sucrose and trehalose) in the ratios below (Table 37).

TABLE 37
High drug load fix-dosed combination
formulation of nintedanib and pirfenidone Glass

| | Nintedanib/ | | | |
|---|---|---|---|---|
| | Pirfenidone | | | |
| | Ratio (weight | % Active | % Shell | % Glass |
| Formulation | by weight) | Drugs | Former^{a} | Former |
| 1 | 4:1 | 100% | N/A | N/A |
| 2 | 4:1 | 90% | 10% L-leucine | N/A |
| 3 | 4:1 | 70% | 10% L-leucine | 20% Sucrose |
| 4 | 4:1 | 70% | 10% L-leucine | 20% |
| | | | | Trehalose |
| 5 | 4:1 | 95% | 5% Trileucine | N/A |
| 6 | 4:1 | 75% | 5% Trileucine | 20% Sucrose |
| 7 | 4:1 | 75% | 5% Trileucine | 20% |
| | | | | Trehalose |
| 8 | 4:1 | 99% | 1% Na Stearate | N/A |
| 9 | 4:1 | 79% | 1% Na Stearate | 20% Sucrose |
| 10 | 4:1 | 79% | 1% Na Stearate | 20% |
| | | | | Trehalose |
| 11 | 3:1 | 100% | N/A | N/A |
| 12 | 3:1 | 90% | 10% L-leucine | N/A |
| 13 | 3:1 | 70% | 10% L-leucine | 20% Sucrose |
| 14 | 3:1 | 70% | 10% L-leucine | 20% |
| | | | | Trehalose |
| 15 | 3:1 | 95% | 5% Trileucine | N/A |
| 16 | 3:1 | 75% | 5% Trileucine | 20% Sucrose |
| 17 | 3:1 | 75% | 5% Trileucine | 20% |
| | | | | Trehalose |
| 18 | 3:1 | 99% | 1% Na Stearate | N/A |
| 19 | 3:1 | 79% | 1% Na Stearate | 20% Sucrose |
| 20 | 3:1 | 79% | 1% Na Stearate | 20% |
| | | | | Trehalose |
| 21 | 2:1 | 100% | N/A | N/A |
| 22 | 2:1 | 90% | 10% L-leucine | N/A |
| 23 | 2:1 | 70% | 10% L-leucine | 20% Sucrose |
| 24 | 2:1 | 70% | 10% L-leucine | 20% |
| | | | | Trehalose |
| 25 | 2:1 | 95% | 5% Trileucine | N/A |
| 26 | 2:1 | 75% | 5% Trileucine | 20% Sucrose |
| 27 | 2:1 | 75% | 5% Trileucine | 20% |
| | | | | Trehalose |
| 28 | 2:1 | 99% | 1% Na Stearate | N/A |
| 29 | 2:1 | 79% | 1% Na Stearate | 20% Sucrose |
| 30 | 2:1 | 79% | 1% Na Stearate | 20% |
| | | | | Trehalose |
| 31 | 1:1 | 100% | N/A | N/A |
| 32 | 1:1 | 90% | 10% L-leucine | N/A |
| 33 | 1:1 | 70% | 10% L-leucine | 20% Sucrose |
| 34 | 1:1 | 70% | 10% L-leucine | 20% |
| | | | | Trehalose |
| 35 | 1:1 | 95% | 5% Trileucine | N/A |
| 36 | 1:1 | 75% | 5% Trileucine | 20% Sucrose |
| 37 | 1:1 | 75% | 5% Trileucine | 20% |
| | | | | Trehalose |
| 38 | 1:1 | 99% | 1% Na Stearate | N/A |
| 39 | 1:1 | 79% | 1% Na Stearate | 20% Sucrose |
| 40 | 1:1 | 79% | 1% Na Stearate | 20% |
| | | | | Trehalose |
| 41 | 1:2.5 | 100% | N/A | N/A |
| 42 | 1:2.5 | 90% | 10% L-leucine | N/A |
| 43 | 1:2.5 | 70% | 10% L-leucine | 20% Sucrose |
| 44 | 1:2.5 | 70% | 10% L-leucine | 20% |
| | | | | Trehalose |
| 45 | 1:2.5 | 95% | 5% Trileucine | N/A |
| 46 | 1:2.5 | 75% | 5% Trileucine | 20% Sucrose |
| 47 | 1:2.5 | 75% | 5% Trileucine | 20% |
| | | | | Trehalose |
| 48 | 1:2.5 | 99% | 1% Na Stearate | N/A |
| 49 | 1:2.5 | 79% | 1% Na Stearate | 20% Sucrose |
| 50 | 1:2.5 | 79% | 1% Na Stearate | 20% |
| | | | | Trehalose |
| 51 | 1:5 | 100% | N/A | N/A |
| 52 | 1:5 | 90% | 10% L-leucine | N/A |
| 53 | 1:5 | 70% | 10% L-leucine | 20% Sucrose |
| 54 | 1:5 | 70% | 10% L-leucine | 20% |
| | | | | Trehalose |
| 55 | 1:5 | 95% | 5% Trileucine | N/A |
| 56 | 1:5 | 75% | 5% Trileucine | 20% Sucrose |
| 57 | 1:5 | 75% | 5% Trileucine | 20% |
| | | | | Trehalose |
| 58 | 1:5 | 99% | 1% Na Stearate | N/A |
| 59 | 1:5 | 79% | 1% Na Stearate | 20% Sucrose |
| 60 | 1:5 | 79% | 1% Na Stearate | 20% |
| | | | | Trehalose |
| 61 | 1:10 | 100% | N/A | N/A |
| 62 | 1:10 | 90% | 10% L-leucine | V/A |
| 63 | 1:10 | 70% | 10% L-leucine | 20% Sucrose |
| 64 | 1:10 | 70% | 10% L-leucine | 20% |
| | | | | Trehalose |
| 65 | 1:10 | 95% | 5% Trileucine | N/A |
| 66 | 1:10 | 75% | 5% Trileucine | 20% Sucrose |
| 67 | 1:10 | 75% | 5% Trileucine | 20% |
| | | | | Trehalose |
| 68 | 1:10 | 99% | 1% Na Stearate | N/A |
| 69 | 1:10 | 79% | 1% Na Stearate | 20% Sucrose |
| 70 | 1:10 | 79% | 1% Na Stearate | 20% |
| | | | | Trehalose |
| 71 | 1:20 | 100% | N/A | N/A |
| 72 | 1:20 | 90% | 10% L-leucine | N/A |
| 73 | 1:20 | 70% | 10% L-leucine | 20% Sucrose |
| 74 | 1:20 | 70% | 10% L-leucine | 20% |
| | | | | Trehalose |
| 75 | 1:20 | 95% | 5% Trileucine | N/A |
| 76 | 1:20 | 75% | 5% Trileucine | 20% Sucrose |
| 77 | 1:20 | 75% | 5% Trileucine | 20% |
| | | | | Trehalose |
| 78 | 1:20 | 99% | 1% Na Stearate | N/A |
| 79 | 1:20 | 79% | 1% Na Stearate | 20% Sucrose |
| 80 | 1:20 | 79% | 1% Na Stearate | 20% |
| | | | | Trehalose |
| 81 | 1:25 | 100% | N/A | N/A |
| 82 | 1:25 | 90% | 10% L-leucine | N/A |
| 83 | 1:25 | 70% | 10% L-leucine | 20% Sucrose |
| 84 | 1:25 | 70% | 10% L-leucine | 20% |
| | | | | Trehalose |
| 85 | 1:25 | 95% | 5% Trileucine | N/A |
| 86 | 1:25 | 75% | 5% Trileucine | 20% Sucrose |
| 87 | 1:25 | 75% | 5% Trileucine | 20% |
| | | | | Trehalose |
| 88 | 1:25 | 99% | 1% Na Stearate | N/A |
| 89 | 1:25 | 79% | 1% Na Stearate | 20% Sucrose |
| 90 | 1:25 | 79% | 1% Na Stearate | 20% |
| | | | | Trehalose |
| 91 | 1:50 | 100% | N/A | N/A |
| 92 | 1:50 | 90% | 10% L-leucine | N/A |
| 93 | 1:50 | 70% | 10% L-leucine | 20% Sucrose |
| 94 | 1:50 | 70% | 10% L-leucine | 20% |
| | | | | Trehalose |
| 95 | 1:50 | 95% | 5% Trileucine | N/A |
| 96 | 1:50 | 75% | 5% Trileucine | 20% Sucrose |
| 97 | 1:50 | 75% | 5% Trileucine | 20% |
| | | | | Trehalose |
| 98 | 1:50 | 99% | 1% Na Stearate | N/A |
| 99 | 1:50 | 79% | 1% Na Stearate | 20% Sucrose |
| 100 | 1:50 | 79% | 1% Na Stearate | 20% |
| | | | | Trehalose |
| 101 | 1:67 | 100% | N/A | N/A |
| 102 | 1:67 | 90% | 10% L-leucine | N/A |
| 103 | 1:67 | 70% | 10% L-leucine | 20% Sucrose |
| 104 | 1:67 | 70% | 10% L-leucine | 20% |
| | | | | Trehalose |
| 105 | 1:67 | 95% | 5% Trileucine | N/A |
| 106 | 1:67 | 75% | 5% Trileucine | 20% Sucrose |
| 107 | 1:67 | 75% | 5% Trileucine | 20% |
| | | | | Trehalose |
| 108 | 1:6 | 99% | 1% Na Stearate | N/A |
| 109 | 1:67 | 79% | 1% Na Stearate | 20% Sucrose |
| 110 | 1:67 | 79% | 1% Na Stearate | 20% |
| | | | | Trehalose |
| 111 | 1:75 | 100% | N/A | N/A |
| 112 | 1:75 | 90% | 10% L-leucine | N/A |
| 113 | 1:75 | 70% | 10% L-leucine | 20% Sucrose |
| 114 | 1:75 | 70% | 10% L-leucine | 20% |
| | | | | Trehalose |
| 115 | 1:75 | 95% | 5% Trileucine | N/A |
| 116 | 1:75 | 75% | 5% Trileucine | 20% Sucrose |
| 117 | 1:75 | 75% | 5% Trileucine | 20% |
| | | | | Trehalose |
| 118 | 1:75 | 99% | 1% Na Stearate | N/A |
| 119 | 1:75 | 79% | 1% Na Stearate | 20% Sucrose |
| 120 | 1:75 | 79% | 1% Na Stearate | 20% |
| | | | | Trehalose |
| 121 | 1:100 | 100% | N/A | N/A |
| 122 | 1:100 | 90% | 10% L-leucine | N/A |
| 123 | 1:100 | 70% | 10% L-leucine | 20% Sucrose |
| 124 | 1:100 | 70% | 10% L-leucine | 20% |
| | | | | Trehalose |
| 125 | 1:100 | 95% | 5% Trileucine | N/A |
| 126 | 1:100 | 75% | 5% Trileucine | 20% Sucrose |
| 127 | 1:100 | 75% | 5% Trileucine | 20% |
| | | | | Trehalose |
| 128 | 1:100 | 99% | 1% Na Stearate | N/A |
| 129 | 1:100 | 79% | 1% Na Stearate | 20% Sucrose |
| 130 | 1:100 | 79% | 1% Na Stearate | 20% |
| | | | | Trehalose |
| 131 | 1:200 | 100% | N/A | N/A |
| 132 | 1:200 | 90% | 10% L-leucine | N/A |
| 133 | 1:200 | 70% | 10% L-leucine | 20% Sucrose |
| 134 | 1:200 | 70% | 10% L-leucine | 20% |
| | | | | Trehalose |
| 135 | 1:200 | 95% | 5% Trileucine | N/A |
| 136 | 1:200 | 75% | 5% Trileucine | 20% Sucrose |
| 137 | 1:200 | 75% | 5% Trileucine | 20% |
| | | | | Trehalose |
| 138 | 1:200 | 99% | 1% Na Stearate | N/A |
| 139 | 1:200 | 79% | 1% Na Stearate | 20% Sucrose |
| 140 | 1:200 | 79% | 1% Na Stearate | 20% |
| | | | | Trehalose |
| 141 | 1:250 | 100% | N/A | N/A |
| 142 | 1:250 | 90% | 10% L-leucine | N/A |
| 143 | 1:250 | 70% | 10% L-leucine | 20% Sucrose |
| 144 | 1:250 | 70% | 10% L-leucine | 20% |
| | | | | Trehalose |
| 145 | 1:250 | 95% | 5% Trileucine | V/A |
| 146 | 1:250 | 75% | 5% Trileucine | 20% Sucrose |
| 147 | 1:250 | 75% | 5% Trileucine | 20% |
| | | | | Trehalose |
| 148 | 1:250 | 99% | 1% Na Stearate | N/A |
| 149 | 1:250 | 79% | 1% Na Stearate | 20% Sucrose |
| 150 | 1:250 | 79% | 1% Na Stearate | 20% |
| | | | | Trehalose |
| 151 | 1:300 | 100% | N/A | N/A |
| 152 | 1:300 | 90% | 10% L-leucine | N/A |
| 153 | 1:300 | 70% | 10% L-leucine | 20% Sucrose |
| 154 | 1:300 | 70% | 10% L-leucine | 20% |
| | | | | Trehalose |
| 155 | 1:300 | 95% | 5% Trileucine | N/A |
| 156 | 1:300 | 75% | 5% Trileucine | 20% Sucrose |
| 157 | 1:300 | 75% | 5% Trileucine | 20% |
| | | | | Trehalose |
| 157 | 1:300 | 99% | 1% Na Stearate | N/A |
| 159 | 1:300 | 79% | 1% Na Stearate | 20% Sucrose |
| 160 | 1:300 | 79% | 1% Na Stearate | 20% |
| | | | | Trehalose |
| 161 | 1:400 | 100% | N/A | N/A |
| 162 | 1:400 | 90% | 10% L-leucine | N/A |
| 163 | 1:400 | 70% | 10% L-leucine | 20% Sucrose |
| 164 | 1:400 | 70% | 10% L-leucine | 20% |
| | | | | Trehalose |
| 165 | 1:400 | 95% | 5% Trileucine | N/A |
| 166 | 1:400 | 75% | 5% Trileucine | 20% Sucrose |
| 167 | 1:400 | 75% | 5% Trileucine | 20% |
| | | | | Trehalose |
| 168 | 1:400 | 99% | 1% Na Stearate | N/A |
| 169 | 1:400 | 79% | 1% Na Stearate | 20% Sucrose |
| 170 | 1:400 | 79% | 1% Na Stearate | 20% |
| | | | | Trehalose |
| 171 | 1:500 | 100% | N/A | N/A |
| 172 | 1:500 | 90% | 10% L-leucine | N/A |
| 173 | 1:500 | 70% | 10% L-leucine | 20% Sucrose |
| 174 | 1:500 | 70% | 10% L-leucine | 20% |
| | | | | Trehalose |
| 175 | 1:500 | 95% | 5% Trileucine | N/A |
| 176 | 1:500 | 75% | 5% Trileucine | 20% Sucrose |
| 177 | 1:500 | 75% | 5% Trileucine | 20% |
| | | | | Trehalose |
| 178 | 1:500 | 99% | 1% Na Stearate | N/A |
| 179 | 1:500 | 79% | 1% Na Stearate | 20% Sucrose |
| 180 | 1:500 | 79% | 1% Na Stearate | 20% |
| | | | | Trehalose |
| 181 | 1:600 | 100% | N/A | N/A |
| 182 | 1:600 | 90% | 10% L-leucine | N/A |
| 183 | 1:600 | 70% | 10% L-leucine | 20% Sucrose |
| 184 | 1:600 | 70% | 10% L-leucine | 20% |
| | | | | Trehalose |
| 185 | 1:600 | 95% | 5% Trileucine | N/A |
| 186 | 1:600 | 75% | 5% Trileucine | 20% Sucrose |
| 187 | 1:600 | 75% | 5% Trileucine | 20% |
| | | | | Trehalose |
| 188 | 1:600 | 99% | 1% Na Stearate | N/A |
| 189 | 1:600 | 79% | 1% Na Stearate | 20% Sucrose |
| 190 | 1:600 | 79% | 1% Na Stearate | 20% |
| | | | | Trehalose |
| 191 | 1:700 | 100% | N/A | N/A |
| 192 | 1:700 | 90% | 10% L-leucine | N/A |
| 193 | 1:700 | 70% | 10% L-leucine | 20% Sucrose |
| 194 | 1:700 | 70% | 10% L-leucine | 20% |
| | | | | Trehalose |
| 195 | 1:700 | 95% | 5% Trileucine | N/A |
| 196 | 1:700 | 75% | 5% Trileucine | 20% Sucrose |
| 197 | 1:700 | 75% | 5% Trileucine | 20% |
| | | | | Trehalose |
| 198 | 1:700 | 99% | 1% Na Stearate | N/A |
| 199 | 1:700 | 79% | 1% Na Stearate | 20% Sucrose |
| 200 | 1:700 | 79% | 1% Na Stearate | 20% |
| | | | | Trehalose |
| 201 | 1:800 | 100% | N/A | N/A |
| 202 | 1:800 | 90% | 10% L-leucine | N/A |
| 203 | 1:800 | 70% | 10% L-leucine | 20% Sucrose |
| 204 | 1:800 | 70% | 10% L-leucine | 20% |
| | | | | Trehalose |
| 205 | 1:800 | 95% | 5% Trileucine | N/A |
| 206 | 1:800 | 75% | 5% Trileucine | 20% Sucrose |
| 207 | 1:800 | 75% | 5% Trileucine | 20% |
| | | | | Trehalose |
| 208 | 1:800 | 99% | 1% Na Stearate | N/A |
| 209 | 1:800 | 79% | 1% Na Stearate | 20% Sucrose |
| 210 | 1:800 | 79% | 1% Na Stearate | 20% |
| | | | | Trehalose |
| 211 | 1:900 | 100% | N/A | N/A |
| 212 | 1:900 | 90% | 10% L-leucine | N/A |
| 213 | 1:900 | 70% | 10% L-leucine | 20% Sucrose |
| 214 | 1:900 | 70% | 10% L-leucine | 20% |
| | | | | Trehalose |
| 215 | 1:900 | 95% | 5% Trileucine | N/A |
| 216 | 1:900 | 75% | 5% Trileucine | 20% Sucrose |
| 217 | 1:900 | 75% | 5% Trileucine | 20% |
| | | | | Trehalose |
| 218 | 1:900 | 99% | 1% Na Stearate | N/A |
| 219 | 1:900 | 79% | 1% Na Stearate | 20% Sucrose |
| 220 | 1:900 | 79% | 1% Na Stearate | 20% |
| | | | | Trehalose |
| 221 | 1:1000 | 100% | N/A | N/A |
| 222 | 1:1000 | 90% | 10% L-leucine | N/A |
| 223 | 1:1000 | 70% | 10% L-leucine | 20% Sucrose |
| 224 | 1:1000 | 70% | 10% L-leucine | 20% |
| | | | | Trehalose |
| 225 | 1:1000 | 95% | 5% Trileucine | N/A |
| 226 | 1:1000 | 75% | 5% Trileucine | 20% Sucrose |
| 227 | 1:1000 | 75% | 5% Trileucine | 20% |
| | | | | Trehalose |
| 228 | 1:1000 | 99% | 1% Na Stearate | N/A |
| 229 | 1:1000 | 79% | 1% Na Stearate | 20% Sucrose |
| 300 | 1:1000 | 79% | 1% Na Stearate | 20% |
| | | | | Trehalose |

^{a}Sodium stearate may be substituted with magnesium stearate

The above formulations are spray dried using a Buchi MiniSpray dryer B-290 (Switzerland) under the following drying conditions: inlet temperature, 100-110° C.; airflow rate, 450-500 L/h; aspirator, 9000 and pump rate, 6.0 mL/min. These conditions resulted in an outlet temperature of 45-50° C. The resulting powder is collected in a cyclone separator. These have smooth spherical shape (particles formed without shell forming agents) or wrinkled surfaces (with shell forming agents) as determined by scanning electron microscopy (SEM).

In vitro aerosol performances (fine particle fraction and MMAD) of the above formulations are tested by Next-Generation Impactor (NGI). Up to 40 mg of the powder is filled into gelatin capsules (the exact amount depends on the required dose) and dispersed using a low resistance Plastiape RS01 DPI device at a flow rate of 100 L/min over 2.4 s to inspire 4 L air. The amounts of nintedanib HBr and pirfenidone on each stage are recovered and analyzed by HPLC. Depending on the formulation, multiple actuations may be required to collect adequate amount of drugs in the impactor stage. The fine particle fraction (FPF), mass median aerodynamic diameter (MMAD) are expected to be ≥50% and ≤5 μm respectively. The amount of formulation to achieve the target nintedanib and pirfenidone, along with the amount to be filled in Size 3 capsules.

### Example 10. Nintedanib and Pirfenidone Ratio Optimization

Selected formulations from the above formulations can be filled into three to four capsules to produce nintedanib and pirfenidone fixed dose combinations in the range of 0.1 mg to 2 mg (freebase) and 50 mg to 100 mg, respectively (Table 38).

### Table 38. Optimized Nintedanib or Salt Thereof and Pirfenidone Combination Formations

A. Sodium Stearate May be Substituted with Magnesium Stearate

TABLE 38
High drug loading fixed dose combination of nintedanib
HBr and pirfenidone with hollow porous particles

| | | Active drugs | | |
|---|---|---|---|---|
| | Nintedanib/ | (Nintedanib or salt | | |
| | Pirfenidone | thereof, including | | |
| Spray Dried | Ratio (weight | hydrobromide/ | Formulation | Formulation |
| Formulation | by weight) | pirfenidone)^{a} | Components | amount (mg) |
| 1 | 4:1 | 100 mg/25 mg | No excipients | 125.00 |
| 2 | 4:1 | 100 mg/25 mg | 10% leucine | 138.89 |
| 3 | 4:1 | 100 mg/25 mg | 10% leucine/20% | 178.57 |
| | | | sucrose | |
| 4 | 4:1 | 100 mg/25 mg | 10% leucine/20% | 178.57 |
| | | | trehalose | |
| 5 | 4:1 | 100 mg/25 mg | 5% T-leucine | 131.58 |
| 6 | 4:1 | 100 mg/25 mg | 5% T- | 166.67 |
| | | | leucine/20% | |
| | | | sucrose | |
| 7 | 4:1 | 100 mg/25 mg | 5% T- | 166.67 |
| | | | leucine/20% | |
| | | | trehalose | |
| 8 | 4:1 | 100 mg/25 mg | 1% Na stearate | 126.26 |
| 9 | 4:1 | 100 mg/25 mg | 1% Na | 158.23 |
| | | | stearate/20% | |
| | | | Sucrose | |
| 10 | 4:1 | 100 mg/25 mg | 1% Na | 158.23 |
| | | | stearate/20% | |
| | | | trehalose | |
| 11 | 3:1 | 100 mg/33.3 mg | No excipients | 133.33 |
| 12 | 3:1 | 100 mg/33.3 mg | 10% leucine | 148.14 |
| 13 | 3:1 | 100 mg/33.3 mg | 10% leucine/20% | 190.47 |
| | | | sucrose | |
| 14 | 3:1 | 100 mg/33.3 mg | 10% leucine/20% | 190.47 |
| | | | trehalose | |
| 15 | 3:1 | 100 mg/33.3 mg | 5% T-leucine | 140.35 |
| 16 | 3:1 | 100 mg/33.3 mg | 5% T- | 177.77 |
| | | | leucine/20% | |
| | | | sucrose | |
| 17 | 3:1 | 100 mg/33.3 mg | 5% T- | 177.77 |
| | | | leucine/20% | |
| | | | trehalose | |
| 18 | 3:1 | 100 mg/33.3 mg | 1% Na stearate | 134.68 |
| 19 | 3:1 | 100 mg/33.3 mg | 1% Na | 168.77 |
| | | | stearate/20% | |
| | | | Sucrose | |
| 20 | 3:1 | 100 mg/33.3 mg | 1% Na | 168.77 |
| | | | stearate/20% | |
| | | | trehalose | |
| 21 | 2:1 | 100 mg/50 mg | No excipients | 150.00 |
| 22 | 2:1 | 100 mg/50 mg | 10% leucine | 166.67 |
| 23 | 2:1 | 100 mg/50 mg | 10% leucine/20% | 214.29 |
| | | | sucrose | |
| 24 | 2:1 | 100 mg/50 mg | 10% leucine/20% | 214.29 |
| | | | trehalose | |
| 25 | 2:1 | 100 mg/50 mg | 5% T-leucine | 157.89 |
| 26 | 2:1 | 100 mg/50 mg | 5% T- | 200.00 |
| | | | leucine/20% | |
| | | | sucrose | |
| 27 | 2:1 | 100 mg/50 mg | 5% T- | 200.00 |
| | | | leucine/20% | |
| | | | trehalose | |
| 28 | 2:1 | 100 mg/50 mg | 1% Na stearate | 151.52 |
| 29 | 2:1 | 100 mg/50 mg | 1% Na | 189.87 |
| | | | stearate/20% | |
| | | | Sucrose | |
| 30 | 2:1 | 100 mg/50 mg | 1% Na | 189.87 |
| | | | stearate/20% | |
| | | | trehalose | |
| 31 | 1:1 | 100 mg/100 mg | No excipients | 200.00 |
| 32 | 1:1 | 100 mg/100 mg | 10% leucine | 166.67 |
| 33 | 1:1 | 100 mg/100 mg | 10% leucine/20% | 214.29 |
| | | | sucrose | |
| 34 | 1:1 | 100 mg/100 mg | 10% leucine/20% | 214.29 |
| | | | trehalose | |
| 35 | 1:1 | 100 mg/100 mg | 5% T-leucine | 157.89 |
| 36 | 1:1 | 100 mg/100 mg | 5% T- | 200.00 |
| | | | leucine/20% | |
| | | | sucrose | |
| 37 | 1:1 | 100 mg/100 mg | 5% T- | 200.00 |
| | | | leucine/20% | |
| | | | trehalose | |
| 38 | 1:1 | 100 mg/100 mg | 1% Na stearate | 151.52 |
| 39 | 1:1 | 100 mg/100 mg | 1% Na | 189.87 |
| | | | stearate/20% | |
| | | | Sucrose | |
| 40 | 1:1 | 100 mg/100 mg | 1% Na | 189.87 |
| | | | stearate/20% | |
| | | | trehalose | |
| 41 | 4:1 | 75 mg/18.75 mg | No excipients | 93.75 |
| 42 | 4:1 | 75 mg/18.75 mg | 10% leucine | 104.17 |
| 43 | 4:1 | 75 mg/18.75 mg | 10% leucine/20% | 133.93 |
| | | | sucrose | |
| 44 | 4:1 | 75 mg/18.75 mg | 10% leucine/20% | 133.93 |
| | | | trehalose | |
| 45 | 4:1 | 75 mg/18.75 mg | 5% T-leucine | 98.68 |
| 46 | 4:1 | 75 mg/18.75 mg | 5% T- | 125.00 |
| | | | leucine/20% | |
| | | | sucrose | |
| 47 | 4:1 | 75 mg/18.75 mg | 5% T- | 125.00 |
| | | | leucine/20% | |
| | | | trehalose | |
| 48 | 4:1 | 75 mg/18.75 mg | 1% Na stearate | 94.70 |
| 49 | 4:1 | 75 mg/18.75 mg | 1% Na | 118.67 |
| | | | stearate/20% | |
| | | | Sucrose | |
| 50 | 4:1 | 75 mg/18.75 mg | 1% Na | 118.67 |
| | | | stearate/20% | |
| | | | trehalose | |
| 51 | 3:1 | 75 mg/25 mg | No excipients | 100.00 |
| 52 | 3:1 | 75 mg/25 mg | 10% leucine | 111.11 |
| 53 | 3:1 | 75 mg/25 mg | 10% leucine/20% | 142.86 |
| | | | sucrose | |
| 54 | 3:1 | 75 mg/25 mg | 10% leucine/20% | 142.86 |
| | | | trehalose | |
| 55 | 3:1 | 75 mg/25 mg | 5% T-leucine | 105.26 |
| 56 | 3:1 | 75 mg/25 mg | 5% T- | 133.33 |
| | | | leucine/20% | |
| | | | sucrose | |
| 57 | 3:1 | 75 mg/25 mg | 5% T- | 133.33 |
| | | | leucine/20% | |
| | | | trehalose | |
| 58 | 3:1 | 75 mg/25 mg | 1% Na stearate | 101.01 |
| 59 | 3:1 | 75 mg/25 mg | 1% Na | 126.58 |
| | | | stearate/20% | |
| | | | Sucrose | |
| 60 | 3:1 | 75 mg/25 mg | 1% Na | 126.58 |
| | | | stearate/20% | |
| | | | trehalose | |
| 61 | 2:1 | 75 mg/37.5 mg | No excipients | 112.50 |
| 62 | 2:1 | 75 mg/37.5 mg | 10% leucine | 125.00 |
| 63 | 2:1 | 75 mg/37.5 mg | 10% leucine/20% | 160.71 |
| | | | sucrose | |
| 64 | 2:1 | 75 mg/37.5 mg | 10% leucine/20% | 160.71 |
| | | | trehalose | |
| 65 | 2:1 | 75 mg/37.5 mg | 5% T-leucine | 118.42 |
| 66 | 2:1 | 75 mg/37.5 mg | 5% T- | 150.00 |
| | | | leucine/20% | |
| | | | sucrose | |
| 67 | 2:1 | 75 mg/37.5 mg | 5% T- | 150.00 |
| | | | leucine/20% | |
| | | | trehalose | |
| 68 | 2:1 | 75 mg/37.5 mg | 1% Na stearate | 113.64 |
| 69 | 2:1 | 75 mg/37.5 mg | 1% Na | 142.41 |
| | | | stearate/20% | |
| | | | Sucrose | |
| 70 | 2:1 | 75 mg/37.5 mg | 1% Na | 142.41 |
| | | | stearate/20% | |
| | | | trehalose | |
| 71 | 1:1 | 75 mg/75 mg | No excipients | 150.00 |
| 72 | 1:1 | 75 mg/75 mg | 10% leucine | 166.67 |
| 73 | 1:1 | 75 mg/75 mg | 10% leucine/20% | 214.29 |
| | | | sucrose | |
| 74 | 1:1 | 75 mg/75 mg | 10% leucine/20% | 214.29 |
| | | | trehalose | |
| 75 | 1:1 | 75 mg/75 mg | 5% T-leucine | 157.89 |
| 76 | 1:1 | 75 mg/75 mg | 5% T- | 200.00 |
| | | | leucine/20% | |
| | | | sucrose | |
| 77 | 1:1 | 75 mg/75 mg | 5% T- | 200.00 |
| | | | leucine/20% | |
| | | | trehalose | |
| 78 | 1:1 | 75 mg/75 mg | 1% Na stearate | 151.52 |
| 79 | 1:1 | 75 mg/75 mg | 1% Na | 189.87 |
| | | | stearate/20% | |
| | | | Sucrose | |
| 80 | 1:1 | 75 mg/75 mg | 1% Na | 189.87 |
| | | | stearate/20% | |
| | | | trehalose | |
| 81 | 4:1 | 50 mg/12.5 mg | No excipients | 62.50 |
| 82 | 4:1 | 50 mg/12.5 mg | 10% leucine | 69.44 |
| 83 | 4:1 | 50 mg/12.5 mg | 10% leucine/20% | 89.29 |
| | | | sucrose | |
| 84 | 4:1 | 50 mg/12.5 mg | 10% leucine/20% | 89.29 |
| | | | trehalose | |
| 85 | 4:1 | 50 mg/12.5 mg | 5% T-leucine | 65.79 |
| 86 | 4:1 | 50 mg/12.5 mg | 5% T- | 83.33 |
| | | | leucine/20% | |
| | | | sucrose | |
| 87 | 4:1 | 50 mg/12.5 mg | 5% T- | 83.33 |
| | | | leucine/20% | |
| | | | trehalose | |
| 88 | 4:1 | 50 mg/12.5 mg | 1% Na stearate | 63.13 |
| 89 | 4:1 | 50 mg/12.5 mg | 1% Na | 79.11 |
| | | | stearate/20% | |
| | | | Sucrose | |
| 90 | 4:1 | 50 mg/12.5 mg | 1% Na | 79.11 |
| | | | stearate/20% | |
| | | | trehalose | |
| 91 | 3:1 | 50 mg/16.67 mg | No excipients | 66.67 |
| 92 | 3:1 | 50 mg/16.67 mg | 10% leucine | 74.07 |
| 93 | 3:1 | 50 mg/16.67 mg | 10% leucine/20% | 95.24 |
| | | | sucrose | |
| 94 | 3:1 | 50 mg/16.67 mg | 10% leucine/20% | 95.24 |
| | | | trehalose | |
| 95 | 3:1 | 50 mg/16.67 mg | 5% T-leucine | 70.18 |
| 96 | 3:1 | 50 mg/16.67 mg | 5% T- | 88.89 |
| | | | leucine/20% | |
| | | | sucrose | |
| 97 | 3:1 | 50 mg/16.67 mg | 5% T- | 88.89 |
| | | | leucine/20% | |
| | | | trehalose | |
| 98 | 3:1 | 50 mg/16.67 mg | 1% Na stearate | 67.34 |
| 99 | 3:1 | 50 mg/16.67 mg | 1% Na | 84.39 |
| | | | stearate/20% | |
| | | | Sucrose | |
| 100 | 3:1 | 50 mg/16.67 mg | 1% Na | 84.39 |
| | | | stearate/20% | |
| | | | trehalose | |
| 101 | 2:1 | 50 mg/25 mg | No excipients | 75.00 |
| 102 | 2:1 | 50 mg/25 mg | 10% leucine | 83.33 |
| 103 | 2:1 | 50 mg/25 mg | 10% leucine/20% | 107.14 |
| | | | sucrose | |
| 104 | 2:1 | 50 mg/25 mg | 10% leucine/20% | 107.14 |
| | | | trehalose | |
| 105 | 2:1 | 50 mg/25 mg | 5% T-leucine | 78.95 |
| 106 | 2:1 | 50 mg/25 mg | 5% T- | 100.00 |
| | | | leucine/20% | |
| | | | sucrose | |
| 107 | 2:1 | 50 mg/25 mg | 5% T- | 100.00 |
| | | | leucine/20% | |
| | | | trehalose | |
| 108 | 2:1 | 50 mg/25 mg | 1% Na stearate | 75.76 |
| 109 | 2:1 | 50 mg/25 mg | 1% Na | 94.94 |
| | | | stearate/20% | |
| | | | Sucrose | |
| 110 | 2:1 | 50 mg/25 mg | 1% Na | 94.94 |
| | | | stearate/20% | |
| | | | trehalose | |
| 111 | 1:1 | 50 mg/50 mg | No excipients | 50.00 |
| 112 | 1:1 | 50 mg/50 mg | 10% leucine | 55.56 |
| 113 | 1:1 | 50 mg/50 mg | 10% leucine/20% | 71.43 |
| | | | sucrose | |
| 114 | 1:1 | 50 mg/50 mg | 10% leucine/20% | 71.43 |
| | | | trehalose | |
| 115 | 1:1 | 50 mg/50 mg | 5% T-leucine | 52.63 |
| 116 | 1:1 | 50 mg/50 mg | 5% T- | 66.67 |
| | | | leucine/20% | |
| | | | sucrose | |
| 117 | 1:1 | 50 mg/50 mg | 5% T- | 66.67 |
| | | | leucine/20% | |
| | | | trehalose | |
| 118 | 1:1 | 50 mg/50 mg | 1% Na stearate | 50.51 |
| 119 | 1:1 | 50 mg/50 mg | 1% Na | 63.29 |
| | | | stearate/20% | |
| | | | Sucrose | |
| 120 | 1:1 | 50 mg/50 mg | 1% Na | 63.29 |
| | | | stearate/20% | |
| | | | trehalose | |
| 121 | 4:1 | 20 mg/5 mg | No excipients | 25.00 |
| 122 | 4:1 | 20 mg/5 mg | 10% leucine | 27.78 |
| 123 | 4:1 | 20 mg/5 mg | 10% leucine/20% | 35.71 |
| | | | sucrose | |
| 124 | 4:1 | 20 mg/5 mg | 10% leucine/20% | 35.71 |
| | | | trehalose | |
| 125 | 4:1 | 20 mg/5 mg | 5% T-leucine | 26.32 |
| 126 | 4:1 | 20 mg/5 mg | 5% T- | 33.33 |
| | | | leucine/20% | |
| | | | sucrose | |
| 127 | 4:1 | 20 mg/5 mg | 5% T- | 33.33 |
| | | | leucine/20% | |
| | | | trehalose | |
| 128 | 4:1 | 20 mg/5 mg | 1% Na stearate | 25.25 |
| 129 | 4:1 | 20 mg/5 mg | 1% Na | 31.65 |
| | | | stearate/20% | |
| | | | Sucrose | |
| 130 | 4:1 | 20 mg/5 mg | 1% Na | 31.65 |
| | | | stearate/20% | |
| | | | trehalose | |
| 131 | 3:1 | 20 mg/6.67 mg | No excipients | 26.67 |
| 132 | 3:1 | 20 mg/6.67 mg | 10% leucine | 29.63 |
| 133 | 3:1 | 20 mg/6.67 mg | 10% leucine/20% | 38.10 |
| | | | sucrose | |
| 134 | 3:1 | 20 mg/6.67 mg | 10% leucine/20% | 38.10 |
| | | | trehalose | |
| 135 | 3:1 | 20 mg/6.67 mg | 5% T-leucine | 28.07 |
| 136 | 3:1 | 20 mg/6.67 mg | 5% T- | 35.56 |
| | | | leucine/20% | |
| | | | sucrose | |
| 137 | 3:1 | 20 mg/6.67 mg | 5% T- | 35.56 |
| | | | leucine/20% | |
| | | | trehalose | |
| 138 | 3:1 | 20 mg/6.67 mg | 1% Na stearate | 26.94 |
| 139 | 3:1 | 20 mg/6.67 mg | 1% Na | 33.76 |
| | | | stearate/20% | |
| | | | Sucrose | |
| 140 | 3:1 | 20 mg/6.67 mg | 1% Na | 33.76 |
| | | | stearate/20% | |
| | | | trehalose | |
| 141 | 2:1 | 20 mg/10 mg | No excipients | 75.00 |
| 142 | 2:1 | 20 mg/10 mg | 10% leucine | 30.00 |
| 143 | 2:1 | 20 mg/10 mg | 10% leucine/20% | 33.33 |
| | | | sucrose | |
| 144 | 2:1 | 20 mg/10 mg | 10% leucine/20% | 42.86 |
| | | | trehalose | |
| 145 | 2:1 | 20 mg/10 mg | 5% T-leucine | 42.86 |
| 146 | 2:1 | 20 mg/10 mg | 5% T- | 31.58 |
| | | | leucine/20% | |
| | | | sucrose | |
| 147 | 2:1 | 20 mg/10 mg | 5% T- | 40.00 |
| | | | leucine/20% | |
| | | | trehalose | |
| 148 | 2:1 | 20 mg/10 mg | 1% Na stearate | 40.00 |
| 149 | 2:1 | 20 mg/10 mg | 1% Na | 30.30 |
| | | | stearate/20% | |
| | | | Sucrose | |
| 150 | 2:1 | 20 mg/10 mg | 1% Na | 37.97 |
| | | | stearate/20% | |
| | | | trehalose | |
| 150 | 1:1 | 20 mg/20 mg | No excipients | 40.00 |
| 152 | 1:1 | 20 mg/20 mg | 10% leucine | 44.44 |
| 153 | 1:1 | 20 mg/20 mg | 10% leucine/20% | 57.14 |
| | | | sucrose | |
| 154 | 1:1 | 20 mg/20 mg | 10% leucine/20% | 57.14 |
| | | | trehalose | |
| 155 | 1:1 | 20 mg/20 mg | 5% T-leucine | 42.11 |
| 156 | 1:1 | 20 mg/20 mg | 5% T- | 53.33 |
| | | | leucine/20% | |
| | | | sucrose | |
| 157 | 1:1 | 20 mg/20 mg | 5% T- | 53.33 |
| | | | leucine/20% | |
| | | | trehalose | |
| 158 | 1:1 | 20 mg/20 mg | 1% Na stearate | 40.40 |
| 159 | 1:1 | 20 mg/20 mg | 1% Na | 50.63 |
| | | | stearate/20% | |
| | | | Sucrose | |
| 160 | 1:1 | 20 mg/20 mg | 1% Na | 50.63 |
| | | | stearate/20% | |
| | | | trehalose | |
| 161 | 4:1 | 10 mg/2.5 mg | No excipients | 12.50 |
| 162 | 4:1 | 10 mg/2.5 mg | 10% leucine | 13.89 |
| 163 | 4:1 | 10 mg/2.5 mg | 10% leucine/20% | 17.86 |
| | | | sucrose | |
| 164 | 4:1 | 10 mg/2.5 mg | 10% leucine/20% | 17.86 |
| | | | trehalose | |
| 165 | 4:1 | 10 mg/2.5 mg | 5% T-leucine | 13.16 |
| 166 | 4:1 | 10 mg/2.5 mg | 5% T- | 16.67 |
| | | | leucine/20% | |
| | | | sucrose | |
| 167 | 4:1 | 10 mg/2.5 mg | 5% T- | 16.67 |
| | | | leucine/20% | |
| | | | trehalose | |
| 168 | 4:1 | 10 mg/2.5 mg | 1% Na stearate | 12.63 |
| 169 | 4:1 | 10 mg/2.5 mg | 1% Na | 15.82 |
| | | | stearate/20% | |
| | | | Sucrose | |
| 170 | 4:1 | 10 mg/2.5 mg | 1% Na | 15.82 |
| | | | stearate/20% | |
| | | | trehalose | |
| 171 | 3:1 | 10 mg/3.33 mg | No excipients | 26.67 |
| 172 | 3:1 | 10 mg/3.33 mg | 10% leucine | 13.33 |
| 173 | 3:1 | 10 mg/3.33 mg | 10% leucine/20% | 14.81 |
| | | | sucrose | |
| 174 | 3:1 | 10 mg/3.33 mg | 10% leucine/20% | 19.04 |
| | | | trehalose | |
| 175 | 3:1 | 10 mg/3.33 mg | 5% T-leucine | 19.04 |
| 176 | 3:1 | 10 mg/3.33 mg | 5% T- | 14.03 |
| | | | leucine/20% | |
| | | | sucrose | |
| 177 | 3:1 | 10 mg/3.33 mg | 5% T- | 17.77 |
| | | | leucine/20% | |
| | | | trehalose | |
| 178 | 3:1 | 10 mg/3.33 mg | 1% Na stearate | 17.77 |
| 179 | 3:1 | 10 mg/3.33 mg | 1% Na | 13.46 |
| | | | stearate/20% | |
| | | | Sucrose | |
| 180 | 3:1 | 10 mg/3.33 mg | 1% Na | 16.87 |
| | | | stearate/20% | |
| | | | trehalose | |
| 181 | 2:1 | 10 mg/5 mg | No excipients | 15.00 |
| 182 | 2:1 | 10 mg/5 mg | 10% leucine | 16.67 |
| 183 | 2:1 | 10 mg/5 mg | 10% leucine/20% | 21.43 |
| | | | sucrose | |
| 184 | 2:1 | 10 mg/5 mg | 10% leucine/20% | 21.43 |
| | | | trehalose | |
| 185 | 2:1 | 10 mg/5 mg | 5% T-leucine | 15.79 |
| 186 | 2:1 | 10 mg/5 mg | 5% T- | 20.00 |
| | | | leucine/20% | |
| | | | sucrose | |
| 187 | 2:1 | 10 mg/5 mg | 5% T- | 20.00 |
| | | | leucine/20% | |
| | | | trehalose | |
| 188 | 2:1 | 10 mg/5 mg | 1% Na stearate | 15.15 |
| 189 | 2:1 | 10 mg/5 mg | 1% Na | 18.99 |
| | | | stearate/20% | |
| | | | Sucrose | |
| 191 | 2:1 | 10 mg/5 mg | 1% Na | 18.99 |
| | | | stearate/20% | |
| | | | trehalose | |
| 190 | 1:1 | 10 mg/10 mg | No excipients | 20.00 |
| 192 | 1:1 | 10 mg/10 mg | 10% leucine | 22.22 |
| 193 | 1:1 | 10 mg/10 mg | 10% leucine/20% | 28.57 |
| | | | sucrose | |
| 194 | 1:1 | 10 mg/10 mg | 10% leucine/20% | 28.57 |
| | | | trehalose | |
| 195 | 1:1 | 10 mg/10 mg | 5% T-leucine | 21.05 |
| 196 | 1:1 | 10 mg/10 mg | 5% T- | 26.67 |
| | | | leucine/20% | |
| | | | sucrose | |
| 197 | 1:1 | 10 mg/10 mg | 5% T- | 26.67 |
| | | | leucine/20% | |
| | | | trehalose | |
| 198 | 1:1 | 10 mg/10 mg | 1% Na stearate | 20.20 |
| 199 | 1:1 | 10 mg/10 mg | 1% Na | 25.32 |
| | | | stearate/20% | |
| | | | Sucrose | |
| 200 | 1:1 | 10 mg/10 mg | 1% Na | 25.32 |
| | | | stearate/20% | |
| | | | trehalose | |
| 201 | 4:1 | 9 mg/2.25 mg | No excipients | 11.25 |
| 202 | 4:1 | 9 mg/2.25 mg | 10% leucine | 12.50 |
| 203 | 4:1 | 9 mg/2.25 mg | 10% leucine/20% | 16.07 |
| | | | sucrose | |
| 204 | 4:1 | 9 mg/2.25 mg | 10% leucine/20% | 16.07 |
| | | | trehalose | |
| 205 | 4:1 | 9 mg/2.25 mg | 5% T-leucine | 11.84 |
| 206 | 4:1 | 9 mg/2.25 mg | 5% T- | 15.00 |
| | | | leucine/20% | |
| | | | sucrose | |
| 207 | 4:1 | 9 mg/2.25 mg | 5% T- | 15.00 |
| | | | leucine/20% | |
| | | | trehalose | |
| 208 | 4:1 | 9 mg/2.25 mg | 1% Na stearate | 11.36 |
| 209 | 4:1 | 9 mg/2.25 mg | 1% Na | 14.24 |
| | | | stearate/20% | |
| | | | Sucrose | |
| 210 | 4:1 | 9 mg/2.25 mg | 1% Na | 14.24 |
| | | | stearate/20% | |
| | | | trehalose | |
| 211 | 3:1 | 9 mg/3 mg | No excipients | 12.00 |
| 212 | 3:1 | 9 mg/3 mg | 10% leucine | 13.33 |
| 213 | 3:1 | 9 mg/3 mg | 10% leucine/20% | 17.14 |
| | | | sucrose | |
| 214 | 3:1 | 9 mg/3 mg | 10% leucine/20% | 17.14 |
| | | | trehalose | |
| 215 | 3:1 | 9 mg/3 mg | 5% T-leucine | 12.63 |
| 216 | 3:1 | 9 mg/3 mg | 5% T- | 16.00 |
| | | | leucine/20% | |
| | | | sucrose | |
| 217 | 3:1 | 9 mg/3 mg | 5% T- | 16.00 |
| | | | leucine/20% | |
| | | | trehalose | |
| 218 | 3:1 | 9 mg/3 mg | 1% Na stearate | 12.12 |
| 219 | 3:1 | 9 mg/3 mg | 1% Na | 15.19 |
| | | | stearate/20% | |
| | | | Sucrose | |
| 220 | 3:1 | 9 mg/3 mg | 1% Na | 15.19 |
| | | | stearate/20% | |
| | | | trehalose | |
| 221 | 2:1 | 9 mg/4.5 mg | No excipients | 13.50 |
| 222 | 2:1 | 9 mg/4.5 mg | 10% leucine | 15.00 |
| 223 | 2:1 | 9 mg/4.5 mg | 10% leucine/20% | 19.29 |
| | | | sucrose | |
| 224 | 2:1 | 9 mg/4.5 mg | 10% leucine/20% | 19.29 |
| | | | trehalose | |
| 225 | 2:1 | 9 mg/4.5 mg | 5% T-leucine | 14.21 |
| 226 | 2:1 | 9 mg/4.5 mg | 5% T- | 18.00 |
| | | | leucine/20% | |
| | | | sucrose | |
| 227 | 2:1 | 9 mg/4.5 mg | 5% T- | 18.00 |
| | | | leucine/20% | |
| | | | trehalose | |
| 228 | 2:1 | 9 mg/4.5 mg | 1% Na stearate | 13.64 |
| 229 | 2:1 | 9 mg/4.5 mg | 1% Na | 17.09 |
| | | | stearate/20% | |
| | | | Sucrose | |
| 230 | 2:1 | 9 mg/4.5 mg | 1% Na | 17.09 |
| | | | stearate/20% | |
| | | | trehalose | |
| 231 | 1:1 | 9 mg/9 mg | No excipients | 18.00 |
| 232 | 1:1 | 9 mg/9 mg | 10% leucine | 20.00 |
| 233 | 1:1 | 9 mg/9 mg | 10% leucine/20% | 25.71 |
| | | | sucrose | |
| 234 | 1:1 | 9 mg/9 mg | 10% leucine/20% | 25.71 |
| | | | trehalose | |
| 235 | 1:1 | 9 mg/9 mg | 5% T-leucine | 18.95 |
| 236 | 1:1 | 9 mg/9 mg | 5% T- | 24.00 |
| | | | leucine/20% | |
| | | | sucrose | |
| 237 | 1:1 | 9 mg/9 mg | 5% T- | 24.00 |
| | | | leucine/20% | |
| | | | trehalose | |
| 238 | 1:1 | 9 mg/9 mg | 1% Na stearate | 18.18 |
| 239 | 1:1 | 9 mg/9 mg | 1% Na | 22.78 |
| | | | stearate/20% | |
| | | | Sucrose | |
| 240 | 1:1 | 9 mg/9 mg | 1% Na | 22.78 |
| | | | stearate/20% | |
| | | | trehalose | |
| 241 | 4:1 | 8 mg/2 mg | No excipients | 10.00 |
| 242 | 4:1 | 8 mg/2 mg | 10% leucine | 11.11 |
| 243 | 4:1 | 8 mg/2 mg | 10% leucine/20% | 14.29 |
| | | | sucrose | |
| 244 | 4:1 | 8 mg/2 mg | 10% leucine/20% | 14.29 |
| | | | trehalose | |
| 245 | 4:1 | 8 mg/2 mg | 5% T-leucine | 10.53 |
| 246 | 4:1 | 8 mg/2 mg | 5% T- | 13.33 |
| | | | leucine/20% | |
| | | | sucrose | |
| 247 | 4:1 | 8 mg/2 mg | 5% T- | 13.33 |
| | | | leucine/20% | |
| | | | trehalose | |
| 248 | 4:1 | 8 mg/2 mg | 1% Na stearate | 10.10 |
| 249 | 4:1 | 8 mg/2 mg | 1% Na | 12.66 |
| | | | stearate/20% | |
| | | | Sucrose | |
| 250 | 4:1 | 8 mg/2 mg | 1% Na | 12.66 |
| | | | stearate/20% | |
| | | | trehalose | |
| 251 | 3:1 | 8 mg/2.67 mg | No excipients | 10.67 |
| 252 | 3:1 | 8 mg/2.67 mg | 10% leucine | 11.86 |
| 253 | 3:1 | 8 mg/2.67 mg | 10% leucine/20% | 15.24 |
| | | | sucrose | |
| 254 | 3:1 | 8 mg/2.67 mg | 10% leucine/20% | 15.24 |
| | | | trehalose | |
| 255 | 3:1 | 8 mg/2.67 mg | 5% T-leucine | 11.23 |
| 256 | 3:1 | 8 mg/2.67 mg | 5% T- | 14.23 |
| | | | leucine/20% | |
| | | | sucrose | |
| 257 | 3:1 | 8 mg/2.67 mg | 5% T- | 14.23 |
| | | | leucine/20% | |
| | | | trehalose | |
| 258 | 3:1 | 8 mg/2.67 mg | 1% Na stearate | 10.78 |
| 259 | 3:1 | 8 mg/2.67 mg | 1% Na | 13.51 |
| | | | stearate/20% | |
| | | | Sucrose | |
| 260 | 3:1 | 8 mg/2.67 mg | 1% Na | 13.51 |
| | | | stearate/20% | |
| | | | trehalose | |
| 261 | 2:1 | 8 mg/4 mg | No excipients | 12.00 |
| 262 | 2:1 | 8 mg/4 mg | 10% leucine | 13.33 |
| 263 | 2:1 | 8 mg/4 mg | 10% leucine/20% | 17.14 |
| | | | sucrose | |
| 264 | 2:1 | 8 mg/4 mg | 10% leucine/20% | 17.14 |
| | | | trehalose | |
| 265 | 2:1 | 8 mg/4 mg | 5% T-leucine | 12.63 |
| 266 | 2:1 | 8 mg/4 mg | 5% T- | 16.00 |
| | | | leucine/20% | |
| | | | sucrose | |
| 267 | 2:1 | 8 mg/4 mg | 5% T- | 16.00 |
| | | | leucine/20% | |
| | | | trehalose | |
| 268 | 2:1 | 8 mg/4 mg | 1% Na stearate | 12.12 |
| 269 | 2:1 | 8 mg/4 mg | 1% Na | 15.19 |
| | | | stearate/20% | |
| | | | Sucrose | |
| 270 | 2:1 | 8 mg/4 mg | 1% Na | 15.19 |
| | | | stearate/20% | |
| | | | trehalose | |
| 271 | 1:1 | 8 mg/8 mg | No excipients | 16.00 |
| 272 | 1:1 | 8 mg/8 mg | 10% leucine | 17.78 |
| 273 | 1:1 | 8 mg/8 mg | 10% leucine/20% | 22.86 |
| | | | sucrose | |
| 274 | 1:1 | 8 mg/8 mg | 10% leucine/20% | 22.86 |
| | | | trehalose | |
| 275 | 1:1 | 8 mg/8 mg | 5% T-leucine | 16.84 |
| 276 | 1:1 | 8 mg/8 mg | 5% T- | 21.33 |
| | | | leucine/20% | |
| | | | sucrose | |
| 277 | 1:1 | 8 mg/8 mg | 5% T- | 21.33 |
| | | | leucine/20% | |
| | | | trehalose | |
| 278 | 1:1 | 8 mg/8 mg | 1% Na stearate | 16.16 |
| 279 | 1:1 | 8 mg/8 mg | 1% Na | 20.25 |
| | | | stearate/20% | |
| | | | Sucrose | |
| 280 | 1:1 | 8 mg/8 mg | 1% Na | 20.25 |
| | | | stearate/20% | |
| | | | trehalose | |
| 281 | 4:1 | 7 mg/1.75 mg | No excipients | 8.75 |
| 282 | 4:1 | 7 mg/1.75 mg | 10% leucine | 9.72 |
| 283 | 4:1 | 7 mg/1.75 mg | 10% leucine/20% | 12.50 |
| | | | sucrose | |
| 284 | 4:1 | 7 mg/1.75 mg | 10% leucine/20% | 12.50 |
| | | | trehalose | |
| 285 | 4:1 | 7 mg/1.75 mg | 5% T-leucine | 9.21 |
| 286 | 4:1 | 7 mg/1.75 mg | 5% T- | 11.67 |
| | | | leucine/20% | |
| | | | sucrose | |
| 287 | 4:1 | 7 mg/1.75 mg | 5% T- | 11.67 |
| | | | leucine/20% | |
| | | | trehalose | |
| 288 | 4:1 | 7 mg/1.75 mg | 1% Na stearate | 8.84 |
| 289 | 4:1 | 7 mg/1.75 mg | 1% Na | 11.08 |
| | | | stearate/20% | |
| | | | Sucrose | |
| 290 | 4:1 | 7 mg/1.75 mg | 1% Na | 11.08 |
| | | | stearate/20% | |
| | | | trehalose | |
| 291 | 3:1 | 7 mg/2.33 mg | No excipients | 9.33 |
| 292 | 3:1 | 7 mg/2.33 mg | 10% leucine | 10.37 |
| 293 | 3:1 | 7 mg/2.33 mg | 10% leucine/20% | 13.33 |
| | | | sucrose | |
| 294 | 3:1 | 7 mg/2.33 mg | 10% leucine/20% | 13.33 |
| | | | trehalose | |
| 295 | 3:1 | 7 mg/2.33 mg | 5% T-leucine | 9.82 |
| 296 | 3:1 | 7 mg/2.33 mg | 5% T- | 12.44 |
| | | | leucine/20% | |
| | | | sucrose | |
| 297 | 3:1 | 7 mg/2.33 mg | 5% T- | 12.44 |
| | | | leucine/20% | |
| | | | trehalose | |
| 298 | 3:1 | 7 mg/2.33 mg | 1% Na stearate | 9.42 |
| 299 | 3:1 | 7 mg/2.33 mg | 1% Na | 11.81 |
| | | | stearate/20% | |
| | | | Sucrose | |
| 300 | 3:1 | 7 mg/2.33 mg | 1% Na | 11.81 |
| | | | stearate/20% | |
| | | | trehalose | |
| 301 | 2:1 | 7 mg/3.5 mg | No excipients | 10.50 |
| 302 | 2:1 | 7 mg/3.5 mg | 10% leucine | 11.67 |
| 303 | 2:1 | 7 mg/3.5 mg | 10% leucine/20% | 15.00 |
| | | | sucrose | |
| 304 | 2:1 | 7 mg/3.5 mg | 10% leucine/20% | 15.00 |
| | | | trehalose | |
| 305 | 2:1 | 7 mg/3.5 mg | 5% T-leucine | 11.05 |
| 306 | 2:1 | 7 mg/3.5 mg | 5% T- | 14.00 |
| | | | leucine/20% | |
| | | | sucrose | |
| 307 | 2:1 | 7 mg/3.5 mg | 5% T- | 14.00 |
| | | | leucine/20% | |
| | | | trehalose | |
| 308 | 2:1 | 7 mg/3.5 mg | 1% Na stearate | 10.61 |
| 309 | 2:1 | 7 mg/3.5 mg | 1% Na | 13.29 |
| | | | stearate/20% | |
| | | | Sucrose | |
| 310 | 2:1 | 7 mg/3.5 mg | 1% Na | 13.29 |
| | | | stearate/20% | |
| | | | trehalose | |
| 311 | 1:1 | 7 mg/7 mg | No excipients | 14.00 |
| 312 | 1:1 | 7 mg/7 mg | 10% leucine | 15.56 |
| 313 | 1:1 | 7 mg/7 mg | 10% leucine/20% | 20.00 |
| | | | sucrose | |
| 314 | 1:1 | 7 mg/7 mg | 10% leucine/20% | 20.00 |
| | | | trehalose | |
| 315 | 1:1 | 7 mg/7 mg | 5% T-leucine | 14.74 |
| 316 | 1:1 | 7 mg/7 mg | 5% T- | 18.67 |
| | | | leucine/20% | |
| | | | sucrose | |
| 317 | 1:1 | 7 mg/7 mg | 5% T- | 18.67 |
| | | | leucine/20% | |
| | | | trehalose | |
| 318 | 1:1 | 7 mg/7 mg | 1% Na stearate | 14.14 |
| 319 | 1:1 | 7 mg/7 mg | 1% Na | 17.72 |
| | | | stearate/20% | |
| | | | Sucrose | |
| 320 | 1:1 | 7 mg/7 mg | 1% Na | 17.72 |
| | | | stearate/20% | |
| | | | trehalose | |
| 321 | 4:1 | 6 mg/1.5 mg | No excipients | 7.50 |
| 322 | 4:1 | 6 mg/1.5 mg | 10% leucine | 8.33 |
| 323 | 4:1 | 6 mg/1.5 mg | 10% leucine/20% | 10.71 |
| | | | sucrose | |
| 324 | 4:1 | 6 mg/1.5 mg | 10% leucine/20% | 10.71 |
| | | | trehalose | |
| 325 | 4:1 | 6 mg/1.5 mg | 5% T-leucine | 7.89 |
| 326 | 4:1 | 6 mg/1.5 mg | 5% T- | 10.00 |
| | | | leucine/20% | |
| | | | sucrose | |
| 327 | 4:1 | 6 mg/1.5 mg | 5% T- | 10.00 |
| | | | leucine/20% | |
| | | | trehalose | |
| 328 | 4:1 | 6 mg/1.5 mg | 1% Na stearate | 7.58 |
| 329 | 4:1 | 6 mg/1.5 mg | 1% Na | 9.49 |
| | | | stearate/20% | |
| | | | Sucrose | |
| 330 | 4:1 | 6 mg/1.5 mg | 1% Na | 9.49 |
| | | | stearate/20% | |
| | | | trehalose | |
| 331 | 3:1 | 6 mg/2 mg | No excipients | 8.00 |
| 332 | 3:1 | 6 mg/2 mg | 10% leucine | 8.89 |
| 333 | 3:1 | 6 mg/2 mg | 10% leucine/20% | 11.43 |
| | | | sucrose | |
| 334 | 3:1 | 6 mg/2 mg | 10% leucine/20% | 11.43 |
| | | | trehalose | |
| 335 | 3:1 | 6 mg/2 mg | 5% T-leucine | 8.42 |
| 336 | 3:1 | 6 mg/2 mg | 5% T- | 10.67 |
| | | | leucine/20% | |
| | | | sucrose | |
| 337 | 3:1 | 6 mg/2 mg | 5% T- | 10.67 |
| | | | leucine/20% | |
| | | | trehalose | |
| 338 | 3:1 | 6 mg/2 mg | 1% Na stearate | 8.08 |
| 339 | 3:1 | 6 mg/2 mg | 1% Na | 10.13 |
| | | | stearate/20% | |
| | | | Sucrose | |
| 340 | 3:1 | 6 mg/2 mg | 1% Na | 10.13 |
| | | | stearate/20% | |
| | | | trehalose | |
| 341 | 2:1 | 6 mg/3 mg | No excipients | 9.00 |
| 342 | 2:1 | 6 mg/3 mg | 10% leucine | 10.00 |
| 343 | 2:1 | 6 mg/3 mg | 10% leucine/20% | 12.86 |
| | | | sucrose | |
| 344 | 2:1 | 6 mg/3 mg | 10% leucine/20% | 12.86 |
| | | | trehalose | |
| 345 | 2:1 | 6 mg/3 mg | 5% T-leucine | 9.47 |
| 346 | 2:1 | 6 mg/3 mg | 5% T- | 12.00 |
| | | | leucine/20% | |
| | | | sucrose | |
| 347 | 2:1 | 6 mg/3 mg | 5% T- | 12.00 |
| | | | leucine/20% | |
| | | | trehalose | |
| 348 | 2:1 | 6 mg/3 mg | 1% Na stearate | 9.09 |
| 349 | 2:1 | 6 mg/3 mg | 1% Na | 11.39 |
| | | | stearate/20% | |
| | | | Sucrose | |
| 350 | 2:1 | 6 mg/3 mg | 1% Na | 11.39 |
| | | | stearate/20% | |
| | | | trehalose | |
| 351 | 1:1 | 6 mg/6 mg | No excipients | 12.00 |
| 352 | 1:1 | 6 mg/6 mg | 10% leucine | 13.33 |
| 353 | 1:1 | 6 mg/6 mg | 10% leucine/20% | 17.14 |
| | | | sucrose | |
| 534 | 1:1 | 6 mg/6 mg | 10% leucine/20% | 17.14 |
| | | | trehalose | |
| 355 | 1:1 | 6 mg/6 mg | 5% T-leucine | 12.63 |
| 356 | 1:1 | 6 mg/6 mg | 5% T- | 16.00 |
| | | | leucine/20% | |
| | | | sucrose | |
| 357 | 1:1 | 6 mg/6 mg | 5% T- | 16.00 |
| | | | leucine/20% | |
| | | | trehalose | |
| 358 | 1:1 | 6 mg/6 mg | 1% Na stearate | 12.12 |
| 359 | 1:1 | 6 mg/6 mg | 1% Na | 15.19 |
| | | | stearate/20% | |
| | | | Sucrose | |
| 360 | 1:1 | 6 mg/6 mg | 1% Na | 15.19 |
| | | | stearate/20% | |
| | | | trehalose | |
| 361 | 4:1 | 5 mg/1.25 mg | No excipients | 6.25 |
| 362 | 4:1 | 5 mg/1.25 mg | 10% leucine | 6.94 |
| 363 | 4:1 | 5 mg/1.25 mg | 10% leucine/20% | 8.93 |
| | | | sucrose | |
| 364 | 4:1 | 5 mg/1.25 mg | 10% leucine/20% | 8.93 |
| | | | trehalose | |
| 365 | 4:1 | 5 mg/1.25 mg | 5% T-leucine | 6.58 |
| 366 | 4:1 | 5 mg/1.25 mg | 5% T- | 8.33 |
| | | | leucine/20% | |
| | | | sucrose | |
| 367 | 4:1 | 5 mg/1.25 mg | 5% T- | 8.33 |
| | | | leucine/20% | |
| | | | trehalose | |
| 368 | 4:1 | 5 mg/1.25 mg | 1% Na stearate | 6.31 |
| 369 | 4:1 | 5 mg/1.25 mg | 1% Na | 7.91 |
| | | | stearate/20% | |
| | | | Sucrose | |
| 370 | 4:1 | 5 mg/1.25 mg | 1% Na | 7.91 |
| | | | stearate/20% | |
| | | | trehalose | |
| 371 | 3:1 | 5 mg/1.67 mg | No excipients | 6.67 |
| 372 | 3:1 | 5 mg/1.67 mg | 10% leucine | 7.41 |
| 373 | 3:1 | 5 mg/1.67 mg | 10% leucine/20% | 9.53 |
| | | | sucrose | |
| 374 | 3:1 | 5 mg/1.67 mg | 10% leucine/20% | 9.53 |
| | | | trehalose | |
| 375 | 3:1 | 5 mg/1.67 mg | 5% T-leucine | 7.02 |
| 376 | 3:1 | 5 mg/1.67 mg | 5% T- | 8.89 |
| | | | leucine/20% | |
| | | | sucrose | |
| 377 | 3:1 | 5 mg/1.67 mg | 5% T- | 8.89 |
| | | | leucine/20% | |
| | | | trehalose | |
| 378 | 3:1 | 5 mg/1.67 mg | 1% Na stearate | 6.74 |
| 379 | 3:1 | 5 mg/1.67 mg | 1% Na | 8.44 |
| | | | stearate/20% | |
| | | | Sucrose | |
| 380 | 3:1 | 5 mg/1.67 mg | 1% Na | 8.44 |
| | | | stearate/20% | |
| | | | trehalose | |
| 381 | 2:1 | 5 mg/2.5 mg | No excipients | 7.50 |
| 382 | 2:1 | 5 mg/2.5 mg | 10% leucine | 8.33 |
| 383 | 2:1 | 5 mg/2.5 mg | 10% leucine/20% | 10.71 |
| | | | sucrose | |
| 384 | 2:1 | 5 mg/2.5 mg | 10% leucine/20% | 10.71 |
| | | | trehalose | |
| 385 | 2:1 | 5 mg/2.5 mg | 5% T-leucine | 7.89 |
| 386 | 2:1 | 5 mg/2.5 mg | 5% T- | 10.00 |
| | | | leucine/20% | |
| | | | sucrose | |
| 387 | 2:1 | 5 mg/2.5 mg | 5% T- | 10.00 |
| | | | leucine/20% | |
| | | | trehalose | |
| 3388 | 2:1 | 5 mg/2.5 mg | 1% Na stearate | 7.58 |
| 389 | 2:1 | 5 mg/2.5 mg | 1% Na | 9.49 |
| | | | stearate/20% | |
| | | | Sucrose | |
| 390 | 2:1 | 5 mg/2.5 mg | 1% Na | 9.49 |
| | | | stearate/20% | |
| | | | trehalose | |
| 391 | 1:1 | 5 mg/5 mg | No excipients | 10.00 |
| 392 | 1:1 | 5 mg/5 mg | 10% leucine | 11.11 |
| 393 | 1:1 | 5 mg/5 mg | 10% leucine/20% | 14.29 |
| | | | sucrose | |
| 394 | 1:1 | 5 mg/5 mg | 10% leucine/20% | 14.29 |
| | | | trehalose | |
| 395 | 1:1 | 5 mg/5 mg | 5% T-leucine | 10.53 |
| 396 | 1:1 | 5 mg/5 mg | 5% T- | 13.33 |
| | | | leucine/20% | |
| | | | sucrose | |
| 397 | 1:1 | 5 mg/5 mg | 5% T- | 13.33 |
| | | | leucine/20% | |
| | | | trehalose | |
| 398 | 1:1 | 5 mg/5 mg | 1% Na stearate | 10.10 |
| 399 | 1:1 | 5 mg/5 mg | 1% Na | 12.66 |
| | | | stearate/20% | |
| | | | Sucrose | |
| 400 | 1:1 | 5 mg/5 mg | 1% Na | 12.66 |
| | | | stearate/20% | |
| | | | trehalose | |
| 401 | 4:1 | 4 mg/1 mg | No excipients | 5.00 |
| 402 | 4:1 | 4 mg/1 mg | 10% leucine | 5.56 |
| 403 | 4:1 | 4 mg/1 mg | 10% leucine/20% | 7.14 |
| | | | sucrose | |
| 404 | 4:1 | 4 mg/1 mg | 10% leucine/20% | 7.14 |
| | | | trehalose | |
| 405 | 4:1 | 4 mg/1 mg | 5% T-leucine | 5.26 |
| 406 | 4:1 | 4 mg/1 mg | 5% T- | 6.67 |
| | | | leucine/20% | |
| | | | sucrose | |
| 407 | 4:1 | 4 mg/1 mg | 5% T- | 6.67 |
| | | | leucine/20% | |
| | | | trehalose | |
| 408 | 4:1 | 4 mg/1 mg | 1% Na stearate | 5.05 |
| 409 | 4:1 | 4 mg/1 mg | 1% Na | 6.33 |
| | | | stearate/20% | |
| | | | Sucrose | |
| 410 | 4:1 | 4 mg/1 mg | 1% Na | 6.33 |
| | | | stearate/20% | |
| | | | trehalose | |
| 411 | 3:1 | 4 mg/1.33 mg | No excipients | 5.33 |
| 412 | 3:1 | 4 mg/1.33 mg | 10% leucine | 5.93 |
| 413 | 3:1 | 4 mg/1.33 mg | 10% leucine/20% | 7.62 |
| | | | sucrose | |
| 414 | 3:1 | 4 mg/1.33 mg | 10% leucine/20% | 7.62 |
| | | | trehalose | |
| 415 | 3:1 | 4 mg/1.33 mg | 5% T-leucine | 5.61 |
| 416 | 3:1 | 4 mg/1.33 mg | 5% T- | 7.11 |
| | | | leucine/20% | |
| | | | sucrose | |
| 417 | 3:1 | 4 mg/1.33 mg | 5% T- | 7.11 |
| | | | leucine/20% | |
| | | | trehalose | |
| 418 | 3:1 | 4 mg/1.33 mg | 1% Na stearate | 5.39 |
| 419 | 3:1 | 4 mg/1.33 mg | 1% Na | 6.75 |
| | | | stearate/20% | |
| | | | Sucrose | |
| 420 | 3:1 | 4 mg/1.33 mg | 1% Na | 6.75 |
| | | | stearate/20% | |
| | | | trehalose | |
| 421 | 2:1 | 4 mg/2 mg | No excipients | 6.00 |
| 422 | 2:1 | 4 mg/2 mg | 10% leucine | 6.67 |
| 423 | 2:1 | 4 mg/2 mg | 10% leucine/20% | 8.57 |
| | | | sucrose | |
| 424 | 2:1 | 4 mg/2 mg | 10% leucine/20% | 8.57 |
| | | | trehalose | |
| 425 | 2:1 | 4 mg/2 mg | 5% T-leucine | 6.32 |
| 426 | 2:1 | 4 mg/2 mg | 5% T- | 8.00 |
| | | | leucine/20% | |
| | | | sucrose | |
| 427 | 2:1 | 4 mg/2 mg | 5% T- | 8.00 |
| | | | leucine/20% | |
| | | | trehalose | |
| 428 | 2:1 | 4 mg/2 mg | 1% Na stearate | 6.06 |
| 429 | 2:1 | 4 mg/2 mg | 1% Na | 7.59 |
| | | | stearate/20% | |
| | | | Sucrose | |
| 430 | 2:1 | 4 mg/2 mg | 1% Na | 7.59 |
| | | | stearate/20% | |
| | | | trehalose | |
| 431 | 1:1 | 4 mg/4 mg | No excipients | 8.00 |
| 432 | 1:1 | 4 mg/4 mg | 10% leucine | 8.89 |
| 433 | 1:1 | 4 mg/4 mg | 10% leucine/20% | 11.43 |
| | | | sucrose | |
| 434 | 1:1 | 4 mg/4 mg | 10% leucine/20% | 11.43 |
| | | | trehalose | |
| 435 | 1:1 | 4 mg/4 mg | 5% T-leucine | 8.42 |
| 436 | 1:1 | 4 mg/4 mg | 5% T- | 10.67 |
| | | | leucine/20% | |
| | | | sucrose | |
| 437 | 1:1 | 4 mg/4 mg | 5% T- | 10.67 |
| | | | leucine/20% | |
| | | | trehalose | |
| 438 | 1:1 | 4 mg/4 mg | 1% Na stearate | 8.08 |
| 439 | 1:1 | 4 mg/4 mg | 1% Na | 10.13 |
| | | | stearate/20% | |
| | | | Sucrose | |
| 440 | 1:1 | 4 mg/4 mg | 1% Na | 10.13 |
| | | | stearate/20% | |
| | | | trehalose | |
| 441 | 4:1 | 3 mg/0.75 mg | No excipients | 3.75 |
| 442 | 4:1 | 3 mg/0.75 mg | 10% leucine | 4.17 |
| 443 | 4:1 | 3 mg/0.75 mg | 10% leucine/20% | 5.36 |
| | | | sucrose | |
| 444 | 4:1 | 3 mg/0.75 mg | 10% leucine/20% | 5.36 |
| | | | trehalose | |
| 445 | 4:1 | 3 mg/0.75 mg | 5% T-leucine | 3.95 |
| 446 | 4:1 | 3 mg/0.75 mg | 5% T- | 5.00 |
| | | | leucine/20% | |
| | | | sucrose | |
| 447 | 4:1 | 3 mg/0.75 mg | 5% T- | 5.00 |
| | | | leucine/20% | |
| | | | trehalose | |
| 448 | 4:1 | 3 mg/0.75 mg | 1% Na stearate | 3.79 |
| 449 | 4:1 | 3 mg/0.75 mg | 1% Na | 4.75 |
| | | | stearate/20% | |
| | | | Sucrose | |
| 450 | 4:1 | 3 mg/0.75 mg | 1% Na | 4.75 |
| | | | stearate/20% | |
| | | | trehalose | |
| 451 | 3:1 | 3 mg/1 mg | No excipients | 4.00 |
| 452 | 3:1 | 3 mg/1 mg | 10% leucine | 4.44 |
| 453 | 3:1 | 3 mg/1 mg | 10% leucine/20% | 5.71 |
| | | | sucrose | |
| 454 | 3:1 | 3 mg/1 mg | 10% leucine/20% | 5.71 |
| | | | trehalose | |
| 455 | 3:1 | 3 mg/1 mg | 5% T-leucine | 4.21 |
| 456 | 3:1 | 3 mg/1 mg | 5% T- | 5.33 |
| | | | leucine/20% | |
| | | | sucrose | |
| 457 | 3:1 | 3 mg/1 mg | 5% T- | 5.33 |
| | | | leucine/20% | |
| | | | trehalose | |
| 458 | 3:1 | 3 mg/1 mg | 1% Na stearate | 4.04 |
| 459 | 3:1 | 3 mg/1 mg | 1% Na | 5.06 |
| | | | stearate/20% | |
| | | | Sucrose | |
| 460 | 3:1 | 3 mg/1 mg | 1% Na | 5.06 |
| | | | stearate/20% | |
| | | | trehalose | |
| 461 | 2:1 | 3 mg/1.5 mg | No excipients | 4.50 |
| 462 | 2:1 | 3 mg/1.5 mg | 10% leucine | 5.00 |
| 463 | 2:1 | 3 mg/1.5 mg | 10% leucine/20% | 6.43 |
| | | | sucrose | |
| 464 | 2:1 | 3 mg/1.5 mg | 10% leucine/20% | 6.43 |
| | | | trehalose | |
| 465 | 2:1 | 3 mg/1.5 mg | 5% T-leucine | 4.74 |
| 466 | 2:1 | 3 mg/1.5 mg | 5% T- | 6.00 |
| | | | leucine/20% | |
| | | | sucrose | |
| 467 | 2:1 | 3 mg/1.5 mg | 5% T- | 6.00 |
| | | | leucine/20% | |
| | | | trehalose | |
| 468 | 2:1 | 3 mg/1.5 mg | 1% Na stearate | 4.55 |
| 469 | 2:1 | 3 mg/1.5 mg | 1% Na | 5.70 |
| | | | stearate/20% | |
| | | | Sucrose | |
| 470 | 2:1 | 3 mg/1.5 mg | 1% Na | 5.70 |
| | | | stearate/20% | |
| | | | trehalose | |
| 471 | 1:1 | 3 mg/3 mg | No excipients | 6.00 |
| 472 | 1:1 | 3 mg/3 mg | 10% leucine | 6.67 |
| 473 | 1:1 | 3 mg/3 mg | 10% leucine/20% | 8.57 |
| | | | sucrose | |
| 474 | 1:1 | 3 mg/3 mg | 10% leucine/20% | 8.57 |
| | | | trehalose | |
| 475 | 1:1 | 3 mg/3 mg | 5% T-leucine | 6.32 |
| 476 | 1:1 | 3 mg/3 mg | 5% T- | 8.00 |
| | | | leucine/20% | |
| | | | sucrose | |
| 477 | 1:1 | 3 mg/3 mg | 5% T- | 8.00 |
| | | | leucine/20% | |
| | | | trehalose | |
| 478 | 1:1 | 3 mg/3 mg | 1% Na stearate | 6.06 |
| 479 | 1:1 | 3 mg/3 mg | 1% Na | 7.59 |
| | | | stearate/20% | |
| | | | Sucrose | |
| 480 | 1:1 | 3 mg/3 mg | 1% Na | 7.59 |
| | | | stearate/20% | |
| | | | trehalose | |
| 481 | 4:1 | 2 mg/0.5 mg | No excipients | 2.50 |
| 482 | 4:1 | 2 mg/0.5 mg | 10% leucine | 2.78 |
| 483 | 4:1 | 2 mg/0.5 mg | 10% leucine/20% | 3.57 |
| | | | sucrose | |
| 484 | 4:1 | 2 mg/0.5 mg | 10% leucine/20% | 3.57 |
| | | | trehalose | |
| 485 | 4:1 | 2 mg/0.5 mg | 5% T-leucine | 2.63 |
| 486 | 4:1 | 2 mg/0.5 mg | 5% T- | 3.33 |
| | | | leucine/20% | |
| | | | sucrose | |
| 487 | 4:1 | 2 mg/0.5 mg | 5% T- | 3.33 |
| | | | leucine/20% | |
| | | | trehalose | |
| 488 | 4:1 | 2 mg/0.5 mg | 1% Na stearate | 2.53 |
| 489 | 4:1 | 2 mg/0.5 mg | 1% Na | 3.16 |
| | | | stearate/20% | |
| | | | Sucrose | |
| 490 | 4:1 | 2 mg/0.5 mg | 1% Na | 3.16 |
| | | | stearate/20% | |
| | | | trehalose | |
| 491 | 3:1 | 2 mg/0.67 mg | No excipients | 2.67 |
| 492 | 3:1 | 2 mg/0.67 mg | 10% leucine | 2.97 |
| 493 | 3:1 | 2 mg/0.67 mg | 10% leucine/20% | 3.81 |
| | | | sucrose | |
| 494 | 3:1 | 2 mg/0.67 mg | 10% leucine/20% | 3.81 |
| | | | trehalose | |
| 495 | 3:1 | 2 mg/0.67 mg | 5% T-leucine | 2.81 |
| 496 | 3:1 | 2 mg/0.67 mg | 5% T- | 3.56 |
| | | | leucine/20% | |
| | | | sucrose | |
| 497 | 3:1 | 2 mg/0.67 mg | 5% T- | 3.56 |
| | | | leucine/20% | |
| | | | trehalose | |
| 498 | 3:1 | 2 mg/0.67 mg | 1% Na stearate | 2.70 |
| 499 | 3:1 | 2 mg/0.67 mg | 1% Na | 3.38 |
| | | | stearate/20% | |
| | | | Sucrose | |
| 500 | 3:1 | 2 mg/0.67 mg | 1% Na | 3.38 |
| | | | stearate/20% | |
| | | | trehalose | |
| 501 | 2:1 | 2 mg/1 mg | No excipients | 3.00 |
| 502 | 2:1 | 2 mg/1 mg | 10% leucine | 3.33 |
| 503 | 2:1 | 2 mg/1 mg | 10% leucine/20% | 4.29 |
| | | | sucrose | |
| 504 | 2:1 | 2 mg/1 mg | 10% leucine/20% | 4.29 |
| | | | trehalose | |
| 505 | 2:1 | 2 mg/1 mg | 5% T-leucine | 3.16 |
| 506 | 2:1 | 2 mg/1 mg | 5% T- | 4.00 |
| | | | leucine/20% | |
| | | | sucrose | |
| 507 | 2:1 | 2 mg/1 mg | 5% T- | 4.00 |
| | | | leucine/20% | |
| | | | trehalose | |
| 508 | 2:1 | 2 mg/1 mg | 1% Na stearate | 3.03 |
| 509 | 2:1 | 2 mg/1 mg | 1% Na | 3.80 |
| | | | stearate/20% | |
| | | | Sucrose | |
| 510 | 2:1 | 2 mg/1 mg | 1% Na | 3.80 |
| | | | stearate/20% | |
| | | | trehalose | |
| 511 | 1:1 | 2 mg/2 mg | No excipients | 4.00 |
| 512 | 1:1 | 2 mg/2 mg | 10% leucine | 4.44 |
| 513 | 1:1 | 2 mg/2 mg | 10% leucine/20% | 5.71 |
| | | | sucrose | |
| 514 | 1:1 | 2 mg/2 mg | 10% leucine/20% | 5.71 |
| | | | trehalose | |
| 515 | 1:1 | 2 mg/2 mg | 5% T-leucine | 4.21 |
| 516 | 1:1 | 2 mg/2 mg | 5% T- | 5.33 |
| | | | leucine/20% | |
| | | | sucrose | |
| 517 | 1:1 | 2 mg/2 mg | 5% T- | 5.33 |
| | | | leucine/20% | |
| | | | trehalose | |
| 518 | 1:1 | 2 mg/2 mg | 1% Na stearate | 4.04 |
| 519 | 1:1 | 2 mg/2 mg | 1% Na | 5.06 |
| | | | stearate/20% | |
| | | | Sucrose | |
| 520 | 1:1 | 2 mg/2 mg | 1% Na | 5.06 |
| | | | stearate/20% | |
| | | | trehalose | |
| 521 | 1:2.5 | 2 mg/5 mg | No excipients | 7.00 |
| 522 | 1:2.5 | 2 mg/5 mg | 10% leucine | 7.78 |
| 523 | 1:2.5 | 2 mg/5 mg | 10% leucine/20% | 10.00 |
| | | | sucrose | |
| 524 | 1:2.5 | 2 mg/5 mg | 10% leucine/20% | 10.00 |
| | | | trehalose | |
| 525 | 1:2.5 | 2 mg/5 mg | 5% T-leucine | 7.37 |
| 526 | 1:2.5 | 2 mg/5 mg | 5% T- | 9.33 |
| | | | leucine/20% | |
| | | | sucrose | |
| 527 | 1:2.5 | 2 mg/5 mg | 5% T- | 9.33 |
| | | | leucine/20% | |
| | | | trehalose | |
| 528 | 1:2.5 | 2 mg/5 mg | 1% Na stearate | 7.07 |
| 529 | 1:2.5 | 2 mg/5 mg | 1% Na | 8.86 |
| | | | stearate/20% | |
| | | | Sucrose | |
| 530 | 1:2.5 | 2 mg/5 mg | 1% Na | 8.86 |
| | | | stearate/20% | |
| | | | trehalose | |
| 531 | 1:5 | 2 mg/10 mg | No excipients | 12.00 |
| 532 | 1:5 | 2 mg/10 mg | 10% leucine | 13.33 |
| 533 | 1:5 | 2 mg/10 mg | 10% leucine/20% | 17.14 |
| | | | sucrose | |
| 534 | 1:5 | 2 mg/10 mg | 10% leucine/20% | 17.14 |
| | | | trehalose | |
| 535 | 1:5 | 2 mg/10 mg | 5% T-leucine | 12.63 |
| 536 | 1:5 | 2 mg/10 mg | 5% T- | 16.00 |
| | | | leucine/20% | |
| | | | sucrose | |
| 537 | 1:5 | 2 mg/10 mg | 5% T- | 16.00 |
| | | | leucine/20% | |
| | | | trehalose | |
| 538 | 1:5 | 2 mg/10 mg | 1% Na stearate | 12.12 |
| 539 | 1:5 | 2 mg/10 mg | 1% Na | 15.19 |
| | | | stearate/20% | |
| | | | Sucrose | |
| 540 | 1:5 | 2 mg/10 mg | 1% Na | 15.19 |
| | | | stearate/20% | |
| | | | trehalose | |
| 541 | 1:10 | 2 mg/20 mg | No excipients | 22.00 |
| 542 | 1:10 | 2 mg/20 mg | 10% leucine | 24.44 |
| 543 | 1:10 | 2 mg/20 mg | 10% leucine/20% | 31.43 |
| | | | sucrose | |
| 544 | 1:10 | 2 mg/20 mg | 10% leucine/20% | 31.43 |
| | | | trehalose | |
| 545 | 1:10 | 2 mg/20 mg | 5% T-leucine | 23.16 |
| 546 | 1:10 | 2 mg/20 mg | 5% T- | 29.33 |
| | | | leucine/20% | |
| | | | sucrose | |
| 547 | 1:10 | 2 mg/20 mg | 5% T- | 29.33 |
| | | | leucine/20% | |
| | | | trehalose | |
| 548 | 1:10 | 2 mg/20 mg | 1% Na stearate | 22.22 |
| 549 | 1:10 | 2 mg/20 mg | 1% Na | 27.85 |
| | | | stearate/20% | |
| | | | Sucrose | |
| 550 | 1:10 | 2 mg/20 mg | 1% Na | 27.85 |
| | | | stearate/20% | |
| | | | trehalose | |
| 55 | 1:20 | 2 mg/40 mg | No excipients | 42.00 |
| 552 | 1:20 | 2 mg/40 mg | 10% leucine | 46.67 |
| 553 | 1:20 | 2 mg/40 mg | 10% leucine/20% | 60.00 |
| | | | sucrose | |
| 554 | 1:20 | 2 mg/40 mg | 10% leucine/20% | 60.00 |
| | | | trehalose | |
| 555 | 1:20 | 2 mg/40 mg | 5% T-leucine | 44.21 |
| 556 | 1:20 | 2 mg/40 mg | 5% T- | 56.00 |
| | | | leucine/20% | |
| | | | sucrose | |
| 557 | 1:20 | 2 mg/40 mg | 5% T- | 56.00 |
| | | | leucine/20% | |
| | | | trehalose | |
| 558 | 1:20 | 2 mg/40 mg | 1% Na stearate | 42.42 |
| 559 | 1:20 | 2 mg/40 mg | 1% Na | 53.16 |
| | | | stearate/20% | |
| | | | Sucrose | |
| 560 | 1:20 | 2 mg/40 mg | 1% Na | 53.16 |
| | | | stearate/20% | |
| | | | trehalose | |
| 561 | 1:25 | 2 mg/50 mg | No excipients | 52.00 |
| 562 | 1:25 | 2 mg/50 mg | 10% leucine | 57.78 |
| 563 | 1:25 | 2 mg/50 mg | 10% leucine/20% | 74.29 |
| | | | sucrose | |
| 564 | 1:25 | 2 mg/50 mg | 10% leucine/20% | 74.29 |
| | | | trehalose | |
| 565 | 1:25 | 2 mg/50 mg | 5% T-leucine | 54.74 |
| 566 | 1:25 | 2 mg/50 mg | 5% T- | 69.33 |
| | | | leucine/20% | |
| | | | sucrose | |
| 567 | 1:25 | 2 mg/50 mg | 5% T- | 69.33 |
| | | | leucine/20% | |
| | | | trehalose | |
| 568 | 1:25 | 2 mg/50 mg | 1% Na stearate | 52.53 |
| 569 | 1:25 | 2 mg/50 mg | 1% Na | 65.82 |
| | | | stearate/20% | |
| | | | Sucrose | |
| 570 | 1:25 | 2 mg/50 mg | 1% Na | 65.82 |
| | | | stearate/20% | |
| | | | trehalose | |
| 571 | 1:50 | 2 mg/100 mg | No excipients | 102.00 |
| 572 | 1:50 | 2 mg/100 mg | 10% leucine | 113.33 |
| 573 | 1:50 | 2 mg/100 mg | 10% leucine/20% | 145.71 |
| | | | sucrose | |
| 574 | 1:50 | 2 mg/100 mg | 10% leucine/20% | 145.71 |
| | | | trehalose | |
| 575 | 1:50 | 2 mg/100 mg | 5% T-leucine | 107.37 |
| 576 | 1:50 | 2 mg/100 mg | 5% T- | 136.00 |
| | | | leucine/20% | |
| | | | sucrose | |
| 577 | 1:50 | 2 mg/100 mg | 5% T- | 136.00 |
| | | | leucine/20% | |
| | | | trehalose | |
| 578 | 1:50 | 2 mg/100 mg | 1% Na stearate | 103.03 |
| 579 | 1:50 | 2 mg/100 mg | 1% Na | 129.11 |
| | | | stearate/20% | |
| | | | Sucrose | |
| 580 | 1:50 | 2 mg/100 mg | 1% Na | 129.11 |
| | | | stearate/20% | |
| | | | trehalose | |
| 581 | 1:67 | 1 mg/67 mg | No excipients | 68 |
| 582 | 1:67 | 1 mg/67 mg | 10% leucine | 75.55 |
| 583 | 1:67 | 1 mg/67 mg | 10% leucine/20% | 97.14 |
| | | | sucrose | |
| 584 | 1:67 | 1 mg/67 mg | 10% leucine/20% | 97.14 |
| | | | trehalose | |
| 585 | 1:67 | 1 mg/67 mg | 5% T-leucine | 75.55 |
| 586 | 1:67 | 1 mg/67 mg | 5% T- | 90.66 |
| | | | leucine/20% | |
| | | | sucrose | |
| 587 | 1:67 | 1 mg/67 mg | 5% T- | 90.66 |
| | | | leucine/20% | |
| | | | trehalose | |
| 588 | 1:67 | 1 mg/67 mg | 1% Na stearate | 103.03 |
| 589 | 1:67 | 1 mg/67 mg | 1% Na | 76.40 |
| | | | stearate/20% | |
| | | | Sucrose | |
| 590 | 1:67 | 1 mg/67 mg | 1% Na | 76.40 |
| | | | stearate/20% | |
| | | | trehalose | |
| 591 | 1:75 | 1.33 mg/100 mg | No excipients | 101.33 |
| 592 | 1:75 | 1.33 mg/100 mg | 10% leucine | 112.59 |
| 593 | 1:75 | 1.33 mg/100 mg | 10% leucine/20% | 144.76 |
| | | | sucrose | |
| 594 | 1:75 | 1.33 mg/100 mg | 10% leucine/20% | 144.76 |
| | | | trehalose | |
| 595 | 1:75 | 1.33 mg/100 mg | 5% T-leucine | 106.66 |
| 596 | 1:75 | 1.33 mg/100 mg | 5% T- | 135.11 |
| | | | leucine/20% | |
| | | | sucrose | |
| 597 | 1:75 | 1.33 mg/100 mg | 5% T- | 135.11 |
| | | | leucine/20% | |
| | | | trehalose | |
| 598 | 1:75 | 1.33 mg/100 mg | 1% Na stearate | 102.35 |
| 599 | 1:75 | 1.33 mg/100 mg | 1% Na | 128.27 |
| | | | stearate/20% | |
| | | | Sucrose | |
| 600 | 1:75 | 1.33 mg/100 mg | 1% Na | 128.27 |
| | | | stearate/20% | |
| | | | trehalose | |
| 601 | 1:100 | 1 mg/100 mg | No excipients | 101.00 |
| 602 | 1:100 | 1 mg/100 mg | 10% leucine | 112.22 |
| 603 | 1:100 | 1 mg/100 mg | 10% leucine/20% | 144.29 |
| | | | sucrose | |
| 604 | 1:100 | 1 mg/100 mg | 10% leucine/20% | 144.29 |
| | | | trehalose | |
| 605 | 1:100 | 1 mg/100 mg | 5% T-leucine | 106.32 |
| 606 | 1:100 | 1 mg/100 mg | 5% T- | 134.67 |
| | | | leucine/20% | |
| | | | sucrose | |
| 607 | 1:100 | 1 mg/100 mg | 5% T- | 134.67 |
| | | | leucine/20% | |
| | | | trehalose | |
| 608 | 1:100 | 1 mg/100 mg | 1% Na stearate | 102.02 |
| 609 | 1:100 | 1 mg/100 mg | 1% Na | 127.85 |
| | | | stearate/20% | |
| | | | Sucrose | |
| 610 | 1:100 | 1 mg/100 mg | 1% Na | 127.85 |
| | | | stearate/20% | |
| | | | trehalose | |
| 611 | 1:200 | 0.5 mg/100 mg | No excipients | 100.50 |
| 612 | 1:200 | 0.5 mg/100 mg | 10% leucine | 111.67 |
| 613 | 1:200 | 0.5 mg/100 mg | 10% leucine/20% | 143.57 |
| | | | sucrose | |
| 614 | 1:200 | 0.5 mg/100 mg | 10% leucine/20% | 143.57 |
| | | | trehalose | |
| 615 | 1:200 | 0.5 mg/100 mg | 5% T-leucine | 105.79 |
| 616 | 1:200 | 0.5 mg/100 mg | 5% T- | 134.00 |
| | | | leucine/20% | |
| | | | sucrose | |
| 617 | 1:200 | 0.5 mg/100 mg | 5% T- | 134.00 |
| | | | leucine/20% | |
| | | | trehalose | |
| 618 | 1:200 | 0.5 mg/100 mg | 1% Na stearate | 101.52 |
| 619 | 1:200 | 0.5 mg/100 mg | 1% Na | 127.22 |
| | | | stearate/20% | |
| | | | Sucrose | |
| 620 | 1:200 | 0.5 mg/100 mg | 1% Na | 127.22 |
| | | | stearate/20% | |
| | | | trehalose | |
| 621 | 1:250 | 0.4 mg/100 mg | No excipients | 100.40 |
| 622 | 1:250 | 0.4 mg/100 mg | 10% leucine | 111.56 |
| 623 | 1:250 | 0.4 mg/100 mg | 10% leucine/20% | 143.43 |
| | | | sucrose | |
| 624 | 1:250 | 0.4 mg/100 mg | 10% leucine/20% | 143.43 |
| | | | trehalose | |
| 625 | 1:250 | 0.4 mg/100 mg | 5% T-leucine | 105.68 |
| 626 | 1:250 | 0.4 mg/100 mg | 5% T- | 133.87 |
| | | | leucine/20% | |
| | | | sucrose | |
| 627 | 1:250 | 0.4 mg/100 mg | 5% T- | 133.87 |
| | | | leucine/20% | |
| | | | trehalose | |
| 628 | 1:250 | 0.4 mg/100 mg | 1% Na stearate | 101.41 |
| 629 | 1:250 | 0.4 mg/100 mg | 1% Na | 127.09 |
| | | | stearate/20% | |
| | | | Sucrose | |
| 630 | 1:250 | 0.4 mg/100 mg | 1% Na | 127.09 |
| | | | stearate/20% | |
| | | | trehalose | |
| 631 | 1:300 | 0.33 mg/100 mg | No excipients | 100.33 |
| 632 | 1:300 | 0.33 mg/100 mg | 10% leucine | 111.48 |
| 633 | 1:300 | 0.33 mg/100 mg | 10% leucine/20% | 143.33 |
| | | | sucrose | |
| 634 | 1:300 | 0.33 mg/100 mg | 10% leucine/20% | 143.33 |
| | | | trehalose | |
| 635 | 1:300 | 0.33 mg/100 mg | 5% T-leucine | 105.61 |
| 636 | 1:300 | 0.33 mg/100 mg | 5% T- | 133.77 |
| | | | leucine/20% | |
| | | | sucrose | |
| 637 | 1:300 | 0.33 mg/100 mg | 5% T- | 133.77 |
| | | | leucine/20% | |
| | | | trehalose | |
| 638 | 1:300 | 0.33 mg/100 mg | 1% Na stearate | 101.34 |
| 639 | 1:300 | 0.33 mg/100 mg | 1% Na | 127.00 |
| | | | stearate/20% | |
| | | | Sucrose | |
| 640 | 1:300 | 0.33 mg/100 mg | 1% Na | 127.00 |
| | | | stearate/20% | |
| | | | trehalose | |
| 641 | 1:400 | 0.25 mg/100 mg | No excipients | 100.25 |
| 642 | 1:400 | 0.25 mg/100 mg | 10% leucine | 111.39 |
| 643 | 1:400 | 0.25 mg/100 mg | 10% leucine/20% | 143.21 |
| | | | sucrose | |
| 644 | 1:400 | 0.25 mg/100 mg | 10% leucine/20% | 143.21 |
| | | | trehalose | |
| 645 | 1:400 | 0.25 mg/100 mg | 5% T-leucine | 105.53 |
| 646 | 1:400 | 0.25 mg/100 mg | 5% T- | 133.67 |
| | | | leucine/20% | |
| | | | sucrose | |
| 647 | 1:400 | 0.25 mg/100 mg | 5% T- | 133.67 |
| | | | leucine/20% | |
| | | | trehalose | |
| 648 | 1:400 | 0.25 mg/100 mg | 1% Na stearate | 101.26 |
| 649 | 1:400 | 0.25 mg/100 mg | 1% Na | 126.90 |
| | | | stearate/20% | |
| | | | Sucrose | |
| 650 | 1:400 | 0.25 mg/100 mg | 1% Na | 126.90 |
| | | | stearate/20% | |
| | | | trehalose | |
| 651 | 1:500 | 0.2 mg/100 mg | No excipients | 100.20 |
| 652 | 1:500 | 0.2 mg/100 mg | 10% leucine | 111.33 |
| 653 | 1:500 | 0.2 mg/100 mg | 10% leucine/20% | 143.14 |
| | | | sucrose | |
| 654 | 1:500 | 0.2 mg/100 mg | 10% leucine/20% | 143.14 |
| | | | trehalose | |
| 655 | 1:500 | 0.2 mg/100 mg | 5% T-leucine | 105.47 |
| 656 | 1:500 | 0.2 mg/100 mg | 5% T- | 133.60 |
| | | | leucine/20% | |
| | | | sucrose | |
| 657 | 1:500 | 0.2 mg/100 mg | 5% T- | 133.60 |
| | | | leucine/20% | |
| | | | trehalose | |
| 658 | 1:500 | 0.2 mg/100 mg | 1% Na stearate | 101.21 |
| 659 | 1:500 | 0.2 mg/100 mg | 1% Na | 126.84 |
| | | | stearate/20% | |
| | | | Sucrose | |
| 660 | 1:500 | 0.2 mg/100 mg | 1% Na | 126.84 |
| | | | stearate/20% | |
| | | | trehalose | |
| 661 | 1:600 | 0.167 mg/100 mg | No excipients | 100.17 |
| 662 | 1:600 | 0.167 mg/100 mg | 10% leucine | 111.30 |
| 663 | 1:600 | 0.167 mg/100 mg | 10% leucine/20% | 143.10 |
| | | | sucrose | |
| 664 | 1:600 | 0.167 mg/100 mg | 10% leucine/20% | 143.10 |
| | | | trehalose | |
| 665 | 1:600 | 0.167 mg/100 mg | 5% T-leucine | 105.44 |
| 666 | 1:600 | 0.167 mg/100 mg | 5% T- | 133.56 |
| | | | leucine/20% | |
| | | | sucrose | |
| 667 | 1:600 | 0.167 mg/100 mg | 5% T- | 133.56 |
| | | | leucine/20% | |
| | | | trehalose | |
| 668 | 1:600 | 0.167 mg/100 mg | 1% Na stearate | 101.18 |
| 669 | 1:600 | 0.167 mg/100 mg | 1% Na | 126.79 |
| | | | stearate/20% | |
| | | | Sucrose | |
| 670 | 1:600 | 0.167 mg/100 mg | 1% Na | 126.79 |
| | | | stearate/20% | |
| | | | trehalose | |
| 671 | 1:700 | 0.143 mg/100 mg | No excipients | 100.14 |
| 672 | 1:700 | 0.143 mg/100 mg | 10% leucine | 111.27 |
| 673 | 1:700 | 0.143 mg/100 mg | 10% leucine/20% | 143.06 |
| | | | sucrose | |
| 674 | 1:700 | 0.143 mg/100 mg | 10% leucine/20% | 143.06 |
| | | | trehalose | |
| 675 | 1:700 | 0.143 mg/100 mg | 5% T-leucine | 105.41 |
| 676 | 1:700 | 0.143 mg/100 mg | 5% T- | 133.52 |
| | | | leucine/20% | |
| | | | sucrose | |
| 677 | 1:700 | 0.143 mg/100 mg | 5% T- | 133.52 |
| | | | leucine/20% | |
| | | | trehalose | |
| 678 | 1:700 | 0.143 mg/100 mg | 1% Na stearate | 101.15 |
| 679 | 1:700 | 0.143 mg/100 mg | 1% Na | 126.76 |
| | | | stearate/20% | |
| | | | Sucrose | |
| 680 | 1:700 | 0.143 mg/100 mg | 1% Na | 126.76 |
| | | | stearate/20% | |
| | | | trehalose | |
| 681 | 1:800 | 0.125 mg/100 mg | No excipients | 100.13 |
| 682 | 1:800 | 0.125 mg/100 mg | 10% leucine | 111.25 |
| 683 | 1:800 | 0.125 mg/100 mg | 10% leucine/20% | 143.04 |
| | | | sucrose | |
| 684 | 1:800 | 0.125 mg/100 mg | 10% leucine/20% | 143.04 |
| | | | trehalose | |
| 685 | 1:800 | 0.125 mg/100 mg | 5% T-leucine | 105.39 |
| 686 | 1:800 | 0.125 mg/100 mg | 5% T- | 133.50 |
| | | | leucine/20% | |
| | | | sucrose | |
| 687 | 1:800 | 0.125 mg/100 mg | 5% T- | 133.50 |
| | | | leucine/20% | |
| | | | trehalose | |
| 688 | 1:800 | 0.125 mg/100 mg | 1% Na stearate | 101.14 |
| 689 | 1:800 | 0.125 mg/100 mg | 1% Na | 126.74 |
| | | | stearate/20% | |
| | | | Sucrose | |
| 690 | 1:800 | 0.125 mg/100 mg | 1% Na | 126.74 |
| | | | stearate/20% | |
| | | | trehalose | |
| 691 | 1:900 | 0.111 mg/100 mg | No excipients | 100.111 |
| 692 | 1:900 | 0.111 mg/100 mg | 10% leucine | 111.234 |
| 693 | 1:900 | 0.111 mg/100 mg | 10% leucine/20% | 143.016 |
| | | | sucrose | |
| 694 | 1:900 | 0.111 mg/100 mg | 10% leucine/20% | 143.016 |
| | | | trehalose | |
| 695 | 1:900 | 0.111 mg/100 mg | 5% T-leucine | 105.380 |
| 696 | 1:900 | 0.111 mg/100 mg | 5% T- | 133.481 |
| | | | leucine/20% | |
| | | | sucrose | |
| 697 | 1:900 | 0.111 mg/100 mg | 5% T- | 133.481 |
| | | | leucine/20% | |
| | | | trehalose | |
| 698 | 1:900 | 0.111 mg/100 mg | 1% Na stearate | 101.122 |
| 699 | 1:900 | 0.111 mg/100 mg | 1% Na | 126.723 |
| | | | stearate/20% | |
| | | | Sucrose | |
| 700 | 1:900 | 0.111 mg/100 mg | 1% Na | 126.723 |
| | | | stearate/20% | |
| | | | trehalose | |
| 701 | 1:1000 | 0.1 mg/100 mg | No excipients | 100.10 |
| 702 | 1:1000 | 0.1 mg/100 mg | 10% leucine | 111.22 |
| 703 | 1:1000 | 0.1 mg/100 mg | 10% leucine/20% | 143.00 |
| | | | sucrose | |
| 704 | 1:1000 | 0.1 mg/100 mg | 10% leucine/20% | 143.00 |
| | | | trehalose | |
| 705 | 1:1000 | 0.1 mg/100 mg | 5% T-leucine | 105.37 |
| 706 | 1:1000 | 0.1 mg/100 mg | 5% T- | 133.47 |
| | | | leucine/20% | |
| | | | sucrose | |
| 707 | 1:1000 | 0.1 mg/100 mg | 5% T- | 133.47 |
| | | | leucine/20% | |
| | | | trehalose | |
| 708 | 1:1000 | 0.1 mg/100 mg | 1% Na stearate | 101.11 |
| 709 | 1:1000 | 0.1 mg/100 mg | 1% Na | 126.71 |
| | | | stearate/20% | |
| | | | Sucrose | |
| 710 | 1:1000 | 0.1 mg/100 mg | 1% Na | 126.71 |
| | | | stearate/20% | |
| | | | trehalose | |

^{a}Nintedanib amounts in nintedanib base or nintedanib base within a nintedanib salt thereof

Alternatively, high drug loading fixed dose combination dry powder formulation of pirfenidone and nintedanib with high drug loading and with porous particles for high aerosol dispersion are prepared from perfluorocarbon emulsion. Under this method, co-sprayed pirfenidone and nintedanib HBr, nintedanib base or other salt thereof hollow particles are prepared by a spray-drying technique with a Buchi MiniSpray dryer (Switzerland) or equivalent under the following spray conditions: aspiration: 100%, inlet temperature: 85° C.; outlet temperature: 61° C.; feed pump: 10%; N flow: 2,800 L/hr. The feed solution is prepared by dissolving pirfenidone and nintedanib in different ratios in 100 grams of water heated to 50° C. in various combinations below. High drug load nintedanib or salt thereof and pirfenidone combination formations are shown in Table 39.

TABLE 39
High drug load nintedanib or salt thereof
and pirfenidone combination formations

| Nintedanib | | | |
|---|---|---|---|
| or salt thereof | | | |
| including | | | |
| hydrobromide | | | Dissolve |
| (expressed on | | | in 50° C. |
| nintedanib base) | Pirfenidone | | water to |
| 40 mg | 2 | g | 100 g |
| 20 mg | 2 | g | 100 g |
| 10 mg | 2 | g | 100 g |
| 2 mg | 2 | g | 100 g |
| 40 mg | 1 | g | 100 g |
| 40 mg | 500 | mg | 100 g |
| 40 mg | 100 | mg | 100 g |

In a separate beaker, 5 g of distearoylphosphatidylcholine (DSPC) is homogenized in 750 g hot water (50° C.) using a tabletop homogenizer. 125 grams of perfluorooctyl bromide is added to the solution dropwise while mixing. This solution is then homogenized through a high pressure homogenizer. This solution is then combined with the solution containing dissolved pirfenidone and nintedanib and the resulting solution is fed through a high pressure homogenizer to make a feed stock solution. This solution is fed into a spray dryer under the condition described above. A free flowing, white powder is collected at the cyclone separator. The hollow porous albuterol sulfate particles had an MMAD) ≤5 μm as determined by cascade impaction method. SEM analysis would show that powders to be spherical and highly porous. The tap density of the resulting power is expected to be less than 0.2 g/cm3.

In vitro aerosol performances (fine particle fraction and MMAD) of the above formulations are tested by Next-Generation Impactor (NGI). About 20-30 mg of the powder is filled into gelatin or HPMC capsules and dispersed using a low resistance Plastiape RS01 DPI device at a flow rate of 100 L/min over 2.4 s to inspire 4 L air. The amounts of nintedanib HBr and pirfenidone on each stage are recovered and analyzed by HPLC. Depending on the formulation, multiple actuations may be required to collect adequate amount of drugs in the impactor stage. The fine particle fraction (FPF), mass median aerodynamic diameter (MMAD) are expected to be ≥50% and ≤5 μm respectively.

### Example 12. Nintedanib/PDE4 Combinations

A fixed dose dry powder combination of nintedanib HBr, base or other salt thereof and PDE4 inhibitor, by example Roflumilast, Apremilast, Crisaborole, BI 101555 and other PDE4 inhibitors, can be prepared by lactose carrier blend. By non-limiting example, nintedanib or salt thereof and roflumilast are micronized using a jet mill to reduce the particle size with D90 ≤5 μm and D50 1-2 μm. Micronized nintedanib and roflumilast are mixed with a coarse lactose carrier (Lactohale 200) and lactose fine (Lactohale 300) and force control agents (L-leucine, magnesium stearate) at various combinations shown in table below. First the force control agent, if applicable), is added to Lactohale 200 in layers in a metal vessel. The powder is mixed in a Turbula tumble blender for 15 minutes at 48 rpm. Roflumilast is added to the resulting powder in layers and mixed for 15 minutes at 48 rpm. Nintedanib is then added to the resulting powder blend in layers and mixed in the Turbula blender for 15 minutes. Once complete the powder formulation is tested for content uniformity with a % RSD ≤15% by obtaining samples from various locations in the blend. Optimized nintedanib or salt thereof and prostacyclin analog combination formations are shown in Table 40.

TABLE 40
Optimized nintedanib or salt thereof and
PDE4 inhibitor combination formations

| | % Active drugs | | |
|---|---|---|---|
| Powder | (Nintedanib or salt | | |
| blend | thereof, including | % | |
| formulation | hydrobromide/PDE4 | Lactohale | |
| number | inhibitor ratio)^{a} | 200 | % Force Control Agent^{b} |
| 1 | 10% (2:1) | 90% | N/A |
| 2 | 10% (2:1) | 85% | 5% Lactohale 300 |
| 3 | 10% (2:1) | 87.5% | 2.5% L-leucine |
| 4 | 10% (2:1) | 87.5% | 2.5% magnesium stearate |
| 5 | 10% (2:1) | N/A | Respitose ML003 |
| 6 | 10% (4:1) | 90% | N/A |
| 7 | 10% (4:1) | 90% | 5% Lactohale 300 |
| 8 | 10% (4:1) | 85% | 2.5% L-leucine |
| 9 | 10% (4:1) | 87.5% | 2.5% magnesium stearate |
| 10 | 10% (4:1) | 87.5% | Respitose ML003 |
| 11 | 10% (20:1) | 90% | N/A |
| 12 | 10% (20:1) | 90% | 5% Lactohale 300 |
| 13 | 10% (20:1) | 85% | 2.5% L-leucine |
| 14 | 10% (20:1) | 87.5% | 2.5% magnesium stearate |
| 15 | 10% (20:1) | 87.5% | Respitose ML003 |
| 16 | 10% (1:1) | 90% | N/A |
| 17 | 10% (1:1) | 90% | 5% Lactohale 300 |
| 18 | 10% (1:1) | 85% | 2.5% L-leucine |
| 19 | 10% (1:1) | 87.5% | 2.5% magnesium stearate |
| 20 | 10% (1:1) | 87.5% | Respitose ML003 |
| 21 | 10% (1:2) | 90% | N/A |
| 22 | 10% (1:2) | 90% | 5% Lactohale 300 |
| 23 | 10% (1:2) | 85% | 2.5% L-leucine |
| 24 | 10% (1:2) | 87.5% | 2.5% magnesium stearate |
| 25 | 10% (1:2) | 87.5% | Respitose ML003 |
| 26 | 10% (1:10) | 90% | N/A |
| 27 | 10% (1:10) | 90% | 5% Lactohale 300 |
| 28 | 10% (1:10) | 85% | 2.5% L-leucine |
| 29 | 10% (1:10) | 87.5% | 2.5% magnesium stearate |
| 30 | 10% (1:10) | 87.5% | Respitose ML003 |

^{a}Nintedanib amounts in nintedanib base or nintedanib base within a nintedanib salt thereof
^{b}Magnesium stearate may be substituted with sodium stearate
Lactohale 200 from DFE Pharma with 9 μm D10, 72 μm D50 and 150 μm D90
Respitose ML003 from DFE Pharma with 4 μm D10, 38 μm D50 and 112 μm D90
Lactohale 300 from DFE Pharma with 5 μm D50 and 10 μm D90

In vitro aerosol performance (fine particle fraction and MMAD) of the above formulations are tested by Next-Generation Impactor (NGI). Approximately 20 mg of the powder is filled into gelatin or HPMC capsules containing up to 2 mg nintedanib and up to and dispersed using a low resistance Plastiape RS01 DPI device at a flow rate of 100 L/min over 2.4 s to inspire 4 L air, a volume generally considered as the normal forced inhalation capacity of an average-sized male. The amount of nintedanib and roflumilast or other PDE4 inhibitor on each stage is recovered and analyzed by HPLC. Depending on the formulation, multiple actuations may be required to collect an adequate amount of drugs in the impactor stage. The fine particle fraction (FPF), mass median aerodynamic diameter (MMAD) are expected to be ≤5 μm and ≥50%, respectively.

### Example 13. Nintedanib/Prostacyclin Analog Combinations

A fixed dose dry powder combination of nintedanib HBr, base or other salt thereof and prostacyclin analog can be prepared by lactose carrier blend. By non-limiting example, the prostacyclin analog may be Selexipag, Epoprostenol, Iloprost or Treprostinil. Nintedanib and prostacyclin analog are micronized using a jet mill to reduce the particle size with D90≤5 μm and D50 1-2 μm. Micronized nintedanib and prostacyclin analog are mixed with a coarse lactose carrier (Lactohale 200), lactose fine (Lactohale 300) and a force control agent (L-leucine, magnesium stearate) at various combinations shown in table below. First, the force control agent, when applicable, is added to Lactohale 200 in 2 or 3 layers in a metal vessel. The powder is mixed in a Turbula tumble blender for 15 minutes at 48 rpm. The prostacyclin analog is added to the resulting powder in 2-3 layers and mixed for 15 minutes at 48 rpm. Nintedanib or salt thereof is then added to the resulting powder blend in layers and mixed in the Turbula blender for 15 minutes. Once complete the powder formulation is tested for content uniformity with a % RSD ≤15% by obtaining samples from various locations in the blend. Optimized nintedanib or salt thereof and prostacyclin analog combination formations are shown in Table 41.

TABLE 41
Optimized nintedanib or salt thereof and
prostacyclin analog combination formations

| | % Active drugs | | |
|---|---|---|---|
| | (Nintedanib or salt | | |
| | thereof, including | | |
| Powder | hydrobromide/ | | |
| blend | selexipag or other | % | |
| formulation | prostacyclin | Lactohale | |
| number | analog ratio)^{a} | 200 | % Force Control Agent |
| 1 | 10% (10:1) | 90% | N/A |
| 2 | 10% (10:1) | 85% | 5% Lactohale 300 |
| 3 | 10% (10:1) | 87.5% | 2.5% L-leucine |
| 4 | 10% (10:1) | 87.5% | 2.5% magnesium stearate |
| 5 | 10% (10:1) | N/A | Respitose ML003 |
| 6 | 10% (20:1) | 90% | N/A |
| 7 | 10% (20:1) | 90% | 5% Lactohale 300 |
| 8 | 10% (20:1) | 85% | 2.5% L-leucine |
| 9 | 10% (20:1) | 87.5% | 2.5% magnesium stearate |
| 10 | 10% (20:1) | 87.5% | Respitose ML003 |
| 11 | 10% (40:1) | 90% | N/A |
| 12 | 10% (40:1) | 90% | 5% Lactohale 300 |
| 13 | 10% (40:1) | 85% | 2.5% L-leucine |
| 14 | 10% (40:1) | 87.5% | 2.5% magnesium stearate |
| 15 | 10% (40:1) | 87.5% | Respitose ML003 |
| 16 | 10% (200:1) | 90% | N/A |
| 17 | 10% (200:1) | 90% | 5% Lactohale 300 |
| 18 | 10% (200:1) | 85% | 2.5% L-leucine |
| 19 | 10% (200:1) | 87.5% | 2.5% magnesium stearate |
| 20 | 10% (200:1) | 87.5% | Respitose ML003 |
| 21 | 10% (5:1) | 90% | N/A |
| 22 | 10% (5:1) | 90% | 5% Lactohale 300 |
| 23 | 10% (5:1) | 85% | 2.5% L-leucine |
| 24 | 10% (5:1) | 87.5% | 2.5% magnesium stearate |
| 25 | 10% (5:1) | 87.5% | Respitose ML003 |
| 26 | 10% (2.5:1) | 90% | N/A |
| 27 | 10% (2.5:1) | 90% | 5% Lactohale 300 |
| 28 | 10% (2.5:1) | 85% | 2.5% L-leucine |
| 29 | 10% (2.5:1) | 87.5% | 2.5% magnesium stearate |
| 30 | 10% (2.5:1) | 87.5% | Respitose ML003 |
| 31 | 10% (0.5:1) | 90% | N/A |
| 32 | 10% (0.5:1) | 90% | 5% Lactohale 300 |
| 33 | 10% (0.5:1) | 85% | 2.5% L-leucine |
| 34 | 10% (0.5:1) | 87.5% | 2.5% magnesium stearate |
| 35 | 10% (0.5:1) | 87.5% | Respitose ML003 |

^{a}Nintedanib amounts in nintedanib base or nintedanib base within a nintedanib salt thereof
Lactohale 200 from DFE Pharma with 9 μm D10, 72 μm D50 and 150 μm D90
Respitose ML003 from DFE Pharma with 4 μm D10, 38 μm D50 and 112 μm D90
Lactohale 300 from DFE Pharma with 5 μm D50 and 10 μm D90

In vitro aerosol performance (fine particle fraction and MMAD) of the above formulations are tested by Next-Generation Impactor (NGI). Approximately 20 mg of the powder blend is filled into each gelatin capsule. A low resistance Plastiape RS01 dry powder inhaler device attached to an NGI operating at 100 L/min for 2.4 second (to inspire 4 L air) to determine the aerodynamic properties (MMAD and FPF) of the formulation. The amount of nintedanib HBr and selexipag on each stage is recovered and analyzed by HPLC. Depending on the formulation, multiple actuations may be required to collect adequate amount of drugs in the impactor stage. The fine particle fraction (FPF), mass median aerodynamic diameter (MMAD) are expected to be ≤5 μm and ≥50%, respectively.

Specific examples for implementation of the present invention include:

The composition can be produced as a dry powder as described above and having 3 important components to create the therapeutically effective dose. The dose has 1% to 20% by weight of a nintedanib base molecule that can also be provided in the salt form using several different salt species as described. Specific salt species include hydrobromide, esylate, and hydrochloride. The composition is provided in defined or portions based on size ranges having a D90 less than about 5 microns, 60% to 90% by weight carrier agent, and 0.01% to 20% by weight force control agent, in this unique formulation the dry powder is designed for aerosol delivery to lungs of the adult human by inhalation. When administered in the defined formulation, each therapeutically effective dose contains 0.005 to 10 mg of the nintedanib base or base within a salt form. Also, the therapeutically effective dose can be defined in terms of an effective daily dose of 0.05 to 40 mg. The dry powder composition can also be defined as having the nintedanib component with a fine particle fraction between 10% and 100%. The delivery of the above composition can also be defined in terms of a fine particle dose between 0.005 mg or 0.05 mg and 10 mg nintedanib base or base within the salt form.

Although there are many measures of lung health, the therapeutic dose of the present invention can improve a number of different measures of lung health. This includes slowing the progression of, or preventing or reducing additional inflammation, fibrosis and/or demyelination. For idiopathic pulmonary fibrosis (IPF), progressive pulmonary fibrosis (PPF) and restrictive allograft syndrome (RAS), a “therapeutic effect” is defined as a reduced level or rate of decline in forced vital capacity (FVC), and/or a patient-reported improvement in quality of life and/or a statistically significant increase in or stabilization of exercise tolerance and associated blood-oxygen saturation, reduced decline in baseline forced vital capacity, decreased incidence in acute exacerbations, increase in progression-free survival, increased time-to-death or disease progression, and/or reduced lung fibrosis. For CLAD, a “therapeutic effect” is defined as a reduced decline in forced expiratory volume in one second (FEV1).

The above disclosure describes the advantages or a dry powder composition having 3 important components to create the therapeutically effective dose. The dose has 1% to 20% by weight of a nintedanib base molecule that can also be provided in the salt form using several different salt species as described above. The composition is provided in defined or portions based on size ranges having a D90 less than about 5 microns, 60% to 90% by weight carrier agent, and 0.01% to 20% by weight force control agent, in this unique formulation the dry powder is designed for aerosol delivery to lungs of the adult human by inhalation. When administered in the defined formulation, each therapeutically effective dose contains 0.005 to 10 mg of the nintedanib base or base within a salt form. Also, the therapeutically effective dose can be defined in terms of an effective daily dose of 0.05 to 40 mg. in any of the desired formulations, the particle size of the nintedanib component is less then 5 microns, sometimes between 1 and 4 microns and can be micronized.

Specifically, for this formulation the options for delivery include containing in pre-filled capsules, pre-filled blister packs or provided in a pre-filled cassette for insertion into a dry powder inhaler device, or contained within the metered device reservoir of a dry powder inhaler.

Dry powder inhalers used with the present invention can have a number of performance parameters. The use of medium resistance or high resistance dry powder inhaler devices depends on the particular physiology or disease state of an individual patient. The preceding formulation has particular advantage when used with medium resistance or high resistance dry powder inhaler devices and is uniquely advantageous for the most challenging victims of interstitial lung disease.

As noted above, one of the key components of the dry powder composition is a carrier agent. In one example, lactose is selected a the carrier agent and provides specific advantages compared to other carrier agents, even though other carrier agents may be modified to take advantage of the same physical and chemical characteristics of lactose.

As is well known in the art, nintedanib is ordinarily delivered as an oral composition. One advantage of using a dry powder composition having 3 important components to create the therapeutically effective dose as described above where the dose has 1% to 20% by weight of a nintedanib base molecule that can also be provided in the salt form using several different salt species and provided in defined or portions based on size ranges having a D90 less than about 5 microns, 60% to 90% by weight carrier agent, and 0.01% to 20% by weight force control agent is that the lung Cmax and/or AUC of nintedanib obtained after a single dose of the dry powder to the human with the dry powder inhaler is about the same or greater than the lung Cmax and/or AUC of nintedanib obtainable after administration of a single dose of orally administered nintedanib to the human at a dose that is from about 80% to about 120% of the dry powder dose. This particular formulation can also be supplemented by adding other species that are useful in creating the dry powder composition having the parameters described above. In particular, additional ingredients include bulking agents, surface modifying agents, taste masking agents, sweeteners, salts, and combinations thereof.

The ability to provide a therapeutically effective dose of nintedanib also creates the opportunity to add additional active pharmaceutical ingredients to address patients suffering from interstitial lung disease. As part of a therapeutic regimen using multiple APIs, a dry powder composition having 3 important components to create the therapeutically effective dose as described above where the dose has 1% to 20% by weight of a nintedanib base molecule that can also be provided in the salt form using several different salt species and is provided in defined or portions based on size ranges having a D90 less than about 5 microns, 60% to 90% by weight carrier agent, and 0.01% to 20% by weight force control agent can be used in regimens that include the additional APIS of any or a combination of a dosage regimen that includes a fixed combination, co-administration, sequential administration, or co-prescribed with pirfenidone or pyridine analog, a PDE4 inhibitor, or a prostacyclin analog.

With respect to any of these species containing nintedanib as the active pharmaceutical ingredient, a number of particular formulations are contemplated. The nintedanib as described in the preceding paragraphs can be provided as nanoparticulates.

Once the utility of a dry powder composition having 3 important components to create the therapeutically effective dose is established. The specific parameters of other elements apart from the nintedanib composition can be defined. In particular, the force control agent can be provided as lactose fines having a D50 less than about 5 microns and a D90 less than about 10 microns. Additional force control agents for use with this nintedanib composition include leucine, trileucine, lecithin, magnesium stearate, sodium stearate, sucrose stearate, polyvinylpyrrolidone, ethyl cellulose, Pluronic F-68, Cremophor RH 40, glyceryl monostearate, and polyethylene glycol 6000 and combinations thereof. the force control agent comprises lactose fines with a particle size distribution defined as having a D50 less than about 5 μm and a D90 less than about 10 μm at a formulation content between more than 0.01% and about 20% on a weight by weight basis. The force control agent may be about 0.1% to about 20% leucine, trileucine, magnesium stearate, sodium stearate and lecithin or combinations thereof.

In particularly preferred examples, the dry powder composition of the therapeutically effective dose has a particle size distribution of nintedanib of a D10 between about 0.1 μm and about 2 μm, a D50 between about 1 μm and about 3 μm, and a D90 between about 1.5 μm and about 5 μm. In preferred examples, the carrier agent is lactose at a formulation content between about 60% and about 99% on a weight by weight basis and the lactose carrier agent has a particle size distribution D10 between about 5 μm to about 15 μm, a D50 between about 50 μm to about 100 μm, and a D90 between about 120 μm to about 160 μm.

1. A dry powder composition comprising:
a therapeutically effective dose comprising 1% to 20% by weight of nintedanib base or base within a salt form, having a particle size distribution defined as having a D90 less than about 5 μm, 60% to 90% by weight carrier agent, and 0.01% to 20% by weight force control agent for aerosol delivery to lungs of the adult human by inhalation, wherein each therapeutically effective dose contains 0.05 to 10 mg of the nintedanib or nintedanib base within a salt form or an effective daily dose of 0.05 to 40 mg, to treat an interstitial lung disease; and
a dose of dry powder pirfenidone that is therapeutically and synergistically effective in combination with the nintedanib dose.
